(19) United States
(12) Patent Application Publication
(10) Pub. No.: US 2010/0021886 A1
(43) Pub. Date: Jan. 28, 2010

(54) Methods and Materials for Identifying the Origin of a Carcinoma of Unknown Primary Origin

(76) Inventors: YIXIN WANG, BASKING RIDGE, NJ (US); ABHIJIT MAZUMDER, BASKING RIDGE, NJ (US); DMITRI TALANTOV, SAN DIEGO, CA (US); TIMOTHY JATKOE, SAN DIEGO, CA (US); JONATHAN F. BADEN, BRIDGEWATER, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/024,134

(22) Filed: Feb. 1, 2008

Related U.S. Application Data
(60) Provisional application No. 60/887,625, filed on Feb. 1, 2007.

(51) Int. Cl. C12Q 1/68; C12Q 1/02; C07H 21/00; C40B 40/06

(52) U.S. Cl. 435/6; 435/29; 536/23.1; 506/16

(57) ABSTRACT

The present invention provides a method of identifying origin of a metastasis of unknown origin by obtaining a sample containing metastatic cells; measuring Biomarkers associated with at least two different carcinomas; combining the data from the Biomarkers into a linear discrimination analysis where the linear discrimination analysis normalizes the Biomarkers against a reference; and imposes a cut-off which optimizes sensitivity and specificity of each Biomarker, weights the prevalence of the carcinomas and selects a tissue of origin determining origin based on highest probability determined by the linear discrimination analysis or determining that the carcinoma is not derived from a particular set of carcinomas; and optionally measuring Biomarkers specific for one or more additional different carcinoma, and repeating the steps for additional Biomarkers.

### STATEMENT REGARDING FEDERALLY SPONSORED RESEARCH OR DEVELOPMENT

No government funds were used to make this invention.

### BACKGROUND OF THE INVENTION

Carcinoma of unknown primary (CUP) is a set of heterogeneous, biopsy-confirmed malignancies wherein metastatic disease presents without an identifiable primary tumor site or tissue of origin (ToO). This problem represents approximately 3-5% of all cancers, making it the seventh most common malignancy. Ghosh et al. (2005); and Mintzer et al. (2004). The prognosis and therapeutic regimen of patients are dependent on the origin of the primary tumor, underscoring the need to identify the site of the primary tumor. Greco et al. (2004); Lembersky et al. (1996); and Schlag et al. (1994).

A variety of methods are currently used to resolve this problem. Several methods followed are diagrammed in FIGS. 1-2. Serum tumor Markers can be used for differential diagnosis. Although they lack adequate specificity, they can be used in combination with pathologic and clinical information. Ghosh et al. (2005). Immunohistochemical (IHC) methods can be used to identify tumor lineage but very few IHC Markers are 100% specific. Therefore, pathologists often use a panel of IHC Markers. Several studies have demonstrated accuracies of 66-88% using four to 14 IHC Markers. Brown et al. (1997); DeYoung et al. (2000); and Dennis et al. (2005a). More expensive diagnostic workups include imaging methods such as chest x-ray, computed tomographic (CT) scans, and positron emission tomographic (PET) scans. Each of these methods can identify the primary in 30 to 50% of cases. Ghosh et al. (2005); and Pavlidis et al. (2003). Despite these sophisticated technologies, the ability to resolve CUP cases is only 20-30% ante mortem. Pavlidis et al. (2003); and Varadhachary et al. (2004).

A promising new approach lies in the ability of genome-wide gene expression profiling to identify the origin of tumors. Ma et al. (2006); Dennis et al. (2005b); Su et al. (2001); Ramaswamy et al. (2001); Bloom et al. (2004); Giordano et al. (2001); and 20060094035. These studies demonstrated the feasibility of tissue of origin identification based on the gene expression profile. In order for these expression profiling technologies to be useful in the clinical setting, two major obstacles must be overcome. First, since gene expression profiling was conducted entirely on primary tissues, gene marker candidates must be validated on metastatic tissues to confirm that their tissue specific expression is preserved in metastasis. Second, the gene expression profiling technology must be able to utilize formalin-fixed, paraffin-embedded (FFPE) tissue, since fixed tissue samples are the standard material in current practice. Formalin fixation results in degradation of the RNA (Lewis et al. (2001); and Masuda et al. (1999)) so existing microarray protocols will not perform as reliably. Bibikova et al. (2004). Additionally, the profiling technology must be robust, reproducible, and easily accessible.

Quantitative RT-PCR (qRT-PCR) has been shown to generate reliable results from FFPE tissue. Abrahamsen et al. (2003); Specht et al. (2001); Godfrey et al. (2000); and Cronin et al. (2004). Therefore, a more practical approach would be to use a genome-wide method as a discovery tool and develop a diagnostic assay based on a more robust technology. Ramaswamy (2004). This paradigm, however, requires a smaller gene set to be developed. Oien and colleagues used serial analysis of gene expression (SAGE) to identify 61 tumor Markers from which they developed a RT-PCR method based on eleven genes for five tumor types. Dennis et al. (2002). Another study which coupled SAGE and qRT-PCR developed a panel of five genes for four tumor types and achieved an accuracy of 81%. Buckhaults et al. (2003). A more recent study coupled microarray profiling with qRT-PCR, but used 79 Markers. Tothill et al. (2005).

### SUMMARY OF THE INVENTION

The present invention provides a method of identifying origin of a metastasis of unknown origin by obtaining a sample containing metastatic cells; measuring Biomarkers associated with at least two different carcinomas; combining the data from the Biomarkers into a classification trees where the classification trees uses biomarkers normalized against a reference; and imposes a cut-off which optimizes sensitivity and specificity of each Biomarker, weights the prevalence of the carcinomas and selects a tissue of origin; determining origin based on highest probability determined by the classification trees or determining that the carcinoma is not derived from a particular set of carcinomas; and optionally measuring Biomarkers specific for one or more additional different carcinoma, and repeating steps as necessary for additional Biomarkers.

### BRIEF DESCRIPTION OF THE DRAWINGS

FIGS. 1-2 depict prior art methods of identifying origin of a metastasis of unknown origin.

FIG. 3 Depicts present CUP classification tree

FIG. 4 depicts microarray data showing intensities of two genes in a panel of tissues. (A) Prostate stem cell antigen (PSCA). (B) Coagulation factor V (F5). The bar graphs show the intensity on the y-axis and the tissue on the x-axis. Panc Ca, pancreatic cancer; Panc N, normal pancreas.

FIG. 5 depicts electropherograms obtained from an Agilent Bioanalyzer. RNA was isolated from FFPE tissue using a three hour (A) or sixteen hour (B) proteinase K digestion. Sample C22 (red) was a one-year old block while sample C23 (blue) was a five-year old block. A size ladder is shown in green.

FIG. 6 depicts a comparison of Ct values obtained from three different qRT-PCR methods: random hexamer priming in the reverse transcription followed by qPCR with the resulting cDNA (RH 2 step), gene-specific (reverse primer) priming in the reverse transcription followed by qPCR with the resulting cDNA (GSP 2 step), or gene-specific priming and qRT-PCR in a one-step reaction (GSP 1 step). RNA from eleven samples was divided into the three methods and RNA levels for three genes were measured: β-actin (A), HUMSPB (B), and TTF (C). The median Ct value obtained with each method is indicated by the solid line.

FIG. 7 depicts CUP assay plate diagrams.

FIG. 8 depicts a univariate analysis tree.

FIG. 9 is a series of graphs depicting the assay performance over a range of RNA concentrations.

FIG. 10 is an experimental workflow diagram showing marker candidate nomination and validation (10A).

FIG. 11 depicts expression of 10 selected tissue specific gene Marker candidates in FFPE metastatic carcinomas and prostate primary adenocarcinoma. For each plot the X axis represents the normalized Marker expression value.

FIG. 12 depicts assay optimization. (A and B) Electropherograms obtained from an Agilent Bioanalyzer. RNA was isolated from FFPE tissue using a three hour (A) or sixteen hour (B) proteinase K digestion. Sample C22 (red) was a one-year old block while sample C23 (blue) was a five-year old block. A size ladder is shown in green. (C and D) Comparison of Ct values obtained from three different qRT-PCR methods: random hexamer priming in the reverse transcription followed by qPCR with the resulting cDNA (RH 2 step), gene-specific (reverse primer) priming in the reverse transcription followed by qPCR with the resulting cDNA (GSP 2 step), or gene-specific priming and qRT-PCR in a one-step reaction (GSP 1 step). RNA from eleven samples was divided into the three methods and RNA levels for two genes were measured: β-actin (C), HUMSPB (D). The median Ct value obtained with each method is indicated by the solid line.

FIG. 13 is a heatmap showing the relative expression levels of the 10 Marker panel across 239 samples. Red indicates higher expression.

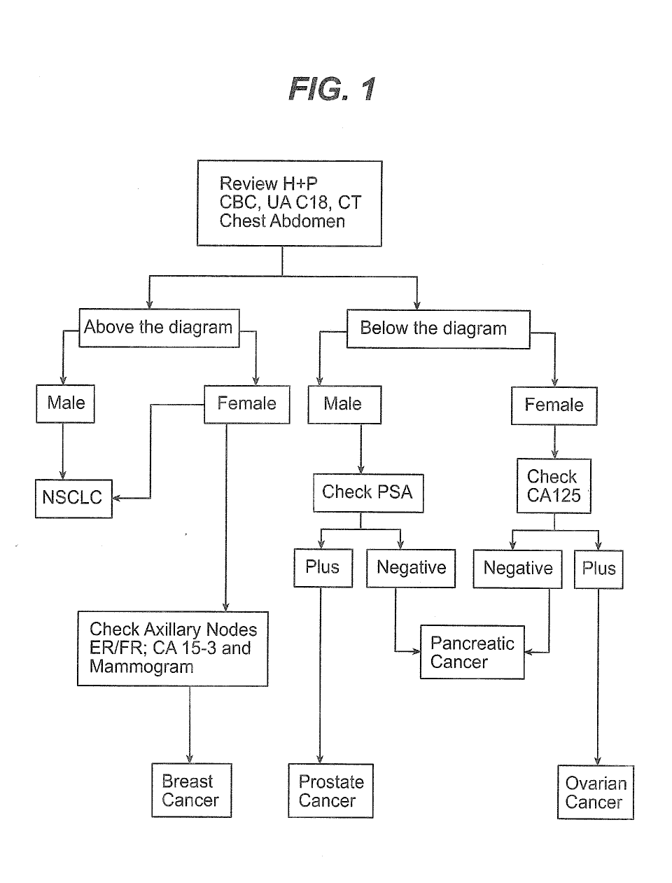

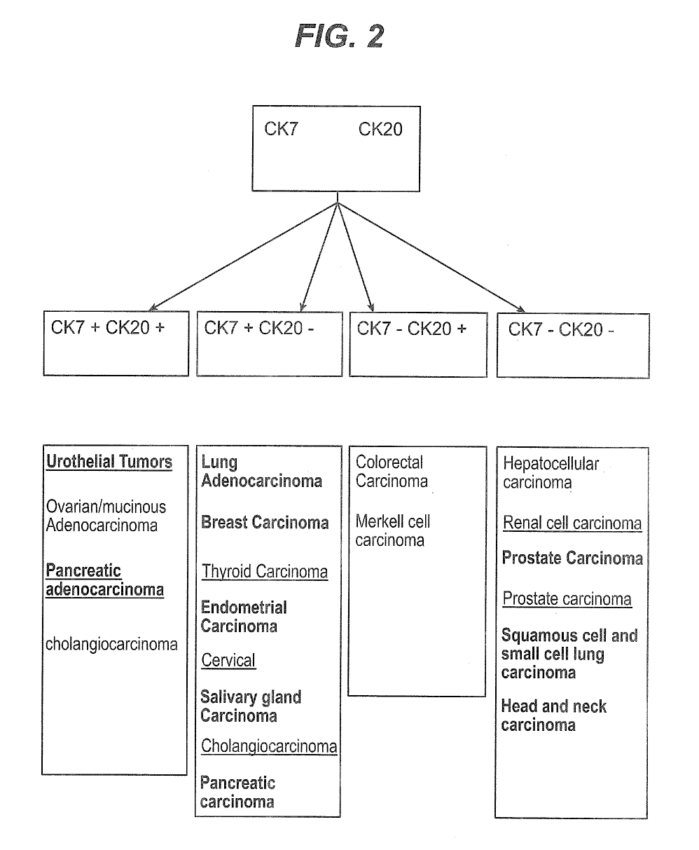

### DETAILED DESCRIPTION

Identifying the primary site in patients with metastatic carcinoma of unknown primary (CUP) origin can enable the application of specific therapeutic regimens and may prolong survival. Marker candidates were then validated by reverse transcriptase polymerase chain reaction (RT-PCR) on 205 FFPE metastatic carcinomas originating from these six tissues as well as metastases originating from other cancer types to determine specificity. A ten-gene signature was selected that predicted the tissue of origin of metastatic carcinomas for these six cancer types. Next, the RNA isolation and qRT-PCR methods were optimized for these ten Markers, and applied the qRT-PCR assay to a set of 260 metastatic tumors, generating an overall accuracy of 78%. Lastly, an independent set of 48 metastatic samples were tested. Importantly, thirty-seven samples in this set had either a known primary or initially presented as CUP but were subsequently resolved, and the assay demonstrated an accuracy of 78%.

A Biomarker is any indicia of the level of expression of an indicated Marker gene. The indicia can be direct or indirect and measure over- or under-expression of the gene given the physiologic parameters and in comparison to an internal control, normal tissue or another carcinoma. Biomarkers include, without limitation, nucleic acids (both over and under-expression and direct and indirect). Using nucleic acids as Biomarkers can include any method known in the art including, without limitation, measuring DNA amplification, RNA, micro RNA, loss of heterozygosity (LOH), single nucleotide polymorphisms (SNPs, Brookes (1999)), microsatellite DNA, DNA hypo- or hyper-methylation. Using proteins as Biomarkers includes any method known in the art including, without limitation, measuring amount, activity, modifications such as glycosylation, phosphorylation, ADP-ribosylation, ubiquitination, etc., or imunohistochemistry (IHC). Other Biomarkers include imaging, cell count and apoptosis Markers.

The indicated genes provided herein are those associated with a particular tumor or tissue type. A Marker gene may be associated with numerous cancer types but provided that the expression of the gene is sufficiently associated with one tumor or tissue type to be identified using the classification tree described herein to be specific for a particular origin, the gene can be used in the claimed invention to determine tissue of origin for a carcinoma of unknown primary origin (CUP). Numerous genes associated with one or more cancers are known in the art. The present invention provides preferred Marker genes and even more preferred Marker gene combinations. These are described herein in detail.

“Origin” as referred to in ‘tissue of origin’ means either the tissue type (lung, colon, etc.) or the histological type (adenocarcinoma, squamous cell carcinoma, etc.) depending on the particular medical circumstances and will be understood by anyone of skill in the art.

A Marker gene corresponds to the sequence designated by a SEQ ID NO when it contains that sequence. A gene segment or fragment corresponds to the sequence of such gene when it contains a portion of the referenced sequence or its complement sufficient to distinguish it as being the sequence of the gene. A gene expression product corresponds to such sequence when its RNA, mRNA, or cDNA hybridizes to the composition having such sequence (e.g. a probe) or, in the case of a peptide or protein, it is encoded by such mRNA. A segment or fragment of a gene expression product corresponds to the sequence of such gene or gene expression product when it contains a portion of the referenced gene expression product or its complement sufficient to distinguish it as being the sequence of the gene or gene expression product.

The inventive methods, compositions, articles, and kits of described and claimed in this specification include one or more Marker genes. “Marker” or “Marker gene” is used throughout this specification to refer to genes and gene expression products that correspond with any gene the over- or under-expression of which is associated with a tumor or tissue type. The preferred Marker genes are described in more detail in Table 1. All sequences discussed herein are described herein and provided in the Sequence Listing.

TABLE 1
CUP panel

| SEQ ID NO: | Gene Name | Affymetrix Chip designation |
|---|---|---|
| 1 | SP-B | 209810_at |
| 2 | TTF1 | 211024_s_at |
| 3 | DSG3 | 205595_at |
| 4 | HPT1 | 209847_at |
| 5 | PSCA | 205319_at |
| 6 | F5 | 204713_s_at |
| 7 | MGB1 | 206378_at |
| 8 | PDEF | 220192_x_at |
| 9 | PSA | 204582_s_at |
| 10 | WT1 | 206067_s_at |

The present invention provides a method of identifying origin of a metastasis of unknown origin by measuring Biomarkers associated with at least two different carcinomas in a sample containing metastatic cells; combining the data from the Biomarkers into a classification trees where the classification trees uses biomarkers normalized against a reference; and imposes a cut-off which optimizes sensitivity and specificity of each Biomarker, weights the prevalence of the carcinomas and selects a tissue of origin; determining origin based on highest probability determined by the classification tree or determining that the carcinoma is not derived from a particular set of carcinomas; and optionally measuring Biomarkers specific for one or more additional different carcinoma, and repeating steps as necessary for additional Biomarkers.

The present invention provides a method of identifying origin of a metastasis of unknown origin by obtaining a sample containing metastatic cells; measuring Biomarkers associated with at least two different carcinomas; combining the data from the Biomarkers into a classification tree where the classification tree i) classification trees uses biomarkers normalized against a reference; and ii) imposes a cut-off which optimizes sensitivity and specificity of each Biomarker, weights the prevalence of the carcinomas and selects a tissue of origin; determining origin based on highest probability determined by the classification tree or determining that the carcinoma is not derived from a particular set of carcinomas; and optionally measuring Biomarkers specific for one or more additional different carcinoma, and repeating steps c) and d) for the additional Biomarkers.

In one embodiment, the Marker genes are selected from i) SP-B, TTF, DSG3, KRT6F, p73H, or SFTPC; ii) F5, PSCA, ITGB6, KLK10, CLDN18, TRIO or FKBP10; and/or iii) CDH17, CDX1 or FABP1. Preferably, the Marker genes are SP-B, TTF, DSG3, KRT6F, p73H, and/or SFTPC. More preferably, the Marker genes are SP-B, TTF and/or DSG3. The Marker genes may further include or be replaced by KRT6F, p73H, and/or SFTPC.

In one embodiment, the Marker genes are F5, PSCA, ITGB6, KLK10, CLDN18, TR10 and/or FKBP10. More preferably, the Marker genes are F5 and/or PSCA. Preferably, the Marker genes can include or be replaced by ITGB6, KLK10, CLDN18, TRI10 and/or FKBP10.

In another embodiment, the Marker genes are CDH17, CDX1 and/or FABP1, preferably, CDH17. The Marker genes can further include or be replaced by CDX1 and/or FABP1.

In one embodiment, gene expression is measured using at least one of SEQ ID NOs: 11-58.

The present invention also encompasses methods that measure gene expression by obtaining and measuring the formation of at least one of the amplicons SEQ ID NOs: 14, 18, 22, 26, 30, 34, 38, 42, 46, 50, 54 and/or 58.

In one embodiment, the Marker genes can be selected from a gender specific Marker selected from at least one of: i) in the case of a male patient KLK3, KLK2, NGEP or NPY; or ii) in the case of a female patient PDEF, MGB, PIP, B305D, B726 or GABA-Pi; and/or WT1, PAX8, STAR or EMX2. Preferably, the Marker gene is KLK2 or KLK3. In this embodiment, the Marker genes can include or be replaced by NGEP and/or NPY. In one embodiment, the Marker genes are PDEF, MGB, PIP, B305D, B726 or GABA-Pi, preferably, PDEF and MGB. In this embodiment, the Marker genes can include or be replaced by PIP, B305D, B726 or GABA-Pi. In one embodiment, the Marker genes are WT1, PAX8, STAR or EMX2, preferably, WT1. In this embodiment, the Marker genes can include or be replaced by PAX8, STAR or EMX2.

The present invention provides methods of obtaining additional clinical information including the site of metastasis to determine the origin of the carcinoma; obtaining optimal biomarker sets for carcinomas comprising the steps of using metastases of know origin, determining Biomarkers therefor and comparing the Biomarkers to Biomarkers of metastases of unknown origin; providing direction of therapy by determining the origin of a metastasis of unknown origin and identifying the appropriate treatment therefor; and providing a prognosis by determining the origin of a metastasis of unknown origin and identifying the corresponding prognosis therefor.

The present invention further provides methods of finding Biomarkers by determining the expression level of a Marker gene in a particular metastasis, measuring a Biomarker for the Marker gene to determine expression thereof, analyzing the expression of the Marker gene according to any of the methods provided herein or known in the art and determining if the Marker gene is effectively specific for the tumor of origin.

The present invention further provides composition containing at least one isolated sequence selected from SEQ ID NOs: 11-58. The present invention further provides kits for conducting an assay according to the methods provided herein and further containing Biomarker detection reagents.

The present invention further provides microarrays or gene chips for performing the methods described herein.

The present invention further provides diagnostic/prognostic portfolios containing isolated nucleic acid sequences, their complements, or portions thereof of a combination of genes as described herein where the combination is sufficient to measure or characterize gene expression in a biological sample having metastatic cells relative to cells from different carcinomas or normal tissue.

Any method described in the present invention can further include measuring expression of at least one gene constitutively expressed in the sample.

Preferably the Markers for pancreatic cancer are coagulation factor V (F5), prostate stem cell antigen (PSCA), integrin, β6 (ITGB6), kallikrein 10 (KLK10), claudin 18 (CLDN18), trio isoform (TR10), and hypothetical protein FLJ22041 similar to FK506 binding proteins (FKBP10). Preferably, Biomarkers for F5 and PSCA are measured together. Biomarkers for ITGB6, KLK10, CLDN18, TR10, and FKBP10 can be measured in addition to or in place of F5 and/or PSCA. F5 is described for instance by 20040076955; 20040005563; and WO2004031412. PSCA is described for instance by WO1998040403; 20030232350; and WO2004063355. ITGB6 is described for instance by WO2004018999; and 6339148. KLK10 is described for instance by WO2004077060; and 20030235820. CLDN18 is described for instance by WO2004063355; and WO2005005601. TR10 is described for instance by 20020055627. FKBP10 is described for instance by WO2000055320.

Preferably the Marker genes for colon cancer are intestinal peptide-associated transporter HPT-1 (CDH17), caudal type homeo box transcription factor 1 (CDX1) and fatty acid binding protein 1 (FABP1). Preferably, a Biomarker for CDH17 is measured alone. Biomarkers for CDX1 and FABP1 can be measured in addition to, or in place of a Biomarker for CDH17. CDH17 is described for instance by Takamura et al. (2004); and WO2004063355. CDX1 is described for instance by Pilozzi et al. (2004); 20050059008; and 20010029020. FABP1 is described for instance by Borchers et al. (1997); Chan et al. (1985); Chen et al. (1986); and Lowe et al. (1985).

Preferably the Marker genes for lung cancer are surfactant protein-B (SP-B), thyroid transcription factor (TTF), desmoglein 3 (DSG3), keratin 6 isoform 6F (KRT6F), p53-related gene (p73H), and surfactant protein C (SFTPC). Preferably, Biomarkers for SP-B, TTF and DSG3 are measured together. Biomarkers for KRT6F, p73H and SFTPC can be measured in addition to, or in place of any of the Biomarkers for SP-B, TTF and/or DSG3. SP-B is described for instance by Pilot-Mathias et al. (1989); 20030219760; and 20030232350. TTF is described for instance by Jones et al. (2005); 20040219575; WO1998056953; WO2002073204; 20030138793; and WO2004063355. DSG3 is described for instance by Wan et al. (2003); 20030232350; WO2004030615; and WO2002101357. KRT6F is described for instance by Takahashi et al. (1995); 20040146862; and 20040219572. p73H is described for instance by Senoo et al. (1998); and 20030138793. SFTPC is described for instance by Glasser et al. (1988).

The Marker genes can be further selected from a gender specific Marker such as, in the case of a male patient KLK3, KLK2, NGEP or NPY; or in the case of a female patient PDEF, MGB, PIP, B305D, B726 or GABA-Pi; and/or WT1, PAX8, STAR or EMX2.

Preferably, the Marker genes for breast cancer are prostate derived epithelial factor (PDEF), mammaglobin (MG), prolactin-inducible protein (PIP), B305D, B726, and GABA-π. Preferably, Biomarkers for PDEF and MG are measured together. Biomarkers for PIP, B305D, B726 and GABA-Pi can be measured in addition to, or in place of Biomarkers for PDEF and/or MG. PDEF is described for instance by WO2004030615; WO2000006589; WO2001073032; Wallace et al. (2005); Feldman et al. (2003); and Oettgen et al. (2000). MG is described for instance by WO2004030615; 20030124128; Fleming et al. (2000); Watson et al. (1996 and 1998); and U.S. Pat. No. 5,668,267. PIP is described for instance by Autiero et al. (2002); Clark et al. (1999); Myal et al. (1991) and Murphy et al. (1987). B305D, B726 and GABA-Pi are described by Reinholz et al. (2005). NGEP is described for instance by Bera et al. (2004).

Preferably the Markers for ovarian cancer are Wilm's tumor 1 (WT1), PAX8, steroidogenic acute regulatory protein (STAR) and EMX2. Preferably, Biomarkers for WT1 are measured. Biomarkers for STAR and EMX2 can be measured in addition to or in place of Biomarkers for WT1. WT1 is described for instance by U.S. Pat. Nos. 5,350,840; 6,232,073; 6,225,051; 20040005563; and Bentov et al. (2003). PAX8 is described for instance by 20050037010; Poleev et al. (1992); Di Palma et al. (2003); Marques et al. (2002); Cheung et al. (2003); Goldstein et al. (2002); Oji et al. (2003); Rauscher et al. (1993); Zapata-Benavides et al. (2002); and Dwight et al. (2003). STAR is described for instance by Gradi et al. (1995); and Kim et al. (2003). EMX2 is described for instance by Noonan et al. (2001).

Preferably the Markers for prostate cancer are KLK3, KLK2, NGEP and NPY. Preferably, Biomarkers for KLK3 are measured. Biomarkers for KLK2, NGEP and NPY can be measured in addition to or in place of KLK3. KLK2 and KLK3 are described for instance by Magklara et al. (2002). KLK2 is described for instance by 20030215835; and U.S. Pat. No. 5,786,148. KLK3 is described for instance by U.S. Pat. No. 6,261,766.

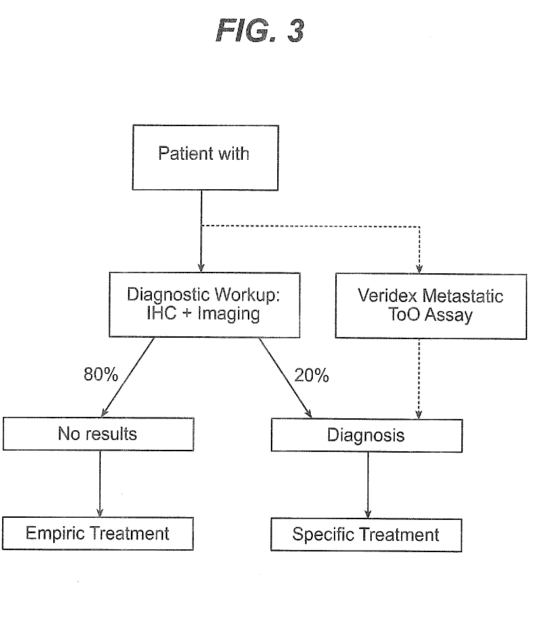

The method can also include obtaining additional clinical information including the site of metastasis to determine the origin of the carcinoma. A flow diagram is provided in FIG. 3.

The invention further provides a method for obtaining optimal biomarker sets for carcinomas by using metastases of know origin, determining Biomarkers therefor and comparing the Biomarkers to Biomarkers of metastases of unknown origin.

The invention further provides a method for providing direction of therapy by determining the origin of a metastasis of unknown origin according to the methods described herein and identifying the appropriate treatment therefor.

The invention further provides a method for providing a prognosis by determining the origin of a metastasis of unknown origin according to the methods described herein and identifying the corresponding prognosis therefor.

The invention further provides a method for finding Biomarkers comprising determining the expression level of a Marker gene in a particular metastasis, measuring a Biomarker for the Marker gene to determine expression thereof, analyzing the expression of the Marker gene according to the methods described herein and determining if the Marker gene is effectively specific for the tumor of origin.

The invention further provides compositions comprising at least one isolated sequence selected from SEQ ID NOs: 11-58.

The invention further provides kits, articles, microarrays or gene chip, diagnostic/prognostic portfolios for conducting the assays described herein and patient reports for reporting the results obtained by the present methods.

The mere presence or absence of particular nucleic acid sequences in a tissue sample has only rarely been found to have diagnostic or prognostic value. Information about the expression of various proteins, peptides or mRNA, on the other hand, is increasingly viewed as important. The mere presence of nucleic acid sequences having the potential to express proteins, peptides, or mRNA (such sequences referred to as “genes”) within the genome by itself is not determinative of whether a protein, peptide, or mRNA is expressed in a given cell. Whether or not a given gene capable of expressing proteins, peptides, or mRNA does so and to what extent such expression occurs, if at all, is determined by a variety of complex factors. Irrespective of difficulties in understanding and assessing these factors, assaying gene expression can provide useful information about the occurrence of important events such as tumorogenesis, metastasis, apoptosis, and other clinically relevant phenomena. Relative indications of the degree to which genes are active or inactive can be found in gene expression profiles. The gene expression profiles of this invention are used to provide a diagnosis and treat patients for CUP.

Sample preparation requires the collection of patient samples. Patient samples used in the inventive method are those that are suspected of containing diseased cells such as cells taken from a nodule in a fine needle aspirate (FNA) of tissue. Bulk tissue preparation obtained from a biopsy or a surgical specimen and laser capture microdissection are also suitable for use. Laser Capture Microdissection (LCM) technology is one way to select the cells to be studied, minimizing variability caused by cell type heterogeneity. Consequently, moderate or small changes in Marker gene expression between normal or benign and cancerous cells can be readily detected. Samples can also comprise circulating epithelial cells extracted from peripheral blood. These can be obtained according to a number of methods but the most preferred method is the magnetic separation technique described in U.S. Pat. No. 6,136,182. Once the sample containing the cells of interest has been obtained, a gene expression profile is obtained using a Biomarker, for genes in the appropriate portfolios.

Preferred methods for establishing gene expression profiles include determining the amount of RNA that is produced by a gene that can code for a protein or peptide. This is accomplished by reverse transcriptase PCR (RT-PCR), competitive RT-PCR, real time RT-PCR, differential display RT-PCR, Northern Blot analysis and other related tests. While it is possible to conduct these techniques using individual PCR reactions, it is best to amplify complementary DNA (cDNA) or complementary RNA (cRNA) produced from mRNA and analyze it via microarray. A number of different array configurations and methods for their production are known to those of skill in the art and are described in for instance, U.S. Pat. Nos. 5,445,934; 5,532,128; 5,556,752; 5,242,974; 5,384,261; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,599,695; 5,624,711; 5,658,734; and 5,700,637.

Microarray technology allows for measuring the steady-state mRNA level of thousands of genes simultaneously providing a powerful tool for identifying effects such as the onset, arrest, or modulation of uncontrolled cell proliferation. Two microarray technologies are currently in wide use, cDNA and oligonucleotide arrays. Although differences exist in the construction of these chips, essentially all downstream data analysis and output are the same. The product of these analyses are typically measurements of the intensity of the signal received from a labeled probe used to detect a cDNA sequence from the sample that hybridizes to a nucleic acid sequence at a known location on the microarray. Typically, the intensity of the signal is proportional to the quantity of cDNA, and thus mRNA, expressed in the sample cells. A large number of such techniques are available and useful. Preferred methods for determining gene expression can be found in U.S. Pat. Nos. 6,271,002; 6,218,122; 6,218,114; and 6,004,755.

Analysis of the expression levels is conducted by comparing such signal intensities. This is best done by generating a ratio matrix of the expression intensities of genes in a test sample versus those in a control sample. For instance, the gene expression intensities from a diseased tissue can be compared with the expression intensities generated from benign or normal tissue of the same type. A ratio of these expression intensities indicates the fold-change in gene expression between the test and control samples.

The selection can be based on statistical tests that produce ranked lists related to the evidence of significance for each gene's differential expression between factors related to the tumor's original site of origin. Examples of such tests include ANOVA and Kruskal-Wallis. The rankings can be used as weightings in a model designed to interpret the summation of such weights, up to a cutoff, as the preponderance of evidence in favor of one class over another. Previous evidence as described in the literature may also be used to adjust the weightings.

In the present invention, 10 markers were chosen that showed significant evidence of differential expression amongst 6 tumor types. The selection process included an ad-hoc collection of statistical tests, mean-variance optimization, and expert knowledge. In an alternative embodiment the feature extraction methods could be automated to select and test markers through supervised learning approaches. As the database grows, the selection of markers can be repeated in order to produce the highest diagnostic accuracy possible at any given state of the database.

A preferred embodiment is to normalize each measurement by identifying a stable control set and scaling this set to zero variance across all samples. This control set is defined as any single endogenous transcript or set of endogenous transcripts affected by systematic error in the assay, and not known to change independently of this error. All markers are adjusted by the sample specific factor that generates zero variance for any descriptive statistic of the control set, such as mean or median, or for a direct measurement. Alternatively, if the premise of variation of controls related only to systematic error is not true, yet the resulting classification error is less when normalization is performed, the control set will still be used as stated. Non-endogenous spike controls could also be helpful, but are not preferred.

Classification trees are constructed using the Matlab function ‘treefit’ which is commercially available from The Mathworks. The function is based on the work described in Breiman, L., Classification and Regression Trees, Chapman & Hall, Boca Raton, 1993.

The code performs the following steps in the following order using Matlab version 7.3.0 (http://www.mathworks.com) with the Statistical Toolbox installed. The term treefit refers to a tree generated from the treefit function in the Statistical Toolbox namespace.
1) CT values for 10 marker genes and 2 controls are stored on a hard drive for all available training set samples.
2) For each sample, subtracting the sample specific average of the controls from each marker normalizes the 10 marker gene values.
3) The training data set is composed of metastasis with known sites of origin where each sample has at least one of its target markers specific for the labeled tissue of origin with a normalized CT rounded to the nearest integer value less than 5.
4) Treefit is used to construct 7 trees from the training data in (3). Each node containing 2 or more classes must have 10 or more observations in order to be split. The criterion for choosing a split is the Gini diversity index. The prior probabilities are equal for each class used in any particular tree. Each tree is pruned to one level below it's minimum cost as determined by the average of 100 10-fold cross-validations. The cost for each pruning level is determined by the Matlab Statistical Toolbox function ‘treetest’. One set of 3 trees is specific for males with a tree corresponding to a background tissue of lung, another tree for a background of colon, and a general tree for any other background that is not colon, lung, pancreas, or prostate. There are four specific female trees: a colon background tree, a lung background tree, an ovarian background tree, and a general tree used with any other background that is not breast, colon, lung, pancreas, or ovary. Each male specific tree will not use WT1, MG, and PDEF as the male specific trees do not attempt to classify samples as breast or ovary.

Likewise, the female specific trees will not use KLK3 as prostate is not used as a class in the female trees. The colon background specific trees do not use HPT1. The lung background specific tree does not use HUMP, TTF1, or DSG3. WT1 is not used in the tree specific for an ovarian background.

In order to test a sample:
1) Read in a test data set.
2) Generate a sample specific average of both controls.
3) For each sample, uses the sample specific average to subtract from each marker.
4) Replace any normalized CT generated from a raw CT of 40 with 40.
5) For each sample in the test set the following are tested.
   a. If the average of both controls are greater than 34 than the sample is labeled as ‘CTR_FAILURE’ with zeros for posterior probabilities.
   b. The backgrounds are checked for colon, ovary, or lung. If a match is found than the gender is checked as well. The background and gender specific tree is then used to evaluate the sample.
   c. If breast, pancreas, lungSCC, or prostate is found as the background label, then a label of ‘FAILURE_ineligible_sample’ is given to the sample, and the posterior probabilities are all set to zero.
   d. The general tree for either male or female is used for all other samples.

The classification results determined at steps 5b-d can be generated by starting at the specified tree's root node and following a path to the terminal leaf based on the cutoff at each encountered node. Alternatively, a program with functionality similar to the Matlab Statistical Toolbox function ‘treeval’ can be used to generate the results and write them to a file.

The present invention includes gene expression portfolios obtained by this process.

Gene expression profiles can be displayed in a number of ways. The most common is to arrange raw fluorescence intensities or ratio matrix into a graphical dendogram where columns indicate test samples and rows indicate genes. The data are arranged so genes that have similar expression profiles are proximal to each other. The expression ratio for each gene is visualized as a color. For example, a ratio less than one (down-regulation) appears in the blue portion of the spectrum while a ratio greater than one (up-regulation) appears in the red portion of the spectrum. Commercially available computer software programs are available to display such data including “GeneSpring” (Silicon Genetics, Inc.) and “Discovery” and “Infer” (Partek, Inc.) Measurements of the abundance of unique RNA species are collected from primary tumors or metastatic tumors from primaries of known origin. These readings along with clinical records including, but not limited to, a patient's age, gender, site of origin of primary tumor, and site of metastasis (if applicable) are used to generate a relational database. The database is used to select RNA transcripts and clinical factors that can be used as marker variables to predict the primary origin of a metastatic tumor.

In the case of measuring protein levels to determine gene expression, any method known in the art is suitable provided it results in adequate specificity and sensitivity. For example, protein levels can be measured by binding to an antibody or antibody fragment specific for the protein and measuring the amount of antibody-bound protein. Antibodies can be labeled by radioactive, fluorescent or other detectable reagents to facilitate detection. Methods of detection include, without limitation, enzyme-linked immunosorbent assay (ELISA) and immunoblot techniques.

Modulated genes used in the methods of the invention are described in the Examples. The genes that are differentially expressed are either up regulated or down regulated in patients with carcinoma of a particular origin relative to those with carcinomas from different origins. Up regulation and down regulation are relative terms meaning that a detectable difference (beyond the contribution of noise in the system used to measure it) is found in the amount of expression of the genes relative to some baseline. In this case, the baseline is determined based on the classification tree. The genes of interest in the diseased cells are then either up regulated or down regulated relative to the baseline level using the same measurement method. Diseased, in this context, refers to an alteration of the state of a body that interrupts or disturbs, or has the potential to disturb, proper performance of bodily functions as occurs with the uncontrolled proliferation of cells. Someone is diagnosed with a disease when some aspect of that person's genotype or phenotype is consistent with the presence of the disease. However, the act of conducting a diagnosis or prognosis may include the determination of disease/status issues such as determining the likelihood of relapse, type of therapy and therapy monitoring. In therapy monitoring, clinical judgments are made regarding the effect of a given course of therapy by comparing the expression of genes over time to determine whether the gene expression profiles have changed or are changing to patterns more consistent with normal tissue.

Genes can be grouped so that information obtained about the set of genes in the group provides a sound basis for making a clinically relevant judgment such as a diagnosis, prognosis, or treatment choice. These sets of genes make up the portfolios of the invention. As with most diagnostic Markers, it is often desirable to use the fewest number of Markers sufficient to make a correct medical judgment. This prevents a delay in treatment pending further analysis as well unproductive use of time and resources.

One method of establishing gene expression portfolios is through the use of optimization algorithms such as the mean variance algorithm widely used in establishing stock portfolios. This method is described in detail in 20030194734. Essentially, the method calls for the establishment of a set of inputs (stocks in financial applications, expression as measured by intensity here) that will optimize the return (e.g., signal that is generated) one receives for using it while minimizing the variability of the return. Many commercial software programs are available to conduct such operations. “Wagner Associates Mean-Variance Optimization Application,” referred to as “Wagner Software” throughout this specification, is preferred. This software uses functions from the “Wagner Associates Mean-Variance Optimization Library” to determine an efficient frontier and optimal portfolios in the Markowitz sense is preferred. Markowitz (1952). Use of this type of software requires that microarray data be transformed so that it can be treated as an input in the way stock return and risk measurements are used when the software is used for its intended financial analysis purposes.

The process of selecting a portfolio can also include the application of heuristic rules. Preferably, such rules are formulated based on biology and an understanding of the technology used to produce clinical results. More preferably, they are applied to output from the optimization method. For example, the mean variance method of portfolio selection can be applied to microarray data for a number of genes differentially expressed in subjects with cancer. Output from the method would be an optimized set of genes that could include some genes that are expressed in peripheral blood as well as in diseased tissue. If samples used in the testing method are obtained from peripheral blood and certain genes differentially expressed in instances of cancer could also be differentially expressed in peripheral blood, then a heuristic rule can be applied in which a portfolio is selected from the efficient frontier excluding those that are differentially expressed in peripheral blood. Of course, the rule can be applied prior to the formation of the efficient frontier by, for example, applying the rule during data pre-selection.

Other heuristic rules can be applied that are not necessarily related to the biology in question. For example, one can apply a rule that only a prescribed percentage of the portfolio can be represented by a particular gene or group of genes. Commercially available software such as the Wagner Software readily accommodates these types of heuristics. This can be useful, for example, when factors other than accuracy and precision (e.g., anticipated licensing fees) have an impact on the desirability of including one or more genes.

The gene expression profiles of this invention can also be used in conjunction with other non-genetic diagnostic methods useful in cancer diagnosis, prognosis, or treatment monitoring. For example, in some circumstances it is beneficial to combine the diagnostic power of the gene expression based methods described above with data from conventional Markers such as serum protein Markers (e.g., Cancer Antigen 27.29 (“CA 27.29”)). A range of such Markers exists including such analytes as CA 27.29. In one such method, blood is periodically taken from a treated patient and then subjected to an enzyme immunoassay for one of the serum Markers described above. When the concentration of the Marker suggests the return of tumors or failure of therapy, a sample source amenable to gene expression analysis is taken. Where a suspicious mass exists, a fine needle aspirate (FNA) is taken and gene expression profiles of cells taken from the mass are then analyzed as described above. Alternatively, tissue samples may be taken from areas adjacent to the tissue from which a tumor was previously removed. This approach can be particularly useful when other testing produces ambiguous results.

Kits made according to the invention include formatted assays for determining the gene expression profiles. These can include all or some of the materials needed to conduct the assays such as reagents and instructions and a medium through which Biomarkers are assayed.

Articles of this invention include representations of the gene expression profiles useful for treating, diagnosing, prognosticating, and otherwise assessing diseases. These profile representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from patient samples. Alternatively, the profiles can be recorded in different representational format. A graphical recordation is one such format. Clustering algorithms such as those incorporated in “DISCOVERY” and “INFER” software from Partek, Inc. mentioned above can best assist in the visualization of such data.

Different types of articles of manufacture according to the invention are media or formatted assays used to reveal gene expression profiles. These can comprise, for example, microarrays in which sequence complements or probes are affixed to a matrix to which the sequences indicative of the genes of interest combine creating a readable determinant of their presence. Alternatively, articles according to the invention can be fashioned into reagent kits for conducting hybridization, amplification, and signal generation indicative of the level of expression of the genes of interest for detecting cancer.

The following examples are provided to illustrate but not limit the claimed invention. All references cited herein are hereby incorporated herein by reference.

### Example 1

### Materials and Methods

### Pancreatic Cancer Markers Gene Discovery.

RNA was isolated from pancreatic tumor, normal pancreatic, lung, colon, breast and ovarian tissues using Trizol. The RNA was then used to generate amplified, labeled RNA (Lipshutz et al. (1999)) which was then hybridized onto Affymetrix U133A arrays. The data were then analyzed in two ways.

### Tissue Samples.

In the first method, this dataset was filtered to retain only those genes with at least two present calls across the entire dataset. This filtering left 14,547 genes. 2,736 genes were determined to be overexpressed in pancreatic cancer versus normal pancreas with a p value of less than 0.05. Forty five genes of the 2,736 were also overexpressed by at least two-fold compared to the maximum intensity found from lung and colon tissues. Finally, six probe sets were found which were overexpressed by at least two-fold compared to the maximum intensity found from lung, colon, breast, and ovarian tissues.

In the second method, this dataset was filtered to retain only those genes with no more than two present calls in breast, colon, lung, and ovarian tissues. This filtering left 4,654 genes. 160 genes of the 4,654 genes were found to have at least two present calls in the pancreatic tissues (normal and cancer). Finally, eight probe sets were selected which showed the greatest differential expression between pancreatic cancer and normal tissues.

A total of 260 FFPE metastasis and primary tissues were acquired from a variety of commercial vendors. The samples tested included: 30 breast metastasis, 30 colorectal metastasis, 56 lung metastasis, 49 ovarian metastasis 43 pancreas metastasis, 18 prostate primary and 2 prostate metastases and 32 other origins (6 stomach, 6 kidney, 3 larynx, 2 liver, 1 esophagus, 1 pharynx, 1 bile duct, 1 pleura, 3 bladder, 5 melanoma, 3 lymphoma).

### RNA Extraction.

RNA isolation from paraffin tissue sections was based on the methods and reagents described in the High Pure RNA Paraffin Kit manual (Roche) with the following modifications. Paraffin embedded tissue samples were sectioned according to size of the embedded metastasis (2-5 mm=9×10 μm, 6-8 mm=6×10 μm, 8-≧10 mm=3×10 μm), and placed in RNase/DNase 1.5 ml Eppendorf tubes. Sections were deparaffinized by incubation in 1 ml of xylene for 2-5 min at room temperature following a 10-20 second vortex. Tubes were then centrifuged and supernatant was removed and the deparaffinization step was repeated. After supernatant was removed, 1 ml of ethanol was added and sample was vortexed for 1 minute, centrifuged and supernatant removed. This process was repeated one additional time. Residual ethanol was removed and the pellet was dried in a 55° C. oven for 5- 10 minutes and resuspended in 100 μl of tissue lysis buffer, 16 μl 10% SDS and 80 μl Proteinase K. Samples were vortexed and incubated in a thermomixer set at 400 rpm for 2 hours at 55° C. 325 μl binding buffer and 325 μl ethanol was added to each sample that was then mixed, centrifuged and the supernatant was added onto the filter column. Filter column along with collection tube were centrifuged for 1 minute at 8000 rpm and flow through was discarded. A series of sequential washes were performed (500 μl Wash Buffer I→500 μl Wash Buffer II→300 μl Wash Buffer II) in which each solution was added to the column, centrifuged and flow through discarded. Column was then centrifuged at maximum speed for 2 minutes, placed in a fresh 1.5 ml tube and 90 μl of elution buffer was added. RNA was obtained after a 1 minute incubation at room temperature followed by a 1 minute centrifugation at 8000 rpm. Sample was DNase treated with the addition of 10 μl DNase incubation buffer, 2 μl of DNase I and incubated for 30 minutes at 37° C. DNase was inactivated following the addition of 20 μl of tissue lysis buffer, 18 μl 10% SDS and 40 μl Proteinase K. Again, 325 μl binding buffer and 325 μl ethanol was added to each sample that was then mixed, centrifuged and supernatant was added onto the filter column. Sequential washes and elution of RNA proceeded as stated above with the exception of 50 μl of elution buffer being used to elute the RNA. To eliminate glass fiber contamination carried over from the column RNA was centrifuged for 2 minutes at full speed and supernatant was removed into a fresh 1.5 ml Eppendorf tube. Samples were quantified by OD 260/280 readings obtained by a spectrophotometer and samples were diluted to 50 ng/1 μl. The isolated RNA was stored in Rnase-free water at −80° C. until use.

### TaqMan® Primer and Probe Design.

Appropriate mRNA reference sequence accession numbers in conjunction with Oligo 6.0 were used to develop TaqMan® CUP assays (lung Markers: human surfactant, pulmonary-associated protein B (SP-B), thyroid transcription factor 1 (TTF1), desmoglein 3 (DSG3), colorectal Marker: cadherin 17 (CDH17), breast Markers: mammaglobin (MG), prostate-derived ets transcription factor (PDEF), ovarian Marker: wilms tumor 1 (WT1), pancreas Markers: prostate stem cell antigen (PSCA), coagulation factor V (F5), prostate Marker kallikrein 3 (KLK3)) and housekeeping assays beta actin (β-Actin), hydroxymethylbilane synthase (PBGD). Primers and hydrolysis probes for each assay are listed in Table 2. Genomic DNA amplification was excluded by designing assays around exon-intron splicing sites. Hydrolysis probes were labeled at the 5′ nucleotide with FAM as the reporter dye and at 3′ nucleotide with BHQ1-TT as the internal quenching dye.

### Quantitative Real-Time Polymerase Chain Reaction.

Quantitation of gene-specific RNA was carried out in a 384 well plate on the ABI Prism 7900HT sequence detection system (Applied Biosystems). For each thermo-cycler run calibrators and standard curves were amplified. Calibrators for each Marker consisted of target gene in vitro transcripts that were diluted in carrier RNA from rat kidney at 1×10⁵ copies. Standard curves for housekeeping Markers consisted of target gene in vitro transcripts that were serially diluted in carrier RNA from rat kidney at 1×10⁷, 1×10⁵ and 1×10³ copies. No target controls were also included in each assay run to ensure a lack of environmental contamination. All samples and controls were run in duplicate. qRT-PCR was performed with general laboratory use reagents in a 10 μl reaction containing: RT-PCR Buffer (50 nM Bicine/KOH pH 8.2, 115 nM KAc, 8% glycerol, 2.5 mM MgCl₂, 3.5 mM MnSO₄, 0.5 mM each of dCTP, dATP, dGTP and dTTP), Additives (2 mM Tris-Cl pH 8, 0.2 mM Albumin Bovine, 150 mM Trehalose, 0.002% Tween 20), Enzyme Mix (2U Tth (Roche), 0.4 mg/μl Ab TP6-25), Primer and Probe Mix (0.2 μM Probe, 0.5 μM Primers). The following cycling parameters were followed: 1 cycle at 95° C. for 1 minute; 1 cycle at 55° C. for 2 minutes; Ramp 5%; 1 cycle at 70° C. for 2 minutes; and 40 cycles of 95° C. for 15 seconds, 58° C. for 30 seconds. After the PCR reaction was completed, baseline and threshold values were set in the ABI 7900HT Prism software and calculated Ct values were exported to Microsoft Excel.

One-Step vs. Two-Step Reaction.

First strand synthesis was carried out using either 100 ng of random hexamers or gene specific primers per reaction. In the first step, 11.5 μl of Mix-1 (primers and 1 ug of total RNA) was heated to 65° C. for 5 minutes and then chilled on ice. 8.5 μl of Mix-2 (1× Buffer, 0.01 mM DTT, 0.5 mM each dNTP's, 0.25 U/μl RNasin®, 10 U/μl Superscript III) was added to Mix-1 and incubated at 50° C. for 60 minutes followed by 95° C. for 5 minutes. The cDNA was stored at −20° C. until ready for use. qRT-PCR for the second step of the two-step reaction was performed as stated above with the following cycling parameters: 1 cycle at 95° C. for 1 minute; 40 cycles of 95° C. for 15 seconds, 58° C. for 30 seconds. qRT-PCR for the one-step reaction was performed exactly as stated in the preceding paragraph. Both the one-step and two-step reactions were performed on 100 ng of template (RNA/cDNA). After the PCR reaction was completed, baseline and threshold values were set in the ABI 7900HT Prism software and calculated Ct values were exported to Microsoft Excel.

### Generation of a Heatmap.

For each sample, a ΔCt was calculated by taking the mean Ct of each CUP Marker and subtracting the mean Ct of an average of the housekeeping Markers (ΔCt=Ct(CUP Marker)−Ct(Ave. HK Marker)). The minimal ΔCt for each tissue of origin Marker set (lung, breast, prostate, colon, ovarian and pancreas) was determined for each sample. The tissue of origin with the overall minimal ΔCt was scored one and all other tissue of origins scored zero. Data were sorted according to pathological diagnosis. Partek Pro was populated with the modified feasibility data and an intensity plot was generated.

### Results.

### Discovery of Novel Pancreatic Tumor of Origin and Cancer Status Markers.

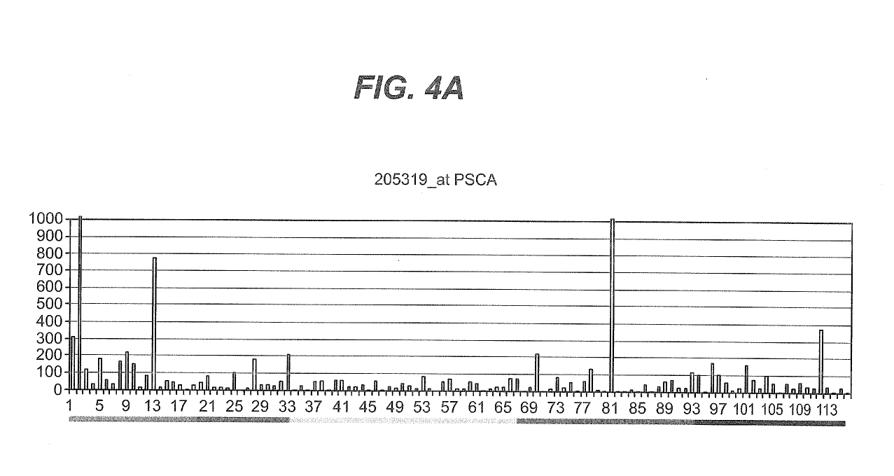

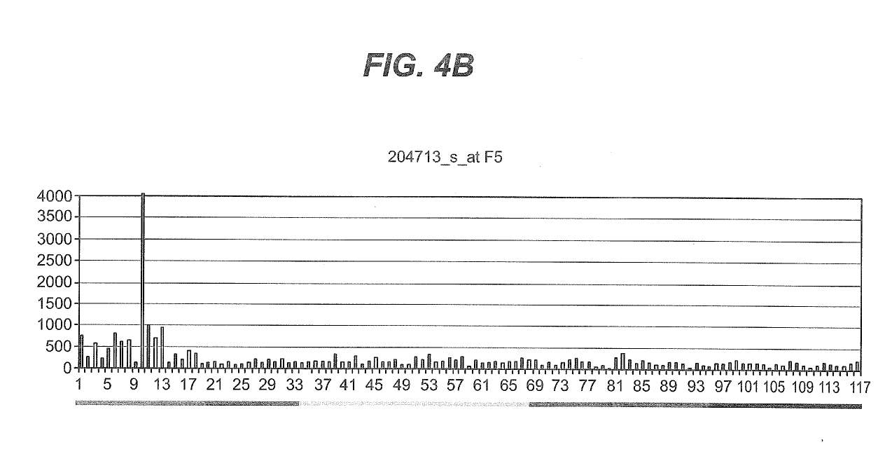

First, five pancreas Marker candidates were analyzed: prostate stem cell antigen (PSCA), serine proteinase inhibitor, clade A member 1 (SERPINA1), cytokeratin 7 (KRT7), matrix metalloprotease 11 (MMP11), and mucin4 (MUC4) (Varadhachary et al. (2004); Fukushima et al. (2004); Argani et al. (2001); Jones et al. (2004); Prasad et al. (2005); and Moniaux et al. (2004)) using DNA microarrays and a panel of 13 pancreatic ductal adenocarcinomas, five normal pancreas tissues, and 98 samples from breast, colorectal, lung, and ovarian tumors. Only PSCA demonstrated moderate sensitivity (six out of thirteen or 46% of pancreatic tumors were detected) at a high specificity (91 out of 98 or 93% were correctly identified as not being of pancreatic origin) (FIG. 4A). In contrast, KRT7, SERPINA1, MMP1, and MUC4 demonstrated sensitivities of 38%, 31%, 85%, and 31%, respectively, at specificities of 66%, 91%, 82%, and 81 %, respectively. These data were in good agreement with qRT-PCR performed on 27 metastases of pancreatic origin and 39 metastases of non-pancreatic origin for all Markers except for MMP11 which showed poorer sensitivity and specificity with qRT-PCR and the metastases. In conclusion, the microarray data on snap frozen, primary tissue serves as a good indicator of the ability of the Marker to identify a FFPE metastasis as being pancreatic in origin using qRT-PCR but that additional Markers may be useful for optimal performance.

Because pancreatic ductal adenocarcinoma develops from ductal epithelial cells that comprise only a small percentage of all pancreatic cells (with acinar cells and islet cells comprising the majority) and because pancreatic adenocarcinoma tissues contain a significant amount of adjacent normal tissue (Prasad et al. (2005); and Ishikawa et al. (2005)), it has been difficult to identify pancreatic cancer Markers (i.e., upregulated in cancer) which would also differentiate this organ from the organs. For use in a CUP panel such differentiation is necessary. The first query method (see Materials and Methods) returned six probe sets: coagulation factor V (F5), a hypothetical protein FLJ22041 similar to FK506 binding proteins (FKBP10), β 6 integrin (ITGB6), transglutaminase 2 (TGM2), heterogeneous nuclear ribonucleoprotein A0 (HNRPA0), and BAX delta (BAX). The second query method (see Materials and Methods) returned eight probe sets: F5, TGM2, paired-like homeodomain transcription factor 1 (PITX1), trio isoform mRNA (TRIO), mRNA for p73H (p73), an unknown protein for MGC:10264 (SCD), and two probe sets for claudin18. F5 and TGM2 were present in both query results and, of the two, F5 looked the most promising (FIG. 4B).

Optimization of Sample Prep and qRT-PCR using FFPE Tissues.

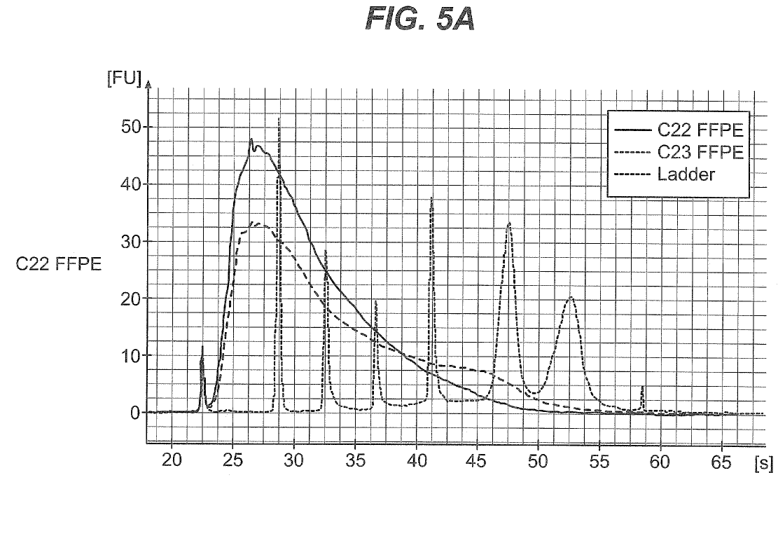

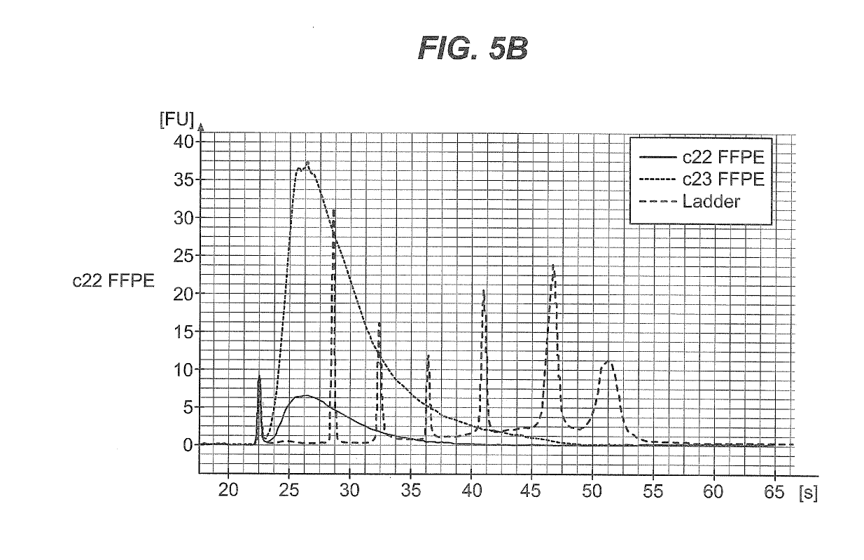

Next the RNA isolation and qRT-PCR methods were optimized using fixed tissues before examining Marker panel performance. First the effect of reducing the proteinase K incubation time from sixteen hours to 3 hours was analyzed. There was no effect on yield. However, some samples showed longer fragments of RNA when the shorter proteinase K step was used (FIG. 5). For example, when RNA was isolated from a one year old block (C22), there was no observed difference in the electropherograms. However, when RNA was isolated from a five year old block (C23), a larger fraction of higher molecular weight RNAs was observed, as assessed by the hump in the shoulder, when the shorter proteinase K digest was used. This trend generally held when other samples were processed, regardless of the organ of origin for the FFPE metastasis. In conclusion, shortening the proteinase K digestion time does not sacrifice RNA yields and may aid in isolating longer, less degraded RNA.

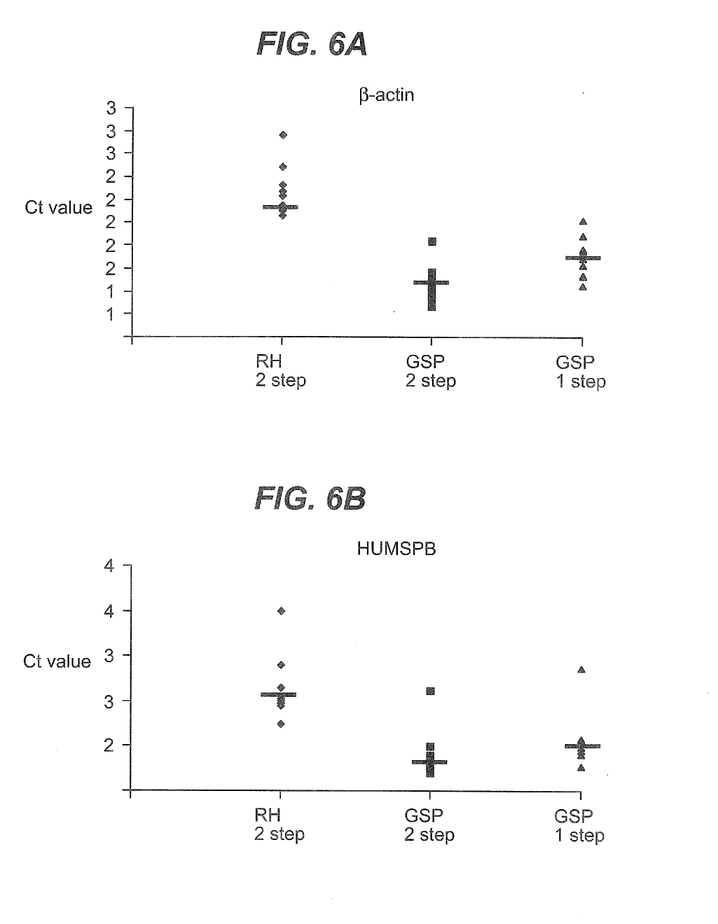

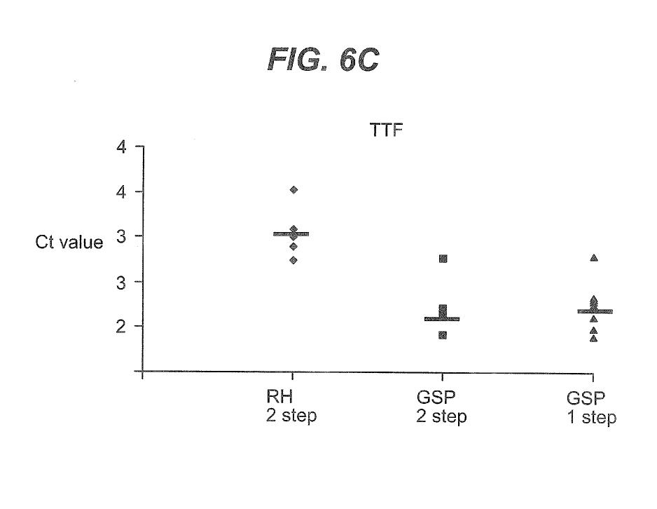

Next, three different methods of reverse transcription were compared: reverse transcription with random hexamers followed by qPCR (two step), reverse transcription with a gene-specific primer followed by qPCR (two step), and a one-step qRT-PCR using gene-specific primers. RNA was isolated from eleven metastases and compared Ct values across the three methods for β-actin, human surfactant protein B (HUMSPB), and thyroid transcription factor (TTF) (FIG. 6). There were statistically significant differences (p<0.001) for all comparisons. For all three genes, the reverse transcription with random hexamers followed by qPCR (two step reaction) gave the highest Ct values while the reverse transcription with a gene-specific primer followed by qPCR (two step reaction) gave slightly (but statistically significant) lower Ct values than the corresponding 1 step reaction. However, the 2 step RT-PCR with gene-specific primers had a longer reverse transcription step. When HUMSPB and TTF Ct values were normalized to the corresponding β-actin value for each sample, there were no differences in the normalized Ct values across the three methods. In conclusion, optimization of the RT-PCR reaction conditions can generate lower Ct values, which may help in analyzing older paraffin blocks (Cronin et al. (2004)), and a one step RT-PCR reaction with gene-specific primers can generate Ct values comparable to those generated in the corresponding two step reaction.

Diagnostic Performance of a CUP qRT-PCR Assay.

Next 12 qRT-PCR reactions (10 Markers and two housekeeping genes) were performed on 239 FFPE metastases. The Markers used for the assay are shown in Table 2. The lung Markers were human surfactant pulmonary-associated protein B (HUMPSPB), thyroid transcription factor 1 (TTF1), and desmoglein 3 (DSG3). The colorectal Marker was cadherin 17 (CDH17). The breast Markers were mammaglobin (MG) and prostate-derived Ets transcription factor (PDEF). The ovarian Marker was Wilms tumor 1 (WT1). The pancreas Markers were prostate stem cell antigen (PSCA) and coagulation factor V (F5), and the prostate Marker was kallikrein 3 (KLK3). For gene descriptions, see Table 28.

TABLE 2
Primer and probe sequences, accession numbers, and amplicon lengths.

| | SEQ | | | SEQ |
|---|---|---|---|---|
| Target | ID NO | Sequence (5′-3′) | Description | ID NO |
| SP-B | 59 | cacagccccgacctttgatga | Forward primer | 11 |
| | | ggtcccagagcccgtctca | Reverse primer | 12 |
| | | agctgtccagctgcaaaggaaaagcc | Probe* | 13 |
| | | cacagccccgacctttgatgagaactcagctgtccagcc | Amplicon | 14 |
| | | tgcaaaggaaaagccaagtgagacgggctctgggacc | | |
| TTF1 | 60 | ccaacccagacccgcgc | Forward primer | 15 |
| | | cgcccatgccgctcatgttca | Reverse primer | 16 |
| | | cccgccatctcccgcttcatg | Probe* | 17 |
| | | ccaacccagacccgcgcttccccgccatctcccgcttca | Amplicon | 18 |
| | | tgggcccggcgagcggcatgaacatgagcggcatgggcg | | |
| DSG3 | 61 | gcagagaaggagaagataactcaa | Forward primer | 19 |
| | | actccagagattcggtaggtga | Reverse primer | 20 |
| | | attgccaagattacttcagattacca | Probe* | 21 |
| | | gcagagaaggagaagataactcaaaaagaaacccaattg | Amplicon | 22 |
| | | ccaagattacttcagattaccaagcaacccagaaaatca | | |
| | | cctaccgaatctctggagt | | |
| CDH17 | 62 | tccctcggcagtggaagctta | Forward primer | 23 |
| | | tcctcaaactctgtgtgcctggta | Reverse primer | 24 |
| | | ccaaaatcaatggtactcatgcccgactg | Probe* | 25 |
| | | tccctcggcagtggaagcttacaaaacgactgggaagtt | Amplicon | 26 |
| | | tccaaaatcaatggtactcatgcccgactgtctaccagg | | |
| | | cacacagagtttgagga | | |
| MG | 63 | agttgctgatggtcctcatgc | Forward primer | 27 |
| | | cacttgtggattgattgtcttgga | Reverse primer | 28 |
| | | ccctctcccagcactgctacgca | Probe* | 29 |
| | | agttgctgatggtcctcatgctggcggccctctcccagc | Amplicon | 30 |
| | | actgctacgcaggctctggctgccccttattggagaatg | | |
| | | tgatttccaagacaatcaatccacaagtg | | |
| PDEF | 64 | cgcccacctggacatctgga | Forward primer | 31 |
| | | cactggtcgaggcacagtagtga | Reverse primer | 32 |
| | | gtcagcggcctggatgaaagagcgg | Probe* | 33 |
| | | cgcccacctggacatctggaagtcagcggcctggatgaa | Amplicon | 34 |
| | | agagcggacttcacctggggcgattcactactgtgcctc | | |
| | | gaccagtg | | |
| WT1 | 65 | gcggagcccaatacagaatacac | Forward primer | 35 |
| | | cggggctactccaggcaca | Reverse primer | 36 |
| | | tcagaggcattcaggatgtgcgacg | Probe* | 37 |
| | | gcggagcccaatacagaatacacacgcacggtgtcttca | Amplicon | 38 |
| | | gaggcattcaggatgtgcgacgtgtgcctggagtagccc | | |
| | | cg | | |
| PSCA | 66 | ctgttgatggcaggcttggc | Forward primer | 39 |
| | | ttgctcacctgggctttgca | Reverse primer | 40 |
| | | gcagccaggcactgccctgct | Probe* | 41 |
| | | ctgttgatggcaggcttggccctgcagccaggcactgcc | Amplicon | 42 |
| | | ctgctgtgctactcctgcaaagcccaggtgagcaa | | |
| F5 | 67 | tgaagaaatatcctgggattattca | Forward primer | 43 |
| | | tatgtggtatcttctggaatatcatca | Reverse primer | 44 |
| | | acaaagggaaacagatattgaagactc | Probe* | 45 |
| | | tgaagaaatatcctgggattattcagaatttgtacaaag | Amplicon | 46 |
| | | ggaaacagatattgaagactctgatgatattccagaaga | | |
| | | taccacata | | |
| KLK3 | 68 | cccccagtgggtcctcaca | Forward primer | 47 |
| | | aggatgaaacaagctgtgccga | Reverse primer | 48 |
| | | caggaacaaaagcgtgatcttgctgg | Probe* | 49 |
| | | cccccagtgggtcctcacagctgcccactgcatcaggaa | Amplicon | 50 |
| | | caaaagcgtgatcttgctgggtcggcacagcttgtttca | | |
| | | tcct | | |
| β actin | 69 | gccctgaggcactcttcca | Forward primer 51 | |
| | | cggatgtccacgtcacacttca | Reverse primer | 52 |
| | | cttccttcctgggcatggagtcctg | Probe* | 53 |
| | | gccctgaggcactcttccagccttccttcctgggcatgg | Amplicon | 54 |
| | | agtcctgtggcatccacgaaactaccttcaactccatca | | |
| | | tgaagtgtgacgtggacatccg | | |
| PBGD | 70 | ccacacacagcctactttccaa | Forward primer | 55 |
| | | tacccacgcgaatcactctca | Reverse primer | 56 |
| | | aacggcaatgcggctgcaacggcggaa | Probe* | 57 |
| | | ccacacacagcctactttccaagcggagccatgtctggt | Amplicon | 58 |
| | | aacggcaatgcggctgcaacggcggaagaaaacagccca | | |
| | | aagatgagagtgattcgcgtgggta | | |

*Probes are 5′FAM-3′BHQ1-TT

Analysis of the normalized Ct values in a heat map revealed the high specificity of the breast and prostate Markers, moderate specificity of the colon, lung, and ovarian, and somewhat lower specificity of the pancreas Markers. Combining the normalized qRT-PCR data with computational refinement improves the performance of the Marker panel. Results were obtained from the combined normalized qRT-PCR data with the linear discrimination analysis and the accuracy of the qRT-PCR assay was determined.

### Discussion.

In this example, microarray-based expression profiling was used on primary tumors to identify candidate Markers for use with metastases. The fact that primary tumors can be used to discover tumor of origin Markers for metastases is consistent with several recent findings. For example, Weigelt and colleagues have shown that gene expression profiles of primary breast tumors are maintained in distant metastases. Weigelt et al. (2003). Italiano and coworkers found that EGFR status, as assessed by IHC, was similar in 80 primary colorectal tumors and the 80 related metastases. Italiano et al. (2005). Only five of the 80 showed discordance in EGFR status. Italiano et al. (2005). Backus and colleagues identified putative Markers for detecting breast cancer metastasis using a genome-wide gene expression analysis of breast and other tissues and demonstrated that mammaglobin and CK19 detected clinically actionable metastasis in breast sentinel lymph nodes with 90% sensitivity and 94% specificity. Backus et al. (2005).

The microarray-based studies with primary tissue confirmed the specificity and sensitivity of known Markers. As a result, with the exception of F5, all of the Markers used have high specificity for the tissues studied here. Argani et al. (2001); Backus et al. (2005); Cunha et al. (2005); Borgono et al. (2004); McCarthy et al. (2003); Hwang et al. (2004); Fleming et al. (2000); Nakamura et al. (2002); and Khoor et al. (1997). A recent study determined that, using IHC, PSCA is overexpressed in prostate cancer metastases. Lam et al. (2005). Dennis et al. (2002) also demonstrated that PSCA could be used as a tumor of origin Marker for pancreas and prostate. As shown herein, strong expression of PSCA is found in some prostate tissues at the RNA level but, because by including PSA in the assay, one can now segregate prostate and pancreatic cancers. A novel finding of this study was the use of F5 as a complementary (to PSCA) Marker for pancreatic tissue of origin. In both the microarray data set with primary tissue and the qRT-PCR data set with FFPE metastases, F5 was found to complement PSCA (FIG. 4 and Table 3)

TABLE 3
feasibility data

| | Breast | Colon | Lung | Other | Ovary | Pancreas | Prostate | Total |
|---|---|---|---|---|---|---|---|---|
| Total tested | 30 | 30 | 56 | 32 | 49 | 43 | 20 | 260 |
| #Correct | 22 | 27 | 45 | 16 | 43 | 31 | 20 | 204 |
| #Other/No test | 1 | 1 | 3 | n/a | 1 | 4 | 0 | 10 |
| #Incorrect | 7 | 2 | 8 | 16 | 5 | 8 | 0 | 46 |
| % Tested | 96.67 | 96.67 | 94.64 | 100 | 97.96 | 90.70 | 100 | 96.15 |
| % Correct of | 75.86 | 93.10 | 84.91 | 0 | 89.58 | 79.49 | 100 | 81.60 |
| tested | | | | | | | | |
| Correct of total | 73.33 | 90.00 | 80.36 | 50.00 | 87.76 | 72.09 | 100 | 78.46 |
| (%) | | | | | | | | |

Previous investigators have generated CUP assays using IHC or microarrays. Su et al. (2001); Ramaswamy et al. (2001); and Bloom et al. (2004). More recently, SAGE has been coupled to a small qRT-PCR Marker panel. Dennis (2002); and Buckhaults et al. (2003). This study is the first to combine microarray-based expression profiling with a small panel of qRT-PCR assays. Microarray studies with primary tissue identified some, but not all, of the same tissue of origin Markers as those identified previously by SAGE studies. Some studies have demonstrated that a modest agreement between SAGE- and DNA microarray-based profiling data exists and that the correlation improves for genes with higher expression levels. van Ruissen et al. (2005); and Kim (2003). For example, Dennis and colleagues identified PSA, MG, PSCA, and HUMSPB while Buckhaults and coworkers (Dennis et al. (2002)) identified PDEF. Executing the CUP assay using qRT-PCR is preferred because it is a robust technology and may have performance advantages over IHC. Al-Mulla et al. (2005); and Haas et al. (2005). As shown herein, the qRT-PCR protocol was improved through the use of gene-specific primers in a one-step reaction. This is the first demonstration of the use of gene-specific primers in a one-step qRT-PCR reaction with FFPE tissue. Other investigators have either done a two step qRT-PCR (cDNA synthesis in one reaction followed by qPCR) or have used random hexamers or truncated gene-specific primers. Abrahamsen et al. (2003); Specht et al. (2001); Godfrey et al. (2000); Cronin et al. (2004); and Mikhitarian et al. (2004).

### Example 2

### CUP FFPE Total RNA Isolation Protocol

### (Highpure kit Cat#3270289)

### Purpose:

Isolation of total RNA from FFPE tissue

### Procedure:

### Preparation of Working Solutions

1. Proteinase K (PK) in kit
   Dissolve lyophilizate in 4.5ml Elution Buffer. Aliquot and store at −20° C., stable for 12 months.

### PK-4×250 mg (cat #3115852)

Dissolve lyophilizate in 12.5 ml of Elution Buffer (1× TE Buffer (pH 7.4-7). Aliquot and store at −20° C.
2. Wash Buffer I
   Add 60 ml absolute ethanol to Wash Buffer I, store at RT.
3. Wash Buffer II
   Add 200 ml absolute ethanol to Wash Buffer II, store at RT.
4. DNase I
   Dissolve lyophilizate in 400 μl Elution Buffer. Aliquot and store at −20° C., stable for 12 months.

Sectioning Paraffin Blocks ˜30-45 Minutes for 12 Blocks (12 Blocks×2 Tubes=24 Tubes)

Sections cut from the block should be processed immediately for RNA extraction
1. Use a clean sharp razor blade on Microtome to cut 6×10 micron thick sections from trimmed tissue blocks (size 3-4×5-10 mm).
   Note: New block-Discard wax sections until obtained tissue section. Used block-Discard first 3 tissue sections
2. Immediately place cut tissue in 1.5 ml microfuge tubes and tightly cap to minimize moisture.
3. The number of sections recommended based on size of tumor are shown in Table 4.

TABLE 4

| Size of MET | Sections/Tube |
|---|---|
| 8-10 mm | 6 |
| 6-8 mm | 12 |
| 2-4 mm | 18 |

### Deparaffinization ˜30-45 Minutes

1. Add 1.0 ml xylene to each sample and vortex vigorously for 10-20 sec and incubate RT 2-5 min. Centrifuge at full speed 2 min. Remove the supernatant carefully.

Note: if the tissue appears to befloating, centrifuge for an additional 2 min.
2. Repeat step 1.
3. Centrifuge at full speed 2 min. Remove the supernatant.
4. Add 1 ml ethanol abs. and vortex vigorously 1 min. Centrifuge at full speed 2 min.

### Remove the Supernatant.

5. Repeat step 4.
6. Blot the tube briefly onto a paper towel to get rid of ethanol residues.
7. Dry the tissue pellet for 5-10 min at 55° C. in oven.
   Note: it is critical that the ethanol is completely removed and the pellets are thoroughly dry, residual ethanol can inhibit PK digestion.
   Note: if PK is in −20° C., warm in RT 20-30 min.

### RNA Extraction ˜2.5-3 Hours

1. Add 100 μl Tissue Lysis Buffer, 16 μl 10% SDS and 80 μl Proteinase K working solution to one tissue pellet, vortex briefly in several intervals and incubate 2 hrs at 55° C. shaking 400 rpm.
2. Add 325 μl Binding Buffer and 325 μl ethanol abs. Mix gently by pipetting up and down.
3. Centrifuge the lysate at full speed for 2 min.
4. Combine the filter tube and the collection tube (12 tubes), and pipet the lysate supernatant into the filter.
5. Centrifuge for 30 sec at 8000 rpm and discard the flowthrough.
   Note: Step 4-5 can be repeated, if RNA needs to be pooled with 2 more tissue pellet preparations.
6. Repeat the centrifugation at 8000 rpm for 30 sec to dry the filter.
7. Add 500 μl Wash Buffer I working solution to the column and centrifuge for 15-30 sec at 8000 rpm, discard the flowthrough.
8. Add 500 μl Wash Buffer II working solution. Centrifuge for 15-30 sec at 8000 rpm, discard the flowthrough.
9. Add 300 μl Wash Buffer II working solution, centrifuge for 15-30 sec at 8000 rpm, discard the flowthrough.
10. Centrifuge the High Pure filter for 2 min at maximum speed.
11. Place the High Pure filter tube into a fresh 1.5 ml tube and add 90 μl Elution Buffer.
   Incubate for 1-2 min at room temperature. Centrifuge for 1 min at 8000 rpm.

### DNase I Treatment ˜1.5 Hours

12. Add 10 μl of 10× DNase Incubation Buffer and 1.0 μl DNase I working solution to the eluate and mix. Incubate for 45 min at 37° C. (or 2.0 μl DNase I for 30 min).
13. Add 20 μl Tissue Lysis Buffer, 18 μl 10% SDS and 40 μl Proteinase Kworking solution. Vortex briefly. Incubate for 30 min (30-60 min.) at 55° C.
14. Add 325 μl Binding Buffer and 325 μl ethanol abs. Mix and pipet into a fresh High Pure filter tube with collection tube (12 tubes).
15. Centrifuge for 30 sec at 8000 rpm and discard the flowthrough.
16. Repeat the centrifugation at 8000 rpm for 30 sec to dry the filter.
17. Add 500 μl Wash Buffer I working solution to the column. Centrifuge for 15 sec at 8000 rpm, discard the flowthrough.
18. Add 500 μl Wash Buffer II working solution. Centrifuge for 15 sec at 8000 rpm, discard the flowthrough.
19. Add 300 μl Wash Buffer II working solution. Centrifuge for 15 sec at 8000 rpm, discard the flowthrough.
20. Centrifuge the High Pure filter for 2 min at maximum speed.
21. Place the High Pure filter tube into a fresh 1.5 ml tube. Add 50 μl Elution Buffer; incubate for 1-2 min at room temperature. Centrifuge for 1 min at 8000 rpm to collect the eluated RNA.
22. Centrifuge the eluate for 2 min at full speed and transfer supernatant to a new tube without disturbing glass fibers at the bottom.
23. Take 260/280 OD reading and dilute to 50 ng/μl. Store at −80° C.

CUP ASR Assay Protocol (ABI 7900)

Purpose: Use qRT-PCR to Determine Tissue of Origin of a CUP Sample

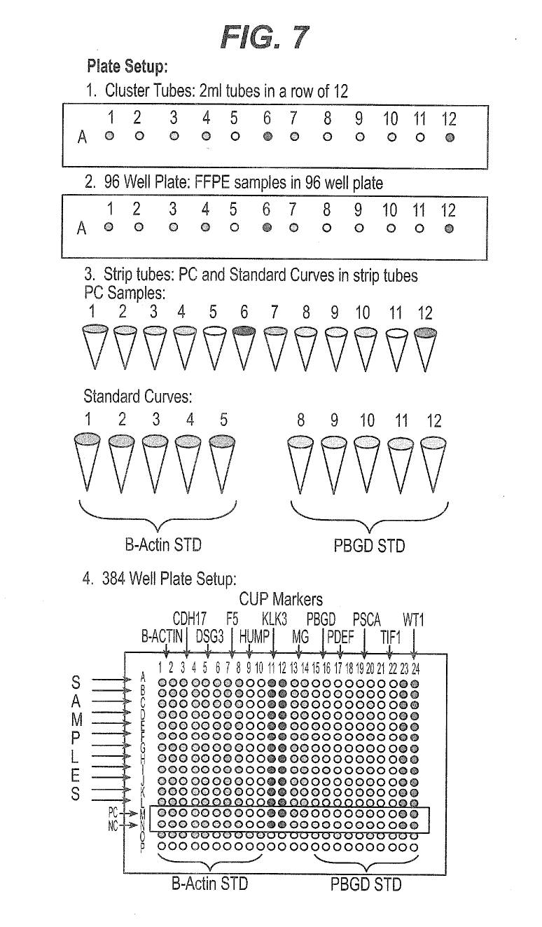

Control Setup:
1. Positive Controls (Refer to Table 5 and Plate C in Plate Setup, FIG. 7)

TABLE 5
Serial dilutions of IVT - 5 μl 1 × 10⁸ into 470 μl water
and 25 μl of 10000 rRNA=

| IVT | | | | |
|---|---|---|---|---|
| Control | CE/μl | Sample | Water | Bkgd rRNA |
| β-actin | 100E+05 | 50 | 425 | 25 |
| CDH17 | 100E+05 | 50 | 425 | 25 |
| DSG3 | 100E+05 | 50 | 425 | 25 |
| F5 | 100E+05 | 50 | 425 | 25 |
| Hump | 100E+05 | 50 | 425 | 25 |
| MG | 100E+05 | 50 | 425 | 25 |
| PBGD | 100E+05 | 50 | 425 | 25 |
| PDEF | 100E+05 | 50 | 425 | 25 |
| PSCA | 100E+05 | 50 | 425 | 25 |
| TTF1 | 100E+05 | 50 | 425 | 25 |
| WT1 | 100E+05 | 50 | 425 | 25 |

1E6. Table 5. Dilute 50,000 CE/μl rRNA to 500 CE/μl−5 μl 50,000 CE/μl+495 μl H₂O
Aliqouts 10 μl per strip tube (2 plates); Place Mix at −80° C. until ready for use.
2. Standard Curves (Refer to Table 6 and Plate C in Plate Setup, FIG. 7)
   Step 1: Standard curve was setup exactly as shown in Table 6.

TABLE 7
Stock Solution - 1 × 10⁸ IVT. Dilute 50,000 CE/μl rRNA
to 500 CE/μl - 5 μl 50,000 CE/μl + 495 μl H₂O

| IVT | | | | |
|---|---|---|---|---|
| Control | CE/μl | Sample | Water | Bkgd rRNA |
| β-actin-1 | 100E+07 | 50 | 425 | 25 |
| β-actin-2 | 100E+06 | 50 | 425 | 25 |
| β-actin-3 | 100E+05 | 50 | 425 | 25 |
| β-actin-4 | 100E+04 | 50 | 425 | 25 |
| β-actin-5 | 100E+03 | 50 | 425 | 25 |
| PBGD-1 | 100E+07 | 50 | 425 | 25 |
| PBGD-2 | 100E+06 | 50 | 425 | 25 |
| PBGD-3 | 100E+05 | 50 | 425 | 25 |
| PBGD-4 | 100E+04 | 50 | 425 | 25 |
| PBGD-5 | 100E+03 | 50 | 425 | 25 |

Aliqouts 10 μl per strip tube (2 plates); Place Mix at −80° C. until ready for use.

### Enzyme Mix:

1. MasterMix: Enzyme (Tth)/Antibody (TP6-25), see Table 7.

TABLE 7

| Reagent | 2x |
|---|---|
| Enzyme Tth (5 U/μl) | 600.00 |
| Antibody: TP6-25 (1 mg/ml) | 600.00 |
| Water | 300.00 |
| Total | 1500.00 |

Aliquot 500 μl/tube and freeze at −20° C.

### CUP Master Mix:

1. 2.5× CUP Master Mix (Tables 8-11):

TABLE 8

| ml | 5x Additives | 2.5x Conc. |
|---|---|---|
| 0.50 | 1M Tris-C1 pH 8 | 5 mM |
| 1.25 | 40 mg/ml Albumin, bovine | 500 μg/ml |
| 37.50 | 1M stock Trehalose | 375 mM |
| 2.5 | 20% v Tween 20 | 0.50% |
| 7.00 | ddH₂O | |
| 48.75 | | |

Allow reagent to fully mix >15 minutes

TABLE 9

| ml | 5x Additives | 2.5x Conc |
|---|---|---|
| 12.50 | 1M Bicine/Potassium Hydroxide pH 8.2 | 125 mM |
| 5.75 | 5M Potassium Acetate | 287.5 mM |
| 20.00 | Glycerol (V × D = M -> 19.6 × 1.26 = 24.6 g) | 20% |
| 1.25 | 500 mM Magnesium Chloride | 6.25 mM |
| 1.75 | 500 mM Manganese Chloride | 8.75 mM |
| 5.00 | ddH₂O | |
| 46.25 | | |

Allow reagent to fully mix >15 minutes; Combine above mixes into sterile container—add the following from Table 10

TABLE 10

| ml | 5x Additives | 2.5x Conc. |
|---|---|---|
| 1.25 | 100 mM dATP | 1.25 mM |
| 1.25 | 100 mM dCTP | 1.25 mM |
| 1.25 | 100 mM dTTP | 1.25 mM |
| 1.25 | 100 mM dGTP | 1.25 mM |
| 100.00 | | |

Allow reagent to fully mix >15 minutes; Aliquot 1.8 ml/tube and freeze at −20° C.

TABLE 11

| Primer/Probe | Stock (μM) | FC (μM) | μl |
|---|---|---|---|
| Forward Primer | 100 | 10 | 100.0 |
| Reverse Primer | 100 | 10 | 100.0 |
| Probe | 100 | 4 | 40.0 |
| (5′FAM/3′BHQ1-TT) | | | |
| DI Water | | | 760.0 |
| Total | | | 1000.0 |

### Primer and Probe Mix:

Aliquot 250 μl/tube and freeze at −20° C.

### Reaction Mix:

1. CUP Master Mix (CMM): (Refer to Tables 12-14 and Plate A in Plate Setup, FIG. 7)

TABLE 12

| Reagent | FC | X1 (10 μl) | 450 |
|---|---|---|---|
| 2.5 x CUP Master Mix | 1X | 4.00 | 1800 |
| ROX | 1x | 0.20 | 90 |
| 2x TthAb Mix | 2U | 1.00 | 450 |
| Water | | 2.3 | 1035 |
| Total | | 7.50 | 3375 |

Preferably, each run/plate will have no more than 356 reactions: 12 samples with 12 Markers (288 reactions with 2 replicates for each)+10 std curve controls in duplicate (20)+2 positive and 2 negative controls for each Marker. (4×12=48)

Adjust water for sample volume—4.3 μl Sample MAX; Mix Well

TABLE 13

| Reagent | FC | X1 (10 μl) | 34 |
|---|---|---|---|
| Primers | 0.5 μM/0.2 μM | 0.50 | 17 |
| 10 μM/Probe 4 μM | | | |
| CMM | 1x | 7.50 | 255 |
| Total | | 8.00 | 272 |

2. ToO Markers: Mix Well

TABLE 14

| Reagent | FC | X1 (10 μl) | 44 |
|---|---|---|---|
| Primers | 0.5 μM/0.2 μM | 0.50 | 22 |
| 10 μM/Probe 4 μM | | | |
| CMM | 1x | 7.50 | 330 |
| Total | | 8.00 | 352 |

3. β-Actin and PBGD Markers: Mix Well

### Sample Setup:

TABLE 15

| | | | |
|---|---|---|---|
| Sample | Sample ID | Conc | Water Added = 50 ng/μl |

A1
A2
A3
A4
A5
A6
A7
A8
A9
A10
A11
A12

1. CUP Samples: 12 samples in 96 well plate: A1-A12 (Refer to Table 16 and Plate B in Plate Setup, FIG. 7); Aliquot 50 μl of 50 ng/μl (2 μl/rxn)

### Load Plate:

1. 384 Well Plate Setup: (Refer to Plate D in Plate Setup, FIG. 7)

2 μl of sample and 8 μl of CMM are loaded onto the plate (sample=50 ng/μl)

4 μl of sample and 6 μl of CMM are loaded on to the plate (sample=25 ng/μl)

The plate is sealed and labeled. Centrifuge at 2000 rpm for 1 min.

ABI 7900HT Setup: Place in the ABI 7900. Select the program “CUP 384” and hit start.

TABLE 16
Thermocycling conditions

95° C. × 60 s
55° C. × 2 m
RAMP 5%
70° C. × 2 m
40 cycles of
95° C. × 15 s
58° C. × 30 s

ROX Turned On

Data are analyzed, Ct's extracted and inserted in classification tree

### Example 3

### Classification Tree

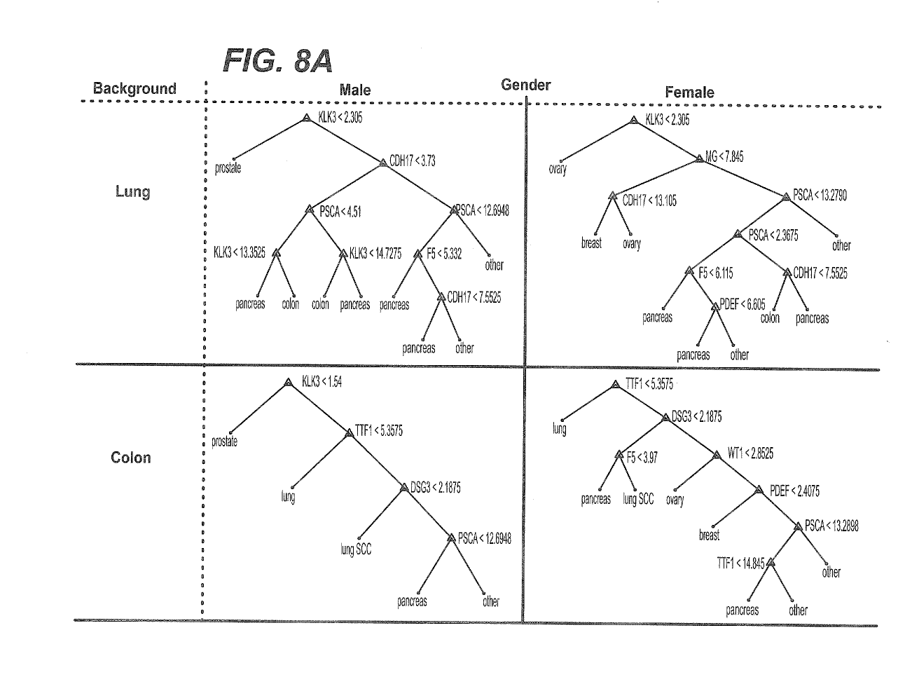

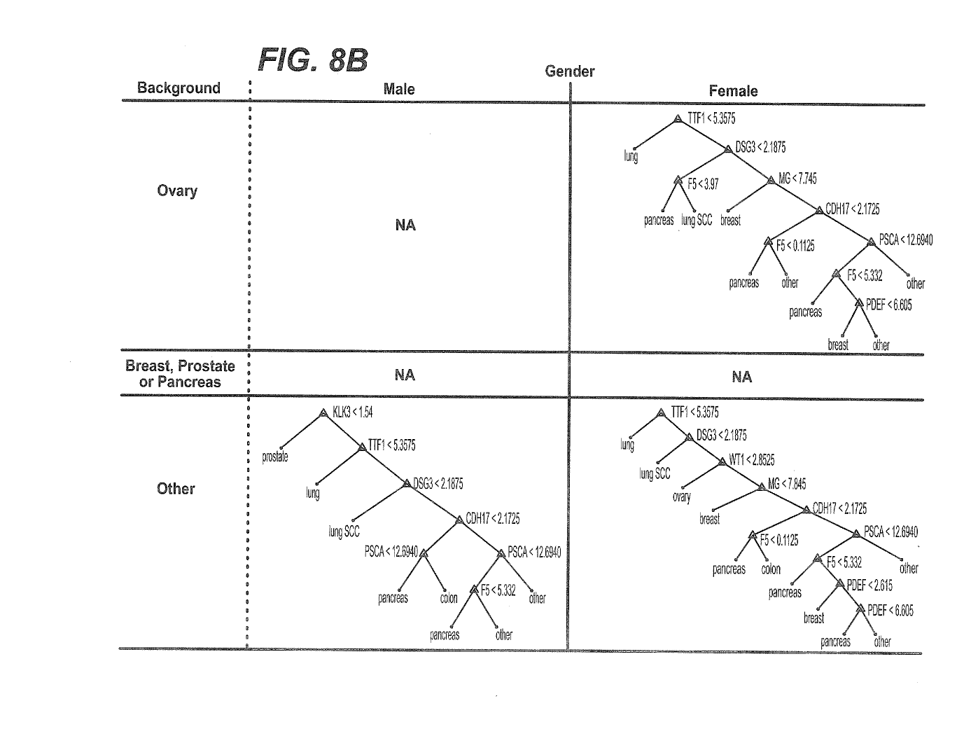

The classification tree is depicted in FIG. 8.

### Example 5

### CUP Assay Limits

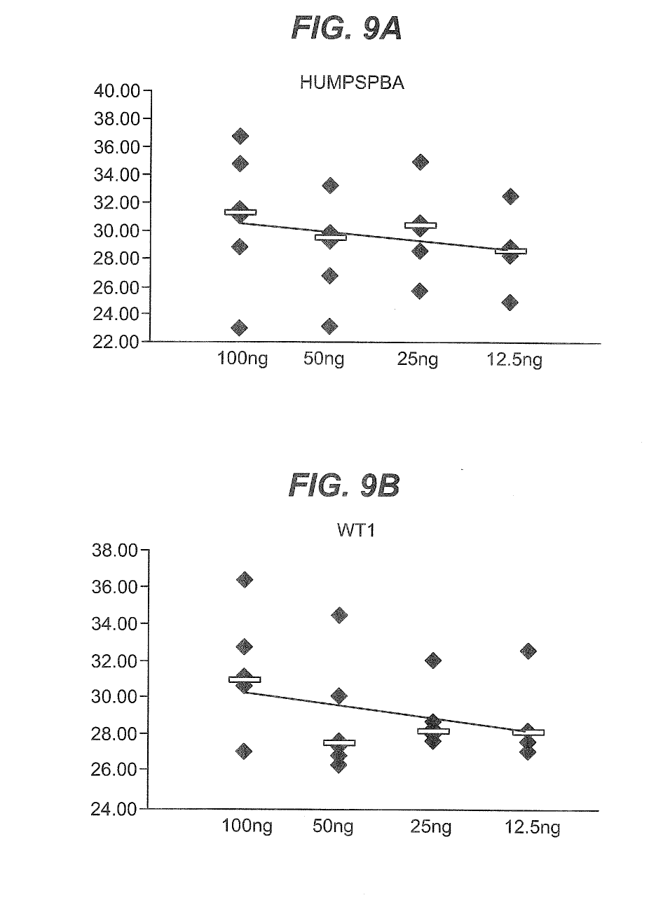

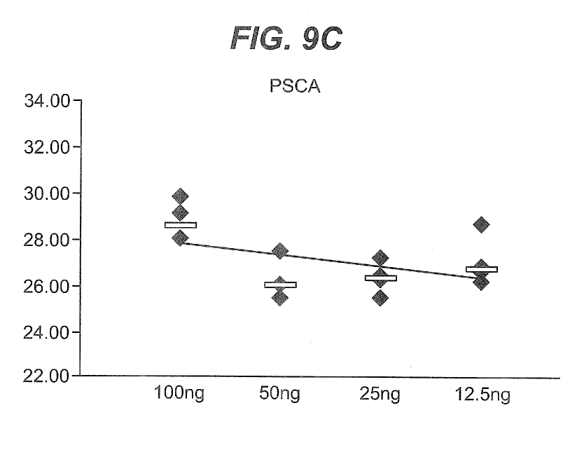

FIG. 9 depicts the results obtained, using the methods described in Examples 1-3, to determine the limits of the CUP assays. Assay performance was tested over a range of RNA concentrations and it was found that CUP assays are efficient in the range of from 100-12.5 ng RNA.

### Example 6

qRT-PCR Assay

### Materials and Methods.

### Frozen Tissue Samples for Microarray Analysis.

A total of 700 frozen primary human tissues were used for gene expression microarray profiling. Samples were obtained from variety of academic institutions, including Washington University (St. Louis, Mo.), Erasmus Medical Center (Rotterdam, Netherlands), and commercial tissue bank companies, including Genomics Collaborative, Inc. (Cambridge, Mass.), Asterand (Detroit, Mich.), Oncomatrix (La Jolla, Calif.) and Clinomics Biosciences (Pittsfield, Mass.). For each specimen, patient demographic, clinical and pathology information was collected as well. The histopathological features of each sample were reviewed to confirm diagnosis, and to estimate sample preservation and tumor content.

### RNA Extraction and Affymetrix GeneChip Hybridization.

Frozen cancer samples with greater than 70% tumor cells, benign and normal samples were dissected and homogenized with mechanical homogenizer (UltraTurrex T8, Germany) in Trizol reagent (Invitrogen, Carlsbad, Calif.). Tissue was homogenized in Trizol reagent by following the standard Trizol protocol for RNA isolation from frozen tissues (Invitrogen, Carlsbad, Calif.). After centrifugation the top liquid phase was collected and total RNA was precipitated with isopropyl alcohol at −20° C. RNA pellets were washed with 75% ethanol, resolved in water and stored at −80° C. until use.

RNA quality was examined with an Agilent 2100 Bioanalyzer RNA 6000 Nano Assay (Agilent Technologies, Palo Alto, Calif.). Labeled cRNA was prepared and hybridized with the high-density oligonucleotide array Hu133A Gene Chip (Affymetrix, Santa Clara, Calif.) containing a total of 22,000 probe sets according to standard manufacturer protocol. Arrays were scanned using Affymetrix protocols and scanners. For subsequent analysis, each probe set was considered a separate gene. Expression values for each gene were calculated using Affymetrix Gene Chip analysis software MAS 5.0. All chips met three quality control standards: the percent “present” call for the array was greater than 35%, the scale factor was less than 12 when scaled to a global target intensity of 600, and the average background level was less than 150.

### Marker Candidate Selection.

For selection of tissue of origin (ToO) Marker candidates for lung, colon, breast, ovarian, and prostate tissues, expression levels of the probe sets were measured in the RNA samples covering a total of 682 normal, benign, and cancerous tissues from breast, colon, lung, ovarian, prostate. Tissue specific Marker candidates were selected based on number of statistical queries.

In order to generate pancreatic candidates, gene expression profiles of 13 primary pancreas ductal adenocarcinoma, 5 pancreas normal and 98 lung, colon, breast and ovarian cancer specimens was used to select pancreas adenocarcinoma Markers. Two queries were performed. In the first query, data set containing 14547 genes with at least 2 “present” calls in pancreas samples was created. A total of 2736 genes that overexpressed in pancreas cancer compare to normal was identified by T-test (p<0.05) were identified. Genes which minimal expression at 11th percentile of pancreas cancer was at least 2 fold higher that the maximum in colon and lung cancer was selected, making 45 probe sets. As a final step, 6 genes with maximum expression at least 2 fold higher than maximum expression in colon, lung, breast, and ovarian cancers were selected. In a second query, data set of 4654 probe sets with at most 2 “present” calls in all breast, colon, lung and ovarian specimens was created. A total of 160 genes that have at least 2 “present” calls in pancreas normal and cancer samples were selected. Out of 160 genes, 10 genes were selected after comparing their expression level between pancreas and normal tissues. Results of both pancreas queries were combined.

In addition to gene expression profiles analysis, a few Markers were selected from literature. Results of all queries were combined to make a short list of ToO Marker candidates for each tissue type. Sensitivity and specificity of each Marker were estimated. Markers that demonstrated the best ability to differentiate tissues by their origin were nominated for RT-PCR testing based on Markers redundancy and complementarity.

FFPE Metastatic Carcinoma of known Origin and CUP Tissues.

A total of 386 FFPE metastatic carcinomas (Stage III-IV) of known origin and 24 FFPE prostate primary adenocarcinomas were acquired from a variety of commercial vendors, including Proteogenex (Los Angeles, Calif.), Genomics Collaborative, Inc. (Cambridge, Mass.), Asterand (Detroit, Mich.), Ardais (Lexington, Mass.) and Oncomatrix (La Jolla, Calif.). An independent set of 48 metastatic carcinoma of known primary and CUP tissues was obtained from Albany Medical College (Albany, N.Y.). For each specimen, patient demographic, clinical and pathology information was collected as well. The histopathological features of each sample were reviewed to confirm diagnosis, and to estimate sample preservation and tumor content. For metastatic samples, diagnoses of metastatic carcinoma and tissue of origin were unequivocally established based on patient's clinical history and histological evaluation of metastatic carcinoma in comparison to corresponding primaries.

RNA Isolation from FFPE Samples.

RNA isolation from paraffin tissue sections was as described in the High Pure RNA Paraffin Kit manual (Roche) with the following modifications. Paraffin embedded tissue samples were sectioned according to size of the embedded metastasis (2-5 mm=9×10 μm, 6-8 mm=6×10 μm, 8-≧10 mm=3×10 μm). Sections were deparaffinized as described by Kit manual, the tissue pellet was dried in a 55° C. oven for 5-10 minutes and resuspended in 100 μl of tissue lysis buffer, 16 μl 10% SDS and 80 μl Proteinase K. Samples were vortexed and incubated in a thermomixer set at 400 rpm for 2 hours at 55° C. Subsequent sample processing was performed according High Pure RNA Paraffin Kit manual. Samples were quantified by OD 260/280 readings obtained by a spectrophotometer and samples were diluted to 50 ng/μl. The isolated RNA was stored in RNase-free water at −80° C. until use.

qRT-PCR for Marker Candidates Pre-Screening.

One μg total RNA from each sample was reverse-transcribed with random hexamers using Superscript II reverse transcriptase according to the manufacturer's instructions (Invitrogen, Carlsbad, Calif.). Primers and MGB-probes for the tested gene Marker candidates and the control gene ACTB were designed using Primer Express software (Applied Biosystems, Foster City, Calif.) either ABI Assay-on-Demand (Applied Biosystems, Foster City, Calif.) were used. All in-house designed primers and probes were tested for optimal amplification efficiency above 90%. RT-PCR amplification was carried out in a 20 ml reaction mix containing 200 ng template cDNA, 2×TaqMan® universal PCR master mix (10 ml) (Applied Biosystems, Foster City, Calif.), 500 nM forward and reverse primers, and 250 nM probe. Reactions were run on an ABI PRISM 7900HT Sequence Detection System (Applied Biosystems, Foster City, Calif.). The cycling conditions were: 2 min of AmpErase UNG activation at 50° C., 10 min of polymerase activation at 95° C. and 50 cycles at 95° C. for 15 sec and annealing temperature (60° C.) for 60 sec. In each assay, “no-template” control along with template cDNA was included in duplicate for both the gene of interest and the control gene. The relative expression of each target gene was represented as ΔCt, which is equal to Ct of the target gene subtracted by Ct of the control gene (ACTB).

Optimized One-Step qRT-PCR.

Appropriate mRNA reference sequence accession numbers in conjunction with Oligo 6.0 were used to develop TaqMan® CUP assays (lung Markers: human surfactant, pulmonary-associated protein B (SP-B), thyroid transcription factor 1 (TTF1), desmoglein 3 (DSG3), colorectal Marker: cadherin 17 (CDH17), breast Markers: mammaglobin (MG), prostate-derived ets transcription factor (PDEF), ovarian Marker: wilms tumor 1 (WT1), pancreas Markers: prostate stem cell antigen (PSCA), coagulation factor V (F5), prostate Marker kallikrein 3 (KLK3)) and housekeeping assays beta actin (β-Actin), hydroxymethylbilane synthase (PBGD). Gene specific primers and hydrolysis probes for the optimized one-step qRT-PCR assay are listed in Table 2 (SEQ ID NOs: 11-58). Genomic DNA amplification was excluded by designing the assays around exon-intron splicing sites. Hydrolysis probes were labeled at the 5′ nucleotide with FAM as the reporter dye and at 3′ nucleotide with BHQ 1 -TT as the internal quenching dye.

Quantitation of gene-specific RNA was carried out in a 384 well plate on the ABI Prism 7900HT sequence detection system (Applied Biosystems). For each thermo-cycler run calibrators and standard curves were amplified. Calibrators for each Marker consisted of target gene in vitro transcripts that were diluted in carrier RNA from rat kidney at 1×10⁵ copies. Standard curves for housekeeping Markers consisted of target gene in vitro transcripts that were serially diluted in carrier RNA from rat kidney at 1×10⁷, 1×10⁵ and 1×10³ copies. No target controls were also included in each assay run to ensure a lack of environmental contamination. All samples and controls were run in duplicate. qRT-PCR was performed with general laboratory use reagents in a 10 μl reaction containing: RT-PCR Buffer (50 nM Bicine/KOH pH 8.2, 115 nM KAc, 8% glycerol, 2.5 mM MgCl₂, 3.5 mM MnSO₄, 0.5 mM each of dCTP, DATP, dGTP and dTTP), Additives (2 mM Tris-Cl pH 8, 0.2 mM Albumin Bovine, 150 mM Trehalose, 0.002% Tween 20), Enzyme Mix (2 U Tth (Roche), 0.4 mg/μl Ab TP6-25), Primer and Probe Mix (0.2 μM Probe, 0.5 μM Primers). The following cycling parameters were followed: 1 cycle at 95° C. for 1 minute; 1 cycle at 55° C. for 2 minutes; Ramp 5%; 1 cycle at 70° C. for 2 minutes; and 40 cycles of 95° C. for 15 seconds, 58° C. for 30 seconds. After the PCR reaction was completed, baseline and threshold values were set in the ABI 7900HT Prism software and calculated Ct values were exported to Microsoft Excel.

One-Step vs. Two-Step Reaction.

For comparison of two-step with one-step RT-PCR reactions, first strand synthesis of two-step reaction was carried out using either 100 ng of random hexamers or gene specific primers per reaction. In the first step, 11.5 μl of Mix-1 (primers and 1 μg of total RNA) was heated to 65° C. for 5 minutes and then chilled on ice. 8.5 ∞l of Mix-2 (1× Buffer, 0.01 mM DTT, 0.5 mM each dNTP's, 0.25 U/μl RNasin®, 10 U/μl Superscript III) was added to Mix-1 and incubated at 50° C. for 60 minutes followed by 95° C. for 5 minutes. The cDNA was stored at −20° C. until ready for use. qRT-PCR for the second step of the two-step reaction was performed as stated above with the following cycling parameters: 1 cycle at 95° C. for 1 minute; 40 cycles of 95° C. for 15 seconds, 58° C. for 30 seconds. qRT-PCR for the one-step reaction was performed exactly as stated in the preceding paragraph. Both the one-step and two-step reactions were performed on 100 ng of template (RNA/cDNA). After the PCR reaction was completed baseline and threshold values were set in the ABI 7900HT Prism software and calculated Ct values were exported to Microsoft Excel.

### Results.

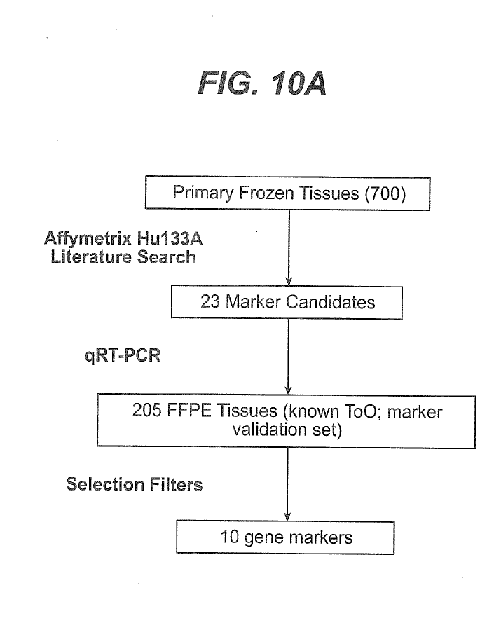

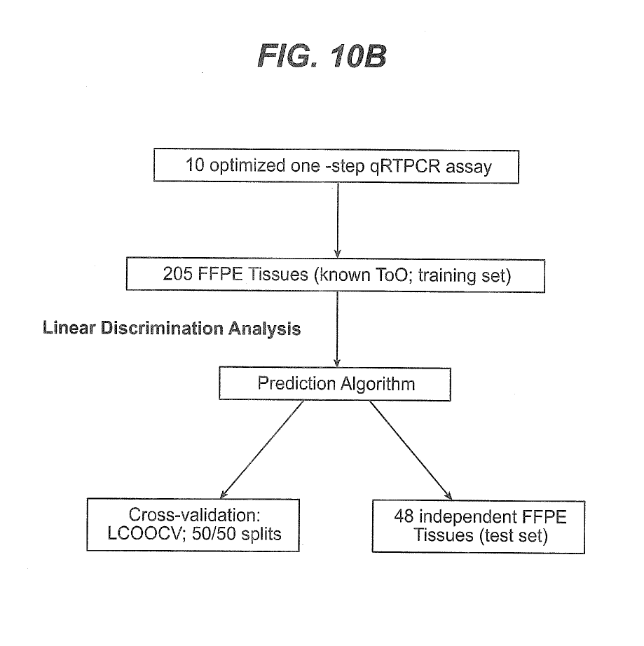

The goal of this study was to develop a qRT-PCR assay to predict metastatic carcinoma tissue of origin. The experimental work consisted of two major parts. The first part included tissue-specific Marker candidates nomination, their validation on FFPE metastatic carcinoma tissues, and selection of ten Markers for the assay (FIG. 10A.). The second part included qRT-PCR assay optimization followed by assay implementation on another set of FFPE metastatic carcinomas, building of a classification tree and validation on an independent sample set. (FIG. 10B).

### Sample Characteristics.

RNA from a total of 700 frozen primary tissue samples was used for the gene expression profiling and tissue type specific gene identification. Samples included 545 primary carcinomas (29 lung, 13 pancreas, 315 breast, 128 colorectal, 38 prostate, 22 ovarian), 37 benign lesions (1 lung, 4 colorectal, 6 breast, 26 prostate) and 118 (36 lung, 5 pancreas, 36 colorectal, 14 breast, 3 prostate, 24 ovarian) normal tissues.

A total of 375 metastatic carcinomas of known origin (Stage III-IV) and 26 prostate primary adenocarcinoma samples were used in the study. The metastatic carcinomas originated from lung, pancreas, colorectal, ovarian, prostate as well as other cancers. The “other” sample category consisted of metastasis derived from tissues other than lung, pancreas, colon, breast, ovary and prostate. Patients' characteristics are summarized in Table 17.

TABLE 17

| | | Metastatic | |
|---|---|---|---|
| | | CUP | Sample Set |
| Total Number | | 401 | 48 |
| Average Age | | 57.8 ± 11* | 62.13 ± 11.7 |
| Gender | Female | 241 | 20 |
| | Male | 160 | 28 |

| | | |
|---|---|---|
| Tissue of Origin | | |
| Lung | 65 | 9 |
| Pancreas | 63 | 2 |
| Colorectal | 61 | 4 |
| Breast | 63 | 5 |
| Ovarian | 82 | 2 |
| Prostate | 27 | 2 |
| Kidney | 8 | 8 |
| Stomach | 7 | 0 |
| Other** | 25 | 5 |
| Carcinoma of Unknown Primary | | 11 |
| Histopathological Diagnosis | | |
| Adenocarcinoma, moderately/well | 306 | 27 |
| differentiated | | |
| Adenocarcinoma, poorly differentiated | 49 | 4 |
| Squamous cell carcinoma | 16 | 5 |
| Poorly differentiated carcinoma | 16 | 10 |
| Small cell carcinoma | 3 | |
| Melanoma | 5 | |
| Lymphoma | 3 | |
| Hepatocellular carcinoma | 2 | |
| Mesothelioma | 1 | |
| Other*** | 14 | 2 |
| Metastatic Site | | |
| Lymph Nodes | 73 | 1 |
| Brain | 17 | 14 |
| Lung | 20 | 7 |
| Liver | 75 | 11 |
| Pelvic region (ovary, bladder, fallopian | 53 | 2 |
| tubes) | | |
| Abdomen (Omentum (omentum, mesentery, | 91 | 5 |
| colon, peritoneum) | | |
| Other (skin, thyroid, chest wall, umbilicus) | 44 | 8 |
| Unknown | 2 | |
| Primary (prostate) | 26 | |

*Age is unknown for 26 patients
**esophagus, bladder, pleura, liver gallbladder, bile ducts, larynx, pharynx, Non-Hodgkin lymphoma
***small cell, mesothelioma, hepatocellular, melanoma, lymphoma

Samples were separated into two sets: the validation set (205 specimens) that was used to validate Marker candidates' tissue-specific differential expression and the training set (260 specimens) that was used for testing of the optimized one-step qRT-PCR procedure and training of a classification tree. The first set of 205 samples included 25 lung, 41 pancreas, 31 colorectal, 33 breast, 33 ovarian, 1 prostate, 23 other cancer metastasis and 18 prostate primary cancers. The second set consisted of 260 samples included 56 lung, 43 pancreas, 30 colorectal, 30 breast, 49 ovarian, 32 other cancer metastasis and 20 primary prostate cancers. Sixty-four specimens, including 16 lung, 21 pancreas, 15 other metastatic, and 12 prostate primary carcinomas were from the same patient in both sets.

The independent sample set obtained from Albany Medical College was comprised of 33 CUP specimens with a primary suggested for 22 of them, and 15 metastatic carcinomas of known origin. For CUPs having a suggested primary, a diagnosis was rendered based on morphological features, and/or results of testing with a panel of IHC Markers. Patient demographic, clinical and pathology characteristics are presented in Table 17.

### Marker Candidate Selection.

Analysis of gene expression profiles of 5 primary tissues types (lung, colon, breast, ovary, prostate) resulted in nomination of 13 tissue specific Marker candidates for qRT-PCR testing. Top candidates have been identified in previous studies of cancers in situ. Argani et al. (2001); Backus et al. (2005); Cunha et al. (2005); Borgono et al. (2004); McCarthy et al. (2003); Hwang et al. (2004); Fleming et al. (2000); Nakamura et al. (2002); and Khoor et al. (1997). In addition to the analysis of the microarray data, two Markers were selected from the literature, including a complementary lung squamous cell carcinoma Marker DSG3 and the breast Marker PDEF. Backus et al. (2005). The microarray data confirmed the high sensitivity and specificity of these Markers.

A special approach was used to identify pancreas specific Markers. First, five pancreas Marker candidates were analyzed: prostate stem cell antigen (PSCA), serine proteinase inhibitor, dade A member 1 (SERPINA1), cytokeratin 7 (KRT7), matrix metalloprotease 11 (MMP11), and mucin 4 (MUC4) (Varadhachary et al. (2004); Argani et al. (2001); Jones et al. (2004); Prasad et al. (2005); and Moniaux et al. (2004)) using DNA microarrays and a panel of 13 pancreatic ductal adenocarcinomas, five normal pancreas tissues, and 98 samples from breast, colorectal, lung, and ovarian tumors. Only PSCA demonstrated moderate sensitivity (six out of thirteen or 46% of pancreatic tumors were detected) at a high specificity (91 out of 98 or 93% were correctly identified as not being of pancreatic origin). In contrast, KRT7, SERPINA1, MMP11, and MUC4 demonstrated sensitivities of 38%, 31%, 85%, and 31%, respectively, at specificities of 66%, 91%, 82%, and 81%, respectively. These data were in good agreement with qRT-PCR performed on 27 metastases of pancreatic origin and 39 metastases of non-pancreatic origin for all Markers except for MMP11 which showed poorer sensitivity and specificity with qRT-PCR and the metastases. In conclusion, the microarray data on snap frozen, primary tissue serves as a good indicator of the ability of the Marker to identify a FFPE metastasis as being pancreatic in origin using qRT-PCR but that additional Markers may be useful for optimal performance.

Pancreatic ductal adenocarcinoma develops from ductal epithelial cells that comprise only a small percentage of all pancreatic cells (with acinar and islet cells comprising the majority) in the normal pancreas. Furthermore, pancreatic adenocarcinoma tissues contain a significant amount of adjacent normal tissue. Prasad et al. (2005); and Ishikawa et al. (2005). Because of this the candidate pancreas Markers were enriched for genes elevated in pancreas adenocarcinoma relative to normal pancreas cells. The first query method returned six probe sets: coagulation factor V (F5), a hypothetical protein FLJ22041 similar to FK506 binding proteins (FKBP10), beta 6 integrin (ITGB6), transglutaminase 2 (TGM2), heterogeneous nuclear ribonucleoprotein A0 (HNRPA0), and BAX delta (BAX). The second query method (see Materials and Methods section for details) returned eight probe sets: F5, TGM2, paired-like homeodomain transcription factor 1 (PITX1), trio isoform mRNA (TRIO), mRNA for p73H (p73), an unknown protein for MGC: 10264 (SCD), and two probe sets for claudin18.

A total of 23 tissue specific Marker candidates were selected for further RT-PCR validation on metastatic carcinoma FFPE tissues by qRT-PCR. Marker candidates were tested on 205 FFPE metastatic carcinomas, from lung, pancreas, colon, breast, ovary, prostate and prostate primary carcinomas. Table 18 provides the gene symbols of the tissue specific Markers selected for RT-PCR validation and also summarizes the results of testing performed with these Markers.

TABLE 18

| | | | | Marker selection filters | | | |
|---|---|---|---|---|---|---|---|
| | SEQ | ID method | | Low exp | | | |
| Tissue | ID | Micro | | corres met | Marker | Tissue cross | Marker |
| type | NOs | array | Lit | tissue | redundancy | reactivity | adequate? |
| Lung | 1/59 | X | X | | | | X |
| | 60 | X | X | | | | X |
| | 61 | | X | | X | | X |
| Pancreas | 66 | | X | | | | X |
| | 67 | X | | | | | X |
| | 71 | X | | | X | | |
| | 72 | X | | X | | | |
| | 73 | | X | | | | |
| | 74 | | X | | | | |
| | 75 | | X | | | | |
| | 76 | | X | | | | |
| Colon | 4/85 | X | X | | | | X |
| | 77 | X | X | | | | |
| | 78 | X | X | | X | | |
| | 79 | X | X | | X | | |
| Prostate | 9/86 | X | X | | | | X |
| | 80 | X | X | | X | | |
| Breast | 63 | X | X | | | | X |
| | 81 | X | X | | | X | |
| | 64 | | X | | | | X |
| Ovarian | 82 | X | X | | | X | |
| | 83 | X | X | | | X | |
| | 65 | X | X | | | | X |

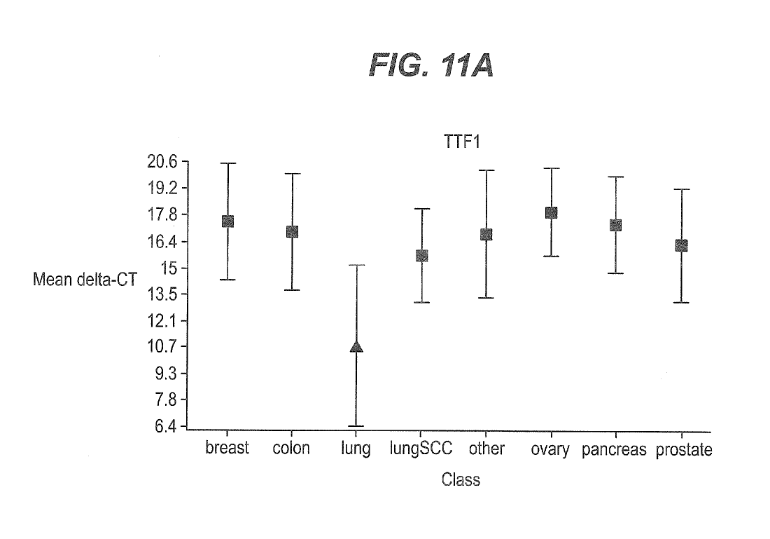

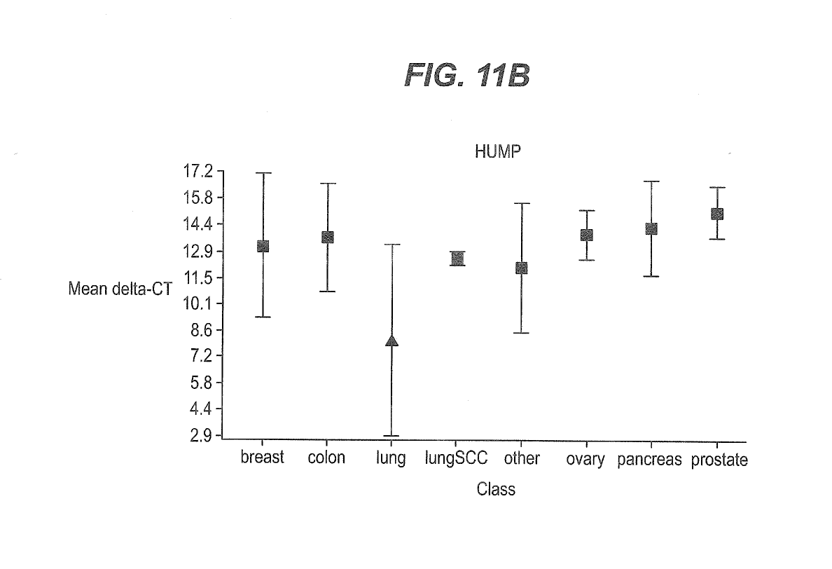

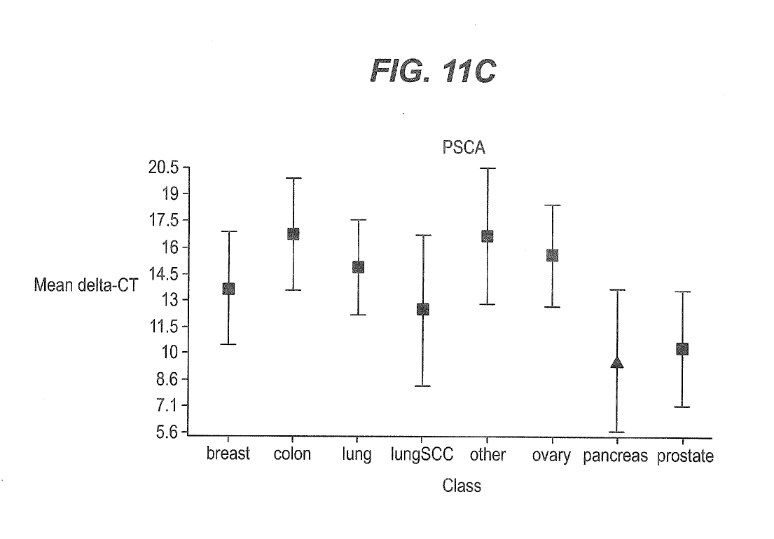

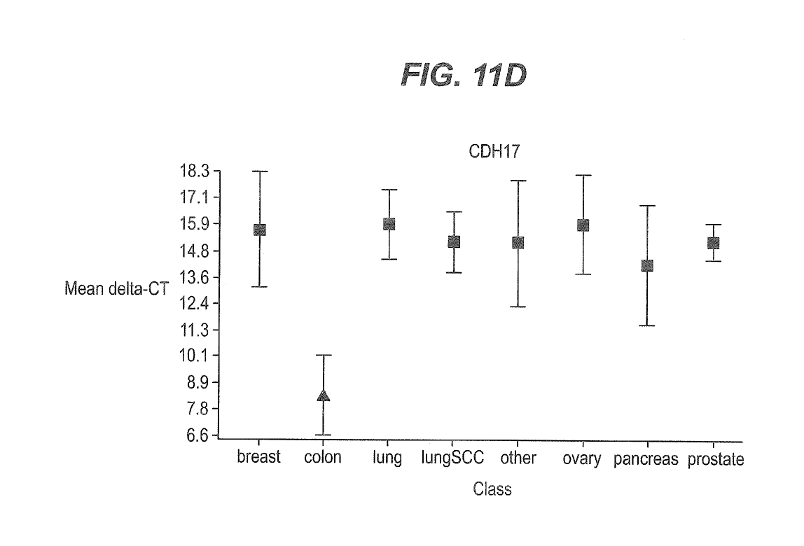

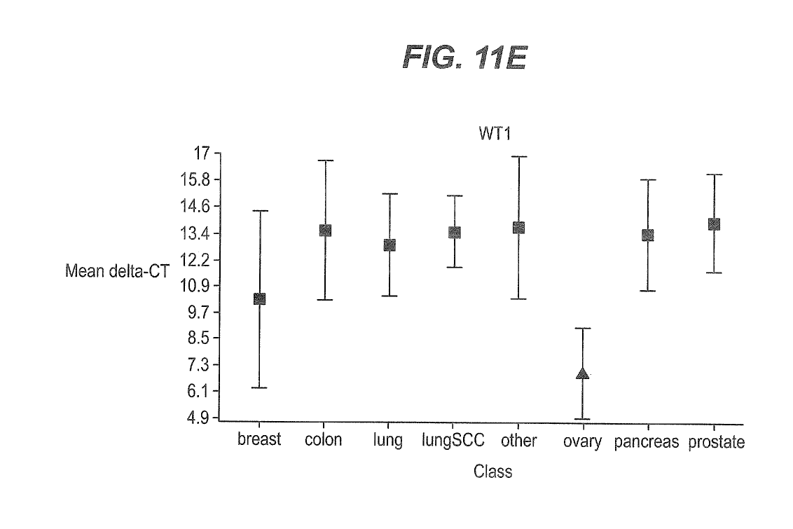

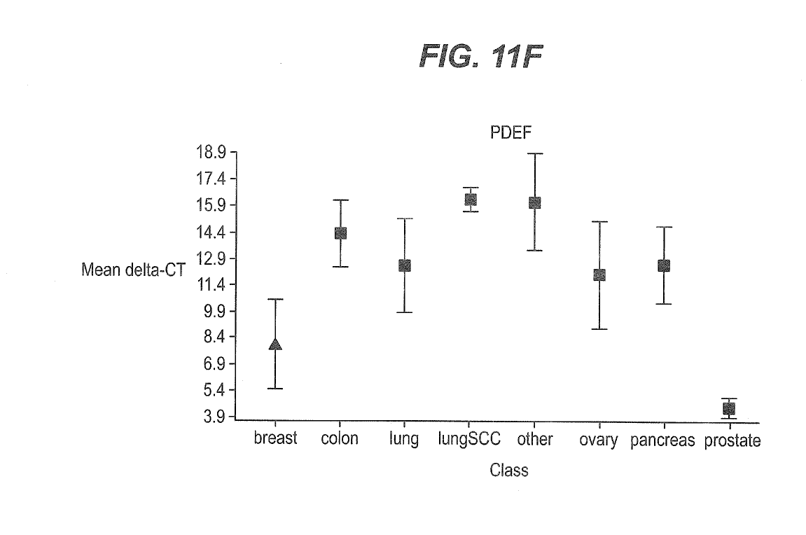

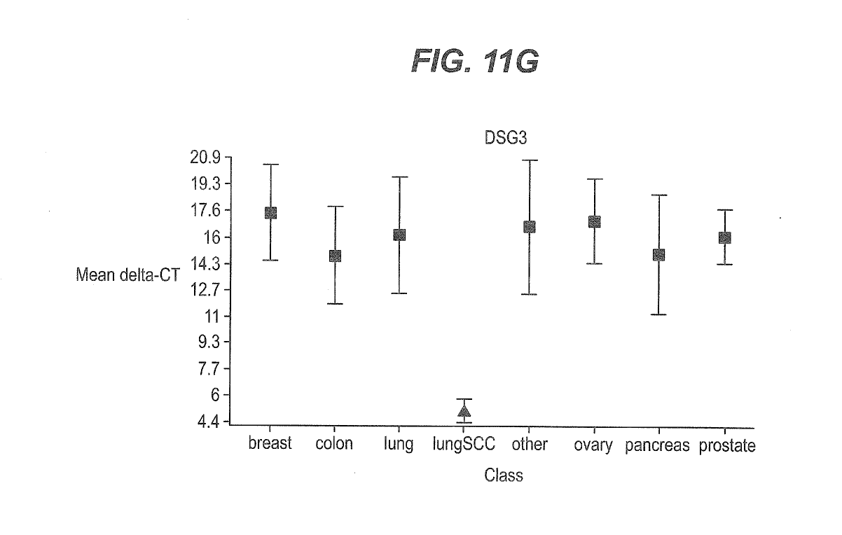

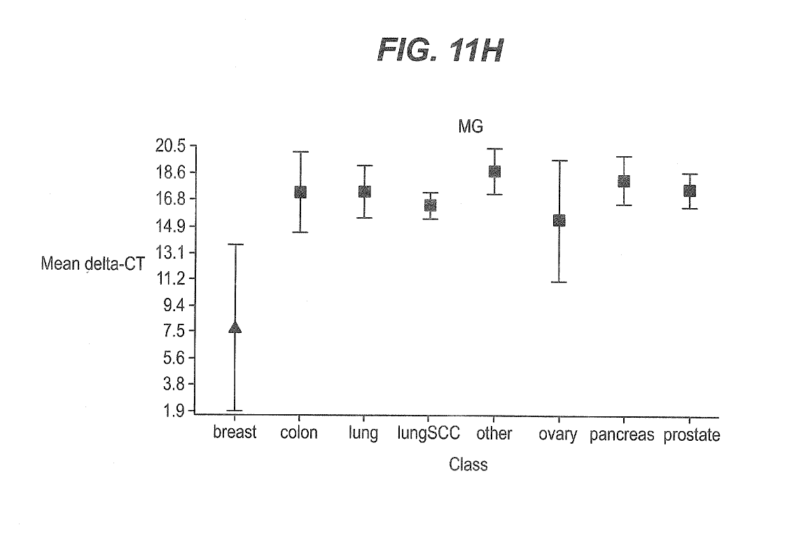

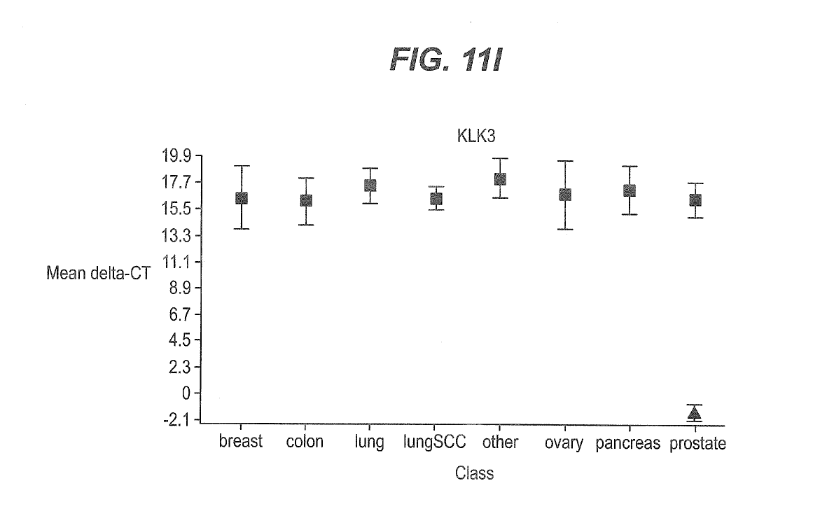

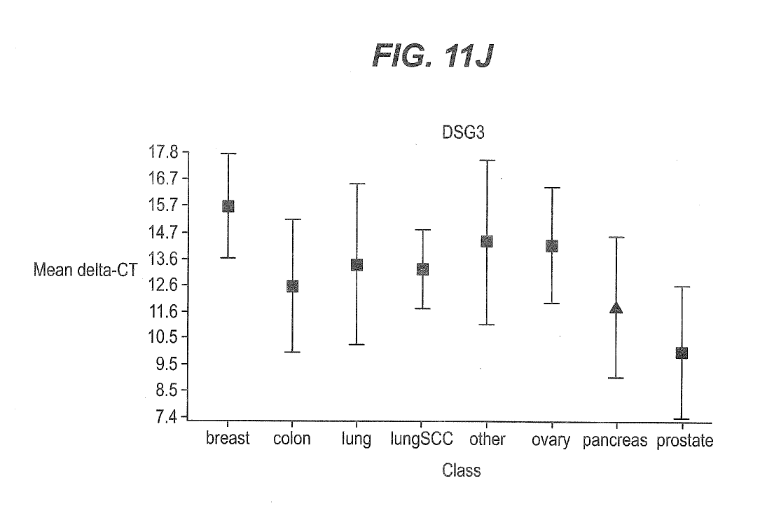

Out of 23 tested Markers, thirteen were rejected based on their cross reactivity, low expression level in the corresponding metastatic tissues, or redundancy. Ten Markers were selected for the final version of assay. The lung Markers were human surfactant pulmonary-associated protein B (HUMPSPB), thyroid transcription factor 1 (TTF1), and desmoglein 3 (DSG3). The pancreas Markers were prostate stem cell antigen (PSCA) and coagulation factor V (F5), and the prostate Marker was kallikrein 3 (KLK3). The colorectal Marker was cadherin 17 (CDH17). Breast Markers were mammaglobin (MG) and prostate-derived Ets transcription factor (PDEF). The ovarian Marker was Wilms tumor 1 (WT1). Mean normalized relative expression values of selected Markers in different metastatic tissues are presented on FIG. 11.

Optimization of Sample Preparation and qR T-PCR using FFPE Tissues.

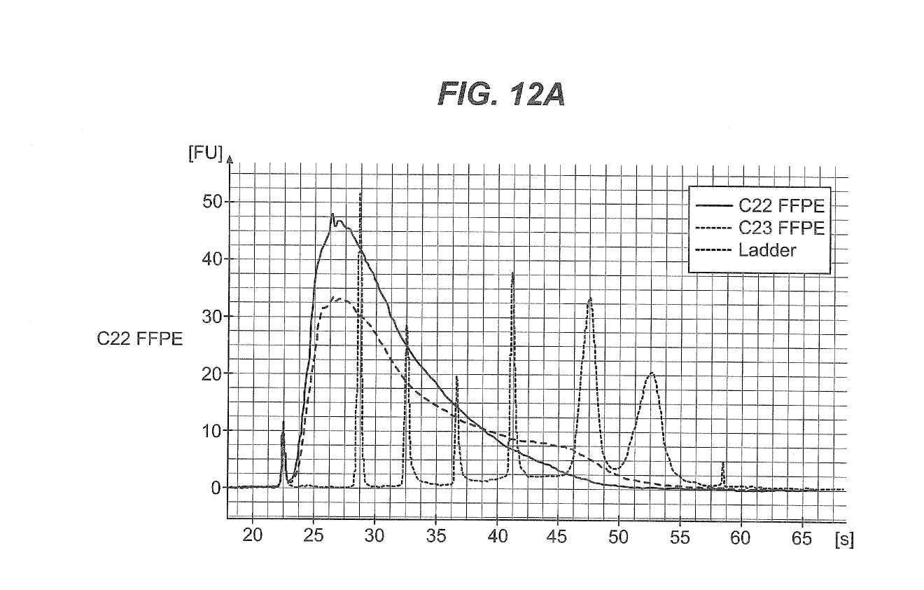

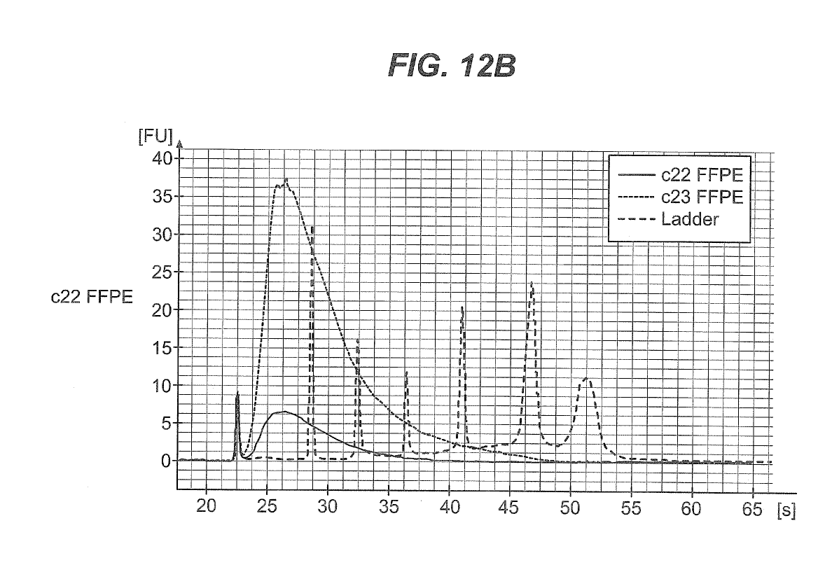

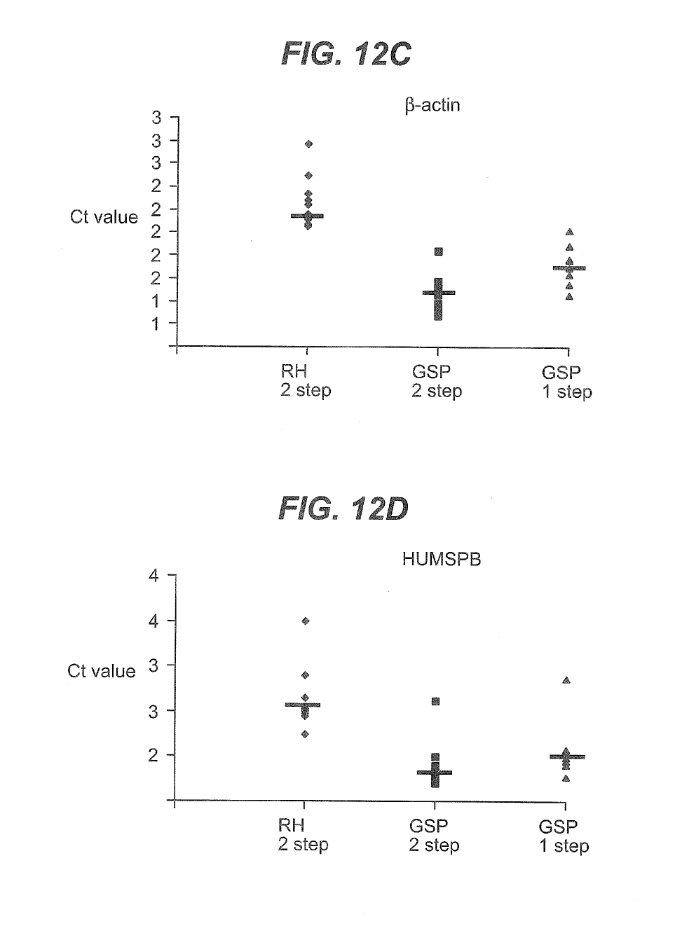

Next the RNA isolation and qRT-PCR methods were optimized using fixed tissues before examining the performance of the Marker panel. First the effect of reducing the proteinase K incubation time from sixteen hours to 3 hours was analyzed. There was no effect on yield. However, some samples showed longer fragments of RNA when the shorter proteinase K step was used (FIGS. 12A, B). For example, when RNA was isolated from a one-year-old block (C22), no difference was observed in the electropherograms. However, when RNA was isolated from a five-year-old block (C23), a larger fraction of higher molecular weight RNAs were observed, as assessed by the hump in the shoulder, when the shorter proteinase K digest was used. This trend generally held when other samples were processed, regardless of the organ of origin for the FFPE metastasis. In conclusion, shortening the proteinase K digestion time does not sacrifice RNA yields and may aid in isolating longer, less degraded RNA.

Next three different methods of reverse transcription were compared: reverse transcription with random hexamers followed by qPCR (two step), reverse transcription with a gene-specific primer followed by qPCR (two step), and a one-step qRT-PCR using gene-specific primers. RNA was isolated from eleven metastases and compared Ct values across the three methods for β-actin, HUMSPB (FIGS. 12C, D) and TTF. The results showed statistically significant differences (p<0.001) for all comparisons. For both genes, the reverse transcription with random hexamers followed by qPCR (two step reaction) gave the highest Ct values while the reverse transcription with a gene-specific primer followed by qPCR (two-step reaction) gave slightly (but statistically significant) lower Ct values than the corresponding 1 step reaction. However, the two-step RT-PCR with gene-specific primers had a longer reverse transcription step. When HUMSPB Ct values were normalized to the corresponding β-actin value for each sample, there were no differences in the normalized Ct values across the three methods. In conclusion, optimization of the RT-PCR reaction conditions can generate lower Ct values, which aids in analyzing older paraffin blocks (Cronin et al. (2004)), and a one step RT-PCR reaction with gene-specific primers can generate Ct values comparable to those generated in the corresponding two step reaction.

Diagnostic Performance of Optimized qRT-PCR Assay.

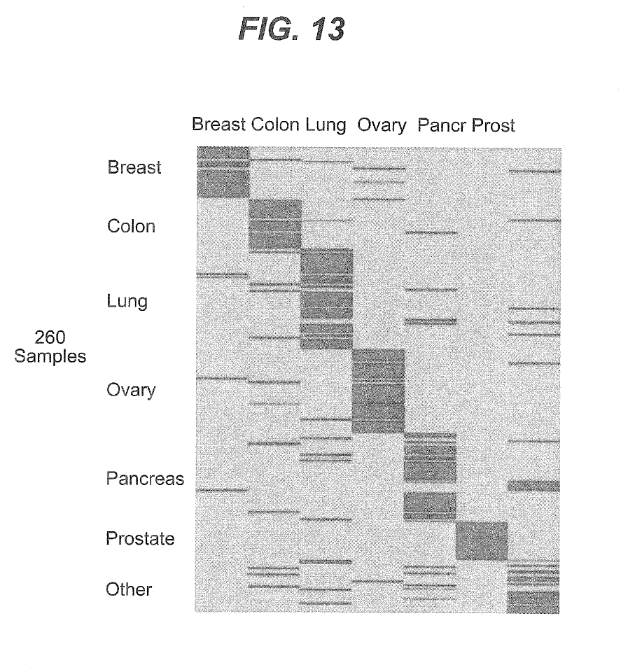

Twelve qRT-PCR reactions (10 Markers and 2 housekeeping genes) were performed on new set of 260 FFPE metastases. Twenty-one samples gave high Ct values for the housekeeping genes so only 239 were used in a heat map analysis. Analysis of the normalized Ct values in a heat map revealed the high specificity of the breast and prostate Markers, moderate specificity of the colon, lung, and ovarian, and somewhat lower specificity of the pancreas Markers (FIG. 13). Combining the normalized qRT-PCR data with computational refinement improves performance of the Marker panel.

Using expression values, normalized to average of expression of two housekeeping genes, a linear discrimination analysis to predict metastasis tissue of origin was developed by combining the normalized qRT-PCR data with the classification tree and determined the accuracy of the qRT-PCR assay to be 74%.

| | | | Lung | Ovar- | | | |
|---|---|---|---|---|---|---|---|
| | Breast | Colon | (SSC) | ian | Pancreas | Prostate | Other |
| Correct | 4 | 2 | 9 | 1 | 0 | 2 | 11 |
| Total | 5 | 5 | 12 | 2 | 1 | 2 | 12 |

### Discussion.

In this study, microarray-based expression profiling on primary tumors was used to identify candidate Markers for use with metastases. The fact that primary tumors can be used to discover tumor of origin Markers for metastases is consistent with several recent findings. For example, Weigelt and colleagues have shown that gene expression profiles of primary breast tumors are maintained in distant metastases. Weigelt et al. (2003). Backus and colleagues identified putative Markers for detecting breast cancer metastasis using a genome-wide gene expression analysis of breast and other tissues and demonstrated that mammaglobin and CK19 detected clinically actionable metastasis in breast sentinel lymph nodes with 90% sensitivity and 94% specificity. Backus et al. (2005).

During the development of the assay, selection was focused on six cancer types, including lung, pancreas and colon which are among the most prevalent in CUP (Ghosh et al. (2005); and Pavlidis et al. (2005)) and breast, ovarian and prostate for which treatment could be potentially most beneficial for patients. Ghosh et al. (2005). However, additional tissue types and Markers can be added to the panel as long as the overall accuracy of the assay is not compromised and, if applicable, the logistics of the RT-PCR reactions are not encumbered.

The microarray-based studies with primary tissue confirmed the specificity and sensitivity of known Markers. As a result, the majority of tissue specific Markers have high specificity for the tissues studied here. A recent study found that, using IHC, PSCA is overexpressed in prostate cancer metastases. Lam et al. (2005). Dennis et al. (2002) also demonstrated that PSCA could be used as a tumor of origin Marker for pancreas and prostate. Strong expression of PSCA in some prostate tissues at the RNA level was present but, because due to inclusion of PSA in the assay, prostate and pancreatic cancers can now be segregated. A novel finding of this study was the use of F5 as a complementary (to PSCA) Marker for pancreatic tissue of origin. In both the microarray data set with primary tissue and the qRT-PCR data set with FFPE metastases, F5 was found to complement PSCA.

Previous investigators have generated CUP assays using IHC (Brown et al. (1997); DeYoung et al. (2000); and Dennis et al. (2005a)) or microarrays. Su et al. (2001); Ramaswamy et al. (2001); and Bloom et al. (2004). More recently, SAGE has been coupled to a small qRT-PCR Marker panel. Dennis et al. (2002); and Buckhaults et al. (2003). This study is the first to combine microarray-based expression profiling with a small panel of qRT-PCR assays. The microarray studies with primary tissue identified some, but not all, of the same tissue of origin Markers as those identified previously by SAGE studies. This finding is not surprising given studies that have demonstrated that a modest agreement between SAGE- and DNA microarray-based profiling data exists and that the correlation improves for genes with higher expression levels. van Ruissen et al. (2005); and Kim et al. (2003). For example, Dennis and colleagues identified PSA, MG, PSCA, and HUMSPB while Buckhaults and coworkers (Buckhaults et al. (2003)) identified PDEF. Execution of the CUP assay is preferably by qRT-PCR because it is a robust technology and may have performance advantages over IHC. Al-Mulla et al. (2005); and Haas et al. (2005). Further, as shown herein, the qRT-PCR protocol has been improved through the use of gene-specific primers in a one-step reaction. This is the first demonstration of the use of gene-specific primers in a one-step qRT-PCR reaction with FFPE tissue. Other investigators have either done a two-step qRT-PCR (cDNA synthesis in one reaction followed by qPCR) or have used random hexamers or truncated gene-specific primers. Abrahamsen et al. (2003); Specht et al. (2001); Godfrey et al. (2000); Cronin et al. (2004); and Mikhitarian et al. (2004).

In summary, the 78% overall accuracy of the assay for six tissue types compares favorably to other studies. Brown et al. (1997); DeYoung et al. (2000); Dennis et al. (2005a); Su et al. (2001); Ramaswamy et al. (2001); and Bloom et al. (2004).

### Example 7

In this study classifier using gene marker portfolios were built by choosing from MVO and using this classifier to predict tissue origin and cancer status for five major cancer types including breast, colon, lung, ovarian and prostate. Three hundred and seventy eight primary cancer, 23 benign proliferative epithelial lesions and 103 normal snap-frozen human tissue specimens were analyzed by using Affymetrix human U133A GeneChip. Leukocyte samples were also analyzed in order to subtract gene expression potentially masked by co-expression in leukocyte background cells. A novel MVO-based bioinformatics method was developed to select gene marker portfolios for tissue of origin and cancer status. The data demonstrated that a panel of 26 genes could be used as a classifier to accurately predict the tissue of origin and cancer status among the 5 cancer types. Thus a multi-cancer classification method is obtainable by determining gene expression profiles of a reasonably small number of gene markers.

Table 19 shows the Markers identified for the tissue origins indicated. For gene descriptions see Table 28.

TABLE 19

| Tissue | SEQ ID NO: | Name |
|---|---|---|
| Lung | 59 | SP-B |
| | 60 | TTF1 |
| | 61 | DSG3 |
| Pancreas | 66 | PSCA |
| | 67 | F5 |
| | 71 | ITGB6 |
| | 72 | TGM2 |
| | 80 | HNRPA0 |
| Colon | 81 | HPT1 |
| | 73 | FABP1 |
| | 74 | CDX1 |
| | 75 | GUCY2C |
| Prostate | 82 | PSA |
| | 76 | hKLK2 |
| Breast | 63 | MGB1 |
| | 77 | PIP |
| | 64 | PDEF |
| Ovarian | 78 | HE4 |
| | 79 | PAX8 |
| | 65 | WT1 |

The sample set included a total of 299 metastatic colon, breast, pancreas, ovary, prostate, lung and other carcinomas and primary prostate cancer samples. QC based on histological evaluation, RNA yield and expression of control gene beta-actin was implemented. Other samples category included metastasis originated from stomach (5), kidney (6), cholangio/gallbladder (4), liver (2), head and neck (4), ileum (1) carcinomas and one mesothelioma. Table 20 summarizes the results.

TABLE 20

| | | Histology | | ACTB |
|---|---|---|---|---|
| Tissue type | Collected | QC | RNA isolation QC | Cut-off QC |
| Lung | 41 | 37 | 36 | 25 |
| Pancreas | 63 | 57 | 49 | 41 |
| Colon | 45 | 42 | 42 | 31 |
| Breast | 40 | 35 | 35 | 34 |
| Ovarian | 37 | 36 | 35 | 33 |
| Prostate | 27 | 27 | 25 | 19 |
| Other | 46 | 34 | 29 | 23 |
| Total | 299 | 268 | 251 | 205 |

Testing the above samples resulted in the narrowing of the Marker set to those in Table 21 with the results seen in Table 22.

TABLE 21
Final Marker Table

| | | |
|---|---|---|
| Lung | surfactant-associated protein | SP-B |
| | thyroid transcription factor 1 | TTF1 |
| | desmoglein 3 | DSG3 |
| Pancreas | prostate stem cell antigen | PSCA |
| | coagulation factor 5 | F5 |
| Colon | intestinal peptide-associated | HPT1 |
| | transporter | |
| Prostate | prostate-specific antigen | PSA |
| Breast | Mammaglobin | MGB |
| | Ets transcription factor | PDEF |
| Ovary | Wilms tumor 1 | WT1 |

TABLE 22

| | | | | Sensitivity | | |
|---|---|---|---|---|---|---|
| Cancer | Samples # | Marker | Correct | % | Wrong | Spec % |
| Lung | 25/180 | SP-B | 13/25 | 52 | 0/180 | 100 |
| | | TTF | 12/25 | 48 | 1/180 | 99 |
| | | DSG3 | 5/25 | 20 | 0/180 | 100 |
| Pancreas | 41/164 | PSCA | 24/41 | 59 | 6/164 | 96 |
| | | F5 | 6/41 | 15 | 4/164 | 98 |
| Colon | 31/174 | HPT1 | 22/31 | 71 | 2/174 | 99 |
| Breast | 33/172 | MGB | 23/33 | 70 | 3/172 | 98 |
| | | PDEF | 16/33 | 48 | 1/172 | 99 |
| Prostate | 19/186 | PSA | 19/19 | 100 | 0/186 | 100 |
| | | PDEF | 19/19 | 100 | 2/186 | 99 |
| Ovarian | 33/172 | WT1 | 24/33 | 71 | 1/172 | 99 |
| Total | 205 | | | | | |

The results showed that out of 205 paraffin embedded metastatic tumors; 166 samples (81%) had conclusive assay results, Table 23.

TABLE 23

| | Candidate | Correct | Incorrect | No | Accuracy (%) |
|---|---|---|---|---|---|
| Lung | SP-B + TFF + | 19 | 0 | 6 | 76 |
| | DSG3 | | | | |
| Pancreas | PSCA + F5 | 27 | 1 | 13 | 66 |
| Colon | HPT1 | 24 | 2 | 5 | 78 |
| Prostate | PSA | 19 | 0 | 0 | 100 |
| Breast | MGB + PDEF | 23 | 3 | 7 | 70 |
| Ovarian | WT1 | 23 | 2 | 8 | 70 |
| Other | | 20 | 3 | | 87 |
| Overall | | 155 | 11 | 39 | 76 |

Of the false positive results, many false derived from histologically and embryologically similar tissues, Table 24.

TABLE 24

| Sample ID | Diagnosis | Predicted |
|---|---|---|
| OV_26 | Ovarian | Breast |
| Br_24 | Breast | Colon |
| Br_37 | Breast | Colon |
| CRC_25 | Colon | Ovarian |
| Pn_59 | Pancreas | Colon |
| Cont_27 | Stomach | pancreas |
| Cont_34 | Stomach | Colon |
| Cont_35 | Stomach | Colon |
| Cont_43 | Bile duct | Pancreas |
| Cont_44 | Bile duct | Pancreas |
| Cong_25 | Liver | pancreas |

The following parameters were considered for the model development:

Separate markers on female and male sets and calculate CUP probability separately for male and female patients. The male set included: SP_B, TTF1, DSG3, PSCA, F5, PSA, HPT1; the female set included: SP_B, TTF1, DSG3, PSCA, F5, HPT1, MGB, PDEF, WT1. Background expression was excluded from the assay results: Lung: SP_B, TTF1, DSG3; Ovary: WT1; and Colon: HPT1.

The CUP model was adjusted to the CUP prevalence (%): lung 23, pancreas 16, colorectal 9, breast 3, ovarian 4, prostate 2, other 43. The prevalence for breast and ovarian adjusted to 0% for male patients, and prostate adjusted to 0% for female patients.

The following steps were taken:

Place markers on similar scale.

Reduce number of variables from 12 to 8 by selecting minimum value from each tissue specific set.

Leave out 1 sample. Build model from remaining samples. Test left out sample. Repeat until 100% of samples are tested.

Randomly leave out ˜50% of samples (˜50% per tissue). Build model from remaining samples. Test ˜50% of samples. Repeat for 3 different random splits.

Classification accuracy was adjusted to cancer types prevalence To produce the results summarized in Table 25 with the raw data shown in Table 26.

TABLE 25

| | Breast | Colon | Lung | Other | Ovary | Pancreas | Prostate | Overall | Adjusted |
|---|---|---|---|---|---|---|---|---|---|
| Correct | 23 | 29 | 22 | 19 | 24 | 35 | 19 | 171 | |
| NoTest | 3 | 2 | 2 | | 2 | 3 | 0 | 12 | |
| Incorrect | 7 | 0 | 1 | 4 | 7 | 3 | 0 | 22 | |
| Prevalence | 0.03 | 0.09 | 0.23 | 0.43 | 0.04 | 0.16 | 0.02 | | |
| Tested/total % | 91 | 94 | 92 | 100 | 94 | 93 | 100 | 94 | 95 |
| Correct/total % | 70 | 94 | 88 | 83 | 73 | 85 | 100 | 89 | 89 |
| NoTest % | 9 | 6 | 8 | n/a | 6 | 7 | 0 | 6 | 5 |
| Correct | 23 | 25 | 19 | 20 | 20 | 24 | 19 | 150 | |
| NoTest % | 7 | 6 | 5 | | 10 | 15 | 0 | 43 | |
| Incorrect | 3 | 0 | 1 | 3 | 3 | 2 | 0 | 12 | |
| Prevalence | 0.03 | 0/09 | 0.23 | 0.43 | 0.04 | 0.16 | 0.02 | | |
| Tested/total % | 79 | 81 | 80 | 100 | 70 | 63 | 100 | 79 | 83 |
| Correct/total % | 70 | 81 | 76 | 87 | 61 | 59 | 100 | 73 | 76 |
| Correct/tested % | 88 | 100 | 95 | 87 | 87 | 92 | 100 | 93 | 91 |
| NoTest % | 21 | 19 | 20 | n/a | 30 | 37 | 0 | 21 | 17 |

TABLE 26

| Pt # | Gender | Class2 | Met.Site.Lite | b | Prediction | |
|---|---|---|---|---|---|---|
| 101 | m | lung | uk | ‘uk’ | ‘other’ | wrong |
| 106 | m | lung | uk | ‘uk’ | ‘lung’ | correct |
| 110 | m | lung | uk | ‘uk’ | ‘lung’ | correct |
| 112 | m | lung | uk | ‘uk’ | ‘other’ | wrong |
| 114 | f | liver | lung | ‘lung’ | ‘pancreas’ | wrong |
| 128 | f | breast | lung | ‘lung’ | ‘pancreas’ | wrong |
| 129 | m | CUP | lung | ‘lung’ | ‘other’ | correct |
| | | (renal) | | | | |
| 134 | f | breast | uk | ‘uk’ | ‘breast’ | correct |
| 136 | m | prostate | lung | ‘lung’ | ‘prostate’ | correct |
| 148 | f | ovary | uk | ‘uk’ | ‘pancreas’ | wrong |
| 163 | f | Colorectal | uk | ‘uk’ | ‘pancreas’ | wrong |
| 166 | f | Breast | uk | ‘uk’ | ‘breast’ | correct |
| 179 | f | renal | uk | ‘uk’ | ‘other’ | correct |
| 184 | m | colorectal | uk | ‘uk’ | ‘colon’ | correct |
| 194 | m | Head/ | uk | ‘uk’ | ‘other’ | correct |
| | | Neck | | | | |
| 199 | f | CUP SSC | uk | ‘uk’ | ‘lungSCC’ | correct |
| 200 | m | CUP SSC | uk | ‘uk’ | ‘lungSCC’ | correct |
| 302 | f | renal | colon | ‘colon’ | ‘breast’ | correct |
| 305 | m | renal | uk | ‘uk’ | ‘other’ | correct |
| 313 | m | lung | uk | ‘uk’ | ‘other’ | wrong |
| 317 | m | GI | uk | ‘uk’ | ‘pancreas’ | correct |
| 325 | m | lung | uk | ‘uk’ | ‘lungSCC’ | correct |
| 331 | f | breast | ovary | ‘ovary’ | ‘breast’ | correct |
| 333 | f | renal | uk | ‘uk’ | ‘other’ | correct |
| 334 | m | renal | uk | ‘uk’ | ‘other’ | correct |
| 335 | m | lung | uk | ‘uk’ | ‘lung’ | correct |
| 339 | f | colon | uk | ‘uk’ | ‘other’ | wrong |
| 342 | f | duodenum | uk | ‘uk’ | ‘other’ | correct |
| 346 | m | colon | lung | ‘lung’ | ‘pancreas’ | wrong |
| 347 | m | SCC | uk | ‘uk’ | ‘lungSCC’ | correct |
| | | lung, H+ | | | | |
| 354 | f | ovarian | uk | ‘uk’ | ‘ovary’ | correct |
| 356 | f | breast | uk | ‘uk’ | ‘breast’ | correct |
| 363 | m | colon | uk | ‘uk’ | ‘colon’ | correct |
| 374 | m | lung | uk | ‘uk’ | ‘lung’ | correct |
| 382 | m | renal | uk | ‘uk’ | ‘other’ | correct |
| 385 | f | SCC | uk | ‘uk’ | ‘lung’ | correct |
| | | lung, H+ | | | | |
| 404 | m | renal | uk | ‘uk’ | ‘other’ | correct |
| 407 | m | prostate | lung | ‘lung’ | ‘prostate’ | correct |
| 417 | f | pancreas | uk | ‘uk’ | ‘colon’ | wrong |

### Example 8

### Prospective Gene Signature Study of Metastatic Cancer of Unknown Primary Site CUP to Predict the Tissue of Origin

The specific aim of this study was to determine the ability of the 10-gene signature to predict tissue of origin of metastatic carcinoma in patients with carcinoma of unknown primary (CUP).

Primary objective: Confirm the feasibility of conducting gene analysis from core biopsy samples in consecutive patients with CUP.

Secondary objective: Correlate the results of the 10-gene signature RT-PCR assay with diagnostic work-up done at M.D. Anderson Cancer Center (MDACC). Third objective: Correlate prevalence of 6 cancer types predicted by assay with the prevalence derived from the literature and MDACC experience.

The method described herein was used to perform a microarray gene expression analysis of 700 frozen primary carcinoma, and benign and normal specimens and identified gene marker candidates, specific for lung, pancreas, colon, breast, prostate and ovarian carcinomas. Gene marker candidates were tested by RT-PCR on 205 formalin-fixed, paraffin-embedded (FFPE) specimens of metastatic carcinoma (Stage III-IV) originated from lung, pancreas, colon, breast, ovary and prostate as well as metastasis originated from other cancer types for specificity control. Other metastatic cancer types included gastric, renal cell, hepatocellular, cholangio/gallbladder and head and neck carcinomas. Results allowed selecting of 10-gene signature that predicted tissue of origin of metastatic carcinoma and gave an overall accuracy of 76%. The average CV for repeated measurements in RT-PCR experiments is 1.5%, calculated based on 4 replicate date points. Beta-actin (ACTB) was used as housekeeping gene and its median expression was the similar in metastatic samples of different origin (CV=5.6%).

Specific aim for this study was to validate the ability of 10-gene signature to predict metastatic carcinoma tissue of origin in the CUP patients compared to comprehensive diagnostic workup.

### Patient Eligibility

Patient must be at least 18 years old with a ECOG performance status of 0-2. Patients with diagnosis adenocarcinoma or poorly differentiated carcinoma diagnosis were accepted. Adenocarcinoma patient's group include well, moderate and poor differentiated tumors.

Patients have fulfilled the criteria for CUP: no primary detected after a complete evaluation which is defined as complete history and physical examination, detailed laboratory examination, imaging studies and symptom or sign directed invasive studies. Only untreated patients were allowed on the study.

If a patient has been treated with chemotherapy or radiation, participation in the study is allowed if prior (to treatment) tissue is available as archived blocks within 10 years time period.

Patients provided written consent/authorization to participate in this study.

### Study Design

Patients with diagnosis of CUP who have undergone a core needle or excision biopsy of the most accessible metastatic lesion were allowed on the study. Patients with FNA biopsy only were not eligible. The first 60 consecutive presenting patients who met the inclusion criteria and consent to the study were enrolled. If repeated biopsy is required at MDACC for diagnostic purposes for their treatment, additional tissue was obtained for the study if patient consented. All participants were registered on the protocol in the institutional Protocol Data Management System (PDMS).

Complete diagnostic work-up, including clinical and pathological assessments, was performed on all enrolled patients according MDACC standards. Pathology part of diagnostic work-up may have included immunohistochemistry (IHC) assays with markers including CK-7, CK-20, TTF-1 and other as deemed indicated by the pathologist. This is part of routine work up of all patients who present with CUP.

### Tissue Sample Collection

Study included formalin-fixed paraffin embedded metastatic carcinoma specimens collected from CUP patients.

Six 10 μm sections were used for RNA isolation, smaller tissue specimens will require nine 10 μm sections. Histopathology diagnosis and tumor content were confirmed for each sample used for RNA isolation on an additional section stained with hematoxylin and eosin (HE). The tumor sample should have had a greater than 30% of tumor content in the HE section.

Clinical data were anonymously supplied to Veridex and include patient age, gender, tumor histology by light microscopy, tumor grade (differentiation), site of metastasis, date of specimen collection, description of the diagnostic workup performed for individual patient.

### Tissue Processing and RT-PCR Experiments

Total RNA was extracted from each tissue sample using the protocol described above. Only samples that yielded more than 1 μm of total RNA out of standard amount of tissue were used for subsequent RT-PCR testing. Samples with less RNA yield were considered degraded and excluded from subsequent experiments. RNA integrity control based on housekeeping expression were implemented in order to exclude samples with degraded RNA, according the standard Veridex procedure.

RT-PCR assay that includes panel of 10 genes and 1-2 control genes was used for the analysis of the RNA samples. The reverse transcription and the PCR assay are completed using the protocols described above.

Relative expression value for each tested gene presented as ΔCt, which is equal to Ct of the target gene subtracted by Ct of the control genes, was calculated and used for the tissue of origin prediction.

### Sample Size and Data Interpretation

A limited sample size of 60 patients were studied due to the exploratory nature of the pilot study. Up to the date, 22 patients have been tested. One patient samples failed to yield enough RNA for RT-PCR test and 3 failed to pass QC control assessed by RT-PCR with control genes. A total of 18 patients were used for determine probability of patient's metastatic lesion.

The statistical model was used to determine probability of metastatic carcinoma tissue of origin of following seven categories: lung, pancreas, colon, breast, prostate, ovarian and no test (other). For each sample, the probability for each category are calculated from a linear classification model. Assay results are summarized in Table 27.

The probability of a patient's metastatic lesion (with known primaries) coming from one of these 6 sites (colon, pancreas, lung, prostate, ovary, breast) is about 76%. This number is derived from literature given the incidence of various cancers and potential for spread and unpublished data generated at M.D. Anderson from tumor registry. For the tested samples, prevalence of 6 sites was 67% (12 out 18 tested samples), which very close consistent with previous observations.

TABLE 27

| Patient data | | | ToO posterior probability (%) | | | | | | | |
|---|---|---|---|---|---|---|---|---|---|---|
| ID | M/F | prediction | Breast | Colon | Lung | LungSCC | Other | Ovary | Pancreas | prostate |
| 1 | M | Other | 0.00 | 0.00 | 0.81 | 0.00 | 98.68 | 0.00 | 0.51 | 0.00 |
| 4 | F | Colon | 0.00 | 99.70 | 0.00 | 0.00 | 0.09 | 0.20 | 0.01 | 0.00 |
| 5 | M | Lung | 0.00 | 33.29 | 52.27 | 0.01 | 13.30 | 0.00 | 1.13 | 0.00 |
| 6 | F | Colon | 0.00 | 99.91 | 0.00 | 0.00 | 0.09 | 0.00 | 0.00 | 0.00 |
| 2 | M | Colon | 0.00 | 93.19 | 0.01 | 0.00 | 2.90 | 0.00 | 3.90 | 0.00 |
| 10 | F | Other | 0.02 | 2.04 | 0.03 | 0.03 | 61.43 | 1.12 | 35.34 | 0.00 |
| 16 | F | Colon | 0.00 | 48.59 | 0.01 | 1.57 | 47.62 | 0.17 | 2.05 | 0.00 |
| 22 | M | LungSCC | 0.00 | 8.85 | 0.01 | 71.69 | 11.84 | 0.00 | 7.62 | 0.00 |
| 23 | M | Colon | 0.00 | 99.27 | 0.01 | 0.00 | 0.72 | 0.00 | 0.00 | 0.00 |
| 24 | F | Colon | 0.00 | 90.59 | 0.00 | 0.00 | 2.36 | 0.00 | 7.04 | 0.00 |
| 26 | F | Lung | 0.00 | 0.00 | 99.93 | 0.00 | 0.06 | 0.00 | 0.01 | 0.00 |
| 17 | M | Other | 0.00 | 0.07 | 0.02 | 0.09 | 94.06 | 0.00 | 5.77 | 0.00 |
| 19 | F | Other | 0.02 | 0.11 | 0.04 | 0.22 | 76.36 | 23.24 | 0.01 | 0.00 |
| 21 | F | Pancreas | 0.00 | 6.97 | 0.00 | 0.00 | 2.37 | 8.43 | 82.23 | 0.00 |
| 27 | F | Other | 0.00 | 0.04 | 0.04 | 0.59 | 99.06 | 0.14 | 0.13 | 0.00 |
| 11 | M | Other | 0.00 | 0.23 | 0.07 | 0.09 | 99.52 | 0.00 | 0.09 | 0.00 |
| 32 | F | Ovary | 0.00 | 0.01 | 0.00 | 0.00 | 7.23 | 92.63 | 0.13 | 0.00 |
| 34 | M | LungSCC | 0.00 | 0.03 | 0.00 | 65.64 | 7.96 | 0.00 | 26.38 | 0.00 |
| 3 | F | ctr failure | 0.00 | 0.00 | 0.00 | 0.00 | 0.00 | 0.00 | 0.00 | 0.00 |
| 8 | M | ctr failure | 0.00 | 0.00 | 0.00 | 0.00 | 0.00 | 0.00 | 0.00 | 0.00 |
| 20 | F | ctr failure | 0.00 | 0.00 | 0.00 | 0.00 | 0.00 | 0.00 | 0.00 | 0.00 |

Although the foregoing invention has been described in some detail by way of illustration and example for purposes of clarity of understanding, the descriptions and examples should not be construed as limiting the scope of the invention.

TABLE 28

| | SEQ ID | | |
|---|---|---|---|
| Name | NOs | Accession | Description |
| CDH17 | 62 | NM_004063 | Cadherin 17 |
| CDX1 | 74 | NM_001804 | Homeo box transcription factor 1 |
| DSG3 | 61/3 | NM_001944 | Desmoglein 3 |
| F5 | 67/6 | NM_000130 | Coagulation factor V |
| FABP1 | 73 | NM_001443 | Fatty acid binding protein 1, liver |
| GUCY2C | 75 | NM_004963 | Guanylate cyclase 2C |
| HE4 | 78 | NM_006103 | Putative ovarian carcinoma marker |
| KLK2 | 76 | BC005196 | Kallikrein 2, prostatic |
| HNRPA0 | 80 | NM_006805 | Heterogeneous nuclear ribonucleoprotein A0 |
| HPT1 | 81/4 | U07969 | Intestinal peptide-associated transporter |
| ITGB6 | 71 | NM_000888 | Integrin, beta 6 |
| KLK3 | 68 | NM_001648 | Kallikrein 3 |
| MGB1 | 63/7 | NM_002411 | Mammaglobin 1 |
| PAX8 | 79 | BC001060 | Paired box gene 8 |
| PBGD | 70 | NM_000190 | Hydroxymethylbilane synthase |
| PDEF | 64/8 | NM_012391 | Domain containing Ets transcription factor |
| PIP | 77 | NM_002652 | Prolactin-induced protein |
| PSA | 82/9 | U17040 | Prostate specific antigen precursor |
| PSCA | 66/5 | NM_005672 | Prostate stem cell antigen |
| SP-B | 59/1 | NM_198843 | Pulmonary surfactant-associated protein B |
| TGM2 | 72 | NM_004613 | Transglutaminase 2 |
| TTF1 | 60/2 | NM_003317 | Similar to thyroid transcription factor 1 |
| WT1 | 65/10 | NM_024426 | Wilms tumor 1 |
| β-actin | 69 | NM_001101 | β-actin |
| KRT6F | 83 | L42612 | keratin 6 isoform K6f |
| p73H | 84 | AB010153 | p53-related protein |
| SFTPC | 85 | NM_003018 | surfactant, pulmonary-associated protein C |
| KLK10 | 86 | NM_002776 | Kallikrein 10 |
| CLDN18 | 87 | NM_016369 | Claudin 18 |
| TR10 | 88 | BD280579 | Tumor necrosis factor receptor |
| B305D | 89 | AC018804 | BAC clone RP11-397H17 from 2 |
| B726 | 90 | AL357148 | |
| GABA-pi | 91 | BC109105 | gamma-aminobutyric acid A receptor, pi |
| StAR | 92 | NM_001007243 | steroidogenic acute regulator |
| EMX2 | 93 | NM_004098 | empty spiracles homolog 2 (Drosophila) |
| NGEP | 94 | AY617079 | NGEP long variant |
| NPY | 95 | NM_000905 | Neuropeptide Y |
| SERPINA1 | 96 | NM_000295 | serpin peptidase inhibitor, clade A member 1 |
| KRT7 | 97 | NM_005556 | Keratin 7 |
| MMP11 | 98 | NM_005940 | matrix metallopeptidase 11 (stromelysin 3) |
| MUC4 | 99 | NM_018406 | Mucin 4 cell-surface associated |
| FLJ22041 | 100 | AK025694 | |
| BAX | 101 | NM_138763 | BCL2-assoc X protein transcript variant Δ |
| PITX1 | 102 | NM_002653 | paired-like homeodomain trans factor 1 |
| MGC: 10264 | 103 | BC005807 | stearoyl-CoA desaturase (Δ-9-desaturase) |

### REFERENCES

US patent application publications and patents

5,242,974
5,350,840
5,384,261
5,405,783
5,412,087
5,424,186
5,429,807
5,436,327
5,445,934
5,472,672
5,527,681
5,529,756
5,532,128
5,545,531
5,554,501
5,556,752
5,561,071
5,571,639
5,593,839
5,599,695
5,624,711
5,658,734
5,668,267
5,700,637
5,786,148
6,004,755
6,136,182
6,218,114
6,218,122
6,225,051
6,232,073
6,261,766
6,271,002
6,339,148
20010029020
20020055627
20030215835
20030219760
20030232350
20030235820
20040005563
20040076955
20040146862
20040219572
20040219575
20050037010
20050059008
20060094035

Foreign patent publications and patents

WO1998040403
WO1998056953
WO2000006589
WO2000055320
WO2001073032
WO2002073204
WO2002101357
WO2004018999
WO2004030615
WO2004031412
WO2004063355
WO2004077060
WO2005005601

### Journal Articles

Abrahamsen et al. (2003) Towards quantitative mRNA analysis in paraffin-embedded tissues using real-time reverse transcriptase-polymerase chain reaction J Mol Diag 5:34-41
Al-Mulla et al. (2005) BRCA1 gene expression in breast cancer: a correlative study between real-time RT-PCR and immunohistochemistry J Histochem Cytochem 53:621-629
Argani et al. (2001) Discovery of new Markers of cancer through serial analysis of gene expression: prostate stem cell antigen is overexpressed in pancreatic adenocarcinoma Cancer Res 61:4320-4324
Autiero et al. (2002) Intragenic amplification and formation of extrachromosomal small circular DNA molecules from the PIP gene on chromosome 7 in primary breast carcinomas Int J Cancer 99:370-377
Backus et al. (2005) Identification and characterization of optimal gene expression Markers for detection of breast cancer metastasis J Mol Diagn 7:327-336
Bentov et al. (2003) The WT1 Wilms' tumor suppressor gene: a novel target for insulin-like growth factor-I action Endocrinol 144:4276-4279
Bera et al. (2004) NGEP, a gene encoding a membrane protein detected only in prostate cancer and normal prostate Proc Natl Acad Sci USA 101:3059-3064
Bibikova et al. (2004) Quantitative gene expression profiling in formalin-fixed, paraffin-embedded tissues using universal bead arrays Am j Pathol 165:1799-1807
Bloom et al. (2004) Multi-platform, multi-site, microarray-based human tumor classification Am J Pathol 164:9-16
Borchers et al. (1997) Heart-type fatty acid binding protein—involvement in growth inhibition and differentiation Prostaglandins Leukot Essent Fatty Acids 57:77-84
Borgono et al. (2004) Human tissue kallikreins: physiologic roles and applications in cancer Mol Cancer Res 2:257-280
Brookes (1999) The essence of SNPs Gene 23:177-186
Brown et al. (1997) Immunohistochemical identification of tumor Markers in metastatic adenocarcinoma. A diagnostic adjunct in the determination of primary site Am J Clin Pathol 107:12-19
Buckhaults et al. (2003) Identifying tumor origin using a gene expression-based classification map Cancer Res 63:4144-4149
Chan et al. (1985) Human liver fatty acid binding protein cDNA and amino acid sequence. Functional and evolutionary implications J Biol Chem 260:2629-2632
Chen et al. (1986) Human liver fatty acid binding protein gene is located on chromosome 2 Somat Cell Mol Genet 12:303-306
Cheung et al. (2003) Detection of the PAX8-PPAR gamma fusion oncogene in both follicular thyroid carcinomas and adenomas J Clin Endocrinol Metab 88:354-357
Clark et al. (1999) The potential role for prolactin-inducible protein (PIP) as a Marker of human breast cancer micrometastasis Br J Cancer 81:1002-1008
Cronin et al. (2004) Measurement of gene expression in archival paraffin-embedded tissue Am J Pathol 164:35-42
Cunha et al. (2006) Tissue-specificity of prostate specific antigens: Comparative analysis of transcript levels in prostate and non-prostatic tissues Cancer Lett 236:229-238
Dennis et al. (2002) Identification from public data of molecular Markers of adenocarcinoma characteristic of the site of origin Can Res 62:5999-6005
Dennis et al. (2005a) Hunting the primary: novel strategies for defining the origin of tumors J Pathol 205:236-247
Dennis et al. (2005b) Markers of adenocarcinoma characteristic of the site of origin: development of a diagnostic algorithm Clin Can Res 11:3766-3772
DeYoung et al. (2000) Immunohistologic evaluation of metastatic carcinomas of unknown origin: a linear discrimination analysisic approach Semin Diagn Pathol 17:184-193
Di Palma et al. (2003) The paired domain-containing factor Pax8 and the homeodomain-containing factor TTF-1 directly interact and synergistically activate transcription Biol Chem 278:3395-3402
Dwight et al. (2003) Involvement of the PAX8 peroxisome proliferator-activated receptor gamma rearrangement in follicular thyroid tumors J Clin Endocrinol Metab 88:4440-4445
Feldman et al. (2003) PDEF expression in human breast cancer is correlated with invasive potential and altered gene expression Cancer Res 63:4626-4631
Fleming et al. (2000) Mammaglobin, a breast-specific gene, and its utility as a Marker for breast cancer Ann N Y Acad Sci 923:78-89
Fukushima et al. (2004) Characterization of gene expression in mucinous cystic neoplasms of the pancreas using oligonucleotide microarrays Oncogene 23:9042-9051
Ghosh et al. (2005) Management of patients with metastatic cancer of unknown primary Curr Probl Surg 42:12-66
Giordano et al. (2001) Organ-specific molecular classification of primary lung, colon, and ovarian adenocarcinomas using gene expression profiles Am J Pathol.159:1231-1238
Glasser et al. (1988) cDNA, deduced polypeptide structure and chromosomal assignment of human pulmonary surfactant proteolipid, SPL(pVal) J Biol Chem 263:9-12
Godfrey et al. (2000) Quantitative mRNA expression analysis from formalin-fixed, paraffin-embedded tissues using 5′ nuclease quantitative reverse transcription-polymerase chain reaction J Mol Diag 2:84-91
Goldstein et al. (2002) WT1 immunoreactivity in uterine papillary serous carcinomas is different from ovarian serous carcinomas Am J Clin Pathol 117:541-545
Gradi et al. (1995) The human steroidogenic acute regulatory (StAR) gene is expressed in the urogenital system and encodes a mitochondrial polypeptide Biochim Biophys Acta 1258:228-233
Greco et al. (2004) Carcinoma of unknown primary site: sequential treatment with paclitaxel/carboplatin/etoposide and gemcitabine/irinotecan: A Minnie Pearl cancer research network phase II trial The Oncologist 9:644-652
Haas et al. (2005) Combined application of RT-PCR and immunohistochemistry on paraffin embedded sentinel lymph nodes of prostate cancer patients Pathol Res Pract 200:763-770
Hwang et al. (2004) Wilms tumor gene product: sensitive and contextually specific Marker of serous carcinomas of ovarian surface epithelial origin Appl Immunohistochem Mol Morphol 12:122-126
Ishikawa et al. (2005) Experimental trial for diagnosis of pancreatic ductal carcinoma based on gene expression profiles of pancreatic ductal cells Cancer Sci 96:387-393
Italiano et al. (2005) Epidermal growth factor receptor (EGFR) status in primary colorectal tumors correlates with EGFR expression in related metastatic sites: biological and clinical implications Ann Oncol 16:1503-1507
Jones et al. (2004) Comprehensive analysis of matrix metalloproteinase and tissue inhibitor expression in pancreatic cancer: increased expression of matrix metalloproteinase-7 predicts poor survival Clin Cancer Res 10:2832-2845
Jones et al. (2005) Thyroid transcription factor 1 expression in small cell carcinoma of the urinary bladder: an immunohistochemical profile of 44 cases Hum Pathol 36:718-723
Khoor et al. (1997) Expression of surfactant protein B precursor and surfactant protein B mRNA in adenocarcinoma of the lung Mod Pathol 10:62-67
Kim (2003) Comparison of oligonucleotide-microarray and serial analysis of gene expression (SAGE) in transcript profiling analysis of megakaryocytes derived from CD34+ cells Exp Mol Med 35:460-466
Kim et al. (2003) Steroidogenic acute regulatory protein expression in the normal human brain and intracranial tumors Brain Res 978:245-249
Lam et al. (2005) Prostate stem cell antigen is overexpressed in prostate cancer metastases Clin Can Res 11:2591-2596
Lembersky et al. (1996) Metastases of unknown primary site Med Clin North Am. 80:153-171
Lewis et al. (2001) Unlocking the archive-gene expression in paraffin-embedded tissue J Pathol 195:66-71
Lipshutz et al. (1999) High density synthetic oligonucleotide arrays Nature Genetics 21:S20-24
Lowe et al. (1985) Human liver fatty acid binding protein. Isolation of a full length cDNA and comparative sequence analyses of orthologous and paralogous proteins J Biol Chem 260:3413-3417
Ma et al. (2006) Molecular classification of human cancers using a 92-gene real-time quantitative polymerase chain reaction assay Arch Pathol Lab med 130:465-473
Magklara et al. (2002) Characterization of androgen receptor and nuclear receptor co-regulator expression in human breast cancer cell lines exhibiting differential regulation of kallikreins 2 and 3 Int J Cancer 100:507-514
Markowitz (1952) Portfolio Selection J Finance 7:77-91
Marques et al. (2002) Expression of PAX8-PPAR gamma 1 rearrangements in both follicular thyroid carcinomas and adenomas J Clin Endocrinol Metab 87:3947-3952
Masuda et al. (1999) Analysis of chemical modification of RNA from formalin-fixed samples and optimization of molecular biology applications for such samples Nucl Acids Res 27:4436-4443
McCarthy et al. (2003) Novel Markers of pancreatic adenocarcinoma in fine-needle aspiration: mesothelin and prostate stem cell antigen labeling increases accuracy in cytologically borderline cases Appl Immunohistochem Mol Morphol 11:238-243
Mikhitarian et al. (2004) Enhanced detection of RNA from paraffin-embedded tissue using a panel of truncated gene-specific primers for reverse transcription BioTechniques 36:1-4
Mintzer et al. (2004) Cancer of unknown primary: changing approaches, a multidisciplinary case presentation from the Joan Karnell Cancer Center of Pennsylvania Hospital The Oncologist 9:330-338
Moniaux et al. (2004) Multiple roles of mucins in pancreatic cancer, a lethal and challenging malignancy Br J Cancer 91:1633-1638
Murphy et al. (1987) Isolation and sequencing of a cDNA clone for a prolactin-inducible protein (PIP). Regulation of PIP gene expression in the human breast cancer cell line, T-47D J Biol Chem 262:15236-15241
Myal et al. (1991) The prolactin-inducible protein (PIPGCDFP-15) gene: cloning, structure and regulation J Mol Cell Endocrinol 80:165-175
Nakamura et al. (2002) Expression of thyroid transcription factor-1 in normal and neoplastic lung tissues Mod Pathol 15:1058-1067
Noonan et al. (2001) Characterization of the homeodomain gene EMX2: sequence conservation, expression analysis, and a search for mutations in endometrial cancers Genomics 76:37-44
Oettgen et al. (2000) PDEF, a novel prostate epithelium-specific Ets transcription factor, interacts with the androgen receptor and activates prostate-specific antigen gene expression J Biol Chem 275:1216-1225
Oji et al. (2003) Overexpression of the Wilms' tumor gene WT1 in head and neck squamous cell carcinoma Cancer Sci 94:523-529
Pavlidis et al. (2003) Diagnostic and therapeutic management of cancer of an unknown primary Eur J Can 39: 990-2005
Pilot-Mathias et al. (1989) Structure and organization of the gene encoding human pulmonary surfactant proteolipid SP-B DNA 8:75-86
Pilozzi et al. (2004) CDX1 expression is reduced in colorectal carcinoma and is associated with promoter hypermethylation J Pathol 204:289-295
Poleev et al. (1992) PAX8, a human paired box gene: isolation and expression in developing thyroid, kidney and Wilms' tumors Development 116:611-623
Prasad et al. (2005) Gene expression profiles in pancreatic intraepithelial neoplasia reflect the effects of Hedgehog signaling on pancreatic ductal epithelial cells Cancer Res 65:1619-1626
Ramaswamy (2004) Translating cancer genomics into clinical oncology N Engl J Med 350:1814-1816
Ramaswamy et al. (2001) Multiclass cancer diagnosis using tumor gene expression signatures Proc Natl Acad Sci USA 98:15149-15154
Rauscher (1993) The WT1 Wilms tumor gene product: a developmentally regulated transcription factor in the kidney that functions as a tumor suppressor FASEB J 7:896-903
Reinholz et al. (2005) Evaluation of a panel of tumor Markers for molecular detection of circulating cancer cells in women with suspected breast cancer Clin Cancer Res 11:3722
Schlag et al. (1994) Cancer of unknown primary site Ann Chir Gynaecol 83:8-12
Senoo et al. (1998) A second p53-related protein, p73L, with high homology to p73 Biochem Biophys Res Comm 248:603-607
Specht et al. (2001) Quantitative gene expression analysis in microdissected archival formalin-fixed and paraffin-embedded tumor tissue Amer J Pathol 158:419-429
Su et al. (2001) Molecular classification of human carcinomas by use of gene expression signatures Cancer Res 61:7388-7393
Takahashi et al. (1995) Cloning and characterization of multiple human genes and cDNAs encoding highly related type 11 keratin 6 isoforms J Biol Chem 270:18581-18592
Takamura et al. (2004) Reduced expression of liver-intestine cadherin is associated with progression and lymph node metastasis of human colorectal carcinoma Cancer Lett 212:253-259
Tothill et al. (2005) An expression-based site of origin diagnostic method designed for clinical application to cancer of unknown origin Can Res 65:4031-4040
van Ruissen et al. (2005) Evaluation of the similarity of gene expression data estimated with SAGE and Affymetrix GeneChips BMC Genomics 6:91
Varadhachary et al. (2004) Diagnostic strategies for unknown primary cancer Cancer 100:1776-1785
Wallace et al. (2005) Accurate Molecular detection of non-small cell lung cancer metastases in mediastinal lymph nodes sampled by endoscopic ultrasound-guided needle aspiration Cest 127:430-437
Wan et al. (2003) Desmosomal proteins, including desmoglein 3, serve as novel negative Markers for epidermal stem cell-containing population of keratinocytes J Cell Sci 116:4239-4248
Watson et al. (1996) Mammaglobin, a mammary-specific member of the uteroglobin gene family, is overexpressed in human breast cancer Cancer Res 56:860-865
Watson et al. (1998) Structure and transcriptional regulation of the human mammaglobin gene, a breast cancer associated member of the uteroglobin gene family localized to chromosome 11q13 Oncogene 16:817-824
Weigelt et al. (2003) Gene expression profiles of primary breast tumors maintained in distant metastases Proc Natl Acad Sci USA 100:15901-15905
Zapata-Benavides et al. (2002) Downregulation of Wilms' tumor 1 protein inhibits breast cancer proliferation Biochem Biophys Res Commun 295:784-790

1. A method of identifying origin of a metastasis of unknown origin comprising the steps of
a. obtaining a sample containing metastatic cells;
b. measuring Biomarkers associated with at least two different carcinomas;
c. combining the data from the Biomarkers into a linear discrimination analysis where the linear discrimination analysis
i. normalizes the Biomarkers against a reference; and
ii. imposes a cut-off which optimizes sensitivity and specificity of each Biomarker, weights the prevalence of the carcinomas and selects a tissue of origin;
d. determining origin based on highest probability determined by the linear discrimination analysis or determining that the carcinoma is not derived from a particular set of carcinomas; and
e. optionally measuring Biomarkers specific for one or more additional different carcinoma, and repeating steps c) and d) for the additional Biomarkers.

2. The method of claim 1 wherein the Marker genes are selected from at least one from a group corresponding to:
i. SP-B, TTF, DSG3, KRT6F, p73H, or SFTPC;
ii. F5, PSCA, ITGB6, KLK10, CLDN18, TR10 or FKBP10; or
iii. CDH17, CDX1 or FABP1.

3. The method of claim 2 wherein the Marker genes are SP-B, TTF, DSG3, KRT6F, p73H, or SFTPC.

4. The method according to claim 3 wherein the Marker genes are SP-B, TTF and DSG3.

5. The method according to claim 4 wherein the Marker genes further comprise or are replaced by KRT6F, p73H, and/or SFTPC.

6. The method of claim 2 wherein the Marker genes are F5, PSCA, ITGB6, KLK10, CLDN18, TR10 or FKBP10.

7. The method of claim 6 wherein the Marker genes are F5 and PSCA.

8. The method of claim 7 wherein the Marker genes further comprise or are replaced by ITGB6, KLK10, CLDN18, TR10 and/or FKBP10.

9. The method of claim 1 wherein the Marker genes are CDH17, CDX1 or FABP1.

10. The method of claim 9 wherein the Marker gene is CDH17.

11. The method of claim 10 wherein the Marker gene further comprises or are replaced by CDX1 and/or FABP1.

12. The method of one of claims 1-11 wherein gene expression is measured using at least one of SEQ ID NOs: 11-58.

13. The method of claim 2 wherein the Marker genes are further selected from a gender specific Marker selected from at least one of
i. in the case of a male patient KLK3, KLK2, NGEP or NPY; or
ii. in the case of a female patient PDEF, MGB, PIP, B305D, B726 or GABA-Pi;
and/or WT1, PAX8, STAR or EMX2.

14. The method of claim 13 wherein the Marker gene is KLK2.

15. The method of claim 14 wherein the Marker gene is KLK3.

16. The method of claim 15 wherein the Marker gene further comprises or are replaced by NGEP and/or NPY.

17. The method of claim 13 wherein the Marker genes are PDEF, MGB, PIP, B305D, B726 or GABA-Pi.

18. The method of claim 17 wherein the Marker genes are PDEF and MGB.

19. The method of claim 18 wherein the Marker genes further comprise or are replaced by PIP, B305D, B726 or GABA-Pi.

20. The method of claim 13 wherein the Marker genes are WT1, PAX8, STAR or EMX2.

21. The method of claim 20 wherein the Marker gene is WT1.

22. The method of claim 21 wherein the Marker gene further comprises or is replaced by PAX8, STAR or EMX2.

23. The method of one of claims 13-22 wherein gene expression is measured using at least one of SEQ ID NOs: 11-58.

24. The method of claim 1 or 2 comprising further obtaining additional clinical information including the site of metastasis to determine the origin of the carcinoma.

25. A method of obtaining optimal biomarker sets for carcinomas comprising the steps of using metastases of know origin, determining Biomarkers therefor and comparing the Biomarkers to Biomarkers of metastases of unknown origin.

26. A method of providing direction of therapy by determining the origin of a metastasis of unknown origin according to one of claims 1-3 and identifying the appropriate treatment therefor.

27. A method of providing a prognosis by determining the origin of a metastasis of unknown origin according to one of claims 1-3 and identifying the corresponding prognosis therefor.

28. A method of finding Biomarkers comprising determining the expression level of a Marker gene in a particular metastasis, measuring a Biomarker for the Marker gene to determine expression thereof, analyzing the expression of the Marker gene according to claim 1 and determining if the Marker gene is effectively specific for the tumor of origin.

29. A composition comprising at least one isolated sequence selected from SEQ ID NOs: 11-58.

30. A kit for conducting an assay according to one of claims 1-3 comprising: Biomarker detection reagents.

31. A microarray or gene chip for performing the method of one of claims 1-3.

32. A diagnostic/prognostic portfolio comprising isolated nucleic acid sequences, their complements, or portions thereof of a combination of genes according to one of claims 2-11, or 13-22 where the combination is sufficient to measure or characterize gene expression in a biological sample having metastatic cells relative to cells from different carcinomas or normal tissue.

33. A method according to one of claims 2-11, or 13-22 further comprising measuring expression of at least one gene constitutively expressed in the sample.


SEQUENCE LISTING (104 sequences)

SEQ ID NO: 1 - DNA, 476 bp, human
gaaaaaccagccactgctttacaggacagggggttgaagctgagccccgcctcacacccacccccatgcactcaaagattggattttacagctacttgcaattcaaaattcagaagaataaaaaatgggaacatacagaactctaaaagatagacatcagaaattgttaagttaagctttttcaaaaaatcagcaattccccagcgtagtcaagggtggacactgcacgctctggcatgatgggatggcgaccgggcaagctttcttcctcgagatgctctgctgcttgagagctattgctttgttaagatataaaaaggggtttctttttgtctttctgtaaggtggacttccagattttgattgaaagtcctagggtgattctatttctgctgtgatttatctgctgaaagctcagctggggttgtgcaagctagggacccattcctgtgtaatacaatgtctgcaccaatgct

SEQ ID NO: 2 - DNA, 493 bp, human
gtgattcaaatgggttttccacgctagggcggggcacagattggagagggctctgtgctgacatggctctggactctaaagaccaaacttcactctgggcacactctgccagcaaagaggactcgcttgtaaataccaggatttttttttttttttgaagggaggacgggagctggggagaggaaagagtcttcaacataacccacttgtcactgacacaaaggaagtgccccctccccggcaccctctggccgcctaggctcagcggcgaccgccctccgcgaaaatagtttgtttaatgtgaacttgtagctgtaaaacgctgtcaaaagttggactaaatgcctagtttttagtaatctgtacattttgttgtaaaaagaaaaaccactcccagtccccagcccttcacattttttatgggcattgacaaatctgtgtatattatttggcagtttggtatttgcggcgtcagtctttttctgttgtaact

SEQ ID NO: 3 - DNA, 545 bp, human
ccatcccatagaagtccagcagacaggatttgttaagtgccagactttgtcaggaagtcaaggagcttctgctttgtccgcctctgggtctgtccagccagctgtttccatccctgaccctctgcagcatggtaactatttagtaacggagacttactcggcttctggttccctcgtgcaaccttccactgcaggctttgatccacttctcacacaaaatgtgatagtgacagaaagggtgatctgtcccatttccagtgttcctggcaacctagctggcccaacgcagctacgagggtcacatactatgctctgtacagaggatccttgctcccgtctaatatgaccagaatgagctggaataccacactgaccaaatctggatctttggactaaagtattcaaaatagcatagcaaagctcactgtattgggctaataatttggcacttattagcttctctcataaactgatcacgattataaattaaatgtttgggttcataccccaaaagcaatatgttgtcactcctaattctcaagtac

SEQ ID NO: 4 - DNA, 284 bp, human
ctgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtc

SEQ ID NO: 5 - DNA, 394 bp, human
  misc_feature (58)..(58): n is a, c, g, or t
ttcctgaggcacatcctaacgcaagtttgaccatgtatgtttgcaccccttttccccnaaccctgaccttcccatgggccttttccaggattccnaccnggcagatcagttttagtganacanatccgcntgcagatggcccctccaaccntttntgttgntgtttccatggcccagcattttccacccttaaccctgtgttcaggcacttnttcccccaggaagccttccctgcccaccccatttatgaattgagccaggtttggtccgtggtgtcccccgcacccagcaggggacaggcaatcaggagggcccagtaaaggctgagatgaagtggactgagtagaactggaggacaagagttgacgtgagttcctgggagtttccagagatg

SEQ ID NO: 6 - DNA, 470 bp, human
  misc_feature (61)..(61): n is a, c, g, or t
atcctctacagccagatgtcacagggatacgtctactttcacttggtgctggagaattcanaagtcaagaacatgctaagcntaagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggaaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaag

SEQ ID NO: 7 - DNA, 396 bp, human
gcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttact

SEQ ID NO: 8 - DNA, 491 bp, human
gagtggggcccttaaactggattcaaaaaatgctctaaacataggaatggttgaagaggtcttgcagtcttcagatgaaactaaatctctagaagaggcacaagaatggctaaagcaattcatccaagggccaccggaagtaattagagctttgaaaaaatctgtttgttcaggcagagagctatatttggaggaagcattacagaacgaaagagatcttttaggaacagtttggggtgggcctgcaaatttagaggctattgctaagaaaggaaaatttaataaataattggtttttcgtgtggatgtactccaagtaaagctccagtgactaatatgtataaatgttaaatgatattaaatatgaacatcagttaaaaaaaaaattctttaaggctactattaatatgcagacttacttttaatcatttgaaatctgaactcatttacctcatttcttgccaattactcccttgggtatttactgcgta

SEQ ID NO: 9 - DNA, 265 bp, human
tggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcc

SEQ ID NO: 10 - DNA, 441 bp, human
atagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagat

SEQ ID NO: 11 - DNA, 21 bp, human
cacagccccgacctttgatga

SEQ ID NO: 12 - DNA, 19 bp, human
ggtcccagagcccgtctca

SEQ ID NO: 13 - DNA, 26 bp, human
agctgtccagctgcaaaggaaaagcc

SEQ ID NO: 14 - DNA, 75 bp, human
cacagccccgacctttgatgagaactcagctgtccagctgcaaaggaaaagccaagtgagacgggctctgggacc

SEQ ID NO: 15 - DNA, 17 bp, human
ccaacccagacccgcgc

SEQ ID NO: 16 - DNA, 21 bp, human
cgcccatgccgctcatgttca

SEQ ID NO: 17 - DNA, 21 bp, human
cccgccatctcccgcttcatg

SEQ ID NO: 18 - DNA, 78 bp, human
ccaacccagacccgcgcttccccgccatctcccgcttcatgggcccggcgagcggcatgaacatgagcggcatgggcg

SEQ ID NO: 19 - DNA, 24 bp, human
gcagagaaggagaagataactcaa

SEQ ID NO: 20 - DNA, 22 bp, human
actccagagattcggtaggtga

SEQ ID NO: 21 - DNA, 26 bp, human
attgccaagattacttcagattacca

SEQ ID NO: 22 - DNA, 97 bp, human
gcagagaaggagaagataactcaaaaagaaacccaattgccaagattacttcagattaccaagcaacccagaaaatcacctaccgaatctctggagt

SEQ ID NO: 23 - DNA, 21 bp, human
tccctcggcagtggaagctta

SEQ ID NO: 24 - DNA, 24 bp, human
tcctcaaactctgtgtgcctggta

SEQ ID NO: 25 - DNA, 29 bp, human
ccaaaatcaatggtactcatgcccgactg

SEQ ID NO: 26 - DNA, 95 bp, human
tccctcggcagtggaagcttacaaaacgactgggaagtttccaaaatcaatggtactcatgcccgactgtctaccaggcacacagagtttgagga

SEQ ID NO: 27 - DNA, 21 bp, human
agttgctgatggtcctcatgc

SEQ ID NO: 28 - DNA, 24 bp, human
cacttgtggattgattgtcttgga

SEQ ID NO: 29 - DNA, 23 bp, human
ccctctcccagcactgctacgca

SEQ ID NO: 30 - DNA, 107 bp, human
agttgctgatggtcctcatgctggcggccctctcccagcactgctacgcaggctctggctgccccttattggagaatgtgatttccaagacaatcaatccacaagtg

SEQ ID NO: 31 - DNA, 20 bp, human
cgcccacctggacatctgga

SEQ ID NO: 32 - DNA, 23 bp, human
cactggtcgaggcacagtagtga

SEQ ID NO: 33 - DNA, 25 bp, human
gtcagcggcctggatgaaagagcgg

SEQ ID NO: 34 - DNA, 86 bp, human
cgcccacctggacatctggaagtcagcggcctggatgaaagagcggacttcacctggggcgattcactactgtgcctcgaccagtg

SEQ ID NO: 35 - DNA, 23 bp, human
gcggagcccaatacagaatacac

SEQ ID NO: 36 - DNA, 19 bp, human
cggggctactccaggcaca

SEQ ID NO: 37 - DNA, 25 bp, human
tcagaggcattcaggatgtgcgacg

SEQ ID NO: 38 - DNA, 80 bp, human
gcggagcccaatacagaatacacacgcacggtgtcttcagaggcattcaggatgtgcgacgtgtgcctggagtagccccg

SEQ ID NO: 39 - DNA, 20 bp, human
ctgttgatggcaggcttggc

SEQ ID NO: 40 - DNA, 20 bp, human
ttgctcacctgggctttgca

SEQ ID NO: 41 - DNA, 21 bp, human
gcagccaggcactgccctgct

SEQ ID NO: 42 - DNA, 74 bp, human
ctgttgatggcaggcttggccctgcagccaggcactgccctgctgtgctactcctgcaaagcccaggtgagcaa

SEQ ID NO: 43 - DNA, 25 bp, human
tgaagaaatatcctgggattattca

SEQ ID NO: 44 - DNA, 27 bp, human
tatgtggtatcttctggaatatcatca

SEQ ID NO: 45 - DNA, 27 bp, human
acaaagggaaacagatattgaagactc

SEQ ID NO: 46 - DNA, 87 bp, human
tgaagaaatatcctgggattattcagaatttgtacaaagggaaacagatattgaagactctgatgatattccagaagataccacata

SEQ ID NO: 47 - DNA, 19 bp, human
cccccagtgggtcctcaca

SEQ ID NO: 48 - DNA, 22 bp, human
aggatgaaacaagctgtgccga

SEQ ID NO: 49 - DNA, 26 bp, human
caggaacaaaagcgtgatcttgctgg

SEQ ID NO: 50 - DNA, 82 bp, human
cccccagtgggtcctcacagctgcccactgcatcaggaacaaaagcgtgatcttgctgggtcggcacagcttgtttcatcct

SEQ ID NO: 51 - DNA, 22 bp, human
cggatgtccacgtcacacttca

SEQ ID NO: 52 - DNA, 25 bp, human
cttccttcctgggcatggagtcctg

SEQ ID NO: 53 - DNA, 100 bp, human
gccctgaggcactcttccagccttccttcctgggcatggagtcctgtggcatccacgaaactaccttcaactccatcatgaagtgtgacgtggacatccg

SEQ ID NO: 54 - DNA, 22 bp, human
ccacacacagcctactttccaa

SEQ ID NO: 55 - DNA, 21 bp, human
tacccacgcgaatcactctca

SEQ ID NO: 56 - DNA, 27 bp, human
aacggcaatgcggctgcaacggcggaa

SEQ ID NO: 57 - DNA, 103 bp, human
ccacacacagcctactttccaagcggagccatgtctggtaacggcaatgcggctgcaacggcggaagaaaacagcccaaagatgagagtgattcgcgtgggta

SEQ ID NO: 58 - DNA, 2724 bp, human
ggtgccatggctgagtcacacctgctgcagtggctgctgctgctgctgcccacgctctgtggcccaggcactgctgcctggaccacctcatccttggcctgtgcccagggccctgagttctggtgccaaagcctggagcaagcattgcagtgcagagccctagggcattgcctacaggaagtctggggacatgtgggagccgatgacctatgccaagagtgtgaggacatcgtccacatccttaacaagatggccaaggaggccattttccaggacacgatgaggaagttcctggagcaggagtgcaacgtcctccccttgaagctgctcatgccccagtgcaaccaagtgcttgacgactacttccccctggtcatcgactacttccagaaccagactgactcaaacggcatctgtatgcacctgggcctgtgcaaatcccggcagccagagccagagcaggagccagggatgtcagaccccctgcccaaacctctgcgggaccctctgccagaccctctgctggacaagctcgtcctccctgtgctgcccggggccctccaggcgaggcctgggcctcacacacaggatctctccgagcagcaattccccattcctctcccctattgctggctctgcagggctctgatcaagcggatccaagccatgattcccaagggtgcgctagctgtggcagtggcccaggtgtgccgcgtggtacctctggtggcgggcggcatctgccagtgcctggctgagcgctactccgtcatcctgctcgacacgctgctgggccgcatgctgccccagctggtctgccgcctcgtcctccggtgctccatggatgacagcgctggcccaaggtcgccgacaggagaatggctgccgcgagactctgagtgccacctctgcatgtccgtgaccacccaggccgggaacagcagcgagcaggccataccacaggcaatgctccaggcctgtgttggctcctggctggacagggaaaagtgcaagcaatttgtggagcagcacacgccccagctgctgaccctggtgcccaggggctgggatgcccacaccacctgccaggccctcggggtgtgtgggaccatgtccagccctctccagtgtatccacagccccgacctttgatgagaactcagctgtccagaaaaagacaccgtcctttaaagtgctgcagtatggccagacgtggtggctcacacctgcaatcccagcaccttaggaggccgaggcaggaggatccttgaggtcaggagttcgagaccagcctcgccaacatggtgaaaccccatttctactaaaaatacaaaaaattagccaagtgtggtggcatatgcctgtaatcccaactactcagaaggccgaggcaggagaattacttgaacgcaggagaatcactgcagcccaggaggcagaggttgcagtgagccgagattgcaccactgcactccagcctgggtgacagagcaagactccatctcagtaaataaataaataaataaaaagcgctgcagtagctgtggcctcaccctgaagtcagcgggcccaggcctacctcactctctcccttggcagagaagcagacgtccatagctcctctccctcacaagcgctcccagcctgccctccagctgctgctctcccctcccagtctctactcactgggatgaggttaggtcatgaggacaccaaaaacctaaaaataaacaaaaagccaaacaagccttagcttttcttaaagactgaaatgcctggaagtgtccctttatttataaaataacttttgtcatatttcttatacatgtttcttgtaagaaattcagaaactacagacaaagagagtggaaattacccactgtcaggcctctgagcccaagctaagccatcatatcccctgtgccctgcacgtatacacccagatggcctgaagcaactgaagatccacaaaagaagtgaaaatagccagttcctgccttaactgatgacattccaccattgtgatttgttcctgccccaccctaactgatcaattgaccttgtgacaatacaccttccccacccttgagaaggtgctttgtaatattctccccacccaccccacgcccgcacccccgcacccttaagaaggtattttgtaatattctctccgccattgagaatgtgctttgtaagatccaccccctgcccacaaaaaattgctcctaactccaccgcctatcccaaacctacaagaactaatgataatcccaccaccctttgctgactctttttggactcagcccacctgcacccaggtgattaaaaagctttattgttcacacaaagcctgtttggtagtctcttcacagggaagcatgtgacacccacaatcccacctagcccaggagagagctacggcagggtgtgtgttttgacactgagcttggggctttttccatcttctccccacagcctctggctccacacctccaccgttcaagcgccagaaagagctgtctatgcagcctgctcttgggcctggggatgagacacacaattcattggctcctggattttaagtagacatttgtaaatctatagctaactactgtccttaaagccattgtttccattacaaaatccaactctctgagagaaaagggtgttttaaatttaaaaaaataaaaacaaaaaagtttgattgagaaaaaaaaaaaaaaa

SEQ ID NO: 59 - DNA, 2352 bp, human
gaaacttaaaggtgtttaccttgtcatcagcatgtaagctaattatctcgggcaagatgtaggcttctattgtcttgttgctttagcgcttacgccccgcctctggtggctgcctaaaacctggcgccgggctaaaacaaacgcgaggcagcccccgagcctccactcaagccaattaaggaggactcggtccactccgttacgtgtacatccaacaagatcggcgttaaggtaacaccagaatatttggcaaagggagaaaaaaaaagcagcgaggcttcgccttccccctctcccttttttttcctcctcttccttcctcctccagccgccgccgaatcatgtcgatgagtccaaagcacacgactccgttctcagtgtctgacatcttgagtcccctggaggaaagctacaagaaagtgggcatggagggcggcggcctcggggctccgctggcggcgtacaggcagggccaggcggcaccgccaacagcggccatgcagcagcacgccgtggggcaccacggcgccgtcaccgccgcctaccacatgacggcggcgggggtgccccagctctcgcactccgccgtggggggctactgcaacggcaacctgggcaacatgagcgagctgccgccgtaccaggacaccatgaggaacagcgcctctggccccggatggtacggcgccaacccagacccgcgcttccccgccatctcccgcttcatgggcccggcgagcggcatgaacatgagcggcatgggcggcctgggctcgctgggggacgtgagcaagaacatggccccgctgccaagcgcgccgcgcaggaagcgccgggtgctcttctcgcaggcgcaggtgtacgagctggagcgacgcttcaagcaacagaagtacctgtcggcgccggagcgcgagcacctggccagcatgatccacctgacgcccacgcaggtcaagatctggttccagaaccaccgctacaaaatgaagcgccaggccaaggacaaggcggcgcagcagcaactgcagcaggacagcggcggcggcgggggcggcgggggcaccgggtgcccgcagcagcaacaggctcagcagcagtcgccgcgacgcgtggcggtgccggtcctggtgaaagacggcaaaccgtgccaggcgggtgcccccgcgccgggcgccgccagcctacaaggccacgcgcagcagcaggcgcagcaccaggcgcaggccgcgcaggcggcggcagcggccatctccgtgggcagcggtggcgccggccttggcgcacacccgggccaccagccaggcagcgcaggccagtctccggacctggcgcaccacgccgccagccccgcggcgctgcagggccaggtatccagcctgtcccacctgaactcctcgggctcggactacggcaccatgtcctgctccaccttgctatacggtcggacctggtgagaggacgccgggccggccctagcccagcgctctgcctcaccgcttccctcctgcccgccacacagaccaccatccaccgctgctccacgcgcttcgacttttcttaacaacctggccgcgtttagaccaaggaacaaaaaaaccacaaaggccaaactgctggacgtctttctttttttccccccctaaaatttgtgggtttttttttttaaaaaaagaaaatgaaaaacaaccaagcgcatccaatctcaaggaatctttaagcagagaagggcataaaacagctttggggtgtctttttttggtgattcaaatgggttttccacgctagggcggggcacagattggagagggctctgtgctgacatggctctggactctaaagaccaaacttcactctgggcacactctgccagcaaagaggactcgcttgtaaataccaggatttttttttttttttgaagggaggacgggagctggggagaggaaagagtcttcaacataacccacttgtcactgacacaaaggaagtgccccctccccggcaccctctggccgcctaggctcagcggcgaccgccctccgcgaaaatagtttgtttaatgtgaacttgtagctgtaaaacgctgtcaaaagttggactaaatgcctagtttttagtaatctgtacattttgttgtaaaaagaaaaaccactcccagtccccagcccttcacattttttatgggcattgacaaatctgtgtatattatttggcagtttggtatttgcggcgtcagtctttttctgttgtaacttatgtagatatttggcttaaatatagttcctaagaagcttctaataaattatacaaattaaaaagattctttttctgattaaaaaaaaaaaaaaaaaa

SEQ ID NO: 60 - DNA, 5561 bp, human
aaagcagcagagacgctgcagagggcttttcttagacatcaactgcagacggctggcaggatagaagcagcggctcacttggactttttcaccagggaaatcagagacaatgatggggctcttccccagaactacaggggctctggccatcttcgtggtggtcatattggttcatggagaattgcgaatagagactaaaggtcaatatgatgaagaagagatgactatgcaacaagctaaaagaaggcaaaaacgtgaatgggtgaaatttgccaaaccctgcagagaaggagaagataactcaaaaagaaacccaattgccaagattacttcagattaccaagcaacccagaaaatcacctaccgaatctctggagtgggaatcgatcagccgccttttggaatctttgttgttgacaaaaacactggagatattaacataacagctatagtcgaccgggaggaaactccaagcttcctgatcacatgtcgggctctaaatgcccaaggactagatgtagagaaaccacttatactaacggttaaaattttggatattaatgataatcctccagtattttcacaacaaattttcatgggtgaaattgaagaaaatagtgcctcaaactcactggtgatgatactaaatgccacagatgcagatgaaccaaaccacttgaattctaaaattgccttcaaaattgtctctcaggaaccagcaggcacacccatgttcctcctaagcagaaacactggggaagtccgtactttgaccaattctcttgaccgagagcaagctagcagctatcgtctggttgtgagtggtgcagacaaagatggagaaggactatcaactcaatgtgaatgtaatattaaagtgaaagatgtcaacgataacttcccaatgtttagagactctcagtattcagcacgtattgaagaaaatattttaagttctgaattacttcgatttcaagtaacagatttggatgaagagtacacagataattggcttgcagtatatttctttacctctgggaatgaaggaaattggtttgaaatacaaactgatcctagaactaatgaaggcatcctgaaagtggtgaaggctctagattatgaacaactacaaagcgtgaaacttagtattgctgtcaaaaacaaagctgaatttcaccaatcagttatctctcgataccgagttcagtcaaccccagtcacaattcaggtaataaatgtaagagaaggaattgcattccgtcctgcttccaagacatttactgtgcaaaaaggcataagtagcaaaaaattggtggattatatcctgggaacatatcaagccatcgatgaggacactaacaaagctgcctcaaatgtcaaatatgtcatgggacgtaacgatggtggatacctaatgattgattcaaaaactgctgaaatcaaatttgtcaaaaatatgaaccgagattctactttcatagttaacaaaacaatcacagctgaggttctggccatagatgaatacacgggtaaaacttctacaggcacggtatatgttagagtacccgatttcaatgacaattgtccaacagctgtcctcgaaaaagatgcagtttgcagttcttcaccttccgtggttgtctccgctagaacactgaataatagatacactggcccctatacatttgcactggaagatcaacctgtaaagttgcctgccgtatggagtatcacaaccctcaatgctacctcggccctcctcagagcccaggaacagatacctcctggagtataccacatctccctggtacttacagacagtcagaacaatcggtgtgagatgccacgcagcttgacactggaagtctgtcagtgtgacaacaggggcatctgtggaacttcttacccaaccacaagccctgggaccaggtatggcaggccgcactcagggaggctggggcctgccgccatcggcctgctgctccttggtctcctgctgctgctgttggccccccttctgctgttgacctgtgactgtggggcaggttctactgggggagtgacaggtggttttatcccagttcctgatggctcagaaggaacaattcatcagtggggaattgaaggagcccatcctgaagacaaggaaatcacaaatatttgtgtgcctcctgtaacagccaatggagccgatttcatggaaagttctgaagtttgtacaaatacgtatgccagaggcacagcggtggaaggcacttcaggaatggaaatgaccactaagcttggagcagccactgaatctggaggtgctgcaggctttgcaacagggacagtgtcaggagctgcttcaggattcggagcagccactggagttggcatctgttcctcagggcagtctggaaccatgagaacaaggcattccactggaggaaccaataaggactacgctgatggggcgataagcatgaattttctggactcctacttttctcagaaagcatttgcctgtgcggaggaagacgatggccaggaagcaaatgactgcttgttgatctatgataatgaaggcgcagatgccactggttctcctgtgggctccgtgggttgttgcagttttattgctgatgacctggatgacagcttcttggactcacttggacccaaatttaaaaaacttgcagagataagccttggtgttgatggtgaaggcaaagaagttcagccaccctctaaagacagcggttatgggattgaatcctgtggccatcccatagaagtccagcagacaggatttgttaagtgccagactttgtcaggaagtcaaggagcttctgctttgtccacctctgggtctgtccagccagctgtttccatccctgaccctctgcagcatggtaactatttagtaacggagacttactcggcttctggttccctcgtgcaaccttccactgcaggctttgatccacttctcacacaaaatgtgatagtgacagaaagggtgatctgtcccatttccagtgttcctggcaacctagctggcccaacgcagctacgagggtcacatactatgctctgtacagaggatccttgctcccgtctaatatgaccagaatgagctggaataccacactgaccaaatctggatctttggactaaagtattcaaaatagcatagcaaagctcactgtattgggctaataatttggcacttattagcttctctcataaactgatcacgattataaattaaatgtttgggttcataccccaaaagcaatatgttgtcactcctaattctcaagtactattcaaattgtagtaaatcttaaagtttttcaaaaccctaaaatcatattcgccaggaaattttcctaaacattcttaagcttctatttttcccctgccaaaggaaggtgtttatcattttaaaatgcaatgtgatttagtggattaagcaggagcgctggttcttgtctccattgccttttcttatatcattgataatgatgtaagaatcacaaggggccgggcgcggtggctcacgcctgtaatcccagcactttgggaggccgaggcaggtggatcatgaggtcaggagatcgagaccatcctggctaacaaggtgaaaccccgtctctactaaaaatacaaaaaattagccgggcgcagtggcgggcgcctgtagtcccagctactcgggaggctgaggcaggagaatggcatgaacccgggaagcggagcttgcagtgagccgagattgcgccactgcagtccgcagtccggcctgggcgacagagcgagactccgtctcaaaaaaaaaaaaaaaaaaagaatcacaaggtatttgctaaagcattttgagctgcttggaaaaagggaagtagttgcagtagagtttcttccatcttcttggtgctgggaagccatatatgtgtcttttactcaagctaaggggtataagcttatgtgttgaatttgctacatctatatttcacatattctcacaataagagaattttgaaatagaaatatcatagaacatttaagaaagtttagtataaataatattttgtgtgttttaatccctttgaagggatctatccaaagaaaatattttacactgagctccttcctacacgtctcagtaacagatcctgtgttagtctttgaaaatagctcattttttaaatgtcagtgagtagatgtagcatacatatgatgtataatgacgtgtattatgttaacaatgtctgcagattttgtaggaatacaaaacatggccttttttataagcaaaacgggccaatgactagaataacacatagggcaatctgtgaatatgtattataagcagcattccagaaaagtagttggtgaaataattttcaagtcaaaaagggatatggaaagggaattatgagtaacctctattttttaagccttgcttttaaattaaacagctacagccatttaagccttgaggataataaagcttgagagtaataatgttaggttagcaaaggtttagatgtatcacttcatgcatgctaccatgatagtaatgcagctcttcgagtcatttctggtcattcaagatattcacccttttgcccatagaaagcaccctacctcacctgcttactgacattgtcttagctgatcacaagatcattatcagcctccattattccttactgtatataaaatacagagttttatattttcctttcttcgtttttcaccatattcaaaacctaaatttgtttttgcagatggaatgcaaagtaatcaagtgtttgtgctttcacctagaagggtgtggtcctgaaggaaagaggtcccctaaatatcccccaccctggtgctcctccctctccctggtaccctgactaccaggaagtcaggtgctagagcagctggagaagtgcaggcagcctgtgcttccacagatgggggtgctgctgcaacaaggctttcaatgtgcccatcttaggtgggagaagctagatcctgtgcagcagcctggtaagtcctgaggaggttccattgctcttcctgctgctgtcctttgcttctcaacggtggctcgctctacagtctagagcacatgcagctaacttgtgcctctgcttatgcatgagggttaaattaacaaccataaccttcatttgaagttcaaaggtgtattcaggatcctcaaagcattttaaccttgccgcttaaaacccaatttaccgtgaaatgggaattttgctgcattgttaaactgtagtggaaaccatgctatagtaataaaggttatataagagagaaattgaaattaaatgtgtttttaaatttcaaaaaaaaatcaatctttaggatgacttaaaaattgatttgccatgtaaaatgtatctgcattttttacacaaaacttgttttaagcataaaattttaaaactgtactacttgatgtattatacattttgaaccatatgtattaaaccataaacagtataatgttgttataataaaacaggcaataaatttataaataaaagctgaaaaaaaaaaaa

SEQ ID NO: 61 - DNA, 3697 bp, human
agggagtgttcccgggggagatactccagtcgtagcaagagtctcgaccactgaatggaagaaaaggacttttaaccaccattttgtgacttacagaaaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccatagaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttgagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagagtttgaggagagggagtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaacgtgaggactcaggaaaaataaataaataaaagaacagccttttcccttagtattaacagaaatgtttctgtgtcattaaccatctttaatcaatgtgacatgttgctctttggctgaaattcttcaacttggaaatgacacagacccacagaaggtgttcaaacacaacctactctgcaaaccttggtaaaggaaccagtcagctggccagatttcctcactacctgccatgcatacatgctgcgcatgttttcttcattcgtatgttagtaaagttttggttattatatatttaacatgtggaagaaaacaagacatgaaaagagtggtgacaaatcaagaataaacactggttgtagtcagttttgtttgttaa

SEQ ID NO: 62 - DNA, 502 bp, human
acagcggcttccttgatccttgccacccgcgactgaacaccgacagcagcagcctcaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttattttaactttctgcaagacctttggctcacagaactgcagggtatggtgagaaaccaactacggattgctgcaaaccacaccttctctttcttatgtctttttactacaaactacaagacaattgttgaaacctgctatacatgtttattttaataaattgatggca

SEQ ID NO: 63 - DNA, 1894 bp, human
gtctgacttcctcccagcacattcctgcactctgccgtgtccacactgccccacagacccagtcctccaagcctgctgccagctccctgcaagcccctcaggttgggccttgccacggtgccagcaggcagccctgggctgggggtaggggactccctacaggcacgcagccctgagacctcagagggccaccccttgagggtggccaggcccccagtggccaacctgagtgctgcctctgccaccagccctgctggcccctggttccgctggccccccagatgcctggctgagacacgccagtggcctcagctgcccacacctcttcccggcccctgaagttggcactgcagcagacagctccctgggcaccaggcagctaacagacacagccgccagcccaaacagcagcggcatgggcagcgccagcccgggtctgagcagcgtatcccccagccacctcctgctgccccccgacacggtgtcgcggacaggcttggagaaggcggcagcgggggcagtgggtctcgagagacgggactggagtcccagtccacccgccacgcccgagcagggcctgtccgccttctacctctcctactttgacatgctgtaccctgaggacagcagctgggcagccaaggcccctggggccagcagtcgggaggagccacctgaggagcctgagcagtgcccggtcattgacagccaagccccagcgggcagcctggacttggtgcccggcgggctgaccttggaggagcactcgctggagcaggtgcagtccatggtggtgggcgaagtgctcaaggacatcgagacggcctgcaagctgctcaacatcaccgcagatcccatggactggagccccagcaatgtgcagaagtggctcctgtggacagagcaccaataccggctgccccccatgggcaaggccttccaggagctggcgggcaaggagctgtgcgccatgtcggaggagcagttccgccagcgctcgcccctgggtggggatgtgctgcacgcccacctggacatctggaagtcagcggcctggatgaaagagcggacttcacctggggcgattcactactgtgcctcgaccagtgaggagagctggaccgacagcgaggtggactcatcatgctccgggcagcccatccacctgtggcagttcctcaaggagttgctactcaagccccacagctatggccgcttcattaggtggctcaacaaggagaagggcatcttcaaaattgaggactcagcccaggtggcccggctgtggggcatccgcaagaaccgtcccgccatgaactacgacaagctgagccgctccatccgccagtattacaagaagggcatcatccggaagccagacatctcccagcgcctcgtctaccagttcgtgcaccccatctgagtgcctggcccagggcctgaaacccgccctcaggggcctctctcctgcctgccctgcctcagccaggccctgagatgggggaaaacgggcagtctgctctgctgctctgaccttccagagcccaaggtcagggaggggcaaccaactgccccagggggatatgggtcctctggggccttcgggaccatggggcaggggtgcttcctcctcaggcccagctgctcccctggaggacagagggagacagggctgctccccaacacctgcctctgaccccagcatttccagagcagagcctacagaagggcagtgactcgacaaaggccacaggcagtccaggcctctctctgctccatccccctgcctcccattctgcaccacacctggcatggtgcagggagacatctgcacccctgagttgggcagccaggagtgcccccgggaatggataataaagatactagagaactg

SEQ ID NO: 64 - DNA, 3029 bp, human
ccaggcagctggggtaaggagttcaaggcagcgcccacacccgggggctctccgcaacccgaccgcctgtccgctcccccacttcccgccctccctcccacctactcattcacccacccacccacccagagccgggacggcagcccaggcgcccgggccccgccgtctcctcgccgcgatcctggacttcctcttgctgcaggacccggcttccacgtgtgtcccggagccggcgtctcagcacacgctccgctccgggcctgggtgcctacagcagccagagcagcagggagtccgggacccgggcggcatctgggccaagttaggcgccgccgaggccagcgctgaacgtctccagggccggaggagccgcggggcgtccgggtctgagccgcagcaaatgggctccgacgtgcgggacctgaacgcgctgctgcccgccgtcccctccctgggtggcggcggcggctgtgccctgcctgtgagcggcgcggcgcagtgggcgccggtgctggactttgcgcccccgggcgcttcggcttacgggtcgttgggcggccccgcgccgccaccggctccgccgccacccccgccgccgccgcctcactccttcatcaaacaggagccgagctggggcggcgcggagccgcacgaggagcagtgcctgagcgccttcactgtccacttttccggccagttcactggcacagccggagcctgtcgctacgggcccttcggtcctcctccgcccagccaggcgtcatccggccaggccaggatgtttcctaacgcgccctacctgcccagctgcctcgagagccagcccgctattcgcaatcagggttacagcacggtcaccttcgacgggacgcccagctacggtcacacgccctcgcaccatgcggcgcagttccccaaccactcattcaagcatgaggatcccatgggccagcagggctcgctgggtgagcagcagtactcggtgccgcccccggtctatggctgccacacccccaccgacagctgcaccggcagccaggctttgctgctgaggacgccctacagcagtgacaatttataccaaatgacatcccagcttgaatgcatgacctggaatcagatgaacttaggagccaccttaaagggagttgctgctgggagctccagctcagtgaaatggacagaagggcagagcaaccacagcacagggtacgagagcgataaccacacaacgcccatcctctgcggagcccaatacagaatacacacgcacggtgtcttcagaggcattcaggatgtgcgacgtgtgcctggagtagccccgactcttgtacggtcggcatctgagaccagtgagaaacgccccttcatgtgtgcttacccaggctgcaataagagatattttaagctgtcccacttacagatgcacagcaggaagcacactggtgagaaaccataccagtgtgacttcaaggactgtgaacgaaggttttctcgttcagaccagctcaaaagacaccaaaggagacatacaggtgtgaaaccattccagtgtaaaacttgtcagcgaaagttctcccggtccgaccacctgaagacccacaccaggactcatacaggtaaaacaagtgaaaagcccttcagctgtcggtggccaagttgtcagaaaaagtttgcccggtcagatgaattagtccgccatcacaacatgcatcagagaaacatgaccaaactccagctggcgctttgaggggtctccctcggggaccgttcagtgtcccaggcagcacagtgtgtgaactgctttcaagtctgactctccactcctcctcactaaaaaggaaacttcagttgatcttcttcatccaacttccaagacaagataccggtgcttctggaaactaccaggtgtgcctggaagagttggtctctgccctgcctacttttagttgactcacaggccctggagaagcagctaacaatgtctggttagttaaaagcccattgccatttggtgtggattttctactgtaagaagagccatagctgatcatgtccccctgacccttcccttctttttttatgctcgttttcgctggggatggaattattgtaccattttctatcatggaatatttataggccagggcatgtgtatgtgtctgctaatgtaaactttgtcatggtttccatttactaacagcaacagcaagaaataaatcagagagcaaggcatcgggggtgaatcttgtctaacattcccgaggtcagccaggctgctaacctggaaagcaggatgtagttctgccaggcaacttttaaagctcatgcatttcaagcagctgaagaaaaaatcagaactaaccagtacctctgtatagaaatctaaaagaattttaccattcagttaattcaatgtgaacactggcacactgctcttaagaaactatgaagatctgagatttttttgtgtatgtttttgactcttttgagtggtaatcatatgtgtctttatagatgtacatacctccttgcacaaatggaggggaattcattttcatcactgggagtgtccttagtgtataaaaaccatgctggtatatggcttcaagttgtaaaaatgaaagtgactttaaaagaaaataggggatggtccaggatctccactgataagactgtttttaagtaacttaaggacctttgggtctacaagtatatgtgaaaaaaatgagacttactgggtgaggaaatccattgtttaaagatggtcgtgtgtgtgtgtgtgtgtgtgtgtgtgtgtgtgttgtgttgtgttttgttttttaagggagggaatttattatttaccgttgcttgaaattactgtgtaaatatatgtctgataatgatttgctctttgacaactaaaattaggactgtataagtactagatgcatcactgggtgttgatcttacaagatattgatgataacacttaaaattgtaacctgcatttttcactttgctctcaattaaagtctattcaaaag

SEQ ID NO: 65 - DNA, 1064 bp, human
tttgaggccatataaagtcacctgaggccctctccaccacagcccaccagtgaccatgaaggctgtgctgcttgccctgttgatggcaggcttggccctgcagccaggcactgccctgctgtgctactcctgcaaagcccaggtgagcaacgaggactgcctgcaggtggagaactgcacccagctgggggagcagtgctggaccgcgcgcatccgcgcagttggcctcctgaccgtcatcagcaaaggctgcagcttgaactgcgtggatgactcacaggactactacgtgggcaagaagaacatcacgtgctgtgacaccgacttgtgcaacgccagcggggcccatgccctgcagccggctgctgccatccttgcgctgctccctgcactcggcctgctgctctggggacccggccagctctaggctctggggggccccgctgcagcccacactgggtgtggtgccccaggcctctgtgccactcctcacacacccggcccagtgggagcctgtcctggttcctgaggcacatcctaacgcaagtctgaccatgtatgtctgcgcccctgtcccccaccctgaccctcccatggccctctccaggactcccacccggcagatcggctctattgacacagatccgcctgcagatggcccctccaaccctctctgctgctgtttccatggcccagcattctccacccttaaccctgtgctcaggcacctcttcccccaggaagccttccctgcccaccccatctatgacttgagccaggtctggtccgtggtgtcccccgcacccagcaggggacaggcactcaggagggcccggtaaaggctgagatgaagtggactgagtagaactggaggacaggagtcgacgtgagttcctgggagtctccagagatggggcctggaggcctggaggaaggggccaggcctcacattcgtggggctccctgaatggcagcctcagcacagcgtaggcccttaataaacacctgttggataagccaaaaaaaaaaaaaaaaaaaaaaaaaaaaaaaaaaaaaaaaaaaaa

SEQ ID NO: 66 - DNA, 9179 bp, human
gcaagaactgcaggggaggaggacgctgccacccacagcctctagagctcattgcagctgggacagcccggagtgtggttagcagctcggcaagcgctgcccaggtcctggggtggtggcagccagcgggagcaggaaaggaagcatgttcccaggctgcccacgcctctgggtcctggtggtcttgggcaccagctgggtaggctgggggagccaagggacagaagcggcacagctaaggcagttctacgtggctgctcagggcatcagttggagctaccgacctgagcccacaaactcaagtttgaatctttctgtaacttcctttaagaaaattgtctacagagagtatgaaccatattttaagaaagaaaaaccacaatctaccatttcaggacttcttgggcctactttatatgctgaagtcggagacatcataaaagttcactttaaaaataaggcagataagcccttgagcatccatcctcaaggaattaggtacagtaaattatcagaaggtgcttcttaccttgaccacacattccctgcggagaagatggacgacgctgtggctccaggccgagaatacacctatgaatggagtatcagtgaggacagtggacccacccatgatgaccctccatgcctcacacacatctattactcccatgaaaatctgatcgaggatttcaactcggggctgattgggcccctgcttatctgtaaaaaagggaccctaactgagggtgggacacagaagacgtttgacaagcaaatcgtgctactatttgctgtgtttgatgaaagcaagagctggagccagtcatcatccctaatgtacacagtcaatggatatgtgaatgggacaatgccagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaagctgggatgcaggcttacattgacattaaaaactgcccaaagaaaaccaggaatcttaagaaaataactcgtgagcagaggcggcacatgaagaggtgggaatacttcattgctgcagaggaagtcatttgggactatgcacctgtaataccagcgaatatggacaaaaaatacaggtctcagcatttggataatttctcaaaccaaattggaaaacattataagaaagttatgtacacacagtacgaagatgagtccttcaccaaacatacagtgaatcccaatatgaaagaagatgggattttgggtcctattatcagagcccaggtcagagacacactcaaaatcgtgttcaaaaatatggccagccgcccctatagcatttaccctcatggagtgaccttctcgccttatgaagatgaagtcaactcttctttcacctcaggcaggaacaacaccatgatcagagcagttcaaccaggggaaacctatacttataagtggaacatcttagagtttgatgaacccacagaaaatgatgcccagtgcttaacaagaccatactacagtgacgtggacatcatgagagacatcgcctctgggctaataggactacttctaatctgtaagagcagatccctggacaggcgaggaatacagagggcagcagacatcgaacagcaggctgtgtttgctgtgtttgatgagaacaaaagctggtaccttgaggacaacatcaacaagttttgtgaaaatcctgatgaggtgaaacgtgatgaccccaagttttatgaatcaaacatcatgagcactatcaatggctatgtgcctgagagcataactactcttggattctgctttgatgacactgtccagtggcacttctgtagtgtggggacccagaatgaaattttgaccatccacttcactgggcactcattcatctatggaaagaggcatgaggacaccttgaccctcttccccatgcgtggagaatctgtgacggtcacaatggataatgttggaacttggatgttaacttccatgaattctagtccaagaagcaaaaagctgaggctgaaattcagggatgttaaatgtatcccagatgatgatgaagactcatatgagatttttgaacctccagaatctacagtcatggctacacggaaaatgcatgatcgtttagaacctgaagatgaagagagtgatgctgactatgattaccagaacagactggctgcagcattaggaatcaggtcattccgaaactcatcattgaatcaggaagaagaagagttcaatcttactgccctagctctggagaatggcactgaattcgtttcttcaaacacagatataattgttggttcaaattattcttccccaagtaatattagtaagttcactgtcaataaccttgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcaaaagtcaagaacatgctaagcataagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggaaggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctacaagtaagacaggatggaggaaagagtagactgaagaaaagccagtttctcattaagacacgaaaaaagaaaaaagagaagcacacacaccatgctcctttatctccgaggacctttcaccctctaagaagtgaagcctacaacacattttcagaaagaagacttaagcattcgttggtgcttcataaatccaatgaaacatctcttcccacagacctcaatcagacattgccctctatggattttggctggatagcctcacttcctgaccataatcagaattcctcaaatgacactggtcaggcaagctgtcctccaggtctttatcagacagtgcccccagaggaacactatcaaacattccccattcaagaccctgatcaaatgcactctacttcagaccccagtcacagatcctcttctccagagctcagtgaaatgcttgagtatgaccgaagtcacaagtccttccccacagatataagtcaaatgtccccttcctcagaacatgaagtctggcagacagtcatctctccagacctcagccaggtgaccctctctccagaactcagccagacaaacctctctccagacctcagccacacgactctctctccagaactcattcagagaaacctttccccagccctcggtcagatgcccatttctccagacctcagccatacaaccctttctccagacctcagccatacaaccctttctttagacctcagccagacaaacctctctccagaactcagtcagacaaacctttctccagccctcggtcagatgcccctttctccagacctcagccatacaaccctttctctagacttcagccagacaaacctctctccagaactcagccatatgactctctctccagaactcagtcagacaaacctttccccagccctcggtcagatgcccatttctccagacctcagccatacaaccctttctctagacttcagccagacaaacctctctccagaactcagtcaaacaaacctttccccagccctcggtcagatgcccctttctccagaccccagccatacaaccctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggatctcagccagatatcacctcctccagaccttgatcagatattctacccttctgaatctagtcagtcattgcttcttcaagaatttaatgagtcttttccttatccagaccttggtcagatgccatctccttcatctcctactctcaatgatacttttctatcaaaggaatttaatccactggttatagtgggcctcagtaaagatggtacagattacattgagatcattccaaaggaagaggtccagagcagtgaagatgactatgctgaaattgattatgtgccctatgatgacccctacaaaactgatgttaggacaaacatcaactcctccagagatcctgacaacattgcagcatggtacctccgcagcaacaatggaaacagaagaaattattacattgctgctgaagaaatatcctgggattattcagaatttgtacaaagggaaacagatattgaagactctgatgatattccagaagataccacatataagaaagtagtttttcgaaagtacctcgacagcacttttaccaaacgtgatcctcgaggggagtatgaagagcatctcggaattcttggtcctattatcagagctgaagtggatgatgttatccaagttcgttttaaaaatttagcatccagaccgtattctctacatgcccatggactttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagaaaaagatattcactcaggcttgataggtcccctcctaatctgccaaaaaggaatactacataaggacagcaacatgcctatggacatgagagaatttgtcttactatttatgacctttgatgaaaagaagagctggtactatgaaaagaagtcccgaagttcttggagactcacatcctcagaaatgaaaaaatcccatgagtttcacgccattaatgggatgatctacagcttgcctggcctgaaaatgtatgagcaagagtgggtgaggttacacctgctgaacataggcggctcccaagacattcacgtggttcactttcacggccagaccttgctggaaaatggcaataaacagcaccagttaggggtctggccccttctgcctggttcatttaaaactcttgaaatgaaggcatcaaaacctggctggtggctcctaaacacagaggttggagaaaaccagagagcagggatgcaaacgccatttcttatcatggacagagactgtaggatgccaatgggactaagcactggtatcatatctgattcacagatcaaggcttcagagtttctgggttactgggagcccagattagcaagattaaacaatggtggatcttataatgcttggagtgtagaaaaacttgcagcagaatttgcctctaaaccttggatccaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatgtattttaatggcaattcagatgcctctacaataaaagagaatcagtttgacccacctattgtggctagatatattaggatctctccaactcgagcctataacagacctacccttcgattggaactgcaaggttgtgaggtaaatggatgttccacacccctgggtatggaaaatggaaagatagaaaacaagcaaatcacagcttcttcgtttaagaaatcttggtggggagattactgggaacccttccgtgcccgtctgaatgcccagggacgtgtgaatgcctggcaagccaaggcaaacaacaataagcagtggctagaaattgatctactcaagatcaagaagataacggcaattataacacagggctgcaagtctctgtcctctgaaatgtatgtaaagagctataccatccactacagtgagcagggagtggaatggaaaccatacaggctgaaatcctccatggtggacaagatttttgaaggaaatactaataccaaaggacatgtgaagaactttttcaaccccccaatcatttccaggtttatccgtgtcattcctaaaacatggaatcaaagtattgcacttcgcctggaactctttggctgtgatatttactagaattgaacattcaaaaacccctggaagagactctttaagacctcaaaccatttagaatgggcaatgtattttacgctgtgttaaatgttaacagttttccactatttctctttcttttctattagtgaataaaattttatacaagaagcttttataatgtaactccttgctaccagtaagtaagataatggctattacttctgcattaatttgaatacaggtaggaaaatatcaagaaccaacaagaaaagggcttatctttcttaatgattgaaaatgctatgaagtaatatttatgtagttaaaatgcttcattataactcttttaaatcctttacacactagtaaaacagatattactttaaataataattgatagacctggataactttcacaaacacatgattttttaatggtttttcttgagtgaagagaaaaacaatattatcaaatgaaataagtacttaaaatatcctgtctttcccatataacaatgatttttctgactttccatgagtaaaaaaacagccaagcatctttccagtagccccattgaaattgtgaatccgtcctggtctccctaaggactgcacacattgatattcaaggttggtggtcattagatatggaacagaactgaaataaccatggtagaactgaatgtgtaatgttggctttattctagctggtactacatggcacacagtttcaaaacataatttcacctactggaaagctcagacctgtaaaacagagcatgggaactgctggtctaaatgcagttgttcctgctcaaagagacctctggccaaactggcaagcagttaaagttttctttcagggccttcctctctatggcctcaacttcctcctctctcttcttccagcaacttcccctttcatcattcctttccctggggacttggcattcagtgatcctgtagatattgcacaactggggaacctttagacatccttaaaatcacatgagatagacagtcatttggggtgtctgaaataaaccaccccaaaacttagtgttaaaagagcaaccaaaaaaaatttatgtgagattatggatttgttacttagcttgatttaatcatcctgtaacgtgtacatatatcaaaatgttatgtataccataaatatataaaattttatcaacgaaattcataacaatctctcagaccacagagaaatcaaattagaactgaggactaagaaactcactcgaaaccacacaactacatggaaactgaacaacctgctcctgaatgactactgggtaaataatgaaattaaggcagaaataaataagttccttaaaaccaatgagaacaaagagacaacataccagaatctctaggagacagggctttgcttttgctgcattctattcgttgtgaacacaaattacaggccagtctcgattcagtgtagaagggaactgcataaggaccacataccaggaggcataattcactgggagcatctttagaaactaccagagttacctgttgcccataccagtggggtaagccctatgaatgtatatgagagtttcaaacatccacaaaacattggctttctaatattcgtattcccactattcctttcttttcatgattcatgtcattgtcccatcaacatttctaagatttccattccgttaagagcaaaagagaatgttggaaggtgggggaaaacatttctttgttttctacagggccagcttcttggatgtgtgtgatctgttcagttgcaaagggtcacatgctcagaaggaccgcatgctaaatttaatgctttgcagttaccctcttgaaatcctttattttttaagaaggaattcgacatttccatttttcaatgagccccacaaattacgcagctagtcctgggcttctctactctgaaattgggcaggatctctcttgatctagaatttactaaggcataataggggcaagaaaatcttatgaaataatggggggtagggaagagatgggaatggagcatgagatccagcttcgttattctctacttgagaaaaataaggccccaaagattaaacaacttgcccaaggatattgcttgttagtgtcagaactgaaaccagaaaccaaatgatcatatccctagacttttagtctgctttctcttccataaaatgaaacttataatgtttctaatccattgctcagacaggtagacatgaatattaattgataatgactattaattgatctggaaaatacttgtttggggatcaataatatgtttgggctattatctaatgctgtgtagaaatattaaaacccctgttattttgaaataaaaaagatacccactttt

SEQ ID NO: 67 - DNA, 1464 bp, human
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 68 - DNA, 1793 bp, human
cgcgtccgccccgcgagcacagagcctcgcctttgccgatccgccgcccgtccacacccgccgccagctcaccatggatgatgatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaagatcattgctcctcctgagcgcaagtactccgtgtggatcggcggctccatcctggcctcgctgtccaccttccagcagatgtggatcagcaagcaggagtatgacgagtccggcccctccatcgtccaccgcaaatgcttctaggcggactatgacttagttgcgttacaccctttcttgacaaaacctaacttgcgcagaaaacaagatgagattggcatggctttatttgttttttttgttttgttttggttttttttttttttttggcttgactcaggatttaaaaactggaacggtgaaggtgacagcagtcggttggagcgagcatcccccaaagttcacaatgtggccgaggactttgattgcacattgttgtttttttaatagtcattccaaatatgagatgcattgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttccccctttttgtcccccaacttgagatgtatgaaggcttttggtctccctgggagtgggtggaggcagccagggcttacctgtacactgacttgagaccagttgaataaaagtgcacacctta

SEQ ID NO: 69 - DNA, 1526 bp, human
ccggaagtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtgtaaaaaaaaaaaaaaaaa

SEQ ID NO: 70 - DNA, 2397 bp, human
gcaagaactgaaacgaatggggattgaactgctttgcctgttctttctatttctaggaaggaatgatcacgtacaaggtggctgtgccctgggaggtgcagaaacctgtgaagactgcctgcttattggacctcagtgtgcctggtgtgctcaggagaattttactcatccatctggagttggcgaaaggtgtgataccccagcaaaccttttagctaaaggatgtcaattaaacttcatcgaaaaccctgtctcccaagtagaaatacttaaaaataagcctctcagtgtaggcagacagaaaaatagttctgacattgttcagattgcgcctcaaagcttgatccttaagttgagaccaggtggtgcgcagactctgcaggtgcatgtccgccagactgaggactacccggtggatttgtattacctcatggacctctccgcctccatggatgacgacctcaacacaataaaggagctgggctcccggctttccaaagagatgtctaaattaaccagcaactttagactgggcttcggatcttttgtggaaaaacctgtatcccctttcgtgaaaacaacaccagaagaaattgccaacccttgcagtagtattccatacttctgtttacctacatttggattcaagcacattttgccattgacaaatgatgctgaaagattcaatgaaattgtgaagaatcagaaaatttctgctaatattgacacacccgaaggtggatttgatgcaattatgcaagctgctgtgtgtaaggaaaaaattggctggcggaatgactccctccacctcctggtctttgtgagtgatgctgattctcattttggaatggacagcaaactagcaggcatcgtcattcctaatgacgggctctgtcacttggacagcaagaatgaatactccatgtcaactgtcttggaatatccaacaattggacaactcattgataaactggtacaaaacaacgtgttattgatcttcgctgtaacccaagaacaagttcatttatatgagaattacgcaaaacttattcctggagctacagtaggtctacttcagaaggactccggaaacattctccagctgatcatctcagcttatgaagaactgcggtctgaggtggaactggaagtattaggagacactgaaggactcaacttgtcatttacagccatctgtaacaacggtaccctcttccaacaccaaaagaaatgctctcacatgaaagtgggagacacagcttccttcagcgtgactgtgaatatcccacactgcgagagaagaagcaggcacattatcataaagcctgtggggctgggggatgccctggaattacttgtcagcccagaatgcaactgcgactgtcagaaagaagtggaagtgaacagctccaaatgtcaccacgggaacggctctttccagtgtggggtgtgtgcctgccaccctggccacatggggcctcgctgtgagtgtggcgaggacatgctgagcacagattcctgcaaggaggccccagatcatccctcctgcagcggaaggggtgactgctactgtgggcagtgtatctgccacttgtctccctatggaaacatttatgggccttattgccagtgtgacaatttctcctgcgtgagacacaaagggctgctctgcggaggtaacggcgactgtgactgtggtgaatgtgtgtgcaggagcggctggactggcgagtactgcaactgcaccaccagcacggactcctgcgtctctgaagatggagtgctctgcagcgggcgcggggactgtgtttgtggcaagtgtgtttgcacaaaccctggagcctcaggaccaacctgtgaacgatgtcctacctgtggtgacccctgtaactctaaacggagctgcattgagtgccacctgtcagcagctggccaagcccgagaagaatgtgtggacaagtgcaaactagctggtgcgaccatcagtgaagaagaagatttctcaaaggatggttctgtttcctgctctctgcaaggagaaaatgaatgtcttattacattcctaataactacagataatgaggggaaaaccatcattcacagcatcaatgaaaaagattgtccgaagcctccaaacattcccatgatcatgttaggggtttccctggctattcttctcatcggggttgtcctactgtgcatctggaagctactggtgtcatttcatgatcgtaaagaagttgccaaatttgaagcagaacgatcaaaagccaagtggcaaacgggaaccaatccactctacagaggatccacaagtacttttaaaaatgtaacttataaacacagggaaaaacaaaaggtagacctttccacagattgctagaactactttatgca

SEQ ID NO: 71 - DNA, 3937 bp, human
ataagttagcgccgctctccgcctcggcagtgccagccgccagtggtcgcacttggagggtctcgccgccagtggaaggagccaccgcccccgcccgaccatggccgaggagctggtcttagagaggtgtgatctggagctggagaccaatggccgagaccaccacacggccgacctgtgccgggagaagctggtggtgcgacggggccagcccttctggctgaccctgcactttgagggccgcaactacgaggccagtgtagacagtctcaccttcagtgtcgtgaccggcccagcccctagccaggaggccgggaccaaggcccgttttccactaagagatgctgtggaggagggtgactggacagccaccgtggtggaccagcaagactgcaccctctcgctgcagctcaccaccccggccaacgcccccatcggcctgtatcgcctcagcctggaggcctccactggctaccagggatccagctttgtgctgggccacttcattttgctcttcaacgcctggtgcccagcggatgctgtgtacctggactcggaagaggagcggcaggagtatgtcctcacccagcagggctttatctaccagggctcggccaagttcatcaagaacataccttggaattttgggcagtttgaagatgggatcctagacatctgcctgatccttctagatgtcaaccccaagttcctgaagaacgccggccgtgactgctcccgccgcagcagccccgtctacgtgggccgggtggtgagtggcatggtcaactgcaacgatgaccagggtgtgctgctgggacgctgggacaacaactacggggacggcgtcagccccatgtcctggatcggcagcgtggacatcctgcggcgctggaagaaccacggctgccagcgcgtcaagtatggccagtgctgggtcttcgccgccgtggcctgcacagtgctgaggtgcctgggcatccctacccgcgtcgtgaccaactacaactcggcccatgaccagaacagcaaccttctcatcgagtacttccgcaatgagtttggggagatccagggtgacaagagcgagatgatctggaacttccactgctgggtggagtcgtggatgaccaggccggacctgcagccggggtacgagggctggcaggccctggacccaacgccccaggagaagagcgaagggacgtactgctgtggcccagttccagttcgtgccatcaaggagggcgacctgagcaccaagtacgatgcgccctttgtctttgcggaggtcaatgccgacgtggtagactggatccagcaggacgatgggtctgtgcacaaatccatcaaccgttccctgatcgttgggctgaagatcagcactaagagcgtgggccgagacgagcgggaggatatcacccacacctacaaatacccagaggggtcctcagaggagagggaggccttcacaagggcgaaccacctgaacaaactggccgagaaggaggagacagggatggccatgcggatccgtgtgggccagagcatgaacatgggcagtgactttgacgtctttgcccacatcaccaacaacaccgctgaggagtacgtctgccgcctcctgctctgtgcccgcaccgtcagctacaatgggatcttggggcccgagtgtggcaccaagtacctgctcaacctcaacctggagcctttctctgagaagagcgttcctctttgcatcctctatgagaaataccgtgactgccttacggagtccaacctcatcaaggtgcgggccctcctcgtggagccagttatcaacagctacctgctggctgagagggacctctacctggagaatccagaaatcaagatccggatccttggggagcccaagcagaaacgcaagctggtggctgaggtgtccctgcagaacccgctccctgtggccctggaaggctgcaccttcactgtggagggggccggcctgactgaggagcagaagacggtggagatcccagaccccgtggaggcaggggaggaagttaaggtgagaatggacctgctgccgctccacatgggcctccacaagctggtggtgaacttcgagagcgacaagctgaaggctgtgaagggcttccggaatgtcatcattggccccgcctaagggacccctgctcccagcctgctgagagcccccaccttgatcccaatccttatcccaagctagtgagcaaaatatgccccttcttgggccccagaccccagggcagggtgggcagcctatgggggctctcggaaatggaatgtgcccctggcccatctcagcctcctgagcctgtgggtccccactcaccccctttgctgtgaggaatgctctgtgccagaaacagtgggagccctgaccttggctgactggggctggggtgagagaggaaagacctacattccctctcctgcccagatgccctttggaaagccattgaccacccaccatattgtttgatctacttcatagctccttggagcaggcaaaaaagggacagcatgccccttggctggatcagggaatccagctccctagactgcatcccgtacctcttcccatgactgcacccagctccaggggcccttgggacagccagagctgggtggggacagtgataggcccaaggtcccctccacatcccagcagcccaagcttaatagccctccccctcaacctcaccattgtgaagcacctactatgtgctgggtgcctcccacacttgctggggctcacggggcctccaacccatttaatcaccatgggaaactgttgtgggcgctgcttccaggataaggagactgaggcttagagagaggaggcagccccctccacaccagtggcctcgtggttattagcaaggctgggtaatgtgaaggcccaagagcagagtctgggcctctgactctgagtccactgctccatttataaccccagcctgacctgagactgtcggagaggctgtctggggcctttatcaaaaaaagactcagccaagacaaggaggtagagaggggactgggggactgggagtcagagccctggctgggttcaggtcccacgtctggccaggcactgccttctcctctctgggcctttgtttccttgttggtcagaggagtgattgaaccagctcatctccaaggatcctctccactccatgtttgcaatgcttttatatggcccagccttgtaaataaccacaaggtccactccctgctccacgaagccttaagccataggcccaggatatttctgagagtgaaaccatgactgtgaccaccttctgtccccagccctgtcctggttccttcctatgcccaggtaccacccttcagaccccagttctaggggagaagagccctggacacccctgctctacccatgagcctgcccgctgcaatgcctagacttcccaacagccttagctgccagtgctggtcactaaccaacaaggttggcaccccagctaccccttctttgcagggctaaggcccccaaacatagcccctgccccggaggaagcttggggaacccatgagttgtcagctttgactttatctcctgctctttctacatgactgggcctcccttgggctggaagaattggggattctctattggaggtgagatcacagcctccagggccccccaaatcccagggaaggacttggagagaatcatgctgttgcatttagaactttctgctttgcacaggaaagagtcacacaattaatcaacatgtatattttctctatacatagagctctatttctctacggttttataaaagccttgggttccaaccaggcagtagatgtgcttctgaaccgcaaggagcaaacactgaaataaaatagtttatttttcacactcaaaaaaaaaaaaaaaaaa

SEQ ID NO: 72 - DNA, 489 bp, human
agagccgcaggtcagtcgtgaagagggagctctattgccaccatgagtttctccggcaagtaccaactgcagagccaggaaaactttgaagccttcatgaaggcaatcggtctgccggaagagctcatccagaaggggaaggatatcaagggggtgtcggaaatcgtgcagaatgggaagcacttcaagttcaccatcaccgctgggtccaaagtgatccaaaacgaattcacggtgggggaggaatgtgagctggagacaatgacaggggagaaagtcaagacagtggttcagttggaaggtgacaataaactggtgacaactttcaaaaacatcaagtctgtgaccgaactcaacggcgacataatcaccaataccatgacattgggtgacattgtcttcaagagaatcagcaagagaatttaaacaagtctgcatttcatattattttagtgtgtaaaattaatgtaataaagtgaactttgtttt

SEQ ID NO: 73 - DNA, 1772 bp, human
aggacagccgagttcaggtgagcggttgctcgtcgtcggggcggccggcagcggcggctccagggcccagcatgcgcgggggaccccgcggccaccatgtatgtgggctatgtgctggacaaggattcgcccgtgtaccccggcccagccaggccagccagcctcggcctgggcccgcaagcctacggccccccggccccgcccccggcgcccccgcagtaccccgacttctccagctactctcacgtggagccggcccccgcgcccccgacggcctggggggcgcccttccctgcgcccaaggacgactgggccgccgcctacggcccgggccccgcggcccctgccgccagcccagcttcgctggcattcgggccccctccagactttagcccggtgccggcgccccctgggcccggcccgggcctcctggcgcagcccctcgggggcccgggcacaccgtcctcgcccggagcgcagaggccgacgccctacgagtggatgcggcgcagcgtggcggccggaggcggcggtggcagcggtaagactcggaccaaggacaagtaccgcgtggtctacaccgaccaccaacgcctggagctggagaaggagtttcattacagccgttacatcacaatccggcggaaatcagagctggctgccaatctggggctcactgaacggcaggtgaagatctggttccaaaaccggcgggcaaaggagcgcaaagtgaacaagaagaaacagcagcagcaacagcccccacagccgccgatggcccacgacatcacggccaccccagccgggccatccctggggggcctgtgtcccagcaacaccagcctcctggccacctcctctccaatgcctgtgaaagaggagtttctgccatagccccatgcccagcctgtgcgccgggggacctggggactcgggtgctgggagtgtggctcctgtgggcccaggaggtctggtccgagtctcagccctgaccttctgggacatggtggacagtcacctatccaccctctgcatccccttggcccatctgtgcagtaagcctgttggataaagaccttccagctcctgtgttctagacctctgggggataagggagtccagggtggatgatctcaatctcccgtgggcatctcaagccccaaatggttgggggaggggcctagacaaggctccaggccccacctcctcctccatacgttcagaggtgcagctggaggctgctgtggggaccacactgatcctggagaaaagggatggagctgaaaaagatggaatgcttgcagagcatgacctgaggagggaggaacgtggtcaactcacacctgcctcttcctgcagcctcacttctacctgcccccatcataagggcactgagcccttcccaggctggatactaagcacaaagcccatagcactgggctctgatggctgctccactgggttacagaatcacagccctcatgatcattctcagtgagggctctggattgagagggaggccctgggaggagagaagggggcagagtcttccctaccaggtttctacacccccgccaggctgcccatcagggcccagggagcccccagaggactttattcggaccaagcagagctcacagctggacaggtgttgtatatagagtggaatctcttggatgcagcttcaagaataaatttttcttctcttttcaaaaatgtataaaaatcattatacatagcattaaagaaacatttttgagaagtacaaatca

SEQ ID NO: 74 - DNA, 3744 bp, human
gcaaagcaagtgggcacaaggagtatggttctaacgtgattggggtcatgaagacgttgctgttggacttggctttgtggtcactgctcttccagcccgggtggctgtcctttagttcccaggtgagtcagaactgccacaatggcagctatgaaatcagcgtcctgatgatgggcaactcagcctttgcagagcccctgaaaaacttggaagatgcggtgaatgaggggctggaaatagtgagaggacgtctgcaaaatgctggcctaaatgtgactgtgaacgctactttcatgtattcggatggtctgattcataactcaggcgactgccggagtagcacctgtgaaggcctcgacctactcaggaaaatttcaaatgcacaacggatgggctgtgtcctcatagggccctcatgtacatactccaccttccagatgtaccttgacacagaattgagctaccccatgatctcagctggaagttttggattgtcatgtgactataaagaaaccttaaccaggctgatgtctccagctagaaagttgatgtacttcttggttaacttttggaaaaccaacgatctgcccttcaaaacttattcctggagcacttcgtatgtttacaagaatggtacagaaactgaggactgtttctggtaccttaatgctctggaggctagcgtttcctatttctcccacgaactcggctttaaggtggtgttaagacaagataaggagtttcaggatatcttaatggaccacaacaggaaaagcaatgtgattattatgtgtggtggtccagagttcctctacaagctgaagggtgaccgagcagtggctgaagacattgtcattattctagtggatcttttcaatgaccagtacttggaggacaatgtcacagcccctgactatatgaaaaatgtccttgttctgacgctgtctcctgggaattcccttctaaatagctctttctccaggaatctatcaccaacaaaacgagactttgctcttgcctatttgaatggaatcctgctctttggacatatgctgaagatatttcttgaaaatggagaaaatattaccacccccaaatttgctcatgctttcaggaatctcacttttgaagggtatgacggtccagtgaccttggatgactggggggatgttgacagtaccatggtgcttctgtatacctctgtggacaccaagaaatacaaggttcttttgacctatgatacccacgtaaataagacctatcctgtggatatgagccccacattcacttggaagaactctaaacttcctaatgatattacaggccggggccctcagatcctgatgattgcagtcttcaccctcactggagctgtggtgctgctcctgctcgtcgctctcctgatgctcagaaaatatagaaaagattatgaacttcgtcagaaaaaatggtcccacattcctcctgaaaatatctttcctctggagaccaatgagaccaatcatgttagcctcaagatcgatgatgacaaaagacgagatacaatccagagactacgacagtgcaaatacgacaaaaagcgagtgattctcaaagatctcaagcacaatgatggtaatttcactgaaaaacagaagatagaattgaacaagttgcttcagattgactattacaacctgaccaagttctacggcacagtgaaacttgataccatgatcttcggggtgatagaatactgtgagagaggatccctccgggaagttttaaatgacacaatttcctaccctgatggcacattcatggattgggagtttaagatctctgtcttgtatgacattgctaagggaatgtcatatctgcactccagtaagacagaagtccatggtcgtctgaaatctaccaactgcgtagtggacagtagaatggtggtgaagatcactgattttggctgcaattccattttacctccaaaaaaggacctgtggacagctccagagcacctccgccaagccaacatctctcagaaaggagatgtgtacagctatgggatcatcgcacaggagatcattctgcggaaagaaaccttctacactttgagctgtcgggaccggaatgagaagattttcagagtggaaaattccaatggaatgaaacccttccgcccagatttattcttggaaacagcagaggaaaaagagctagaagtgtacctacttgtaaaaaactgttgggaggaagatccagaaaagagaccagatttcaaaaaaattgagactacacttgccaagatatttggactttttcatgaccaaaaaaatgaaagctatatggataccttgatccgacgtctacagctatattctcgaaacctggaacatctggtagaggaaaggacacagctgtacaaggcagagagggacagggctgacagacttaactttatgttgcttccaaggctagtggtaaagtctctgaaggagaaaggctttgtggagccggaactatatgaggaagttacaatctacttcagtgacattgtaggtttcactactatctgcaaatacagcacccccatggaagtggtggacatgcttaatgacatctataagagttttgaccacattgttgatcatcatgatgtctacaaggtggaaaccatcggtgatgcgtacatggtggctagtggtttgcctaagagaaatggcaatcggcatgcaatagacattgccaagatggccttggaaatcctcagcttcatggggacctttgagctggagcatcttcctggcctcccaatatggattcgcattggagttcactctggtccctgtgctgctggagttgtgggaatcaagatgcctcgttattgtctatttggagatacggtcaacacagcctctaggatggaatccactggcctccctttgagaattcacgtgagtggctccaccatagccatcctgaagagaactgagtgccagttcctttatgaagtgagaggagaaacatacttaaagggaagaggaaatgagactacctactggctgactgggatgaaggaccagaaattcaacctgccaacccctcctactgtggagaatcaacagcgtttgcaagcagaattttcagacatgattgccaactctttacagaaaagacaggcagcagggataagaagccaaaaacccagacgggtagccagctataaaaaaggcactctggaatacttgcagctgaataccacagacaaggagagcacctatttttaaacctaaatgaggtataaggactcacacaaattaaaatacagctgcactgaggcagcgacctcaagtgtcctgaaagcttacattttcctgagacctcaatgaagcagaaatgtacttaggcttggctgccctgtctggaacatggactttcttgcatgaatcagatgtgtgttctcagtgaaataactaccttccactctggaaccttattccagcagttgttccagggagcttctacctggaaaagaaaagaaatgaatagactatctagaacttgagaagattttattcttatttcatttattttttgtttgtttatttttatcgtttttgtttactggctttccttctgtattcataagattttttaaattgtcataattatattttaaatacccatcttcattaaagtatatttaactcataatttttgcagaaaatatgctatatattaggcaagaataaaagctaaagg

SEQ ID NO: 75 - DNA, 901 bp, human
agccccaaactcaccacctggccgtggacacctgtgtcagcatgtgggacctggttctctccatcgccttgtctgtggggtgcactggtgccgtgcccctcatccagtctcggattgtgggaggctgggagtgtgagaagcattcccaaccctggcaggtggctgtgtacagtcatggatgggcacactgtgggggtgtcctggtgcacccccagtgggtgctcacagctgcccattgcctaaagaagaatagccaggtctggctgggtcggcacaacctgtttgagcctgaagacacaggccagagggtccctgtcagccacagcttcccacacccgctctacaatatgagccttctgaagcatcaaagccttagaccagatgaagactccagccatgacctcatgctgcttcgcctgtcagagcctgccaagatcacagatgttgtgaaggtcctgggcctgcccacccaggagccagcactggggaccacctgctacgcctcaggctggggcagcatcgaaccagaggagttcttgcgccccaggagtcttcagtgtgtgagcctccatctcctgtccaatgacatgtgtgctagagcttactctgagaaggtgacagagttcatgttgtgtgctgggctctggacaggtggtaaagacacttgtgggggtgattctgggggtccacttgtctgtaatggtgtgcttcaaggtatcacatcatggggccctgagccatgtgccctgcctgaaaagcctgctgtgtacaccaaggtggtgcattaccggaagtggatcaaggacaccatcgcagccaacccctgagtgcccctgtcccacccctacctctagtaaatttaagtccacctcaaaaaaaaaaaaaaaaaaaaaaaaaaaaa

SEQ ID NO: 76 - DNA, 591 bp, human
cttctctgggacacattgccttctgttttctccagcatgcgcttgctccagctcctgttcagggccagccctgccaccctgctcctggttctctgcctgcagttgggggccaacaaagctcaggacaacactcggaagatcataataaagaattttgacattcccaagtcagtacgtccaaatgacgaagtcactgcagtgcttgcagttcaaacagaattgaaagaatgcatggtggttaaaacttacctcattagcagcatccctctacaaggtgcatttaactataagtatactgcctgcctatgtgacgacaatccaaaaaccttctactgggacttttacaccaacagaactgtgcaaattgcagccgtcgttgatgttattcgggaattaggcatctgccctgatgatgctgctgtaatccccatcaaaaacaaccggttttatactattgaaatcctaaaggtagaataatggaagccctgtctgtttgccacacccaggtgatttcctctaaagaaacttggctggaatttctgctgtggtctataaaataaacttcttaacatgcttaaaaaaaaaaaaaaa

SEQ ID NO: 77 - DNA, 570 bp, human
cacctgcaccccgcccgggcatagcaccatgcctgcttgtcgcctaggcccgctagccgccgccctcctcctcagcctgctgctgttcggcttcaccctagtctcaggcacaggagcagagaagactggcgtgtgccccgagctccaggctgaccagaactgcacgcaagagtgcgtctcggacagcgaatgcgccgacaacctcaagtgctgcagcgcgggctgtgccaccttctgctctctgcccaatgataaggagggttcctgcccccaggtgaacattaactttccccagctcggcctctgtcgggaccagtgccaggtggacagccagtgtcctggccagatgaaatgctgccgcaatggctgtgggaaggtgtcctgtgtcactcccaatttctgagctccagccaccaccaggctgagcagtgaggagagaaagtttctgcctggccctgcatctggttccagcccacctgccctcccctttttcgggactctgtattccctcttgggctgaccacagcttctccctttcccaaccaataaagtaaccactttcagcaaaaa

SEQ ID NO: 78 - DNA, 2698 bp, human
cttcagaaggaggagagacaccgggcccagggcaccctcgcgggcggacccaagcagtgagggcctgcagccggccggccagggcagcggcaggcgcggcccggacctacgggaggaagccccgagccctcggcgggctgcgagcgactccccggcgatgcctcacaactccatcagatctggccatggagggctgaaccagctgggaggggcctttgtgaatggcagacctctgccggaagtggtccgccagcgcatcgtagacctggcccaccagggtgtaaggccctgcgacatctctcgccagctccgcgtcagccatggctgcgtcagcaagatccttggcaggtactacgagactggcagcatccggcctggagtgatagggggctccaagcccaaggtggccacccccaaggtggtggagaagattggggactacaaacgccagaaccctaccatgtttgcctgggagatccgagaccggctcctggctgagggcgtctgtgacaatgacactgtgcccagtgtcagctccattaatagaatcatccggaccaaagtgcagcaaccattcaacctccctatggacagctgcgtggccaccaagtccctgagtcccggacacacgctgatccccagctcagctgtaactcccccggagtcaccccagtcggattccctgggctccacctactccatcaatgggctcctgggcatcgctcagcctggcagcgacaagaggaaaatggatgacagtgatcaggatagctgccgactaagcattgactcacagagcagcagcagcggaccccgaaagcaccttcgcacggatgccttcagccagcaccacctcgagccgctcgagtgcccatttgagcggcagcactacccagaggcctatgcctcccccagccacaccaaaggcgagcagggcctctacccgctgcccttgctcaacagcaccctggacgacgggaaggccaccctgaccccttccaacacgccactggggcgcaacctctcgactcaccagacctaccccgtggtggcagatcctcactcacccttcgccataaagcaggaaacccccgaggtgtccagttctagctccaccccttcctctttatctagctccgcctttttggatctgcagcaagtcggctccggggtcccgcccttcaatgcctttccccatgctgcctccgtgtacgggcagttcacgggccaggccctcctctcagggcgagagatggtggggcccacgctgcccggatacccaccccacatccccaccagcggacagggcagctatgcctcctctgccatcgcaggcatggtggcaggaagtgaatactctggcaatgcctatggccacaccccctactcctcctacagcgaggcctggcgcttccccaactccagcttgctgagttccccatattattacagttccacatcaaggccgagtgcaccgcccaccactgccacggcctttgaccatctgtagttgccatggggacagtgggagcgactgagcaacaggaggactcagcctgggacaggccccagagagtcacacaaaggaatctttatttattacatgaaaaataaccacaagtccagcattgcggcacactccctgtgtggttaatttaatgaaccatgaaagacaggatgaccttggacaaggccaaactgtcctccaagactccttaatgaggggcaggagtcccagggaaagagaaccatgccatgctgaaaaagacaaaattgaagaagaaatgtagcccccagccggtacccaccaaaggagagaagaagcaatagccgaggaacttggggggatggcgaatggttcctgcccgggcccaaggggtgcacagggcacctccatggctccattattaacacaactctagcaattatggaccataagcacttccctccagcccacaagtcacagcctggtgccgaggctctcctcaccagccacccagggagtcacctccctcagcctcccgcctgccccacacggaggctctggctgtcctctttctccactccatttgcttggctctttctacacctccctcttgggcatgggctgagggctggagcgagtccctcagaaattccaccaggctgtcagctgacctcttttgcctgctgctgtgaaggtatagcaccaccccaggtcctcctgcagtgcggcatccccttggcagctgccgtcagccaggccagccccagggagcttaaaacagacattccacagggcctgggcccctgggaggtgaggtgtggtgtgcggcttcacccagggcagaacaaggcagaatcgcaggaaacccgcttccccttcctgacagctcctgccaagccaaatgtgcttcctgcagctcacgcccaccagctactgaagggacccaaggcaccccctgaagccagcgatagagggtccctctctgctccccagcagctcctgcccccaaggcctgactgtatatactgtcaatgaaactttgtttgggtcaagcttccttctttctaccccccagactttggcctctgagtgaaatgtctctctttgccctgtggggcttctctccttgatgcttctttctttttttaaagacaacctgccattaccacatgactcaataaaccattgctcttcaaaaaaaaaaaaaaaaaa

SEQ ID NO: 79 - DNA, 2983 bp, human
gcccagataggggagcggaggtggcggcggcggcggtagcggtggccttggttgtcttccagtctcctcggctcgccctttagccggcaccgctccccttccctcccccttcctctcttccttccttccctccccttccctttttcccttccccgtcggtgagcggcgggggtggctccagcaacggctgggcccaagctgtgtagaggccttaaccaacgataacggcggcgacggcgaaacctcggagctcgcagggcgggggcaaggcccgggccttggagatggagaattctcagttgtgtaagctgttcatcggcggcctcaatgtgcagacgagtgagtcgggcctgcgcggccactttgaggcctttgggactctgacggactgcgtggtggtggtgaatccccagaccaagcgctcccgttgctttggcttcgtgacctactccaatgtggaggaggcggacgccgccatggccgcctcgccccatgccgtggacggcaacactgtggagctgaagcgggcggtgtcccgggaggattcggcgcggcccggtgcccacgccaaggttaagaagctctttgtcggaggccttaaaggagacgtggctgagggcgacctgatcgagcacttctcgcagtttggcaccgtggaaaaggccgagattattgccgacaagcagtccggcaagaagcgtggattcggcttcgtgtatttccagaatcacgacgcggcagacaaggccgcggtggtcaagttccatccgattcagggccatcgcgtggaggtgaagaaagcagtccccaaggaggatatctactccggtgggggtggaggcggctcccgatcctcccggggcggccgaggcggccgggggcgcggcggtggtcgagaccagaacggcctttccaagggcggcggcggcggttacaacagctacggtggttacggcggcggcggaggcggcggctacaatgcctacggaggcggcggcggcggttcgtcctacggtgggagcgactacggtaacggcttcggcggcttcggcagctacagccagcatcagtcctcctatgggcccatgaagagcggcggcggcggcggcggtggaggcagtagctggggcggtcgcagtaatagtggaccttacagaggcggctatggcggtgggggtggctatggaggcagctccttctaaaagaaaatttaaaatgcctgggagtggctataggggtagctctttccaacagcccaagtggggtcaactcctaagccccaccccctcacacacaccgccttccctgttttgcccttgggggagccacttctaaggctgcttacccttgggggtgttcctctatttgcctgccacctctcttgtctctccctctgaagatggactcggccccacatacacatttttgtgttacagtcattgatggactctatttttttattattacttggaccttggtcgtttttatactagcaaaatgtcttgttttaatttgtgttttttggggggagggagggagtgaacttgctgattctgtagcaaaacctgggtgggggttggggtggggggtagtttactttgttgtaaggacttgataacctggctacagcgttttctatgaaatctacttggatcccatgcctgaaatttggaagcatatgtacaaaaatcatttttacgttttatttttaataaatcattgtgtttgaccgtacatgtctaacattttttttctaggatccattccgtaccgttttttaagggatatttgtttaagactttacgtgttaattctttattcttgatgtgtacttagagaaacttaagaggtcctgtggtttttttcccctctcctgttgccctgctagttgcgtgttgaattatatcccttacaggcaaaacttttgaagtggtggatgtggctttttaaactcttaagtttctgtgcatccatctcttgtactaagcgaattgtttatcatcttgacatggttggtcatttctatgacaatttacttcaaactgtgtactgtgtagttctatatagtttgtgttaagcatgtcattcatataaactgtttaaaatttttcagatggcctagtttcatccctcttactggtttgtctgtaatgaatggttaaaaataagggttatattttaccctcaaatgcgtttttgtactttcagagcaggtttaaacgttttttttttttttttcctatatccgaactgttggcctcatggaaatccctttcccgatctttgtagcaccatctactggcagaatggcagagtagctgcgaaacaatttgtttaaaaacttgcttaagacaattgcatcagatttggaagttttgccatcaaaattctttgcagaattggaagttaacacatttgcttgtaactgagatgggcttcacaggaatgtagttgccagttcatatcacaatagccctttctatatgaggtttgaaaatgtaaactgctatgcatagcttgggcaatagccctaaattgctatgacaactaatgaaccagctacgtatactggtattttaggtgcaagttgtaaagcaaaatatctgtgtattctgcttggttaacaaatgtatatttgtagccctttcctgcaatagcattcaagttgttgtttataagagaagaacaaaagtgataataggtgaaaattgcctttctggatagaaatagagaatagcaacgtttatggatatcacaaataaagaattcaattctttacatgattgagtgagagtatgtataacctggtgggtgggttcagagtaccttttaatctagtatgcttaacttgatgttaatatttaacttaaatatttgacttacatgttgacgttgaaggctcaaagctatactaagaagctttctgaaagattgggctttaaaataaaataatattttaatattgaaaaaaaaaaaaaaaaaa

SEQ ID NO: 80 - DNA, 3345 bp, human
gaattccgtctcgaccactgaatggaagaaaaggacttttaaccaccattttgtgacttacagaaaggaatttgaataaagaaaactatgatacttcaggcccatcttcactccctgtgtcttcttatgctttatttggcaactggatatggccaagaggggaagtttagtggacccctgaaacccatgacattttctatttatgaaggccaagaaccgagtcaaattatattccagtttaaggccaatcctcctgctgtgacttttgaactaactggggagacagacaacatatttgtgatagaacgggagggacttctgtattacaacagagccttggacagggaaacaagatctactcacaatctccaggttgcagccctggacgctaatggaattatagtggagggtccagtccctatcaccatagaagtgaaggacatcaacgacaatcgacccacgtttctccagtcaaagtacgaaggctcagtaaggcagaactctcgcccaggaaagcccttcttgtatgtcaatgccacagacctggatgatccggccactcccaatggccagctttattaccagattgtcatccagcttcccatgatcaacaatgtcatgtactttcagatcaacaacaaaacgggagccatctctcttacccgagagggatctcaggaattgaatcctgctaagaatccttcctataatctggtgatctcagtgaaggacatgggaggccagagtgagaattccttcagtgataccacatctgtggatatcatagtgacagagaatatttggaaagcaccaaaacctgtggagatggtggaaaactcaactgatcctcaccccatcaaaatcactcaggtgcggtggaatgatcccggtgcacaatattccttagttgacaaagagaagctgccaagattcccattttcaattgaccaggaaggagatatttacgtgactcagcccttggaccgagaagaaaaggatgcatatgttttttatgcagttgcaaaggatgagtacggaaaaccactttcatatccgctggaaattcatgtaaaagttaaagatattaatgataatccacctacatgtccgtcaccagtaaccgtatttgaggtccaggagaatgaacgactgggtaacagtatcgggacccttactgcacatgacagggatgaagaaaatactgccaacagttttctaaactacaggattgtggagcaaactcccaaacttcccatggatggactcttcctaatccaaacctatgctggaatgttacagttagctaaacagtccttgaagaagcaagatactcctcagtacaacttaacgatagaggtgtctgacaaagatttcaagaccctttgttttgtgcaaatcaacgttattgatatcaatgatcagatccccatctttgaaaaatcagattatggaaacctgactcttgctgaagacacaaacattgggtccaccatcttaaccatccaggccactgatgctgatgagccatttactgggagttctaaaattctgtatcatatcataaagggagacagtgagggacgcctgggggttgacacagatccccataccaacaccggatatgtcataattaaaaagcctcttgattttgaaacagcagctgtttccaacattgtgttcaaagcagaaaatcctgagcctctagtgtttggtgtgaagtacaatgcaagttcttttgccaagttcacgcttattgtgacagatgtgaatgaagcacctcaattttcccaacacgtattccaagcgaaagtcagtgaggatgtagctataggcactaaagtgggcaatgtgactgccaaggatccagaaggtctggacataagctattcactgaggggagacacaagaggttggcttaaaattgaccacgtgactggtgagatctttagtgtggctccattggacagagaagccggaagtccatatcgggtacaagtggtggccacagaagtaggggggtcttccttaagctctgtgtcagagttccacctgatccttatggatgtgaatgacaaccctcccaggctagccaaggactacacgggcttgttcttctgccatcccctcagtgcacctggaagtctcattttcgaggctactgatgatgatcagcacttatttcggggtccccattttacattttccctcggcagtggaagcttacaaaacgactgggaagtttccaaaatcaatggtactcatgcccgactgtctaccaggcacacagactttgaggagagggcgtatgtcgtcttgatccgcatcaatgatgggggtcggccacccttggaaggcattgtttctttaccagttacattctgcagttgtgtggaaggaagttgtttccggccagcaggtcaccagactgggatacccactgtgggcatggcagttggtatactgctgaccacccttctggtgattggtataattttagcagttgtgtttatccgcataaagaaggataaaggcaaagataatgttgaaagtgctcaagcatctgaagtcaaacctctgagaagctgaatttgaaaaggaatgtttgaatttatatagcaagtgctatttcagcaacaaccatctcatcctattacttttcatctaacgtgcattataattttttaaacagatattccctcttgtcctttaatatttgctaaatatttcttttttgaggtggagtcttgctctgtcgcccaggctggagtacagtggtgtgatcccagctcactgcaacctccgcctcctgggttcacatgattctcctgcctcagcttcctaagtagctgggtttacaggcacccaccaccatgcccagctaatttttgtatttttaatagagacggggtttcgccatttggccaggctggtcttgaactcctgacgtcaagtgatctgcctgccttggtctcccaatacaggcatgaaccactgcacccacctacttagatatttcatgtgctatagacattagagagatttttcatttttccatgacatttttcctctctgcaaatggcttagctacttgtgtttttcccttttggggcaagacagactcattaaatattctgtacattttttctttatcaaggagatatatcagtgttgtctcatagaactgcctggattccatttatgttttttctgattccatcctgtgtccccttcatccttgactcctttggtatttcactgaatttcaaacatttgtcagagaagaaaaaagtgaggactcaggaaaaataaataaataaaagaacagccttttgcggccgcgaattc

SEQ ID NO: 81 - DNA, 990 bp, human
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttctta

SEQ ID NO: 82 - DNA, 1840 bp, human
gcgcacactctcctaagccctctcatctcctggaaccatggccagcacatccaccaccatcaggagccacagcagcagccgccggggtttcagtgccaactcagccaggctccctggggtcagccgctctggcttcagcagcatctccgtgtcccgctccaggggcagtggtggcctgggtggcgcatgtggaggagctggctttggcagccgcagtctgtatggcctggggggctccaagaggatctccattggagggggcagctgtgccatcagtggcggctatggcagcagagccggaggcagctatggctttggtggcgccgggagtggatttggtttcggtggtggagccggcattggctttggtctgggtggtggagccggccttgctggtggctttgggggccctggcttccctgtgtgcccccctggaggcatccaagaggtcactgtcaaccagagtctcctgactcccctcaacctgcaaattgaccccgccatccagcgggtgcgggccgaggagcgtgagcagatcaagaccctcaacaacaagtttgcctccttcatcgacaaggtgcggttcctagagcagcagaacaaggttctggacaccaagtggaccctgctgcaggagcagggcaccaagactgtgaggcagaacctggagccgttgttcgagcagtacatcaacaacctcaggaggcagctggacaacatcgtgggggaacggggtcgtctggactcggagctgagaaacatgcaggacctggtggaggacctcaagaacaaatatgaggatgaaatcaacaagcgcacagcagcagagaatgaatttgtgactctgaagaaggatgtggatgctgcctacatgaacaaggttgaactgcaagccaaggcagacactcttacagatgagatcaacttcctgagagccttgtatgatgcagagctgtcccagatgcagacccacatctcagacacatccgtggtgctatccatggacaacaaccgcaacctggacctggacagcatcatcgctgaggtcaaggcccaatatgaggagattgctcagaggagcagggctgaggctgagtcctggtaccagacaaagtacgaggagctgcagatcacagcaggcagacatggggacgacctgcgcaacaccaagcaggagattgctgagatcaaccgcatgatccagaggctgagatctgagatcgaccacgtcaagaagcagtgtgccaacctacaggccgccattgctgatgctgagcagcgtggggagatggccctcaaggatgctaagaacaagctggaagggctggaggatgccctgcagaaggccaagcaggacctggcccggctgctgaaggagtaccaggagctgatgaacgtcaagctggccctggacgtggagatcgccacctaccgcaagctgctggagggcgaggagtgcaggctgaatggcgaaggcgttggacaagtcaacatctctgtagtgcagtccaccgtctccagtggctatggcggtgccagcggtgtcggcagtggcttaggcctgggtggaggaagcagctactcctatggcagtggtcttggcgttggaggcggctttagttccagcagcggcagagccactgggggtggcctcagctctgttggaggcggcagttccaccatcaagtacaccaccacctcctcctccagcaggaagagctacaagcactgaagtcgtgccgccagctctcagtcccacagctctcaggcccctctctggcagcagagccctctcctcaggttgcttgtcctcccctggcctccagtctcccctgccctc

SEQ ID NO: 83 - DNA, 2820 bp, human
tggcaaaatcctggagccagaagaaaggacagcagcattgatcaatcttacagctaacatgttgtacctggaaaacaatgcccagactcaatttagtgagccacagtacacgaacctggggctcctgaacagcatggaccagcagattcggaacggctcctcgtccaccagtccctataacacagaccacgcgcagaacagcgtcacggcgccctcgccctacgcacagcccagccccaccttcgatgctctctctccatcacccgccatcccctccaacaccgactacccaggcccgcacagttccgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgagttcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtgagcctcaccatgtgagctcttcctatccctctcctaactgccagccccctaaaagcactcctgcttaatcttcaaagccttctccctagctcctccccttcctcttgtctgatttcttaggggaaggagaagtaagaggctacctcttacctaacatctgacctggcatctaattctgattctggctttaagccttcaaaactatagcttgcagaactgtagctgccatggctaggtagaagtgagcaaaaaagagttgggtgtctccttaagctgcagagatttctcattgacttttataaagcatgttcacccttatagtctaagactatatatataaatgtataaatatacagtatagatttttgggtggggggcattgagtattgtttaaaatgtaatttaaatgaaagaaaattgagttgcacttattgaccattttttaatttacttgttttggatggcttgtctatactccttcccttaaggggtatcatgtatggtgataggtatctagagcttaatgctacatgtgagtgcgatgatgtacagattctttcagttctttggattctaaatacatgccacatcaaacctttgagtagatccatttccattgcttattatgtaggtaagactgtagatatgtattcttttctcagtgttggtatattttatattactgacatttcttctagtgatgatggttcacgttggggtgatttaatccagttataagaagaagttcatgtccaaacggtcctctttagtttttggttgggaatgaggaaaattcttaaaaggcccatagcagccagttcaaaaacacccgacgtcatgtatttgagcatatcagtaacccccttaaatttaatacccagataccttatcttacaatgttgattgggaaaacatttgctgcccattacagaggtattaaaactaaatttcactactagattgactaactcaaatacacatttgctactgttgtaagaattc

SEQ ID NO: 84 - DNA, 989 bp, human
ttctggagggccaggaacaaacaggcttcaaagccaagggcttggctggcacacagggggcttggtccttcacctctgtcccctctccctacggacacatataagaccctggtcacacctgggagaggaggagaggagagcatagcacctgcagcaagatggatgtgggcagcaaagaggtcctgatggagagcccgccggactactccgcagctccccggggccgatttggcattccctgctgcccagtgcacctgaaacgccttcttatcgtggtggtggtggtggtcctcatcgtcgtggtgattgtgggagccctgctcatgggtctccacatgagccagaaacacacggagatggttctggagatgagcattggggcgccggaagcccagcaacgcctggccctgagtgagcacctggttaccactgccaccttctccatcggctccactggcctcgtggtgtatgactaccagcagctgctgatcgcctacaagccagcccctggcacctgctgctacatcatgaagatagctccagagagcatccccagtcttgaggctctcactagaaaagtccacaacttccagatggaatgctctctgcaggccaagcccgcagtgcctacgtctaagctgggccaggcagaggggcgagatgcaggctcagcaccctccggaggggacccggccttcctgggcatggccgtgagcaccctgtgtggcgaggtgccgctctactacatctaggacgcctccggtgagcagggtcagtggaagccccaacgggaaaggaaacgccccgggcaaagggtcttttgcagcttttgcagacgggcaagaagctgcttctgcccacaccgcagggacaagccctggagaaatgggagcttggggagaggatgggagtgggcagaggtggcgcccaggggcccgggaactcctgccacaacagaataaagcagcctgattgaaaagcaaaaaaaa

SEQ ID NO: 85 - DNA, 3123 bp, human
catcctgccacccctagccttgctggggacgtgaaccctctccccgcgcctgggaagccttcttggcaccgggacccggagaatccccacggaagccagttccaaaagggatgaaaagggggcgtttcgggcactgggagaagcctgtattccagggcccctcccagagcaggaatctgggacccaggagtgccagcctcacccacgcagatcctggccatgagagctccgcacctccacctctccgccgcctctggcgcccgggctctggcgaagctgctgccgctgctgatggcgcaactctgggccgcagaggcggcgctgctcccccaaaacgacacgcgcttggaccccgaagcctatggctccccgtgcgcgcgcggctcgcagccctggcaggtctcgctcttcaacggcctctcgttccactgcgcgggtgtcctggtggaccagagttgggtgctgacggccgcgcactgcggaaacaagccactgtgggctcgagtaggggatgaccacctgctgcttcttcagggagagcagctccgccggaccactcgctctgttgtccatcccaagtaccaccagggctcaggccccatcctgccaaggcgaacggatgagcacgatctcatgttgctgaagctggccaggcccgtagtgctggggccccgcgtccgggccctgcagcttccctaccgctgtgctcagcccggagaccagtgccaggttgctggctggggcaccacggccgcccggagagtgaagtacaacaagggcctgacctgctccagcatcactatcctgagccctaaagagtgtgaggtcttctaccctggcgtggtcaccaacaacatgatatgtgctggactggaccggggccaggacccttgccagagtgactctggaggccccctggtctgtgacgagaccctccaaggcatcctctcgtggggtgtttacccctgtggctctgcccagcatccagctgtctacacccagatctgcaaatacatgtcctggatcaataaagtcatacgctccaactgatccagatgctacgctccagctgatccagatgttatgctcctgctgatccagatgcccagaggctccatcgtccatcctcttcctccccagtcggctgaactctccccttgtctgcactgttcaaacctctgccgccctccacacctctaaacatctcccctctcacctcattcccccacctatccccattctctgcctgtactgaagctgaaatgcaggaagtggtggcaaaggtttattccagagaagccaggaagccggtcatcacccagcctctgagagcagttactggggtcacccaacctgacttcctctgccactccctgctgtgtgactttgggcaagccaagtgccctctctgaacctcagtttcctcatctgcaaaatgggaacaatgacgtgcctacctcttagacatgttgtgaggagactatgatataacatgtgtatgtaaatcttcatggtgattgtcatgtaaggcttaacacagtgggtggtgagttctgactaaaggttacctgttgtcgtgatctgaccacgtcccggtgaaagcgtgtgtccagggaagaagtgcacagggtagcccccagtcccaaccttccatccccaacccttagggatgatggaagaatcattttcctcaccctagttccaagtcccaggaaacaccttttaaccacttccttctcatctcccactgtttcccacttctggttccacccaacaccagttcctccgagctaggctggccctgagtcattagcaccttctctgccttcatgaggccactgaactcaagggacctcaccttgcttctatcccagcctctaagaccagagggccgagggggtagtgaagattgggaaacccttgccacctcaactgcccagcttgtgccaagaaacctccctctgacatttaggggaaaatctttggtttgtctgttattaattggctgtgagattttcgtcctgaaaacttgggaggaggaattgtttgattctccctgaaattgggggagggaaggggagttaatacacagaatccaggtaaggctaatagaagcttcagtgtccgctgggtgtggtggctcacgcctgtaatcctagcactttgggaggccgaggaaagcagatcatctgaggtcaggtgttcgagaccagtctggccaaaatggtgaaatcccatctctactgaaaatacaaaaactagccaggcatggtggcaggcacctataatcccatctacttgggaggctgaggcagaagaatcacttgaacccggggggcagaggttgcagtgagccaagattgcaccattgcactccatccagcctggacaaaagagcaaaactccatctcaaaaaaagaagaagtttgagtgtctcagcttatcctgaactttgaagcagtaacataagctagctgaatataggcacaacagaaatttcccattaagcacggggtttttgtttgtttgtttgtcctttcagttaccaatttatggagcatctattatgtgccaggcccagtgctgggtgctggggacatacgtaggggtgatcacaaggtcccccaccctgcagagcccacaggaggttgatgtacaaggtctgagcacacagctcccttccgtggcctctttcccattctgcccccattagcacacgcaggaaatgtcagacaggcgtatcagctgggtttgtcatccaaagaccagaagcgaggtcggtggaaacttgaaaactcgattcattattaaaggcaactcacccgtccctttagtcactccacaatgtttatcgagccacgtcttatgccaggcctcaaagatgaatcagaccaggcactaccctcaaggagctgctagttaggtcagggagacaggccagtcttacattcctgtcattcaggtctccactcaaaagtcacctcctccgggaggccttcctgaattgcccaggctataggagtccacttttggtcatccagtcccgatgctctgttttttgtttgtttgtttttttttttaatagcacctattatacctgaaattaaaaaaaaaaaaaaaaaa

SEQ ID NO: 86 - DNA, 3359 bp, human
cacaccttcggcagcaggagggcggcagcttctcgcaggcggcagggcgggcggccaggatcatgtccaccaccacatgccaagtggtggcgttcctcctgtccatcctggggctggccggctgcatcgcggccaccgggatggacatgtggagcacccaggacctgtacgacaaccccgtcacctccgtgttccagtacgaagggctctggaggagctgcgtgaggcagagttcaggcttcaccgaatgcaggccctatttcaccatcctgggacttccagccatgctgcaggcagtgcgagccctgatgatcgtaggcatcgtcctgggtgccattggcctcctggtatccatctttgccctgaaatgcatccgcattggcagcatggaggactctgccaaagccaacatgacactgacctccgggatcatgttcattgtctcaggtctttgtgcaattgctggagtgtctgtgtttgccaacatgctggtgactaacttctggatgtccacagctaacatgtacaccggcatgggtgggatggtgcagactgttcagaccaggtacacatttggtgcggctctgttcgtgggctgggtcgctggaggcctcacactaattgggggtgtgatgatgtgcatcgcctgccggggcctggcaccagaagaaaccaactacaaagccgtttcttatcatgcctcaggccacagtgttgcctacaagcctggaggcttcaaggccagcactggctttgggtccaacaccaaaaacaagaagatatacgatggaggtgcccgcacagaggacgaggtacaatcttatccttccaagcacgactatgtgtaatgctctaagacctctcagcacgggcggaagaaactcccggagagctcacccaaaaaacaaggagatcccatctagatttcttcttgcttttgactcacagctggaagttagaaaagcctcgatttcatctttggagaggccaaatggtcttagcctcagtctctgtctctaaatattccaccataaaacagctgagttatttatgaattagaggctatagctcacattttcaatcctctatttctttttttaaatataactttctactctgatgagagaatgtggttttaatctctctctcacattttgatgatttagacagactccccctcttcctcctagtcaataaacccattgatgatctatttcccagcttatccccaagaaaacttttgaaaggaaagagtagacccaaagatgttattttctgctgtttgaattttgtctccccacccccaacttggctagtaataaacacttactgaagaagaagcaataagagaaagatatttgtaatctctccagcccatgatctcggttttcttacactgtgatcttaaaagttaccaaaccaaagtcattttcagtttgaggcaaccaaacctttctactgctgttgacatcttcttattacagcaacaccattctaggagtttcctgagctctccactggagtcctctttctgtcgcgggtcagaaattgtccctagatgaatgagaaaattattttttttaatttaagtcctaaatatagttaaaataaataatgttttagtaaaatgatacactatctctgtgaaatagcctcacccctacatgtggatagaaggaaatgaaaaaataattgctttgacattgtctatatggtactttgtaaagtcatgcttaagtacaaattccatgaaaagctcactgatcctaattctttccctttgaggtctctatggctctgattgtacatgatagtaagtgtaagccatgtaaaaagtaaataatgtctgggcacagtggctcacgcctgtaatcctagcactttgggaggctgaggaggaaggatcacttgagcccagaagttcgagactagcctgggcaacatggagaagccctgtctctacaaaatacagagagaaaaaatcagccagtcatggtggcctacacctgtagtcccagcattccgggaggctgaggtgggaggatcacttgagcccagggaggttggggctgcagtgagccatgatcacaccactgcactccagccaggtgacatagcgagatcctgtctaaaaaaataaaaaataaataatggaacacagcaagtcctaggaagtaggttaaaactaattctttaaaaaaaaaaaaaagttgagcctgaattaaatgtaatgtttccaagtgacaggtatccacatttgcatggttacaagccactgccagttagcagtagcactttcctggcactgtggtcggttttgttttgttttgctttgtttagagacggggtctcactttccaggctggcctcaaactcctgcactcaagcaattcttctaccctggcctcccaagtagctggaattacaggtgtgcgccatcacaactagctggtggtcagttttgttactctgagagctgttcacttctctgaattcacctagagtggttggaccatcagatgtttgggcaaaactgaaagctctttgcaaccacacaccttccctgagcttacatcactgcccttttgagcagaaagtctaaattccttccaagacagtagaattccatcccagtaccaaagccagataggccccctaggaaactgaggtaagagcagtctctaaaaactacccacagcagcattggtgcaggggaacttggccattaggttattatttgagaggaaagtcctcacatcaatagtacatatgaaagtgacctccaaggggattggtgaatactcataaggatcttcaggctgaacagactatgtctggggaaagaacggattatgccccattaaataacaagttgtgttcaagagtcagagcagtgagctcagaggcccttctcactgagacagcaacatttaaaccaaaccagaggaagtatttgtggaactcactgcctcagtttgggtaaaggatgagcagacaagtcaactaaagaaaaaagaaaagcaaggaggagggttgagcaatctagagcatggagtttgttaagtgctctctggatttgagttgaagagcatccatttgagttgaaggccacagggcacaatgagctctcccttctaccaccagaaagtccctggtcaggtctcaggtagtgcggtgtggctcagctgggtttttaattagcgcattctctatccaacatttaattgtttgaaagcctccatatagttagattgtgctttgtaattttgttgttgttgctctatcttattgtatatgcattgagtattaacctgaatgttttgttacttaaatattaaaaacactgttatcctacagtt

SEQ ID NO: 87 - DNA, 733 bp, human
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 88 - DNA, 195514 bp, human
gaattcctttctcctctctccccatcttctctttgggactttgatatagtagttatgggatgggatctgggaatctgaattttaaacaaacagatgcttagagaaccatactgtccttagagaaacctgttctaaagccatagtctttttaaatatgaagtttgcacaaaaaattcattaggatggattaaagaaatagaactttagttatttttcctaataaaataataaatttcagtttcactttaatagcatttaactacttgagctggccccctctctgagttcttatgtcaaatggccaattcacacgcatggtccaacagatattcttttttttttattatactttaagttttagggtacatgtgcacaatgtgcaggttagttatatatgtatacatgtgccatgttggtgtgctgcacccattaactcatcatttaacattagatatatctcctaatgctatcccaaccccctccccccaccccacaacaggccccggtgtgtgagttccccttcctgtgtccatgtgttctcattgttcaattcccacctatgagtgagaacatgaggtgtttggttttttgaccttgcaatagtttactgagaatgatggtttccagcttcatccctgtccctacaaaggacatcaactcatcattttttatggctgcatagtattccatggtgtatatgtgccacattttcttaatccagtctatcattgttggacatttgggttggttccaagtctttgctattgtgaatagtgccacaataaacatatgtgtgcatgtgtctttatagcagcatgatttataatcctttgggtatatacccagtaatgggatggctgggtcaaatggtatttctagttctagatccctgaggaatcgccacactgacttccactaatggttgaactagtttacagtcccaccaacagtgtaaaagtgttcctatttctccacatcctctccagcacctgttgtttcctgagtttttaacgatcgccattctaactggtgtgagatggtatctcattgtggttttgatttgcatttctctgatggccagtgatggtgagcattttttcatgtgtcttttggctgcataaatgtcttcttttgagaagtgtctgttcatatccttcacccactttttgatggggctgtttttttcttgtaaatttgtttgagttcattgtagattctggatattagccctttgtcagatgagtagattgcaaaaattttctcccattctgtaggttgcctccaacagatattcttgaaagaaagtgtaggtggcacctggtcccctcaccagtgtgtttgctctttaaacactgcatggcaatttccaattatgtgggttataccatatagttgtcacgaaaggaatcctcacaaaatgaacaagtggcatgattatattcctgaagagaagccacagattgaagacagagcactactagatgatgcttttgctatttgtagttcaaggaaaagatttcatcaatcacattatgagtcaaacacaaggatgatctaatcagatatgaaaagcattggctcagtaattttaagtgatcagaaatgttactggaaaatagagatccatacacagtatcagcaatagcaaatcttatcttaattggatattgtacctgcgaattaactgataaaaggatgaggtcggccgggcgcggtggctcaggcctgtaatcccagcactttgggatgctgaagcgggcggatcagaaggtcaggagatcgagaccatcctggctaacatggtgaaaccccgtctctactgaaaatacaaaaaattagccgggcatggtggaaggcacctgtagtcccagctacttgggaggctgaggcgggagaatggcatgaacccgggaggcggagcttgcagtgagcggagatcaggccactgcactccagcctgggtgacagagcgagacatgagactccatctcaaaaaaaaaaaaaaaaaaagatgaggtctttaggatgctgagcacttaaaatctaagcatccagaatattattaaaataattttaattagagatatgatttgtaaataaatgaaaattcacatattgtggtaatatttcaacagcatttttttgctgataaatgtgtatcattaaacatattaagcgtcttctttcaagcagcactattcaaaatgtggtccatgggcttctggtggtcatgaactatttgttacaagttcatgataaatatggcaatgacagtcagcatttagagacttttacagcaatttggtagagaaatttagtgtctattgaatgtaatcataaagaaattaaacttttattttgattgtttgtggcttactattttaattttctactaatttctttttgttttgttttaaccataatagcagtacataatggatgggaaactgcaaaaaaggatttcacacagataatttgaaaaacattgttttaaagcatagaaaacaatcctaatttatgtataatgtataatatcctaatttatgtataatgtataatatcctagtttattttataaattagcttagccttgaaataaaaacctgacaaatatcccacaataagaaacctacaattacattatgagtaaaatattagcaaactcatccagtctcaaattaaaatactaatatgctatgaccaagttgaattttgtttttttttctttaacttttaaactgcagtgatggattaatttttaataatgtgttatttatgttatagtattggcttaaaagagacaataatataattacactgttaaatattcatgaaaaattatttcatgaacaacaaaacaggatattaagcagtaatgagtacaacttttcataatcatatatttacttagtagaaaaaatctgaaatattataaactaaaacatttacagtggctgtttttgttttgatttatattttcttcttttgtttattgctatgttctaaactttcaataatgccttttttttgttgttttttttgagacggagtctcactctgtcgccaggctggagtgcaacggtgtgatctcggctcactgcaacctccacctcccaggttcaagcgattctcctgcctcagcctcccaagtagctgggactacaggtgtgtgtcaccatgcctagctaatttttatatttttagttgagacggggtttcaccatgttgaagagatcaagagatcatgaggtctcccaaagtgattggattacaggcttgagccattgtgccctaccagtacttttttttttttttttaagagacaggatcttgccctgtcacctaggctagagtacaatggcacgattatagctaactgcaaccttgaagtcctgggctcaagtgttcctcttatctcagcctcccaagtagctgggactacaggtgtgtaccaccactcctggctaatttatttatttatttgttgtagagatagggtcttgctatgttgccaaggctgatctcaaactcacagccttaagtgatcctcctgccttgacctcctaaagtgctgggactagaagcgtgagccactgtgcctggactataacatatataattttatgtaataagttaaaaaatgtatagtcattatcggtataaaacattgcgtgtgcctggagtctaaatgcctataaaacatgtaaggatttaatattactcacttacaggtttacacgttttccaccagcttgttttggaagtaatttactcaatggctcattttagtttcatcttcaggaatgagcaatttgtaattgacaaagaaaagcaataaaaaaacccattagcacctctcaaactactgtgcattacatgcatttgcattaacaaagacagattttttgttctctgaaaagtatcaagtctttttttctccccgtgctgtggccttacttatttcccaggtaacagggtccagtcttcttcccttgaaatttccctcccctgatgggtctgccttctagatagttcctttctttgtttatgtgattactctgaacttcccacccttctttactgcctacctgtcgaatgctttttctaatagttctagtcatcgataatcatccttgggtttgactcacgttgtgattgatgaaatcttttttttgaactacaaataggaaaagcatccacttttttctttgaacacttaaatcacatgatagcattatttattataaaatcactaatactctctaaatgtcttgctgaaataatttagttcagtagtccctttcctttcatgtgtatttagactagattccttaaagaaatcataatttcttttagaagaatacttgtcacacacaccaaaaaacaacccaaagtgattgtgttgagtgaaaagaacaaggtgagaaattgtgaatgtatttaatagtattgcatcttcattcgttctctcaaaagaatatttgctgaatatttactatgtgacagttactgccgtaggagctggtgatatgacaaggaacatacaagatgggatctttgccatcaagttgcacgcatgtatacgtacatgcatatgtgcatatgcatagacatagagtaaaacatcagtctgcaacaaaacagattaagaggacagagagtgtatactggttattcttcatcatctatctagatctattctctgccctttgctgcttggctctttgtggctcctgttttctattgctcatgaagagcgaatgccctgttctttaggaaaacattgttgggcttttagacggtggaccaggaaggcagatcgcattctctgttatgggacatttcatcagaagtgagtggagacttggaactaatgggacttttgcagattactgacagttctggggactcaacaaagatggcagtggtgtggcatggtcctcgttcaggagaggggtgccaccttttaaacaaaggaaacagtcaagggaagctgagatttatgttcagtacagaaggctttctgaggaggtgccctgtaaattgaagtctgattgaattgagaaagcgagtcatttaaatatctgagtgaagagtgttccagacagagaaaaccacagtgcaaaggaaagtgaggagcagcaagaaagccaatgtggctgatgaagtgactgagggggtagtgataggagctgagggtggagaggtagccagaagctggctacatagagttttttaggccatattttgaagacttaacaaaaaattatgagtgaggttggaaggcattcaaaggtttcaactttgcgatttgaaagtgacaaactatgacttacattttaaaaagataactgatggctgtgtggcaaatagactgtgaagggcagaagtgaaagccaagaggcctatgacactagaagactaggtgactcaggacagaaagttgtaccggggttgcaagtggtgaaggtcgtgactggtcggatttaggaaatattttgaaggtagagcttgcaggatttggtggattgaatgtagaatgtgagagaaaagtgcgaagttcaggatggctataatgattttggcctgaataactggaaaaatggagttggtatttattgacatggggaaaactatgggaagaatttgggtgggaaatgaaagttccattttggacatattaagtttgagtgcatttcatatattcagtgcacatggagaatagatagttgaatatttgaatctgaagtaaaatggagagattggtgttgaaggtaagatgtgaagcttgttgctgaatgtggcatctaaagccaattgggttggacaagatcatttatagatgagcactgagcaggtgctttgctgcattctgaggatgggaacagggcttcatgggcacaagatctttgcaatcacgcaagacctcttgctcagaagggccccatgctttgttgagtgctctgctctcactatcttgaaacttttaataatttttgaataagagccctgcattttcattctgcactgcaccctgcaaattatgcaacgggttctagttgggaacataaagatccagccaaagaaagagccaatgaagtagaccgaaaaccaagcaagtgtgaggaggtcataaaagccaagggagtaaattgtgtgacatggacagcatgacccacagactcaaaaagatgctgcaaggtcaaattacctcaggactgaacatcaaccattggatttagcaacttgggtgtgcttgttgcccatgctggagtgggttgaagagagaagaaaagaaaaaatgttgaatgtgaacaactcttttttttttttttttttttacaatggccctgcctcccacagctttctttctttcagttttagacgcaaaacaaaaaagtttaaaggaaaatgtgacttgaaaggaaaggaacaaatttccaaagacttgggggagtgaatgcagagcctggtgcggacggacaaggtctgcagacggagggcagaggtggtggaaggggccagggacctgcaggcctccccctggaactgggactggtctccgtcagctgacgtcaaggtcagctcccccgcggagctgacttcaacagcccacaactgtggggcttcagcagccacgccagcccagcctagctctcggtaagtttggtcgttcatgctgaaaaataaatattaaagcctgtcccctgtctactgcctcccccaactgcacagacgccagcctctaggcctgagaggtggaaacactggccaccagcccggcagcccctacaggccccccagatgggctgcctcagtcctcctctgagagctgcagatcctccagctcatcctccggcccctgggccagccgctgcagctccccaggggccagccccgcctctgcgtctgggtctccatcctccaagttgctgctgtcctcctcgccctcctccttgtcctctttatcctcttccaccccatgccgagtgctcaggggccccagtatccctctttccaagaatccctcggtgtcgtcctcttcacagctgttcaggtcaaagaggtctttaaattgcttcctgtcctcatccttcctgtcagccaccttccttcgtttgatctcagggaagttcaggtcttccagccgctctttgccactgatctccagctggatctcccggtcatgcagcttgcgccagtggctgtagtacaaggtcaggggggtcccctcttccagggtcagcttctgccaggcttccactgcctgcagagacaccaaaggaaaccctctggcggcggctgcagatgtgttccgagttctcctgaaccttccccagcagctgctgcacctgccggcagtagttggccaccttgcgctcccggaggaacaacttcagctgcaggaccacaggcagcgccagctccgggaagccgatgcagtgtgcctggctgtgcaggtactccagggtgaggtcgtacagctgctccaccaggccgtcctggtacgccttctcctgcaggttgacattggacagcttcaggatcacggagaagttgatgggcttggagctcatgcgccccggcttcctgctgaagtcgacctgctggaacatctccaggatgaaaggcagcactgggatgaaggcccccgagctccccgagagcagcgtcagggcacggatgcagtgcattcgcagtgggtagaagcgggcagtggggatgagcttgatacagccaatgatgacctgggcaagggagtagaccaagggctggagggcttcgctggggcccacagtgctcaggacccggcaccacaggaagaggcagtgcatgtactgccagttgtacacagacgtttccttcttgcgggtggtcatggcgttgtgcaggtgtatggcgagctggcggatgtagaggaaggcgtgctggtaggccacacccggctccaaggccagcagctccgtcagggtccgctgcatggaactgatgaaggggagggcaccaggcgaggtgaacttgcagttcctcacatacatgatgtacatttgcttgaggatggggccaaggaaagtgtccttcttgtgccggcagactctgctgaggaccaggaaagccagcacccgcagggactccttccctgtgctccatacgaccaccattctcttgagcactgcttggggaaggtcaggaagcagggcaccagcacgctgatgtgccatagcacggccgccaacaccactgtctctgccacacaggacaccagctgtatgaccgagcccaggtacgccttgatgtccacacggagcttcccccagagcaggctgctggacggctgcagcatcctgctgctgtcctttgccacctttccaatcagcagcttctggagacagccagtaaggtctctgatgcagaaggtgaccagagcattgaacacgaccagagcattgaacacagcactgtccgtgacctggaatttgttggcctcagcactttcctggtccccttgggtggtggccacagctgctcggaacgcctgtaccacttcatggaacagctttggagtgaggcgttgctttgctgcctgcttccatctctcaaccatggcgagggtcacaggaacacaattcttcttccccttcagccctctggggactctgtccccatcttccccctcctctgctccatcctcatcctcactggcttcctccagcacatatggcagggagtggaacggctcctcttcctcctcagagctgtccgagtcgctgaagtttagctggctctggtcattctcctgcaggaacttgtagaactcggcgtctctgtccttcagccgagagagctgatctttgtgctcagaggcacggcctttacgccggctggccgaggggctcccgcccggctcatccggactccgggcagcctcgcgtgcctcccgcgtctccgcttctggagagtattcggacttagagtcaaagcccgaagctaggaactcgtccaccgtcagctccgccaggcgcctcctgcggcgccgcccacctgccatgacaccagcccgcagctggcgccccacttccggccccagaatgccgcgcggctacgcacttccgtcgcccaggtcccgccccgttacccgccccgccgcgccacgcccagccgagtggctctacggttctccgaccgcagcgcccgcggcctccgggcgggagggcgcgttcgcgtgctcggtgcgggcagccccggtggggcccagatgcgcctcccgctcggcgcccggcttcgtaggacgcggcgacgccggtgtccgtcctggggaagagagaccaggagtcccgtcgcgcccgcagcccacccggcgttccgagggcatgcgcctgcgagaacgccaggctgcgacggcggggcccctatgcaaagagctcccacaaatcaacaataaaaagcagagggtccaggagaaagcgcaaatggcggtgggaaccgcactgatgttgccagctcgacagaagacgggcgacccgagagccaggctggcttcgcctctgatccgcagagaccaggccagcgccacgaacaccacgcagggcgctccccgtccatggccctttgggtgccgacggcgcctcttctgcggcctttcccggggacgccgcatggggcgaatgcggtggggaggccagcgccttccttcctgctaccagcctgacacccgggcccccgggcctggacagccagaccccgcgacagggtcgccccggggcgcaggcgcggatcccccagacctggcgctgaagggggcgcgtgagccggacgcggccagagtgccctcagggtcccattcggcctcttggtgcaccggggccccgtggcttcccagaggagagcaagttagggggtcgggggagcggggatggcttctgggggaggaggagggcgaggcctaggggtttctcggaggaggaggagggcgaggcttagggggcttcccgggagaggaggagggcgaggcctggggggcttctcggaggaggaggagggcgaggcctgggggggcttcctggaggaggaggaaggcgaggcctaggtggggcttctcagaggacgagggcaaggcctggggggcttcccggaggaggaaggcgaggcttaggggggcttcccggaggaggaggagggctaggcctgaggggggaggttcccagaggaggaggagggctaggcctgggggcggggcttcccggaggaggaggatgcagacccggggcgcagcgctggaaggatccgcgtccaaggtctagtccccaaagcctctcgggaggccgggcgggcggcgccgagcgaatgtgaacaactcttgcagtcacttttcttggaaatgggaagcttaaaattgggtagtagctgagaaagatacaggttcaagagagggcttttttaagatggaggtatgtgttataaatgagggattaagtaccccttgtggagtccttaaggaacattccaataggctgtggagatgactttgggaaggcatatgaataaaggatttactgtactcacaggttccaaagagggaggcacgacataccttgtggggatcacctgggcagagcaccaacaaagcgggctcagccaagcaggtggggagccaagggcaaacaggttcctgtggacagttacccttattgggcatcagggtggggttcaccagcaaaggcaggaggggatatcatgggtgtgcttaaatgtcactaggccatggtcaggggagagtaagaagggggacttgtgacagggaccagccttgtcacattggtgtacctggtcatctgggaaaggtgcccacagcttgtttgtagagatactgaggaatcaggaaaatgggaagtttttagaagttgcaattcaagatgttacggtacatcatttttcttggcaatgactcattaaaaaagaagaaaaaactaatgaggcaggaatggaaataattccaggagcaaagtcctttaggaggagtggggaacaggatccagtacaaagtggggggttggccctagaagccagggaaagccgagtgcatggagacagatgtctgtgcctgctgcattttgtagaatgatccattctgatttctcctgtatcaaaattgaagccttcaggtgagagtgagaagcagagaggaaatgttgaggtttgaataagagaagaatacggaacggtcgtctaggaggatgggcaagcaaggagaccagaactggcaggcagcgcaagggctgctgaacagcccgagcagtgaagtggctgaaataggaacatcaggtgtctctggtgattatttcttttttgtagttttctgattttccagatgttccctaataggcattatcttacttccacagttatccttttttagactgttacagaggggtgaaaaatagaacatataatgtatgagagaggacttaggatttggggaggtgtctggggaaacagaaagagatagaagacctcattctgtttgcctgtaaaaacaatgcctgcaatagatgaaaagtccagttttctgcagacatgtacaaaatgtttttgaccttttattcaggccaacagatttactccatttctaggtctgagccaccaattgggtttcttcattgagtctgagtgtttgataggccaagtggtggaaactgttcccaagttctgaaactaggggagcctccattctgggctctgcagtggtgaggtgcactctcatccctgccacattttagtgggaagcctggtgaggctcattcagggactatgaaaacatctcttacaccagaacctcactaatcttgtaaagtttgcttatttgttaaaatgattaaaaataaacattagaagttgaggtccttgatatctgttttcagagtttcttagccactctctatttattatatcttaatagatttgcctgatttttttaaaatttcaaatcaaggtcactgttgtgaaagggaatttcagagataatacattgttatgaagggccgggcgtggtggcttacacctgtaatcccaacactttgggaggctgaggtgggtggatcatgaggtcaggagtttgagaccagcctggccaacataatgaaaccccgtctctactaaaaatacaaaatttagctgggtgtggttgtgcacacctataatcccagctactcgggaggctgaggcaggagaatcgcttgaacccgggaggtggaggttgcagtgagccgggatcatgccacagcacttcagcctgggcgacaaagccagactgtgtctcaaaaataaataaataaataaataagttcctctgagttatttaccatctttctgttgaaatctttattgaaattataaatctcagctttcagaacaagatgtggtaattaaatcctatgactgttaaaacacacagagctcagctgtgtggacgcagagtaactgcgtggggtgtgaggtagggtgtgtgtttagaactctgcaagttgaagatttcagagacaagggcagctgcagaagtcctgagttttacactaggacaaaagagaatagatttttctcttatggtggaaagaatatttaacccagcaatatgtatttagatgatgtcatactttcttcttgaaatatacaaaccctttgatgaatttctttcatatttaaactctttcataatgaatctcttaaataataaatctttctaatacattttttcagtctttgtattcatacaccaatgtaaattctttttttttttttttttttgagacagagtctcgccctgtcacccaggctggagtgcagtggcacgatctcggctcactgcaagctccgcctcccgggttcaagcgattctcctgcctcagcttcctgagtagctgggactacaggtgcccaccaccatacccggctaatttttagtagagtcggggtttcaccgtgttggccaggatcgtctccatcttctgaccttgtgatccacagaccttggcctcccaaagtgctgggagtataggcttgagccaccacacccgaccgtaaattcttaaaaagacagcatatttattaagttcatgtgttattgctattggaaacaaagacacagtagataaggagtattgcatgagcactggatctgagttgaaaactatgcttggaagacactaacagaactatcttcaagtccgttaaagggaattatttgcagccaggcatttttttccctgaagtaatatctatgacttcttagtagatggtctgaagtggcaaggtaggaacttcagttattgtaggtgaaaacctcaagctatggctgggcatggtggcacatgcctgtagcctgtactcaggaggctgaggcgggaggatctcttgagttgaggagtttgacgctatagtgcactatgatcactcctgtgaatggccgtggcactccagcccgggcaacatagtgagaccccatctctaaaaaaaattgaaaattaaaaaaactttcagatatgcttagaagcagatttttcaaatcccatggggaagggctttaagaagagtatgatcagtaaccagctaaacaatgattaaagcatcatctttagaagatgatgaactaaaggaatctaaatcttattgcctgaaactctttgcagtcaggcagcttaactgggtgtctttgagaaaattattgtctcatttatcatatttgtgcaatgaaaataatacctacattgtacgcttgtggaatttaaataacaaaatattttatatatctacatatctcttggatatagtacaagtttaaaacattgtagtttattatcagtttcctgagagatggcaccaatggcaaaacggtcaccaaagctattcctttagaattagaggacagtccccacttcatggacaacatgtgagtctatgcacccacagcatccgtcagcctgtcctctctccttttttaaagagcaaaacaggaattctgttgtgattgaaggactttctgtccatccctggaatcctagatgctttgagatacctgcatagaagttgcagacttttttcaaacactaatggaataaaatactgtcattgcctctcttttttcatagctagcatagaagactgatcttttgggtgggaattgggtgggtggcaacctcatccaaggatggggggctacataagttgtagaagaccttgccaatccaggtaggttgtaactttaggctctgtgtggctggtcagggagtcagtcactggcttgactctgaccttccaggagcctggcagttgaagaggatctcagggtgatctgatagagaactgcagaaggtgagagtcaagttagcctcttccagacttattacagtcccctaaagcattaatgacctttttgcctaaagctgatacatggacatttttagttcattgaccttcaaataattacagctgtttggaaagctcttggtcatagaaactggtagtacttccaaacttttcagtgatgtttatcatcttttttttttttttttttttttttttttgagatggggttttgctcttgttgtccaggctggagtgcaatggcgcgatcttggctcaccacaacctctgcctccttggttcaagcaattctcctgcctcagcctctggagtagctgggattacaggcatgtgccaccacgcccggctaatattgtatttttagtagagacggggtttctccatgttggtcagattggtctcgaactcccaacatcaggtgatgcctccgcctcggcctcccaaagtgctgggattacaggcgtgagccaccgcacctggctgatgtttatcatcttaatagcatctgtcctttaaccctgcacacacactccaaaaaagttaactaaaagagacaatgattaattttatattctgatgttttcttatgaacacatttgtggccactttagtgtccagcaaagcatttctttggcgagtgatttaaggaggggtggaaagtggaatggaagaagtctctggaaagtctttaacactgagacgtattttctccctgtttgactccaaggaatttccactcatggaatctggcctattagaaagaactttttccttttgtacttgattcaacttaataaattatgttaaccagctatggtgaatgatgagcattcctcaaaccacagaacaataggcttcatcgttgaagctctaaaagagcagggatctatctgttccaattctcttctgctgggcaccatcttctagctctgctgcagtctctcattcataattcttcccctagaccaagttcttcttttttactgacctctaggacacctggcatgcattcctcttctgactgcctttgttggcctgaatgctgaaataacatcactgaactaacatctcctaaagaagacatttgtcatcagaggcaaatcctcagtgaggattaacctgtctttaaaagatcatgggtaaatcagcacaatctgaaagattcagagcttttttacctttctctctgctaggcacagtgattaccatctccagccttattcttcacagccactcaatcgtggtccttgcagttttgcttcactccacatacaccatttcatttcccttcccacagtcacttcctagaacagatatagagggaggccctgctttgcatgctaatgagggaccacagaaatgactgcaaacagaaaatggaacgcgatctcgtgcatcaaaggggaatgttctgactgttctgtgacttttaaaaatatgttgtcaaaacattaaaaactattgtaggttataaatgtacaggaacattttcaaaaatagtaaaactaatatttagtatagtgtaatttaaaacatcagaaacacgaatcaatattttatttctttgtaaaaatcttatggagtatagtttgaatgaagctggccctcctcttcttgtagtatacttacagtgcagagtgagctataccttgacaaactgtcattctcctgtctaagtttgaatcagcttccaactatttattctttgtgctttcaatgtcatgaaatacctctgataattccgttaatgtgagttttgtttgttttgttttgttttgccatgtcactttcttgggacatctttatcctaaaatgaatgaacacatctattttcatttatgttgttaagtctgccttccctaagttcctctggaatgcctcaaacagtggcaaattacctccaatagctatttcttccataactccatttacactcaatttgaatttcactttcagagtatactagtcagtgttctccagaaaacagtatacgcacacacagaggcataaatatttatttcatcttatatatatgaagatttataagatctataaagatttatataagtatatataaagatttattttattatggaaattggcttacacaattagagaggctgagaagttccaccatctgcattctggagaaccaagaaaaccagtggaataattcagtccaaagtccaagtccaaggctggaaaaccacagtgccagtgtccaagtgctggagaagatggatatcccagctcaaacagggggagtacatttgcccttcctccaccttcttgttctatcagatgatgcccacattggtgagggtgatctgatctttactcagtctactcatttacatggtaatgtcttctgcaaacaccctcacagacacaccagaactaatgttttaccagctgtctggacatcccttagccaagttgatgcataaaagtaactagcattcagagttctctatttttatttgctgtgcttttatctttctgacacttcactgttggccagttttctcttttgattagccattattgaaaatgttatgcaggtttatcactggaagaaaaggaaaaaacacagctgcatggtttgttgccagtgtgtgaattgaataaaagatgtggggccgggtgcggtggctcacacctgtaatgccagcactttgggaggctgatgtgggcggatcacgaggttaagagatcgagaccatcctggccaacatggtgaaactctgtctctactaaaaatacaaaaattagctgggcatggtggcacctgcctgcagtcccagctacttgggaggctgaggcaggagaatcacttgaacccaggaggcagaggttgcagtgagccgagattgcgccactgcactccagcctggcgacagagggagacacaagactccgtctcaaaaaaaaaaaaaaaaaaaaaaaaaaagatgtgtggtgaccaatcaccaatagatattgaaataagtgacatgattggtcactgatcataatgcgcctctggtatacatgcagcaatttatgggttgaaggtctagcagtgaagtttctcctttatccaattattcatagttaatagaccatagcaactgatatcagaactgtgttgttgggggactggtgtcattggactaaactgtggtaactgaaatctgtgtaatactggagctgtgctaagcaaggatggactgattattactaggacaactttctggcttctctttttatttttagtgcatctaattcatctgacatttaatcgatccagattttcttccaggatgactaataccttagtcatctaaccactcaaggacctcacaatatcttacatgtagttttcacctaaactagggtataacaattataaccatcttcctggtcatcctaagcatgtatactgcagttatatcctaagatttcatctctattaagctccagggagtaataggtgttctttagccctctgcaggattagagaattgaggctgctttgttactgttgttgttcacaataaacttccataggaagtgagttacaagactctagacctttgttaatcctgaactttctgggcatcttgtctgagtgatatatcttcagtaaagtgtatcctaagagctgtcctttctgtttccttggccttcttcacaagcctgctctccaaatacccccactctgggagactgactagctcccaagttggaacacagagttctcaagcttgttgctttgtaggtgacaccaacagaggaacttctgcctgatgttctatctgctaactccgaaatcagagtctggaaagagtaatcttgccttgcttatttagtcaaggatacctctcttcttggtactgctatagagggatgtaaatgcctcttcttcacagttgacttcggtagctgggaaatacgacaatatctctaaatatttatttgtatgtaacacaaaactcctgcaaaagaataagagataaacaggcagaggctgttctgcctttttatactgagcccccaaagaaaaacctgtggtgtcatgctgggcagaattttgtaactattttgaccaatagagtataatggaagtaatgccatggtgccctccaagatcgatagtaagaacaccttgcacttctgccttgcctacttaggacatttgttcttagaacccatctgctatacatgctgtgaggaggacagaataacccgtgtgcagaggctgtgcagaggcagtttggcctgtagtccatctgaggtcccagccaataatcagcatcaactaccaggcatgtgaatgaagatacacacaggtgattcctgttcccaactgctgtcacacctaaactttgagtcttccaagcaaatgccccagatgtggcatagcagacatataagcagtctgccctgtgtccagactaaactcctgacccataaaatctgggaatacaaatgttgttattttaatacaattgtatggcactgagattggggtggtttgttatgcagcaattataactggaacaagaacacttgaccctgtaagacacttgaaattatatgagttatgacatcagtaaagtatcttttcttatctacactttctgttgtaaccacaatgcatatggataatttgtgaagtgaataaggacatggtgaaggactgattccaagtggctgggagagggaagggtgtgctgcatgaatttcatgttcttagatactagtgagaagaggctgtaacacattaatcactccccttctctcactcatacaatggtagcactagtagcacattatcttttcttgtgaaagtgttgcattaagaaatttcttttggagtagcccccaaactgtaacagaatgtgacgtgataatttcccaccgtcttcaagtactgccaagatgtagaagtctccttactccttgtagacaaatggttgctccttggaggggcctatttgaattttttttgttttgttggtcttttttttttttttttgagacagggtctcactctgttgcccaggctgtagagcagtgatgcaaccacagctcactgatgcctcggccttccaggctcagttcctcccacctcagcctcctgggtagctgggaatacaggcatgcaccacaaagtctggctaattttttgtagtttttcatagagatgaggtttccccatgttgcccaggctggtctcgaactcctgggctcaagctgtccacccacttcagcctcccaaagtgagccacctttcatctcctcatctcatcttatgactctgtgcttgaactggtgtatttaaagattaaggacctttctgaccaatgtcaaatgaaccatccagattgttctggctctcagaaagatcagaggcagtctgaatagcaagtaaggatcaagtcaaccaccaattcaccaaacaaagccaaaacattggcgtcaaggaataagccaattgctgacataagtacctgctgccccagaagttacttgtttcaactatttgagttcagcacattatcgcaattctaaatacttaaggtatcttcagcaagtgaatgcaacttataacttgttcttccatcctcgggtaagtgggggaaccatttgccattttcagagtaaattcatctgagttagaaggaagcccctataacatcaacagaaagcaaattctatagttaagtctctcaaccattaaattttaggattatattctagtggtacttcttagaaaaaagaaagaaagtttcacctatgagataaattagaggacaaagtgttacttcctgtcttcttgcaatcttggccgggcaccgtggctcacgcctgtaatcccagcactttgggaggccgaggcgggcggatcacgaggtcaggagatcgacaccatcctggctaacacggtgaaaccccgtttctactaaaaatacaaaaaaattagctgggcacggtggcgggcgcctgtagtcccagctactccggaggttgaggcaggagaatggcgtgaactcaggaggcggagcttgcagtgagccgagatcgcgcctctgcactccagcctgggcgacagcgagactccgtctcaaaaataataataataaaatataaaaataaaaaattgtcttgaccatcttggggtgaaacagctggagattaaaggaagaaatggctgccgagcacagtggctcacgcctgtaatcccagcactttgcgaggccgaggcggacagatcccgaggtcgggagattgagaccatcctggccaacatggtgaaaccccgtctctactaaaaatacaaaaattagctgggcatggtggcgtgtgcctgtaatcccagctactcgggaggctgaggcaggagaatcgcttgaactagggagtcagaggttgccctgagccgagatcgcaccactgcactccagcctggtgacagagcgacactccatcttaaaaaaaaaaaaaaaaaaaaaaaaaagagcgggggacgggggcggaggggggtggggaagaaatggcaaggctctgggaggaataaaaagaaaaaaaaaaaaaaaaaaaaaaagaaagaaaataaccagaattatatctttgttaaacgaggggccaaaaccagaaagctttgtacaaggttttgtgtcttggcagcaagcattgcccttacaacatagaatgtcattgcaatcatatggtatcagtaaccaatgaattacggccagaacacctaggacctgttcttctagactttagatgtgagttctccttctcacaaatctcaaatgcatgttcattctgaacactggctggtttttttgaccctttttgctgacctgagagggttatatagctagagtaagactgaaactattgcctggaacatccctcagagtggccaaaatatttcctttatgaccataaagcaattccaattcacattgctttctgtaatagagctttcctttccttccttccttcctttctttcttcctttctttctttgttgacagagtctccctctgtcgactaggcttgagtacaatggtgctatctcggctcattgcaacctccgtctcctgggttcaaatgattctcctgcctcagcctctggagtaactgggattacaggtgcacgccaccatgcctggcttgattttggggtttttagtagagatggggtttcaccatgttggcaggctggtctcaaactcctgacctcagatgattcacctgcctcagccacccaaagtgctgagattacaggcatgagccaggcataagccagtgcccagcctgtaatagagctttctggactactgagttctacagtattttcctgtatgattattttcaatttctgtctcaacacttgaggtaggctctttcaataaggcccaaacacactaccaattaatagcattatgctgtgcttctgatttaaactttggtcccagaacccagaaatgggagcctgtgtctattatctgtctcatttttagtggtctttgtggtctgcatggtgaagatgtaaaagctttaactaagatgtaggaatggagaaaagaaaaccaggagaggacttgcttacattctaaaagtgggttgtaggggaagaggaagcaaagctgagacttctgagtaactggacttatgctaagatgcctaactgaggtgggggatgatttagttttggatggttacatggtaaactaaagaaaaatagtattgaagtgagacaaacatctaaaagctttataataccttaatttgattttttaaaaattatactttaagttctggggtaaatgtgcagaatgtgcaggtttgttacataggtatacatgtgccatggtggtttgctgcatccatcaacccatcatctacattaggtatttctcctaatgctatccctcccctagccccccacccccaacaggccccagtgtgtgatgttcccctccctgtgtccatgtgttctcattgttaacctcccacttatgagtgagaacatgcaatatttggttttctgttcctgtgttagtttgctgagaatgatggtttccagcttcatccatgtccttgtaaaggacatgaactcatcctttttaatggctgtgtagtattccatggtgtatatgtgccacattttctttatccagtctatcattgatgagcatttgggttggttccaagtctttgctactgtgaatagtgctgcaatgaacaaacatgtgcatgtgtctttatagtaaaatgatttataatcctttggaaatgtaaatccttataatcccagtaatgggattgctgggtcaaatggtatttcttgatctagatccttgaggaatcaccacactgtgttctacaatggttgaactaatttacagcttcaccaacagtgtaaaagtgttcctacttctccacatcctctccaacatctgttgttttctgactctttaatgatcaccattctaactgacatgacatggtatctcattgtggttttgattttttaaagaattctacattaaacatcttgatgaattactaaaggaatttctgttggtaagagaaaaagcatgactactttcactgagagtattcagtactgttcaggttttattcccagtgcaatatgacaggaaaaatgaaatatacatattagaatgaaacatcaattacacagccactgatataattttcagaaaatgaaattaaaaattaataaaaacaatgagttaagaggacagataaaagataaaatgaaactcaaaagaattctcatgaatcaaactgaatcaaattgaatttgtacaagcaaaaagtttctattcacattgaaaactctaaaatatcttgatatgcacttagaaaaagtgagcattccctatgcagataaatctacagaatgttacttggactacaagagttagttaatggagagacatcatctgactagatgagaatatccagcattatttatatcttgattatctcagcctcctccaaattaaatatgtttaatacaattttaatcaaattattgtttttttttcttcgaacgtcagtgcttgacttcagatttcttttggaagaaaaatgtatacgaataccttatattcgataagaatgtgaaaagatttgctctactatgtatcaaaaaatgtaaagctaatcgaaacaaaattgagggaaaagctatattataaatattgattaaaatcattataaaaagcttctgcaatggaattactattccatggcaaagcatttcaacagacaagtcacaaaaaaagaaagaagagtaggctcttcaaatgaaaagctgctcaactttcttcccacaacaaagatgtgaaacagataattagggtttgatcattggttcttttgttttgttttgttggtttctcttttacatataagaggtttaacaatttgacacccagtgtctttaagacagaaatgtcacttatgtcagtgttaatgtaattttggaggaacaaacattgggttgtatctataaagtgttcttagtttttttgtttgtttgagatggagtttcactcttgttgtccaggctggagtgcaatggcacgatctcggctcactacaacctccgcctcctgggttcaagcgatcctcctgcctcagcctccccagtagctgggattataggcgcacaccctgacgcctggctaatttttgtatttttagtagagacggtttcaccatgtttgccagggtggtctcgaactcctgacctcaaggtgatctgcctgcctcggcctcccaaagtgctgggattacaggcgtgagccaccgtgtctggccaaaagtgttcttagtttttgacctagcaattccacctgtagaatttttttttctataaaacagacatatataaagataaaagataggctgggcacagtggcttatgcctgtaatcccagcactttgggaggctgaggtgggcggatcacctgaggtcaggagtttgagaccagcctgatcaacatggagaaacgccatctctactaaaaatacaaaattagctggacgtggcggcgcatgcctgcaatcccagctacttgggaggctgaggcaggagaatcacttgaatccaggaggcggaggttgcggtgagccgagattgtgccattgcactccagcctggacaacaagagcgaaactccacaaaaaaaaaaaaaaaaaaagatgcttatgatccggcggatcctctcctggggagaccaggccggggagagggcgcgggcgcccagtggcgggggcagcgggcaggcgcagcgaccggggccggggcggggatcctggcggcgaccgccgcggcggcagcgccccgggcccgccgccccctcccctccggcgaggggagccgctggatggggccggtcggggggcggcctgcggcgcagagcggcggcgacggcggcggactccccaggcgggctggggctgagcccggggaccggggcgggggctccggggggaccatgcccggaggccggcgggcagcagcgtggctcacgggcctggcgcgctgatgctcaagtgcgtggtggtcggcgacggggcggtgggcaagacgtgcccactcatgagctatgccaacgaagccttcccggaggagtacgtgcccaccgtcttcgaccactaagcagtcagcgtcactgtggggggcaagtagtacctcctaggactctatgacacggccggacaggaagactatgactgtctgaggcctttatcttacccagtgaccgacgtcttccttatatgcttctcggtggtaaatccagcctcatttcaaaatgtgaaagaggagtgggtaccggaacttaaggaatacgcaccaaatgtaccctttttattaataagaactcagattgatctccgagatgaccccaaaactttagcaagactgaatgatatgaaagaaaaacctatatgtgtggaacaaggacagaaactagcaaaagagataggagcatgctgctatgtggaatgttcagctttaacccagaagggattgaagactgtttttgatgaggctatcatagccattttaactccaaagaaacacactgtaaaaaaaaaaagaataggatcaagatgtataaactgttgtttaattacgtgagaaacatcttcagtggccaaggaaactggccatttctctgagaaagcaaatgaaatgctacagctatacccagaccttttataggtaatgaagcagttcaaaacttgaaagaaaacaaaacctgtccccagaattccataaagtttattaagaatgttccttaaaggtttaagaagcagtaagcagtatctgaagccacaatctactataaatactttatttcaactagaaggtacaatctctcaggggtttcatagtttaaaaagctacaatcacatcatgtaactacgtaaaaaacagagctgtaaatggaactgcttggctttgaccatacacatttctgcacagcccttacagaatctgcacaaagaaatatctccctttgttccagttaattgttcttgtatgtaagttgctttctattccagtatatccagagtggtgaaataacaaggccagccacatagccaaaggtcgctccaagcgtacaggagatgggccatacctgaggagagaatgtatgagatcaaaaaagaacaaatgttttattattacttgagcacaagtgtaacctaaatatttctatattaaagcttaatgtgctttcttaaagaatgcctaaagtgtaataaggtcataactgcatttatcatgaacactaaaaatgtacacattttagttaatgtgcattaaactgtaacaaggcttcgggcaattgtagatttagtttgacgctccccaaagtgcatgagacacatgctaaaattacaaattaaaattttgggtcagactttgccataatgctggactcaatttagctctctgaactagatggtaatttttttttaattcccactttggctgtgtacatcaaatgaaatgagaagtgtgtatgctgaccaaaccacaagaaactttaagttgtgttaaagaggaaagacctagaatccaagcatgttacatgaaaattgtaacagagcagctgcttccacctttcagatatagatgttgggaccacagcagaagttatagagcgagaacttatatacacacctagaatgtaagttaaacaaaataccggcttccggagacccattttctccagccatattacattaggctagaagtaattaatgttgatttatttcatctacaagcagttggtccctaagtgacaggctctgcttcaaaaaaaaaagttggagaaaaattttcatgttcttctgtgaagcttatttggtacactggagccttttctaatctttctctcgggggaacaggctgcagaactgtgttagaggtgaaccatcttaattactagttctattacctaattcagcttccttgtttggtctgctgtggatctgccttattgcatatgccatgcatcagataatggtgttagacaaagcttcattgtgaacaacctaatgcattttagagaaacaatctcatctacacatttttttctagcctttcctacatttaaacttgctgttgcctaaattataattttttaaatgtctttggtgggcttctgttaattcatgtgacttgagcttatagctatgtctactgcacagattgggtaatggaacactaaacttttatacttgaaaatgacagccttaaatgctcatatcagtcacaaatctaggatgtactgtcttgttgtatgtgagctttgtagagatttgtaaaaatataagcatcaccttcgcactgaagagtggagagagtctactggatgactggccaggaactttctctctgaattggacatttggatgtcttctttcttccaagaaatggtggttcacattaaagtatcatggccttatgtatgctcaaatggaatcttatgtaagtttcttaatttaattttggtctgctatttttagataaaattgaaaggaattgtataaatcaattaacatattagctaagttgtccaacacatgttataaatgaattacaacagtaaactattatacatttccaaaaaaaagatatattgcacaatgttcactgctatatagtgtacaatatcaaaaattggaaactatcttatatattataatttatttcagttatgagtggtagaaaaaccattgtagctcttgtcaaaacctgcataggcagtctaaggtaaatataacacttgattttatcagggcccaggttccttctgtcttgtggccactgtatgagccgcaaccccaaggtggaagatggcagtatatgtttatacacagcaatgaggaagggagataggaaagcaaagtgtgtccctttaggaaagatcttggaatctgccatgccatatttctactacatcctgttactaagaacttaggaacatgattgcacctttattggtagtctttattctgggcagccatctgcctagcttaaaatgtggagggtccattaccatagaggaaacagagaatgaattttagcaaacaactagtggtttctgccacatgactaaatgaccaacaaggaaacatattacaagacatcatacaattaacctctattcagttgttacagactcaagcaactctacatgaattggcatgtgaacttcttgcaaaacagggtgtttattaaacagtttttaaaaagtgtgtgaaaagcctgcatatgtccaccgaaagacatgtaaaaatgcttattcctttattatttgtaatatcccaaattttgaaatgccacaaatattcaccagaagtacaatggataagtaaagtgtagcatatttatctagttaaataccataggttatgggaatgaacaaagtgttgcctgatacaataatgtggatgactcctaatgttgcagcaaaagaagacaggcacagaacatataatttacaaccccacttacataaagtttaaaaacaagtaaaattaacctacagtgctagaaagcagaataaagtcaaggaatgctagtgtcaggaaaggagaaagcagccttctgaggggcttctgaggggctgctaatgttctatttctagaactgggtgttggtttttggggcatattcactttgtaaaaattcaccaggttacatgctgtgatttgtgcaatttgttgtatggatgttacacttcagtatgattgcagaagcctagcttatctaactgaggtactgacagtaattctcaaaatggaaggtgggctctcgtgcctggacagatgtcctcactgcaagctggcatgcagggaccaggtgagtggcttctggtcaccaagggcagctgtgtcacttggacatgagggcagaccccaggtgagagcatcagtaggacaatgctgtgttacttcctgcggttctgtgctggcagccacagaaattgatggggacgcttccagcttcagtgcttgtgtttcttgctcactagctcagctggtctgtaataacaaagaaatgaatagtgggatgaagacatattccacaggattcagactctacttacaagttctttacttaaaaagtgcactcttggccgggcgcggtggctcacgcctgtaatcccagcactttcggaggctgaggcagggggatcacgaggtcaggagatcgagaccatactggctaacacggtgaaaccccgtctctcttaaaaatacaaaaaattagccaagcatggtggcacgtgcctgtggtcctagctactcgcgaagctgaggaaggaaaatcacttgaacctgggaggtagagtgagccaagacagcgcctctgcactccagcctgggcaacagagtgagtctctgtctcaaaaaaaaagaaaaagaaaaaaaaatgcactgtgtctggcccagaggttcccgtggttcttattcctggatcccatcacccctcctgtctctacacagacccccactccactcccagctggccgcctacccaagcaccactgcccttggcatccttggctaaagtggtgactgcatgggcaggtggagtctgacaagtcacagtgaccaccagcagtgggaggtcacttggcctcacctgtaattcacagccctgaccacaccctctggcatgtgagtttatccacaagtaacaaagctgagatgagacaatttcactacataacttgctcacgcggcatcctgtggtatgctagcattattaacactggaagttcttttgaagatttagaaagtttaattgtcatgagatttttccaaactaagttcatgatatggatttttaaattgtatctaatttaagtgacatttcaattcaaatctaaatctaaaactgatagacctaatgatttcaggtgattaattcttttttgtttttgtttttgttttgttttgttcaagatggagtctcgctctgtcgcccaggctggagtacagtggcacaatcttggctcactgcaacctctgcctcccaggttcaagtgattctcctgcctcagcctcccaagtagctgggactacagacacccaccaccaagcctggctaatttttgtatttttagtagagacaaggtttcaccatgttggccaggctggtctcaaactcctgacctcaggtgatccacctgccttggcctcccagagtgctgggattacaggggtgagccaccgggctggcctaattagcatcttttaactgaataaaaacatcctatttaaaagaccaaatatattacttcatttgaaaattaaaatatagggttgggcatggtggctcacgcctgtaatcccagcactatgggaggccgagccaggtggatcacccgaggtcaggagttctcatgagatctggttgtttaaaggtgtgtagcatctccccgctctctctcttcctcctgctctggccatgtaagatgtgcctgcttccccttctgctatgattgttaagtttcctgaggcctccccagaaacagaagccggtacagcctgcagaactgtgagccaattaaacctgttggttttttttttttttttaataaattacccagtctcatgtagttcttttttttttctgtcacccaggctgtagtgcagtagcgcaatctcggctcgctgcaacctccatctcccaggttcaagcaattcttctgcttcagcctcccgagtagctgggattacaggcgcccaccaccacgcctggttaatttttgtattttaatagagatggggtttcgccatgttggccaggctggtgtcaaactcctgacctcaggtgctccacccacctcggtctcccaaactgctgagattacaggcgtcggccaccgcgccggcctcaggtagttgtttatagcagtgcgagaatggactaatacacttggctatgaaaatgaaaacaatcacgaaggcagggtccctccccacctctccatatccctgttttgtctggagctgccttgggtaacgctcagccgggaccagcacattacacaaggcaatgtccacgtgccgggatcagcaaggatgagctttctgtctggggtaagagtaaagactctaacccagcaccacctcgagggcttcaggctcactttctgttgcttgaagtccgtgacgtgctcacgacgtctgcactcctttgaggcctcagcaacagtgtccttgagctgagcttccagctacgaaacgaagacgcacttgtcattaacacaaaggaagcacggccttaccagttagatcttccccaaaggacttgtaagagagcaagaccttaaaggacctttgtattttttcaccctttatcaatcttatcacaaattaaagtttcagaaatacaccgcagggctcagctgaagtggttggttagtaataaaacaagttcaaattcaaatttaagttgatattttgagataagatttcaagaaagggtactgatttgtgaaaacaagtacttctgatttctgtagaacactgattcattttttaaatggacaatttcagacacacacagaagaacagaagaagagagagtaatgaacctcctgtgcctggcacctcggctttgacactaaggagaggactttttttttttgacaagattcttgctctgtcgcccaggctggaatgcagtggtgtgatctcagctcactgcaacctctacctcctgggttcaagcgattcttatgcctcagcctccagtttagctgggattacaggcacacaccacctcgcccggctagtttttgtattttttttagtagagatggggtttcaccgtgttggccaggctaatctcaaactccacctcaggtgatccgcccacctcagcctcaccaaagtgctgggattacaggcaagccaccgtagtcggctgacagtgagcagcttgagctgagcttgcttcatctctgcctacgcacaaaccgcttgagaacaaatcccaggcaacgtatcactgcatccgatgtggaatggtaatccttagttaggggtttcattaacttgagttgtaactggctacaactacaaatatatttaggttgcaaaataaactaagaagaaaacaaacagatatcctgtggctaaacagcaaagggccctgaacttgagcatgtgctttcagaccttctcatttgtgggattcattaaataaattttaatagaatttttttgagacagattctcactctgtcgcccaggctggagtacagtggcacaatctcggctcactgcaacctccacctcccaggctcaagtgatcctcccactaagcctcctgagtacttcatgctagatgtgcatgtcaccacacccagctaactttttgtatttttggtagagatggggtttcactatgttgcccagactggtcttgaactcctcagctcaatcgatgggcccacctcggcctcccaaagagctaggattagaggtgtgagcaactgcccctggcctttaatcaaattttaaagtaagcattgcttaaaacatgtcactcatcaacagcagaacattcagaatacctagggcagcagttctcaaattgtgggctgagaaccttgggatcctgagacattgtcagagaataaataccctctacttactgaaaagggaatttttaaaacctagtaccacagtaatctcttttaataattaattagctatctgtgggaaccaggagttcttcatgtacttcatccggaacagtgtatccaaacagattgagtgcagaaacagacatgagaacccgagcacgttctattaagccagatattaaagaaacattaaaaaatgtaaaaatgcggctgggtgcggtggctcacgcctgtaatcccaggactttgggaggctgaggagggcagatcacctgaggccgggagttcgagaccagcctggccaacatggtgaaaccccatctctactaaaaatacaaaaattagccgagaatggtggtgcatgtctgtaatcccagctactcaagaggccgaggcacgagaatcgcttgaacccgggaggtagaggctgcagtgagcggagattgtgccactgcactccagcctgggtgacagagcaagactccatcttaaaataaaataaaaaaaatgtaaaaatgccattcttctcactagatttttaaagaaatagttacttttcattacaaactttacgttaacatgtaatgggtttttgttgtattgaaagaattctcagttttaatttctgccgtggtgcatattgagagctgtaatctacctaagtagaagctctttgggatccccagtaatttgcaagagtgtaaaggagtgcttaggccaagatgcttcagaagcactggtctaaagcaagtggcttgaagcgtcctagactatgatttgcctttctactgccattggccctttatggactgcaggtgctcttattcaattaagccgggccatatcacagctcagcataaataccctctacttacttaaaagggaattttaaaaacttaggactaggaacctcttttaataatttcacaaatagatattgagggatgagcacaaaataaatgcttggcagatcaacttacttgggttcacataaaacttaagatgattttaaagtaatttacataatgcagcccaagttttcaaagatgttttattaaggtcatacaaaagtccagtcaataccatcgctgaaatatctaaatagcttcctccccaccccccctttttttttttttgagacagagttttgctcttgatgcccaggctggagtgcagtggcatgatcttggctcactgcaacctccgcctcccaggttcaagcaattctcctgcttcagcctcccaagtagctaggattacaggcacccaataccatgcctggttagtttttgcatttttaatagagacggggtttcaccatgttggccaggctggtctcaaactcctgacctcaggccatccacccgcctcgacctcccaaagtgctgggattacaggcgtgagccaccacatctggctaaacagcatcctctttctgagcttcttggatcttcagatctcctgccacatcatgtcagctttctgcatggctacttccatctcctcctccttgtcccacaccttctgcttctgggagtagagctctgccatgagctcattgagctccaagaactcctgcagggccagcttttgctgctgatgggcatctttcagttctttggcttgggatttcaatgtctctgaagcttcaaccaattgctgaaaaaaaaattacagatgttttatgtttgcttaacaaaggtctggaaggctggacatttagtcttacactatattaaagagttacaagtgacagccaatgctggttacagattaaagccacagatgggagaaatcccctgcttggaaaaagccctgtgctcacctcaccccagccaaacattcaatgggaagaccacctgggctcaggtgctgagcaacaaccccacaccaagaaagacctcgcaactgcatggagtaacttacagcaggacagctggctctgtaatgccaacctctgcccgaggtcttctacacaattgcaaaggtgaataaagcatttgggcactcaatctagttacttaaacacaagaagaagcagaagcagaaaaaaatacataggcaaagtgctgaagatttaaatcaggccaggcactcaagcctgtcatcccagcactttgggaggctgaggtgggaggatcacttgagcccaggagttcaagaccagcgcctgggcaaaatagtgagacctcatctcttaaaaaaactaaaaaagaaagaaaagaaaattaagaagaaagagatttaaatcaaagactccctgggtggagggccacaagtaggacttaccacaagttcagctgcactgacaacccaaccttaccacttgctgcactgatgaccctgacctccctgcagctgatctggccaacagtctggctgtttagtcactgtcacacttgctggactaagactggaagtgacgagacctggagcaagctggcagggcggctcccacatggggaggaaaggcctccccaggcctctctgggtccagctcagagtcactgtgactgtgtcaagcaatgagaggccgcccagcccacactgcactcccagacaagtcatgcagggggatgataccacagatgatcttaagaagcaatttggttttaaaatctgccccattttagaggacagatcacaaactacaggtccaaggacttcagtgtataaatgaatgtattttagaaaacagagcctatgcagtgagctgagatcgcatgactgcagcctggtgacagagagagactctgtctcaaaaaaaaaaaaaaaaaaagaaaaaagaaaagaaaaaagaacctaattgtgttattactatacttaatggcatttgaatataaagttcagatatcttactaaaaaccaaatttataaaggagttatgggggaatcagccacattttaacatatgtcctcttttgcagttccctgtccttactaagatgccccttctaagagcagggggcatggtgtagagagaaggacgaggtcactggtgtcaggcgcagcgtgtcactgcagataacacagtgccctctggacatcgttcctcattcagaatgggcggggctgggggagttgctgcttactctgcggggttgaactggagatggcagatgatgactgtgaagtgggtgcctgctgcaggcagcacgctgaaaggcacccatggcaaggagccaagcccagctagctgacatgggctgtgtagcaactgggcacagcaggggccctggaaccaggcttggccctgttgtcctgcttgcagcagggacagtccatgagtgggagtcccaaggaggtctgtaaacatctgtatgaaatgaaccttacaaaactctgcaaagcctcttaagcctttctcacccagaaccaaactgcctaccttgtgcatgtcctccttctcctgccacaccatgcggtgctgcttctctggcccccgtagctgctgtgtgaaatcttgtcgaggcgccgccttgtcctcaagttgtcactcaagcctgtttgaatatgagcaaaagaggaaataatttgtgttcctttctgtgtagcaactatatgtaaacgttgttagctgacttattgaaatgatttaataattcacaggactgttttttttagtgctgtatactaaataaaatacacaaggcagcaatacttaggggccagaaacactgtttactgcaagtcaattatggaatcataacttacagtaaaaatgggcatgtccaaggctcaatttttcttttgtcatttacagtagaatagtttgttgctaattttctacactgtctactgggaatatgtgttacttttataatcagaaaaataaatattgcagtcacgtagcatataatgatgtttcggtcaacaacagacagcatatatataagagtggtcccataaagttataataccatatttttcccgtagcttttctgtgtttagatacataaatccttgctgtacaggtctgtagccaaggggctatatgctataccatgtggcctaggtgtgcagtaggctgcaccaccttggttggtctaagaacactctgtgatgttctcacgtcaacgaaattacctgatgatgcattgtcgagaacgtatcccagccgctgaaggatgcgtggctggactgaagagcaccgtgtgatgtgggctacagctgagttctgtgcatgatgtggttggagccctgaggaaggggggctatggactgaatggtgtccccccccgccaatattagacgctgaagctctaatccccaatgtgactgtagttggggacagggcctataaggagatgatgaaggttaagtgaggtcataagggtgggccctgatccaacaggattcatgtccttataagagaccagagcgcttggatgctacctccccgtgggaggacatggtgagaaggtggctgtgtgcaaaccaggaagagaggcctctggggcttttagcctccagaactgtgagaaaataaaaccctgctgtttaagccactcagtcagtggtattttttagggtgcctggggctaatacagaaggattcaccctcctggggagaaggggccagtggcctcctctgattttcccagctgtcggcagaactccgtccctgcccctcctccttttgccaaagcatctttctaagggaagacctgattgcactgttcagaggtccccaaaggccacagaaaagtctcaactcctggacctcgtgagtcccatcccactgggggagctccctctctcttctctgctctcctctcaggagggaaagttaagggcctgcttggccaccctccagggagatgacatggtgggatgctgcatcctggtgatgagctgggggtaagccagcacgtctcctccagggctttccccagaactgaatacacactaagatgatctttagggatggaagactcaaactgggaagaatcacacacctgtagtcccagcctacaaggaaggctgaggcttggggatcacttgaggccaggaatttgagtccagcctggtcaacatagtgagaaccagtgtcaaaaaataaataaataaaaaggaagaatcaggtgactgttccaaagtcctccaaaatgggtattgggtcctgactctgctcttcccgggacaggggtgtgccttcccctttcctgttggccataaacccactccctctcaccaaagaaataagaaaaagtgttcacagcactttgagttatgtgaggtgtgcagaatttatcaggcccagagagacaggaatatggacttcagtcacacgccttgtactcatgccctggggtgactgtttaaaggcattttgttcctgactagctcaacccattatcttcctgttcctggaatttgtgataccaaagacaatgtagacaatgtagagacaataaatagctttttttttttttttgagacagagtctcgctgtgttgcccaggctggagtgcagtggtgttatctcggctcactgcaagctccacctcccgggttcatgccatcctcctgcctcagcatccagagtagctgggactacaggcacctgcagccatgcccggctaattttttgtatttttagtagagacggggtttcaccgtgttagccaggatggtctcgatctcctgacctcgcgatccgcctgacttggcctcccaaagtgctgcgattacaggcgtgagccaccacgccctgccttctttttttttttgagatggagttttactctgttgcccaggctggagtgcaatggcatgatcttggctcactgcaaactctgcctcctgggttcaagtgattatcctacctcagcctcccaagtagctgggactataggtgctcaccaccatacctggctaatttttgtatttttagtagaaacagggtttcatcatgttggccaggctggtctcgaactcctgacctcaagtgatcctcctaccttggcctcccaaagtgttgggattacaggtgtgagccaccacgcctggcccaataaatagcttacgttattttaatataaattcctcaggctaggccagccgaggtaggaggatcccttcaggtcaggagttcaagactagcctggacaacacagcaagacttctctaatttcaaattcttggtaaacaacttaggaactgcctcttcttttcctttaaaaacccacttataactgctgctaatattttaagacaacttgcatctccagggttgcacttctcaaacttggcccaaataaactattttttttttcaagatggagttttgctctgtcacccaggcttgagtgcagtggcgtgatctcagctcactgtaacctctgcctcccgggttcaagggattctcctgcctcagcctcctgagtagctgggattacaggcgcatgccaccatgccttgctaattttattttttgtagaaacgggcaaggtgggactcaccacgttgcccaggctggtctcgaactcctgacctgaagcaatcctcctgccgtggcctcccaaagcactggaattacaggcatgagctggtgtgcctggcccaacctctccacttacgttaattttgcctcctttcttccttttttttttgagacagagtctggctctgttgcccaggctggagtgtgctgtggcccaatctcggctcactgcaacaactgcctcccagtttcaagcgattctcctacctcagcctcccgtgtagctgggattacaggtgcctaccaccagccagggtaatttttgtgtttttagtagagatgggttttcaccatattggccaggctggtctcgggaactcttgacctcaagtgatccacccgcctcggcctcccgaagtgctgggattgcaggcgttagccatcgcgcccggcccgttttctccctttgggtcagaaaagtgattgaatcctgccacttcccgggtgctgggtgggaacagggaagagggggataattgcggtgctgctgtgacttgggtagatggcggcaaggcctctcagaggtggccttgtttgcaaagatctgaattaggcagggagaggagcaggcctggggcaatcagggaggggcagtggcacactgggagcaaggcaatgttccgcgacccacagaggcagcgcagtagctgttccaatcctggtcggaagacaccccggaaatgcctcagcaaccgcgcagcctttgatgaccccgctgccttcccgccaatcctgcatccaatcagagagcatccccactacacgtgttgagcaacggccaatcagaactgggatccggccctcggcctgcctcccaggaactccgagccaatggcggcctggcaccggcaggccaatcctgtgcggcgcgcgtgggcaggtcattgcagttgggcgctcagcagctgtggcagccggttgaggtctggaagtagcgttgggctgaagcagcggagttcgccatggtaagacccgggtcactcccgccccgcagatgcccaggcagacgaagttggcctcgggtggacagagggacgttgttgcgggcctgggcgctgagaggaggccagagaaggacgcagggtctagtgcgggacaggaggacacgggatcgtttcctggatctgcaggtcccagggcagggacggcggcttttgttttagatgaagctgccggcctttagtggacggggaccattgacggtatcccgtcccccagagagcaaatagtggcgtcgcctcctgaagacaggagggcgctgctcccgcttcacacgtctttgtgctactggaaacagctgaggagctgcaggagccttcggtgacgggggttggggctgtggacattggtcagatgaggaattaaggctcagacgctgctgcaacattgccttgttcacctaaaggcaaccgttaggccaggcgcggcggctcacgcctggaatcccagcccttggggaggtggaggcgggcggatcccctgatgtctggagttcaagatcagcccgaccaacatggcgaaacccagtctctacaaaaaaatactaaaattacccgggcatggtgatgcgagtctgtggttccagctactcgggaggctgaggtgggaggatcagccgagcctggaactttgaggctgcagtgagtcgtgatggcgccagtgcactccagcctgggccacagagaccctgcctcaaaaaaccaaacaaaaaaagcagcccattaacataacagattcccaagtcccaagtctgaatagctgtgatttgtcagttatcctttttttcctttctttttcttttttcttttcttttcttttttttttttttttgagacagcactcaataaatgctctgttgcccaggctggaacacagttgcaagatcacggctccctgcagccttgaacccctggcttccagcagtcctcccgcctcagccttccgagtagctggtactacaggtgtgtgccaccacacctggctaatttgtaaatttttcatagggacagggactcactatgttacccaggctggtctcaaactggtcccaagtgatcctctcacctcagccttccaaagtgctgggattacaggaatgagccaccacacccagcttggcagagtttaaagaaaccaccaaaggacagtgcagtgtcccagcactgatgatgtctgggagccattacacccgtaggcttcaaggggtagagagagggcgtggttacaagaaattaggagggtagttttatgaagagggcagcctgccaaaagtgtagccttgagagggattcaggtcacccacagcagttgagcaggaaggggccaaggagggagtaccctggcactgctttcctccagccccccagtatcccactgctgcctgccattgaccaaaccagcaagaagccagagggcgaggaaggcctctgatgctgttctcaagggtcagccttcgagagcacagagcaggtggtgaatgatggacggggtagaactggggggcagagagaggacacacactgagcacctgggcttggaccatggtgacctgggacctactctgtatttccttacccaacccgactaccttatcctttgatcaggacataatcaaaaactgctacttattcttcagagaattactgtgacgggctataatgtggggttgattattgacattaaatggcttccaagttttaaaataacctaagttttgtgtagtggcaaacaggttctagggctacttctagaacttactagctagtatggtttacagcaattagttttcacagcgataaagggatcagtcactaaccctcctaggaaatatcgattgtgaaagcgccagatactgaagcactatataaatattaaattagaatattttgcatgccatatagtttcaagttgccttgaaatgaatgggttcacatttatgttccggttcacagcgcgagtgtatctctatccacgtggggcaggcgggtgtccagatcggcaatgcctgctgggaactgtactgccttgaacatggaattcagcccgatggtcaaatgccaagtgataaaaccattggtggcggggacgactccttcaacacgttcttcagtgagactggagctggcaagcacgtgcccagagcagtgtttgtggacctggagcccactgtggtcggtaggtgcctgggcactggatggcagctttcctgagagggtgggagagcattggtaaagccccgtgtgggctcctttgaatcctcctgctgaaaggatgggatagacaggcatatgcccatggcattgttaggagagaaactgaaaggtttgtgatgaaagtgtctgtgagactcggctcctaatttaggaaaacctgacctacagggaaaagctgctttgccagcagtaagatgggctgtgaagagattccctcatggctgcctcagccctgctcaggtggccctgcctgcagggtgcagtggcattggttccctcccgaatgctgtgcattctcttatgctggtgcacgagagtcttgacctgcatcttaggcatagaaggtaagtacagaacagtcatttgcttccttcagattcgcagcctgaggtagtttccaaggagagctacaaagaggcagcacctttcagtggccccccaggagatgacagcatcacctgaaagcctgtgggacccaggtcatccagggtgccctgatgggactgccctgcagaagtcacactgacctggtgactgcccaggttgtaagagtttttaacttacagcctttgatgtagctacacgtaggaaagaattatgtacctgagtaggagtgcaagagcttatttcctttcactgggaactggtcgtgctgacatttataccacaagctgagtttaacgtcgtagagtcataaattgcatatgcagtttggttatcacacagagacattttctaccagacactgccactgttgacctatgacatgtaggtcatgtactttgaacagcatgtgctctgtagaagtgaacactcctggaatgtgtccatcttggtgagtacaggccttaaaaattcacagtacacactgtctcttttgcagatgaagtgcgcacagggacctacaggcagctcttccacccagagcagctgatcaccgggaaggaagatgcagccagtaattacgccaggggccattacaccatcggcaaggagattgttgacctagtcctggaccggatccgcaaactggtaagaagagaaggtttcatgtggccattgtcttgcatgggaggggtagttcttggaatgtgaaagggaagtcattttatcaacacttagaccagcatcttggccgggcgcggtggctcacacgtgtaatcccagcactttgagaggctaaagcaggtggattacctgaggtcaggagtttgagaccagcctggcctacatggtgaaactctatgtctactaaaaatacgaaaattagctgggtgtggtggcacacacctgtactgccagctacttgagaggctgaggcagaagaattgcttgaactcggaaggtggaggttgcagtgagctgagattgcaccactgcactctaacctgggcaacagagcaagattccgtctcaaacaaacaagcaaaaacttagaccagcatcttgagtcctactgagtctgcaatggtgatttgtccgtagatctttgcttttctttgcttcaaagtgtattgcatgtttattatggatgatttgaatccatatcaactctttagaggcaaagagaagcggccctggcttgttggaggttggtggtgtggcttccacgggcattggctcacgttgtctggtttctctcaggcggatctgtgcacaggactgcagggcttcctcatcttccacagctttgggggcggcactggctctgggttcgcatctctgctcatggagcggctctcagtggattacagcaagaagtccaagctagagtttgccatttacccagccccccaggtctccacagccgtggtggagccctacaactccatcctaaccacccacacgaccctggaacattctgactgtgccttcatggtcgacaatgaagccatctatgacatatgtcggcgcaacctggacattgaacgtcccacgtacaccaacctcaatcgcctgattgggcagatcgtgtcctccatcacggcctccctgcgatttgatggggccctgaatgtggacttgacggaattccagaccaacctcgtgccgtacccccgcatccacttccccctggccacctacgccccagtcatctcagctgagaaggcctaccacgagcagctgtctgtggccgagatcaccaatgcctgcttcgagccagccaatcagatggtcaagtgtgaccctcgccatggcaagtacatggcctgctgcatgttgtacaggggggacgtggtccccaaagacgtcaatgcggccatcgccaccatcaagaccaagcgcactatccagtttgtggattggtgcccgactggatttaaggtatgactgggtgacatggaggcctttcagcaagcagcagatgcacaaaataacactggccttgaaggcccacatcctttggggagactatccctgttccatgggctaggcatgtgggcataagttagtgaaccagagttgataattgattcagtgattttttttttttttttttttttgagacagtcttgctctgtcgaccaggctggagtgcagtggtgcgatctcagctaactgcaacctctgcctcccgggttcaagcaattctctggcctcaccctcccaagtagctgggattacaggtacctgccaccatgcccggctaatttttgtatttttagtagagacggggtttcgccatgttggacaggttggtcttgaactcctgacctcaggtgatctgcctgcctcagcctcccaaagtgctgggattacagtcgtgagccactgcgcccaggtcagtgatgtcttttgaactctttctgagtcatcacactatatatctgtggtgagctttatttatttatttattttttgagacagggtctcgctttgtctctcaggctgtagtagtgtgatcctggctcactgcaacctctgcctcccagactctagcgattctctcacctcagcctcccaagtagctgggaacacagacatgtaccaccatgcccgggtaatttttttttttttttgagacaatcttgctctgtcgtccaggttggaatgcagtggcacgatcttgactcactgcaacctccacctcccgggttcaagcacttctctgcctcagcctcccaagtagctgggattacaggcacctgccaccacgcctggctaatttttgtatttttagtagagatgggattttgccatgttggccaggctggtctcaaacccctggcctcacgtgatctgcccgcctgtctcccaaagtgctggggtcacaggcttgagccaccacacctggcctgatgttgtcatttatagaacgagtttgctgatccctgctctagttgaccctgtgcactgctgccaggacctcaggtcatgttattaccgagttctgaaacttggaatgcttcccctttggttaccaccttggaatgtcaccccctacttaggcaggcacatggaccacttcgaccacttcctgctgctgtggtagcagcattcatatgactcttgacccatgatctgctacctttgcatatccacagtggcttgttttgtgggtcagaagtgggtatgatgttccttgtgatagcatgggcaagagttgtgtactctgaatctatctgggtggaagaagtttagctacagaatttaattgtaatctacaaagaagacaagcagcatacattttcagagggaagactgtcttcacctgggcacctatcacatgtcacaggtactaggacacagccctttaggtagcttgggatcccaccagggtggctacctccaaggttcctttctctattcctcgtctggaactatcaccctgcctggtgcagagaagggcttagtgacgtttgtgaggtgaactgagcattctccgtgtcacctacagggtgtcttctgtttgaggagaagtagctaccatttctaggtttgatataagcttcacggactgctttctttcccttctctggcaggtgggcattaactaccagccccccacagtggtccccgggggagacctggccaaggtgcagcgggccgtgtgcatgctgagcaacaccacggccattgcggaggcctgggcccgcctggtccataagttcgatctcatgtatgccaagtgggcctttgtgcactggtacgtgggcgaaggcatggaagagggagagttctctgaggcccgcgaggacctggcagctctagagaaggattgtgaagaggtgggcgtggattccgtggaagctgaggctgaagaaggcgaagcatactgaggggagggtgtggtgggttctcccctgccacccctaggatggctgctttcaagttgtttgcaattaaagattctgtataaaaccaagacctctgagtgtcatgcagcttagctctgcctacaggagctggtgggaccccagagcctgcatggacagcggtggggtgccagcctgccctgctggcgtggggttgggtgcagcaggactgtacataagcagctgccacagaagtaacctgaccaggggaaaatagtgtttcttcaaagccaggcgttcaatgtatagatttcctaaattctaggaaagctgagtcagggttttcaactgaatttgtgatttttcgggcttctttattgatgggtcatgctggctaaaagggagcatgaagtgttggaggtctgtggatggggcttgtccatgaagtctctaaagctgtgggtcaaatatgtaaggtcaggacattttagagggggaaagaatattaaccgagttccacacagcattcgtgacccacagaaggtcacaacacacccatgctgaccccagtgctccacggtaacctggaaggcagcgttcgccctctgtgcgcacacacagccaccctgtccctcagcactgtcaactgccttggtagaaaacctcccatgaggtgggagatcgagaccaacctagctaacacagtgaaaccccgtctctactaaaaatacaacaaaattagccgggcgtggtgatgggcacctgtaggcccagctacttgggaggctgaggcaggagaatggcgtgaacccaggaggcggagcttgtagtgagccgagatcgcaccactgcactccagcttgggtgacagagcaagactccatttcaaaaaaaccaaaaaaacctcccacatttcacttcaagtgaagcttgttgacgtctatttataaaagacctggcaatccagatgccttttctcagaacccaaatcaggttcaggaagttgacccatggtgaagggacctggttagttcactgtggcacaaccaggaccggcacagaaacttcattctaagtgtgccttaatataaaaaaaaaaacaaaaaaaaaaaacggtagacaacatctgacctggaccctctgcatctcagaaaggtttattatcaattgaaggaggtaacatgtagcctttgctggggacaaagatgcaacaagtctctgccccatcctaggtgctgccccgtgtagggctcttcccaggcccgccccccttgggcagcctggtagcgctgggctggcgtggcatcctctggctggatccttcgcggctgctgcgttccgccagggccaatgctcccccgagggcagcgctgcctttgtttctccctgaggagacacaaagcacaatgattaggaatgggcgtgccccgtaactgcagcacctgctgtactcgtggtccttgggcaacatttgtgtctgcacatttccatcaaagtcccttcagctagaggccctgcatcagccccaagtcctgagactcagaaccactaaagacaggtcacgccccagaagcccttcgcagtgtgcaggggacccttttggatgtgtcctaggagcaaagccatgcaaggagcacactgccaagggaacattttgaaaggacacatgccacgggcccatttagggaaaggtaggtgtgctggtccacacctagcctggtcagcaaacctagtctgccctgatggtgccatcagcggcggcagcagcagggatgcgctgcctcatcctcaaggtgtgttcatgctggactgcacatctgagctgccctagatggtgacaggcaggaagtactgcagtcaccaagtcctggaattgggtttctgattttgattttggaagctctttcattgtttcctacctacccgagtgttctgcagtggtcaagcctcttgctttcgggtttagctgccagattccatgacagggttcctgccacagtctgaatgcttgagactttccttctgaccaccagaggctctcttggatgaagaccactggttcagagtttctttgatttgtaatttgcttactttactctttaagatagccaagaaaaatctgcaaccacacttgggccagacctttattttaatgggatggatttccctgggctcagggtgttgacagcatcggcaagactgagaggaccctccaaggtacaaacgtgcccgggtagcaggcttgcccgtggggtcacagacccccatgtggagtgccttggccagggctctcccctgctctggcctggcagggtctggcctgtgggctctaaacagggcatgtgtcccctgcctgactcagtgttgggtcccccatttcatccccccagtgtttcctttgtcctgactgaggaagtgccttgactactctgacctaatcagctacatgttccccctgcaggctcaaaccctggctggggcttgcatgttaccagacactgatacacgtttaggttgttgcctggaacactgaaagatctgacatgttgctaagcacgtggaaactggtcccaccccagccaaattccttgcaccctcatagaaactccacaccccaccccctcagtgcggtgtgcctaggcacaacctctttcctggctgtccctcgtgagggtacactgcagccccctctgtatgtaagctccctaacaaatgctttggactgaccaccctggcattaagtgtttcttcctttggaatcccaactggctctcatcctggaaaggtttggggcagtcccttgcaggaactcccctgccatcacttttagggtgactccagacaagggttcagccaacagaacacccagcctcaatcctggtccttccctggcctcttatcaagtgtagtcaaataccctgcaatgggaaactggattcctggagaaaagatgcaaccgggtgtctagagatccattaggtcatttctgtttcaccatctgtcaccctgaacaagcagagactcctgtgtctgagatgccgtggacccccgctcttccagaggtacctccagctccctttctatgctaataccagaaaattctccccataccctgcagggataggacggccctccttttgagtttccggggagttcttcctcagagaatgagatctttgctcattcttgcctggtggtggtgagggttacagcggtgtcatgcttggaactctgtcagcttgtcacacttgactcaacaaagatggagtcagggttgccctgagccaaggaggagcgcagtcaggaacacgtggtgggtgatgattggccacctcagcccagcagtgactcgtgaaaaaggcacaaagcagacaatagccaccacgcatctcttgtggtacaaactgccataattctagtccccactgactggtcaaggggcaaggattctgtcttccatgagcagacgggccccgccaccctgcagcagctcccacactgggccagcatcctcctctgaggccacagtccctccgcaggccaggccagacagagtcagatcttctgtagccgcagcctaatctgggtgcttaggtgcccatgcgctctcagctccccacagcacgcagcatggcaggccgcagacttgggcctgactcagagtcggcttcccttcagcttgctgccatctgagcttcctctcctcccctcagcaatgacccagtcccttctggaaacaaggatgagagggaagcagaaaagagcaaataatgcttttttttttttttgagtggtgggggaggggggtttagagactgagtcttgctctgtcacttggtctagagcacagtgatgcaatcacagttcattgctgccccaaactcacggcctggagggatcttcctgcctcagcctccgatgtagctgggactacaagcatggggccacacctggctaataatgcttatctttaaatctgacagggttgacaaaccctggaaatgtgaaaccaacataactttaagccataactgatactaagaaaatcctggaaatcttcaggaattgtgcgctctcagggttagtggggagccctgttcaggcctcctcactttatgaaggatgagaaggcccagaatagaatggcctgcagtgccttgcagtggggaaaccagctttgcttagccctgctccctcgggaggggctactgagtcagacgggctagacagtgggattgaaccaagtgtggctgcaggttggggtgtgagggcggggtgactgatgcccctacgcctggtccctggagggccctcctaaacgccaaagatagtcaatgaccattgaatgttgaaaacagcctggctcagcaggggtcagtggctcatgcctgtaattccactactttgggaggccaaggcgggaggatcctttgaggccagaagctagagaccagctgggacaacacagtgagacctccatctctacaaaaaattaaaaaattagccacgtgtggtagtgtgcacctgtagtcccagctactcaagaggaggtgggaggattgtctgagcccacgaggttgaagctgcagtgagctatgagtgtgccactgcactccagcctgggtgacagaacaagactgtctcaaaaataaataaataaataaaaagtaaaagaaaatgcagaattagctcaaatactttaatctaaaatatccaacgtattatttcaatacgtaatcagtacataagttattcataagatgttgcattcacctctgttctttttcttgtttctttgtagaaatgggtggggtcttgctattttgcccaggctggtctcaaactccgagcttcaagtgatgatcctgcctcagcctcccaaagtgctgggattacaggtgtgaaccaatgcccagcctcattcactttggggttttttgtttatttttgagacagggtctcactctctcacccaggctggagtgcagtggtgcagtcacagctcactgcaacctcaacctcccaggctcaagcaatcctccaacctcagcctcccaagtagctggaactacagatgtgtgccaccatgccctgctaatttttgtgttttgtagagattgggtctccctgtgttgccctggctagtcctgaactgctgagctcaagagatccagtagctgtggactcccaaggtgtggtgggttttttgcattcccttttgtaccggtcatttggcatctgctgtgcataccacacgcagtgcaatgaccatgccagccatgtgtggctcaaggttcctgtatcaggcagcacagctcaggtgagatggcttccacactccaaacgtcatcttacatgcatgctctggacaacttttaaaacgtatagtttctctaaacttaaatagaacgcttgactgaatttatttttccctcaatgactccagactctcaccaagacaagcattgcccctctgtgcttctggggttttctatttccccttccctgtgggtatgagtgaatctggtggtggttgtccccacaggctgtctggatggctgggggacaggcatccagaggacccagggtgcctgctgcagctcagaggcaagcttgagagggcctggcccacagcctggagaactccacacccgagtcctgtgggaaacctatggcctccatgccccccagccggatcactccaacaaccccccttatgtcctccaccccagattctgccattaccagggcagccggagctctgtgtggtcagatgctgtaacatgtgggcctcagagagctctcagcattccatctgtggcctctgcttctcactcagcagcacactcctggtgtggccccacatctgccccattcccctccacctcctcctcaggttccactctatctccacagggcctcggtgccctcatgtcctggctttctgccacccagcctacaggacctgggctatgcttccaggacagagccctcaaggaacaaatgaccctgagggtcccagcactccaggccactcctgtccttgggtaaattctctcggagcccccattcgctcacacagcaccccctaatcctcctggcccctctatgcctgcatttccagtgccacaaccctcacatacatcttgaccgtgcccccccacctgcctcctcccgcccccggcctaccagcacctggacagcgctctgcctcacccacgccctcccactgccccagctcacacgtgtggggctctgccgacccccctcctgctgtaacccttgttacccccctgtgtaacccttgctgctctctgggctctgaagcctcctcccatctccattgctagcggtttttcttttttttttttttttgagacagggtcttgctctgttgcccaggctggagtccagtcacaagctcttggctcacggcaacatccccctcaggctcaggcgatcctcccacctcagcctcccaagtagctgggactacagatgggcgccaccaccatgcccacctggctagtttttttttttttttgagacggagtctcgctctgtcccccaggctggagtgcagtggtgtgatctcggctcactgtaagctccacctcccaagttcacgccattcttctgcctcagcctcccgagtagctgggactacaggcacctgccaccacgcccagctaattttttttgtatttttagtagagacggggtttcaccgtgttagccgggatggtctcaatctcctgacctcgagatccgcccgcctcggcctcccaaagtgctgggattacaggcgtgagccaccgcgcccagccctgcctggctagtttttgtattttttgtagagatggggtcttgctatgttgcccaggctggtcttgaactcctgagctcaagggatccacctgccttgacttcccaaagtgctgggattacaggtgtgagccaccacactggtctcgctggtggttttcctcactgcctctcactgggctctgagtgtgatcatctcacttccctgccacaccctctgccctgagagctaggcctggctcccagagccctgagtgagtgctgagtgagcagcaacatcccctggagtgacaaggactctgttcccagtttctccctcctgctggacacttgcccctcaattgctcatctcagcctgcagtgcatacacccctactccaggcctgtccctcagcccctctccaccctcccttgggtccgagctctgggtggcagtgaggtgggcgctgtcaggaagtgcacgcaggaggtgcaaccaaagacccctgggctctcacaggaccctggctctgagcccctgatcacacctgctgacacctggaaagtgtgctaactcaggagtgaggtgggcaagggggctgagtgctggagtgtggccacggggccagcatatggcatctgtgtggcactgcccaggagactctgggaaggtgggagtctctgtttcagcctccagacttcctgtccagataagggacaacttctaagaggccgaggtgtttgtcacgcctggggctgtgctctccagcacagcacaccgcactctggccggctgtcttcccttggagaggggctctttacactcaccacagttcgcctctggggtgtgggggtgttggtcctgtccccagatgccagcccgactgtggggtctagagactgccacactatgtctctaataaaccgagtcagaaatcgtgccctgtggctaagcggcagggcagcgatggcctgcatggtcctgcccttggagctcaaagctgcttgggcctcctggggccaggcagtttccatggaaacaagggagagcaccaacccccagagataagccaataaacgcaacctgcaaaaggagagaacaggcagcaagagaggcggagggaacccggggggcctggagagagaggcgagtgtgtggcagccgggctgtggagcagaaccggagaggccgcaggctgcgggctggaggaaaaggagggccttgcaggggggctactgttcctggcagagagaaaggaggcctctggaggtcagagagagacacaaagagagcagacagagcgaaggtcagtgaagaggccagcacgcaggtgcccgggccctcttgggtcagcgggtgtgggtgcccaggccctggcactccccccatgaaggcagcctgctctccacgccttctcaggatgcctgtgcgggtctcctcgtgcttccaggcagggagccagctctgggccttcttccattggcctaggaggttgtgctgcatttcagagagagccatcgggagcagaagcaacatgcggagaggatgagcagggacagaggctccaccatgacactctcagtcccagaatctcctcagcccagtcaccacactggagagggaggaggcccttcagaccccgcaggcctctgcaccctgggagttccgcgtctgactctcttcctgcagtcatcagagggaaagctgaccaggactggccaagcccctgcgcaggaactgggcccaggtggggtaaaggcaggaagacctctgcctgccctggtccctgggcacttccctggatgctgtataccacaggacagagatcgagactgtcttttgaaaaacatttagagacaggatctcaccctgtcacccagagtggtctaaaactcatggcctcaaacgatcctcccgccctggggtcccaaagattataggcgtgagccagggcgcccagccccaaactgtcttgattcccgacgttgtaggatgaaggcctgagtgaagaggtggaggggagctaagcggaggagccctacccagcctggaccaccctggggtacgcactctgcgtccacctgacctggacgtgcctcctaaggctggcagcgaagcccaggagcagagcccacggatgggccctgcaggtgctgggcctagaccgacagacaggcctggccactcagggccacgccgccgctgtgccgcgcctgccccctccccgcccgccacccccgccactggggcagggacagcgggcccagctctgacctcgggtctcgggcacgctcgacgtgggcagagacacggccgcagggtcctggggctcgctcgctagcctctgcccggcacacatggacttgagcatctggaagacggcgaggggggccacgttcagcttcagcaggtccaccaggatcctggcggggacagacgcggggccggtgagccctctccgcccggcgcggccacctccctgaagacccgacaaggacgggggcgggggctcctcccgaccagcgggccgggcgtacccggaaagcagaggtcggggggtgtctggctagcatgcggcccccacccgccccgctcacttgaacacgtcggggtcgatagcgccgcccgccgcctgcgccagctcgtacagctccatctcctcggtgctcagcaccttcttccgccgcagcgccagcttctgccgggccgcctccagccccgggggcgccgccgaccccggcccaggccctacgccctgcgccgccatccccgcggcccgcggaaaggtgcgctccgccccgccgcgccccctagcggctgcgcccaccaccctctccctgcagcgtcaacgtctggcctcccgggctgccctggcgggaggggcgggagcgcggggacggggccgcctccgcggcgtcattgaagcccaacagttattgggcgctcacggtgtgctgagcggcgctctaggagccgggagtggcatggcggaccgcaggggtccgctgcttggcgatctgggcctccctgatagactgcattggtcgtgctcgcttaggtggcagcacccagcccagtgccaggcatacattgggcgctcggtaaatgcctctttcaagaaagcgtgcaattttctggtccgcaccggtgcaccactaggggtcgcttttcggggcgggcggggggaaggggggggcactaatcaacaatactgcttacgcgcacgcggattccttgctggggagaaagtaccttggggcgccggaggccgccacaacgcaggcgcattcagctaaggaccactccctcccccgcactcctgcctcgccatttctcttccccgcccggccggccttcgctttgcgcacgcgccttttgaggtaacggcccaaagaggtggaagcgcttttcccgcccggccgcggggcgtggctctgcgcgcagcttgatgacgactttcggcgccgtggcggaatggcggcttccatctctgaggcgagcgacgctatggatcccacaggtgagtggcgcgggatgcgcgcctgagacggtccggcgccctgggggcttccccgagcccctgcggaggccatctccgttcctccgcgagccccaggactcgggccggccagccggctctgcccaggcagggcaggctggggagtggagggccccggttgagtcttctgccacccctctgggccatgcctttgtgccaccttcccgcgcggctcgtacactctgtcccggtccggtaggtgttcgctgaaggttaaccttcgttgaaggaattgagggaaggagagctttgtgttgaagttgagccttgggctcaacaggcggtgtgggtgtaagagccgggtgtgggtcccagctcgacccttcccctgtgtgctccacatctccgagccttggttttggaggctcccgaagcccaattggcaggagagttgggaacaagtgggcgcgtgctgttaagggaggtcttggcatgacagaatttgcttggaatgattagcgttgcccaagggctgtcgctgtcagaaaaacaaaacaaaacaaaaaacaaacggggtccgaagcagctcctggagaaaataggtgttggagagtcgccatgaagtggactctaggtgcatggcacgcagagacctgtgacgggggagggtggccatggcagagtcagataacaggggtgtgggagacacctcttgggaattctcgggtttgaatttcctttcttggaaatttcctagaaattcctctgggcctcattctttcagtcgctgcccatgaaagcttcttctgagcagagatgctctccccttcatcgtctctagagctccgcacttaccgaacagtccacagtttaatgctgctttgatcattttgtagtagtgcagcgttttcttgaccaagtgccttaggactgaggcgtttttctttaacactgcagttgatagctaggttaagaattgaggccgggagcggtgactcacgcctgtaatcccagcactttgggaggcccaggcgggtggatcacgaggtcaggagttcaagaccagcctggccaagatggtgaaacctcgtctctagtaaaaaatacaaaaattagccgggcgtggtagcgggcgcctgtaatcccagccacttgggaggctgagtaagagaattgcttgcacccgcgaggcggaggttgcagtgagctgagatcgcgccactgcactccagcctgtgcgacagagcccagagcgagactccgtctgaaaaaagaaaaagagaaagctggccgggcgcagtggctcacgcttgtaatcctagcactttgggaggctgaggcgggcggatcacgaggtcaggagatcgagaccatcctggctaacacgtgaaaccctgtctactaaaaatacaaaaaacaattagccgggcgtggtggcgggcgcctgtaatcccagctactagggaggctgaggcaggagaatggcgtgaacccgggaggcggagcttgcagtgagcccagatcgcgccactgcactccactctgggcgacagagcgagaccatctcaaaaaaaaaaaaaggagaaaaaagaaaaagcggccgggcatggtgactcacgtctgtaatctctgcactttgggaggccgaggagggctgatcacgaggtcaggagtttgagaccagtctggccaacatagtgaaaccccatctctactaaaaatacaaaaattaactggatgtggtggcatgtgcctgtaattccagctactcgggaggctgaggcaggagaattgcttgaaccagggagtcggaagttgcagtgagccgagatcatgccattggactccagcttggcaacggagcgagactccatctcaaaaaagaattgaaaaagtcggcactttgggaggctgaggcagcggatcacttgaggccaggaatttgagaccagcctggccagcatagggaaacccgcctctactaaaaatagaaaaattaggcagcctgtggtggcaggcccctgtaatcccagctactcgggaggctgaggcacgagaaccgtttgaactttggaggtggaggttgcagtgagatgatggagtgagactcagtcaaaaaaagaaaaaagaggctgggcgcggtgggtcatgcctgtaagcccatcactttgggaggccgaggcaggcggatcacgaggtcaggagatcgagaccatcctggctaatacagtgaaaccccgtctctactaaaaatacaaaaaaattagccgggtgtggtggccagtgcctgtggtcccagctactcgggaggctgaggcaggagaatggcgtgaacccaagcggagcttgcagtgagccaagatagcgccacaggactctagcctgggcaacagagcgagactctgtctcaaaaaaaaaaaaaaaaaaagaattgaaaaaacttgggagatgttcctattgccgagaggcagagattcctgtctcctctcactctacagctgcctacatggcctctttacacccagcctctcttcctccacagactgccaggtgtcaccccagtgcctttacttgtgcagtgggcagagctgggccatgaaaactgtaactagcccaccagtgggacttggcagacttcctgtctgtgaagtctaggtctgagcatttttcatttctgacctgggtgagctgagaggtaggattctggaaagctgatcttagttttctttctttttttttctttttttttttccattttttgagagtttccctctatcaccccggctagagtgcaatggtgtgatcttggctcactgcaacctccgcctcctgggctcaagcatttctcatgcctcagcctcccgagttgctgggactacaggtgcccgccaccacacccagctaatttttatatttttagtagagatggggttttgccacgttggccagactggtctcaaactcctggcctcaagtgatctgcccaccgcggcctcccaaagtgctgggattacaggtgtgagccatggtgcccggcctttgatcctagttttctcatggcttattcaaaggctcccttggcagagcttgctcttcccctccgttcattgcatagtgcacaggtaatatggaggggatttgcccctgtctcagttttgtgaggtggtgtttgtattggaatcttggttctgccacttactagctgggtgggggtaagtgaattatcccatttctgcctctccttcatcatctgtactgacctgcagtcatagtacatctgtacccatcgggttgttgtgaggttggtttttctattcttgtatgacaaacttagcagcttaaaccggcatccatttattatctcatagttatgtatgcagaagtccaggcattacaagattaaaatcaaggtgttggtagctttgctcttatctggagactctagggaagaagctgcttccaggtttatttaggttgctggcagaatccagttccttggagttgtagaactaaggtctttgtttccttgctggctgtcagctggggactactctgtaacttaaggtccactaacttaggactctaattggatgtgcaaaatgccgtcatagcagtacctagatgagtatttgattgaacaaacaggggtcgggaatcttgggctgctatctttagaattctgcctaccactgaggattaaatgaggagatatatagagagcatttagagcgagtcctggtatataacaaatgctgtacaagcattaccgaggattcagtactacaaacttggctctgaagctggccactatgggctgcaggagaacagtatttctttttttcttttttttttttttgagatggagtcttgctcagccttctaggctggagtgcagtggtgtgactcagctcactgcaaccacctctcctgggttcaagtgattctcgggtctcagcctcctgagtagctgggattataggcacccaccatcatgcccggctaatttttgtattttggtagagacgggttttcaccatgttagccaggctggtcttgaactcctgacctcaggtgatcctcctgcctcagcctcccaaagtactaggattacaggtgggagccaccatgcccggctgagaacagcatttctataaaagatgggaattgctggccaggcgtgttggctcacacctgtaatcccagcactttgggaggctgaggcggatagttcgcttgagtctgggagttcaagagcagtctaagcaacatgatgaaacccccatctttacaaaaagtacaaaaattagctgggcttgttgatgtttgcctgtagttcccgctagttgggaggctgacgcaggaggatcacttgagctcaggagtttgaggttgcaatgagctatgatcgcaccactgtactgcagcctgagtgagcaagcaagagcctgtctctaatcaataagtaagacatgagaattggcaatgtaatgtgtgttgcgaattggtaatgtaatgtgtgatattctcctgggcattgtgaaactatgtaatattttgttagggccaggtgcagtggctcacacctgtaaccccaacacttaggaggccaaggcaggaggatcagtggaggccaggatcctgggcatcacagcctgacaaactagcctgggcaacatagcgagaccctgtctcaaaaaagaagaaaattagccaggcatagtggcacgtgcctgtactcaggagactgacgtgggaggattgcttgaacccaggagtttgaggttgcagtgagctatgatcgtgctgctgcatgccagcctgggcaacagaacaagaccctgtctctaaaaaaataatataaaaagattctgctgtgttacagatgttgagattttgtctttgtttttccacagtggtttgctaagaaggccattttcaactctccactggaggctgctatggcgttccctcacctgcagcagcccagctttctactggtaaatatcaactgcttgcatgagtgtcagtgtggccatagttctgtagatgtgggacttgagtgttcttcccattgacttgaagaactctgtagtggttgttttttaggggcatgtgatggaaacaatggcagactccttaacttgactgtgcctccttattactacaggaccttggggttactcaacttctgggctctgggttttctcacacaagttcattcaaattctattttctctacttcccaagggtgtgaggaggaggaagagcaagtgagaaggatgtatatcagaatgttcatgacttgtaaattcttaaatagatatgaagtattctaataatcagtcataactgtcatcagtgaaaaatggagacactgttgtgcgggggcctgctaggttgcactttaagtattaaacatgatgctgagaactgggtgcagtggctcatgcctggagttccagctactgaggagactgaggcaggaggattacttgagcccaagagtttgagaccagcctgggcaacatagtaaagactcaaaaaggaaaaaaaaatgatactgagaagtgagaagttagatatgaggcaagtattggtgatgaaagttcctaattttagttcttaggaggtgtttttgtaaacatttatgtggaaggacattcttcctcagtgcaccccactagagtcaacaggtctggcccatggccagatagctcaggtctccatctctgtctcaatcactgtgaaatccacccttgtttggaggtagacaaggccttagatatcctttggcaaagctctctaggttgaaattctagccgtttctcctgatcttaatccattcgcacagatgccctttttctccttgtagtcagggtgctttcctcacatcccacgactaggtagatttcattagtccttgtcctgagtcttttctggcctctccattgccttcatgattagtgtctacttggctgaagccatctataatttttgtctgccctgtgttttaagatcattggtttagtggttcagtgtctaccgaataaaggacagctcatttaagagagcttctgccatagccgtgatgttgttgggcgttcttactgatgatcacaatggtgtaatcacggtggtccatcaggctggaaatttccagttgatgcgtacaaactcagttatactctgtggtttgtgaggggtgcccttacacagtcaagatcctcttctggctgtggggctgactctgggaccaacccctacttatggattggtggaagtcaatcctgttctgatcatgtgcagcactctgagggggtactgggaaacaaggctgatagttcctggtccagagagcatagaactacggcctctcctagtagtactctttgggccactgacacacatgaggggctggggaaccaggccagaggttatgggtctgactcataacctattgcttggatttctgtttgactcaagccagagtgccactaaaagctgcagagtgaatgtcctcgtgactttttcgctttgttctgtcttctaggctagcctgaaagctgactctataaataagccctttgcacagcagtgccaagacttggttaaagtcattgaggactttccagcaaaggtatagctctgctaacgtctctctttggagcatgtcttatacattcgttgaagaactttattataattcctaatttccctccccgccccaaccccgagcactgcctggcacagaggcctggccattaccatgaccattatgtttacggacacatttaaacatgtgaacttgtagatcaggggttctcagccacagatgctctgttcctcagggacacttggcaatgtcagagacatttttggttgtcacatctggaggtagatggggagtgtgtgctactagcttctaagaagtagaggccagggttgctggaaacattctgcaaagcacaggacagctccctgcgacaaagaagtatctagcccaaaatgtcaacagcaccaagattgagaaaccctgatgtaaatactaaaaatatgtatttaaatcacacctggaattgtcaagcccaggaagctggcctagacaaggagttaataccaaatggcaataggtcagttaggtttcaggggcttcactctggaaaggatgttgctttcctttctgatagagcatccttcaagtcagaagtttccatcactgtttaaagaatcttttttttgtggtggctcatgcctgtaatctcaacactttgggaggccgaggtgggaggatcacttaagcccaggagctcaaaaccagcctaggcaacatactgagaacctatctctacaagaaatttaaaaattagctgagtgtagtggtgtgtgcctatagtcccatttactcaggaagctgaggtggggggattgctcgagcccaggaagtcgaggctgcagtgagccataattgcgccactgcactccagccagggcaacagagtgagaccttgtctcaataaatatgtattttgtttatttttttattttatatttttttattttcattgtttgtttttttttaaatttatttatttttcttaatttaatcttctatcacacacaaaaaatgtattttgatcggaggctgacttacttaactggcccaagtctaaagcaacactgcttggtatgccatgtaccccactggaagaacgtaaagctgctcttgtcgttggcaccatggcaccaggcttcctttgggtgcagggctgatcccataggtcttagccagggagtggcttcctcagagggagctgggcagcaggtggtgcaggactagagccggggcaggagtgggtgctggtcctgcacgcggcttcaccacacattgtttcaggagctgcacaccatcttcccatggctggtagaaagcatttttggcagcctagatggtgtcctcgttggctggaacctccgctgcttacaggggcgcgtgaatcctgtggagtacagcatcgtgatggaatttctcgaccctgggtaggtaggttggtcaccggagagaggctgggtgattcctcatggtgattccacctttaaagcactctctgaatttctcaagattaagaaacaatcaaagcaagataattcgcagttcatttgccctgtttttgtttgtttgttcaatttcgtgttcagtggcccaatgatgaagttggtttataagcttcaagctgaagactataagttcgactttcctgtctcctacttgcctgtaagtaaaccacagtggcgagagtggtgctcggggcagtgggccttgcacagccctgtggtgatgggcaatctcctccagtcctctgagggcaaattgaggagcgattcgttccaggggtgtgagaggcaaaggaaatgggtattgcatgactgctacccttgctgtctgcagctctttatgagatgatggtaggagaagttctaagcaaagactcttgttcaggcactgctcagaggcagggcactgtccccacaggtgggttctgtgtgtgggtctcttccccagcagccttcccaaagtgctgctgtgcatgtggggtactgcgtatcgggcactacgcaccagcacctgcccacaagcctcactgcctctctctgcagggtcctgtgaaggcgtccatccaggagtgcatcctccctgacagtcctctgtaccacaacaaggtccagttcacccctactgggggccttggtctgaacttggccctgagtatcctttggtggccctgaggtagggaggctgtgggtggcccagcacttgacactgagcggggggtggctcatcaaccctgcatgtttgcccctgacgctgctctcagatccgttcgagtattacatattcttctttgccttgagcctcatcactcagaaggtaaggaagggatgccttttgctgggagaggtggggccacggcccagggcttccctgcacttgttggtgcttggtggcctggcagtgtccagagcccatgctaacaactagcttggttttccagccacttcctgtgtccctccacgtccgtacttcagactgtgcctatttcatcctggtggacaggtacctgtcatggttcctgcccaccgaaggcagtgtgcccccaccactctcctccagcccaggggggaccagcccctcaccacctcccaggtaaggctgccctttgttaagacctggcccttccataagggagtggcctggtcacttgcatcgcccacggggacaatgttgccaccctttgtccacggttccccaatttttcttatgcctcattccccatccccagctgtcttagcacagactggagggtggggtatggcgggcaatctgtccttagtgttcagtgtctttggccatcactgaagctatcaggtccctggaggcctccacggcctgcatgggaatgggctgtgttgtgcatggagcagccgtccccacaggcggtgtctgagtcagtgagcagggtggaaaggttgtcctatgagcaaaaaaaataggaatctctatgggccccaggagcagatcttgaaggggaggccctgggccaaagagcctgttttgtcagtccaaacaggctgagcctgatgcgtgcctgcacaggttgggagtgtgaaccaggggctctcatgcctggctgtcctgggtcctcagtgggcactaacgcaggccgacttcccaggagaccctcttgccccagcagcagctgcctgtgcacctctgaccctctttaatctcactggcccatcgccttccccaggaagctgcatgcaggccccttcctgcagatgctggcccaccccagatctgctggcatgtgtaaggcaggccaggcctttacctacaattcacgtgtggacagattccccagagggtggaggggagaaagggagtggcaaggaggagacgctgcccaggtagcggcagtgatcagtgaccctgaaatgttgctgatcctgtggaggactcggaggtgaggcagattcacaccctcttagatgcagaattcagctgccaagcagtttttgatgcctggtttttttgcttgttttaactttttgggttgttttaattttttctattagaaattatatacgtaggctgggagtggtgactgatgcctgtaatcccagcactttgggaggccaaggtggagggatcgcctgaggtcaggaattcgagaccagcctagccaacatggtgaaaccttgtctactaaaaatacaaaaattagctgggcatggtggtgcacacctgtaatcccagctactcaggaggttgaggcgggagaattgcttgaacccgggaggcggaggttgtggtgagctgagattgcaccactgtacacacacacaaaaaaaaaaaagaaaagaaattatatatatatggtttgggtttatttatttattttattttattttattttattttgtgtttggagatggagtctcactctgtcgcccatgctggagtggtgcagtggcatgatcttggctcactgcaacctccatctcctgggttcaagtgattctcccacctcagcctcgcaagtagctgggattacaggcacaggtcagcatgcccgactaatttttgtatttttagtagtaacggggtttcaccatgttggccaggctggcctcaaactcctgacctcaggcaatccgtccaccttggcctcagacaaagtgctgggattacaggtgtgagctactgcacccagcatttttttttttttgagacagggcctggctgggttgctcagcctggagtgcagtgttgtgatcacaactcactgcaacattgacctcctgagctcaagtgatcctcccacttcaacctcccaagtagctagtactataagcatgcacccccacacctagcttttattttatttttttgtaagatgggtcttgctatgttgcccaggctgctctagaactcctgggctcgagcgatcctcctgccttagcctcccaaagttcagagattacaggcatgaaccactacactggtccattgtgtgcttttttttttttttttgacagggtcttactctgttgcccaggctggagtgcagtggcgctgtctctgcttcaagtgattctcctggctcagtcccaagtagctgggactacaggtgcctgtcaccacacccagctaatttttgtatctttagtagagacagggtttcaccatgttgagcagactgatcttgaactccttaccacaagtgatctgcctgcctcgaccccctaaagtgctgggattacaggtgtgagccgccgctcctggcctagccattatgtatttttaaagacggtcttactctgtcacacaggctggagtgcagtgtcataatcatcacttggtggcacaatcatcactcaagccagcctgtcacatcagtgactataggcatgcatcactacagccagctaatttaacttttttttttttttttgcggagatatggggatgcctcgctgtgttgcccaggctggtcttgaactcctggcctcagcaatcttcccgcctcagcttcccaagtagctggaattacagttgcaagtcaccacacctaactttttattttgaaataacttcagatttacagataagtttccacgcataataaaaataacttgcaggctgagcacagtggctcacgcctgtaatcccaacactttgggaggccaaggcgggaggatcacttgaggccaggaattcgagaccagtgtgggcaacgtcgggagacctcatttctacaaaaaaaaaattagccaagtatggtgatgtgcaactatggtcccagctactcaggaggctgaggtgggaggatcacttgtgcccaggagcaatgagctgtgagcgtgccactgcactttagcctggggtaacagaacaagatcctgtctcagggaaaaaaaaaaaaaagaaactttcatagatcttcacccagatttccaaactgttaatactttgctgcatgtgttggtttccacctctattgtatgtgtttttttttctctaagccaatatgagtaagctacaggatatggcatcccttgacctcttaatatttcagtgtatttcctagaagcgaatgcattatcctatatagtcacagtgcctgtaaccacaccaggaagttagtattgccaccaggcctcacactgtgtgcagtgatgtttcacaggctcacccactgtatatagtgatatttctagtccccttcagtcaggaatggtcccttgcctttctgtctttcctgaccttgacccctccagagcatgcggccgccccttaggctgtgtggtatttgctggtgactaggcccagcatgggctttgttgggaggagcaccgtagtgggacacctggtccttgccaggtgcccttttatcactgggttaaaaaggtgccggtcaggtttttccattgtaaaataattactttgtgcccaggttaaatagtaatgaggctgggcatggtggctcatgcctgtaattccaggactttgagaggctgaggtgggcggatcactggagctcaggagtgtgagaccagctcaggcaacatagcaaaatcccatctctaccaaaaatttaaaaataaccaggtgtggtggtgtgcgcctgtaatcccagctacttgagaggctgaggtgggaggattgcttgcacccaggaggcggaggtagcagtgagccaagatcatgcccctgtactccagcctgggctacagagtgagaccgtatctcaaccaaaaaaaaaaaaaaaaaaattaatagtgcctgggcaacatagcatgaccttgtttctactaaaaagaaaaacatcagccaggcgtggtggtgttggtcccagctacttgggaggcctaagtctaggaggttgaggttgcagtgagctgtgattgcatcattgcactccagcctaggcgatagggtacgatcctgtctcaaaaaaaaattgataacgagaatttaccgggaagatgtatggagactatgtaattatcagtagtattaacatccacttttgattctcctattttttcaaattgattttatttgcagacagggtcttgttctcttaccccaggctggagtgcagtggtacgatctctgctcacagcaacctcaacctcctgggctcaagtgatcctcctgtctcaaccccccaagtatctgggactacagggaggcatcaccacacctggctaatttttgtatttttttgtggagacagggttttgccatgttgcccaggctgggaatttcttaattttttagaaacagggtcttgctctgtcaccccagggtggagtgaagttggtacgatcatttgtcactatagcctcaactcgtgggcccaggccattctccctcctcagccttctaagtagctaggactacaggcacatgccaccaggcccagctgttttatgtttcaatgtttttgtggagataggttctcattttgttgcccaggctgatctcaaatgcctggcctcaagaaatcctcctgccttggtctcccaaggtgctggtgtgagcccccatgctcacaggtgtgaacccccatgcccaacctcactgttgattcttgaccaaatcagctattaatagggcggttactaaatggtggttctctaacttcatccctccttcattgatttgtgggtcaatggtcactctgtggtaaggaagacttctctctttgctatttatttattcaggtatatcagtgtggatcagggggctggggggaggtgtgcattgtgctattgaacaagttgtaatctgtccctgctctcgttgattttgatatccagattgcccctgttacagcgggagcgagttacagcgggagcctgtaactcgggcctgtttctgtgtctccctcaggacaccagccataccctttgcttcctatggcctccaccacactagcctcctaaagcgacacatctctcatcagacgtctgtgaatgcagaccccgcctcccacgagatctggaggtcagaaactctgctccaggtgagagttgagctggtcaaaagaaactgttcagccagctgggcatggtggctcgtgcctataatccccagcactttgcgaggcctaggtgggcagatgacctgaggtcaggagtttgggaccaacctggccagcatggtgaaaccacgtctctactaaaaacacaaaaattagttgggcgtggtggcaggcgcctgtaatcccagctactcgggaggctgaggcgggagaatcgcttgaacccaggaggtggaggctgcagtaagctgagatcgtgccattgcactccagcctgggtgacagaactagactccatctcaaaaaaaaaaaaagaaaaagaaaagaaactgttgccaccttccactatgtggggcagagtcccagaggcggcccctcagcccggggctgattctaaattgttgatattttcctgctgtgacatttagaaaacatgaaagtaaatctgcccacccttttaacttgaatgtcattgatggcgtttttcatcccaggatcccctgtccatggaagttgtttcgttttgcctgtggtgatataattgttgcagagtcttttggtttccatgacactgaatggtgaggggtctgtgcagcctccacaggccttccattgattcaggggatccaagtagtaggtgacagatttccttgtctgcccagaggggctccagcctgtcaggatggtatgggctggctcacttcctgagtgctggccaactcccaggtcctggctgtgtcttctaggcacacagagagattcagcaggtgctgcttctgacgctggatcctggggctgacttggtagccacagccttgctttacagcctggcatgtagataagataggattctatctcccatcttatatgtaactcctagtatttggggtgctgcactttttgaagcctccataccatttcacagcatcctcgcagggtggaatggtaagaaccgttgcccctgttcacagaggggaccatgatgcccagatggctcttggctgtgtggaccgtaggctgtgctggccatgaagagggcagaagatcatgacacagcgcttatagggaagaactggaaggttctgctgttccctgggccaaagcattttttttttttttttttttttttttgagatggagtctccctctgttgtccaggctggagtgcagtggctcaatcttggctctgtgcaaccttcacctctcaggttcaggtgattctcccgcctcagccccccgagtagctaggattacaggtgcccactgccatgctcagctaatttttgcatttttagtagagatggggtttcaccatgtctcgaactcctgacatcaagtgatccatttgctttggcctcccaaagtgttgggattacaggcgtgagccaccgtgcccggccctttttgtttttttaactaagaaaaaaatttaattataagatatatataacatgaaacttattgtcttaactatttccaagtgtacagtcagtagtgttgaatacagtcatgttgttgtgcatgcaatctccagaatgcttttcatcttgcaaaactgaaactgaaccagcccgggcgcaggggcctcacacctctaatcttagtgcgttgggaggcccagcaggaaaggattgctcaaggccgggagttcaagaccagcctggccagcatagtgagaccccatctcttctaatgaaaaacaaggaaatcaaaccattaaacaattccccacccagcacctggcaaccactattctgctctctgtttctatggagttgactcctagatctcatacccagagtatgcgtagcacatagtacttatttgtgtgtgagtagcatgcttcacttagcataatggactaaagattaatccatgttacagcatgtgtcagaatttccttgctttttagggctgaatagtgttccattgtgtggatgtaccacattgtgtttattcattcatctctcagtagacacttgagttgctgggttttttttttgttttttttttgagaccgagttttgttcttgttgcccaggttggagtggagtggcacgattttggcttaccacaacctctgcctgttgggttcaagcgattctcctgcctcagcctcctgagtagctgagctggaattacaggcatgcgccaccacgcccggctaattttgtatttttagtagagacactgtttctccatgttgggtcaggctggtctcgaactcctgacctcaggtgatccacctgccttggcctcccaaagtgttgggattacaggtgtgagccactgtgcccagccctttttttttttttttttttttttttttttaaatcagagtctgtctttgttgccaggctagagtgcagtggtgcagtcttggctcactgcagtctcaaactcctgggcacaagcaatcctcccacctcagactcccaagtcgctgggattataggtgtgatgcctcaccacacccagctaatttttgtattttttgtagagacagattttcattatgttggccaggctggtctggaactcctggcctcaggtaatcccacccacctcggcctcccaaagtgctgggatgacagggttgagccaccatgcctgttggttgcctttttactctatctatagtattttttcatgctcaagagtgttgattttgatgaagtctagttgtctattttttttccttttgttgcctgtgctttgggcatcatatctaattaatcattgccaaatctagtgttatgaagttttctcccatgttttcttctaagagtgtatggttttagtgcttatcctcaggtcgcttacccattttgagttattttttggtgatggtgtgaaggaagggcccagcttcactgttctgcacatgggtattctgtgttcccagctctgcttgttggagacactgtcctttccccactggatggtctgacaccattgtcaagaatggtttgactagctaatgcttttttgcaggtttttgttgaaatgtggcttcatcactattccttggagatgtatcaaaaaatgcagtcccctcatgccaaggtatgttttcagtttttcttcccacctcctgcttctgaccctgctcagcctgctgcttcttatcctgcctctgagtgagttttcacaggctggatggtgggaagcagaaacccaccacccacctgtcactcagaagccaaataagaagtgggcatcgttctctagtttccggtggcgggcagggggcattgcaccctgatgtgtagaaacttgaggcctggaagtctggcctctccggggccctccctgtgcagcttcctgagccccctccctggcagcaggaacagatgaggttgtggtgctcttgctctgggagtgggggagctgcgatgcctcagggagtcactttcttgtctgtctcctcgaccatcagcagtgcctttgtgctttcccatgaaggggtggcctggagcctcctttgcctgctcaccctactcccccaccttctccagagcatgagcaagcagcccttctcgtggcgagtggcacctggtgcccccctgcatgcagcctggcttgcctcctctccctccgcctcgcctgtccctgctctctctctctgggcagcacgctgctttcagatgcatggaggtggcggaggccctgggtcattcctgctgttgccgttcctggagctagcaccacccctggaccggtcagagtgagaaagcagctaggtgaaccttagatctccagtcagtcatctccatggagcagcttgactcatgcccatccgtgcccttgcctgaagtggcatcagccacgtagtctggtgcccatggcgtctgtgcatcagtatgcttgggaaactttggctttgtcactgacagaattattgagggcttcctccagaatgtgggtgatggagttaaacttcagaagagcatcctgtcacttttcctctggttctggcaaagagctgtgggtctgcttctgccacagtctgcagccagttccatggccccatgctttgccatgtggaggctctctcagagcatagggtcccccaaatcctcaccctcagaatcacatgggcggagaatggggaaaagctgaggaccccatcttgggcctcttgagtcacaaagagcctgcagtgcccttcctgcttccagagcagacttgctgcatgttcctggccggtgcctgggggcctggttattccctgagcctgctcctcccgtggggctctgggacactcagcactcgtgcatgtggcgtggcgtggcgtggcctggcaggggcagaggccactgcaccgcattgtgttcctgttgctccttctgccttctgagggagtggaagcacacctactttcaagagtcagccagaaaggctctttgaggctgtcacctgtgaggattctgtgtcctcacgggccagaggaagggcagggggctgtccctgtggagggcaggaggtgcagttcccttcttccccacatttgcttcctcttggccagaccttggggtgggtggaccctgctcagaataccttgcagtggccggaccaagtacccagagatgctccactcttcgcctcttccagttcaggcaaaacacaaaacgcaagaaaacttggtgggtgggagtcagagaaaggcagctgtggaggtctgtgtctcccaaggcttcttgccgcttgtccaggcctgtgctacacgtactgccatgcagaaatccctgcccgtccccactagcccttattttcagatgcaggaagtgaggctcctggggtcatcctcctcaccctgcttgagtccaggatgcatgcttgctccccagtggccctgtgggcagtaaggatggccatggcgctgtaggccactgtgttcctgcaagcaagggcagagccacactggggaactatgtgtctgattcctccctgagccccaggtctggcacagaggaaggctgtggagggcaacacctccctgccctgctccttcactccctgctctgcgtgtcatggcgactggcgtgtgttctgatttctcctgtgtggagcccagtgggtgtgctgcttgggcaggaggcatgctgctggcggggcaggatgtgcaccaggccggctgtggctgcactgggctgaaggggtgcttcggcaggccgtggtgctgcagggcagcaggtcggagggtcctggctaggagccagctcagcctcaggttcctgctgcctctgggtgtgtgtggctgtggccagatcctcaggggctcccgcccttgggaacccactgtatctggagggtgggagtttctggtgcggtaggagaggctgcctccaggagtcagagctgatgtgcacaccaccttagctgacgtccagactatgagggtggagggcagggcctggtggcctgagcctgtgctcctcgtctctgtgctacccttggggacctgtcctcgccctcagttctcagagagccagtcatggaggggacattgatttgagttttagacacataggaatcaatcatgtcttataccaatttggtaaatttcttaagacaacccagggaaatccagtcaattccacatgtgttttctgggcagagtgctcctcctggaacatgctccgctggtttcttttctgagcctcaggatcttgaggtcttggtggctggcagcatgtcccagtgagggccctgggctcttggagggctctggtttgcatttgccacacatagtgtgacctcagtcactggggagagtgggtcccgtcttgaagggcagtgagttgggcccttccaaggagctagagaggcctttaggccccagcaatactgaggggccacccaggtgacctggaggcccctctgcccgggccttcaaagcgaggagagtgtggccacagccctggcctggttgtggaggggggtgctgcgctggcccgcggctgctgggactgcaattgtttggattgtcactgaccgtgctttgtttttgtttttttctcctcactcctgggctcgttctcattgctttctttctctctccgcttccccctcccgtgtctggtcctatctctccccttccccaccccattctcttgtcctctgctcttcggcctcttccggcatctctctgtttcttatctcagctggaggttctgcactaccgactcagtgtctccagcgccctctacagccccgcccaacccagcctccaggccctccacgcctaccaagtactggctcattcgttcccttcctcctgcccagagtgcatgtccatgcctggcaaagcaaggggccgccggggccggccgctggtgactcagaggctagttccaagcagaacgaagcccctgtgtcggggggaggcaggggtcagcctccagcctggcttggcccctggctttgtagactagtttggttgggggtgggggtggtggctttgtggcagaacgaggcatgtgggcatgtggccctggctgtccaggctccaggccagtggaggtgtcacaagctctccagtgttggaggcctcgtgcttggccctggtttgaagagctggagtcctcaggcctcagcagtggctgggttcactgcactctgcagggtctggcgcttaggaaggagctgaagacgcagaggcctgggacttacgggtggtgtgggttctgccttgttcagaactgtcacccagcctggaggtgctgtgacccagagcttgcgtgtgaggcagggggtgggaaggacctggggcttgaccctatagcccacagaagctgtcagtccttgacagcccagccaggacccggccttcacctggggagccctggccgctcatacacacctccagaggcagattagagtgggtggtgacctcatagaggttgcagctgcctaggcagaaatgacaagtgtgtggatggatttgaggtcagtgaagcagagttcagacctgtaggggacttcagggatgacagaggaatctgtgagagccgggggagacgggctgtgctcacagccagtgatctgtcccattcacctgggaaagagcacacatgattgaacaggaatttgagcataagatgagaaaatgtgttggctccttagcgctggtgggctgggatggtggccacagcacacaggggcacctcattccgcaggagccactgcagaggagaggagggacctgagggcttgcgccagggagggaggacagtggtccagaaagcatctggcaaaccccatggtgggagggtgaccagcgagacccaggtggggtgagctgaggtgctgtgagtctcagcccagtgggaaatgtgcggctttggggacacatccttaagaagcaagcaggtgactgtggccagggactccatgtgggaacatggcccaggaggggtgcagagctgagggagaagtttggctcagcaaacctatgggaaaatagcctgagagggttccatgtgactgccggcggcaggggcggacagtgggccctccagacccttcggaaggaccaggtgaggtgggtcaagtgcaccagcaggggaaagcagttttccagttcacatgtggaggtcttcccctccccttagctgcaggaaggatgttacggctcaggatcccaggccaggattggagggagggcctgggctgagactggaggaggagaaagaccctggtctaacttcccaccccactggtgcccttctagttgggcagtataggtacaggtattatgtctcatacttttctccttctgtctttttttttttttttttgagatggagtctcactccgttgcccaggctggaatgcattgacatggtctcggctcactgcaatctccacctcccagattgaagtgattcttctgcctcagcctcttgagtagctgggattacaggcatgtgccacccacccggctaatttttgtatttttagtagagatgtagtttcaccatattggccaggctggtctcaaactcctgacctcaagtgatctgcctgccttggcctcccaaagtgttgggattataggcgtgagccaccgcacctggccctttcttccaaaacaatgcaaattcattgcccaaagacttaacaatacagatagagaaaaatagccagccacgatggctcatgcttgtaatcccagcactttgggaggttgaggcaggcggattgcctgagctcaggagtttgagaccagcctgggcaacatggcgaaacccctgtctctacaaatacaaaaattagcctggtgtggtggtgtgtgcgcctgtgttcccagctacccgggaggctgagatgggaggatcccttaagccctggaggcagaggctgcagtgagccgagaccacgacactacagcgcgggtgacagagtgagaccctgtctcaaaaaaaagaaaatgcctcccaccccatagctccatcttcagactcagccacttatctctggggtgtgtcacttggacctgtggggacagtttcctgacggggagacttgagtgggtagcgatatgggcagtggcccgtgcggggtctacagcataccaggatatacctggaggctgggccctgcaccagcctcagcagcacagatgtgttacggcccaaagggctaggggacaagtgtggagaggcgagcatgtccactcagactcagggtcctgccctcagcacccctacttccctgccctcgtctactctcgatgcgcccacctgccctcccaatgctaaacaaccagaggaagcccatggccaccaaaaaggctttcctctccggctagatctccaggatcctgcttgtatgttacaaatcaaaagtttggcctaggctggacacagtggctcacgcctgtattccctctctttgggagacctgaggtcaggccaacatggtgaaaccccatctctatgaaaaataaaaattagctgggcatggtggcacacacttgtcgtcccagctactcaggaggctgagtcaggagaattgcttgaacctggaggcggaggttgcagtgagccgagattgcgccactgtgctccagcctgggcgacagagggagacgttgtctcaagaaaaaaaaaaaaagtttggcctaatatgcttgactgtgcttttgtcttggggttttcctgaccacaggtccctgggcaacagaggaagtgacgcccggggcttctactgggccgagttctcatctggctgtgtccaggatgaggagcaggtgggaggaattggggaggaaagtgaagccatggggttcttatttttgaaccaggtcttactctcacccacgctggagtgcagtggcacgatcctagctcactgcagcctcaaactcctgggctgaagtgatcctcccaccctagcctcctgagtcattaggactataggtgtgcaccaccacacccagctaacttttaaaaattttttgtagagacagggtgtccctatgttgtctaggctaatctcgaacttcttcttagcgatctttccaccttggcctcccaaagtgctggaattacaggtctgaggcaccacacctgggttccatgtgctttctgcacacacttgggaggcaggtgggagaccctggatccagagcttgtgggtgatgctggctttctcctgccctggggatcaagacaggcaccagcgcccaagggcacagcctgtgccacccgctgcggatttgcagccgtgcagaagcagggctgggaggccctctgcagatgtgtctgtcttagtgaggcctccccagggctgtgtgggcgctgggccaagcacttccataccacaagctggtcacagttcagaccaagcagtgcaaggcacatctgggtgccaggcacagtggtgccccatggggtggccccactcctggcctggctcatgcctcccgtgggggcagcagagcaactggctcagggcgggatgtggtcagacctggaggcggagtgctatgggtgccacgctccttcccaggccctcacccctccttctccacccaggagtcgttcacgcctactgaggagcatgtgttggtggtgcgcctgctgctgaagcacctgcacgcctttgccaacagcctgaagccagagcaggcctcaccctccgcccactcccacgccaccagccccctggaggagttcaaacggtgggtggcccttgcggcttcccaccactgccgtctcccccactttcccctcctaaggtgtcgcccacagcgacctctccccagggcatggagggcagggtgaggggagtccctttctgttcacagagcacgtgaggcctctccaggcctaggcaggtgtcctctcaccccgcccagttggctcccagagcctctctctccagactggccagagaggccccattgtgctgccatctcttgcatctcatattcttcaaccctgtcagaagagttggggtggccctgactgccaggcccaccagggccagcagagtgggctgtttgtatgggacggagggtgaggtgtgtgcaggagttgtgctgactcggccgcctgcgtgctgtgtgtccttgggatgttccctaacctctctgaaccttgatttcttcctctgaagaatggggctgaacctcccggaggtgcccagtgctctcccccggcggagccaaccctcactgatgggagccctggggtcatctgtgagcagggcctccttactgggcaagcagcaggtgaccctccccatcccctgcagggctgctgtcccgaggttcgtccagcagaaactctacctcttcttgcagcattgctttggccactggcccctggacgcatcgttcagagctgtgagtgttggccccgtcacacgtgtgcctgtgtcctctgtgtgccttgaggtgggaggtcctttggggcagataaaggaggagagcaagtgttatcacagaggccttggcaaggagggggtcttggagggccacattgttctttctgtttgagtttccaaagccagccctcaggaaagccctgccctgtcccacctgtctggtgcagggcaggttcctacccttacctgaccagggagtactctgccccctgggctgggaaaagcccaccctggcttctggagggccagcaagagccaaacctcacagggctgtgcatgtctctcctgcgccctctggaggaagtgagaagagtcagtcccacccagctgccgcctggtatctgggctccaggccaccgagtatttggcccccagccacggagcccttagcacacacctcccccacaggtcctggagatgtggctgagctacctgcagccgtggcggtacgcgcctgacaagcaggctccgggcagcgactcccagccccggtgtgtgtcggagaaatggtgagcctcagctcctctcacagacatgccactggctcccccaggtggatattgttccagtgggtggaggccaagccagcggcctgtgctggggtcagcctggccctggcccccgtggcctcctgaagtcctctcccagcccctgccgagcggaacacagaccagccttttcctaggctcaactttgccaggccctagaattggtttcttatggaagctgtgggtgacccatcctgccctgtcccctctcccttctgtcccagatgagtgtttagccacccccaaactatcccttgaccttcatcagcacagaacagagcagcggtgatcaagcaggggatgagggaggctgtcttcccctcatcccctgcttgatcctcacagcggacccccaggcttccgtggcacctggcatggccactgatccctcccctccttgcccaaggcattcccctttggaccctctgggggatcctccctgagccccctcaggctctgggctggcctctgccatggtggcctccatccacttggaaaggggcctcagccttgctaaggatgtggcatcctcagagggcctgggtcttacctgcctctgtcctgtcctccctgcctgcccgtggccccctgagctgtcatctgtgacagcttgtcaccttggctaaggagagtctgaaagcccaagggtgagcaagggcattaggggcacggaccgggcattaggggcatgcttccagttggaaggctggccccaacctgccacctcggtccccagggcaccctttgtccaggagaacctgctgatgtacaccaagttgtttgtgggctttctgaaccgcgcgctccgcacagacctggtcagccccaagcacgcgctcatggtgttccgagtggccaaagtctttgcccagcccaacctggctgagatgattcagaaaggtaagtcctcagcctgggccagcccggtatacgtcaacccagccagaccagggccaggcccttagctgggggtggctggtctttaagagggacccacacgcccagagtgggccccggcaaagggtgctgtggagactcctgggacacgtctgtgtggtgaggtctgaaaccggctggatcctgcctgccctgcagccaccaggcactgggtgggggatcgcaggtgtgcccacagagcgggccggcacggtccaagctgcggccccgtgaccccgctgtttctccctggcaactcgtgttggccgcttcaggaggtgagcctggtctctgccttgcattcatcctggctgggttgggcgttggtttctgtcgatcctcctgttgtttacctcctgcagacactgctaggggctctgtgagccagcagctcccccatccgccctccccatgtcagccacctactgtgtgggagatgctgccaggcagggcctcaggaggacacccctgggtcatggctccccagacacctcagagacaccccagaatcctaagggcagcagtcgggtactctgcctccgggaagtgggtgccaggttcgtctcctgccttctaggtgagcagctattcctggagccagagctggtcatcccccaccgccagcaccgactcttcacggcccccacattcactgggagcttcctgtcaccctggccaccagcggtcactgatgcctccttcaaggtgaagagccacgtctacagcctggagggccaggactgcaagtacaccccgatgtttgggcccgaggcccgcaccctggtgagtggttcggcggtgcctgtcctcagggcccctggagccatggggctgagcagaacccgggaagtgctgtgatccagcaggaaggagggaggctgggtatagatttgacgccatatctccttcccccgttttagtaaagtctaattttttcctgataacgaaggcagtgtttcttgggaaatttcaaatgtagaagatcatcctttagtctttaaaagtcttctggcagaagccactcctcctgacgctcagcagtctggctgtgcattgctcttggggctgcctgggtggcagcacaggcctgtcctgctggtgacctgcccgcgcctcccttgcaggtcctgcgcctcgctcagctcatcacacaggccaaacacacagccaagtccatctccgaccagtgtgcggagagcccggctggccactccttcctctcatggctgggctttagctccatggacaccaatggctcctacacagccaacgacctggacgagatggggcaagacagtgtccggaagacagatgaatacctggagaaggccctggagtacctgcgccagatattccgggtacgagctatggggcctgccccaccgccatcagggtccccttccctgaaggatccactccacccagcctgtggtcagccacctaccagtcccaggccccacacatatggacccagtgttgtgggaggcaggcccacaccccgagcgtcatagctgggtccccttgttgccactgttgtaggctggtggggtgaagactggtcccctctcttcatcttgatgctgagggcagtggcatttctctctggcagctcagcgaagcgcagctcaggcagttcacactcgccttgggcaccacccaggatgagaatggaaaaaagcaactccccgactgcatcgtgggtgaggacggactcatccttacgcccctggggcggtaccaggtgagaggcccgtgtctcagaggcgcacaggctgggccctgacccgtccccagctccctcctcccttgtgagcagaggccatcaggctcctctgagttgtgtgtggggctgtgttttctgacccatccccatggcctggctttctagatcatcaatgggctgcgaaggtttgaaattgagtaccagggggacccggagctgcagcccatccggagctatgagatcgccagcttggtccgcacactctttaggctgtcgtctgccatcaaccacagagtgagtgggcaggagggctggcctggcgtcttcagagagggttctagatgctcctgcctggggtgcagtcctggggtggcctgtgagggtggggtactgctgtcctgaggcctgtggtgccaccaccccacatccccgtgcctctgtctgtgcttcagtttgcaggacagatggcggctctgtgttcccgggatgacttcctcggcagcttctgtcgctaccacctcacagaacctgggctggccagcaggcacctgctgagccctgtggggcggaggcaggtggccggccacacccgcggccccaggctcagcctgcgcttcctgggcagttaccggacgctggtctcgctgctgctggccttcttcgtggcctctctgttctgcgtcgggcccctcccatgcacgctgctgctcaccctgggctatgtcctctacgcctctgccatgacactgctgaccgagcgggggaagctgcaccagccctgaaggtgtcagctgccttcagagcaggctggagggatttgccacacagccccacccttgggctgagaggacctgggaagcccctccaggagggaacacggtcatcctcgggcttctggagcggggttcctgcagccgcagaggcatctggaggaaacgcaaccaagaaaggaaggcaggtgggccccagcaaaggagtagctgccagggctcaacagctacgctctgtgacagcgcagagctcagcggcggcctttccctccctccgccaaggactcacggccaagccagctctcggggccttttttccactgcccatttggctactctgctgcaccaagcttgggagccagcctgccaacagccacctgggcctggcctccccactggctggccttgaggttggcagagtgggttgtggcgcttcctctctctgtgtgggaccaggacagtggcttaagtctccactccaggaaagaatcaaagtttctagagttgtgagaaaaccagagagtggctgtcctgattcttcactgtgaggggcgttcttcatgttctcccagctgttccaagactgggccgtagaattccatgtttcaggagcctaagaccctcccagagcccaggggcttcaccgcagaccccaagccattgagcacatcacccaaagcagtggccaacatcgcggacccctgtgccttgtcacagatgggtgctggtcctcaggcgttggggacactgctgggtcgatggggtcggattctgccagtttctgctctgcagccaaagatggtcagaagcattgtcacttcagtaacatcaagtgctcaaagacatggcaaccgttcagtggtacttaagtattcaaaatatacaactacagattctctgacagaaaccagcacggggtcttcaccttcattcaccccacaggcgacatgcgagggagaacagcatctcagtggtgatttccaaaccaagcctttgttttcggtgtggggttttgggggtttgctttaatgtttttgaaattgtaaatgttgggctttgtattttgatgtaaactgagcataatggcattttagggcctgtgaccaaaaatgaagcttgtaacgaccatggatctgaataaacatgtccttgcttctgagtcttctggcacctgggctcagtctctaggaggttcacctgtacagagtccctgagcacacgggggctgcagaaacaactgttgggcaccctgtcggcccctcccgctagtgtccacaggcctggacctctggcttcctatgctttcctctctcagaggcacagtagactttgccctgcctttacaaaagattcaaaaagttaggcatggcggttcacatgtgtggtcccagctacttgggatgctgagtttggaggatcatcagggtccaggaggtcaaggctgtagtgagccttcatcacgccactatactccagcctgggtgacagagtgagaccctgttctcaaagaaaagctgaccaccgggaacatgtgctgccaagtctatggcttccagaagctctgaggattgcgggagttgctgggggctgaactgtctggccaggagcggagggcagacaaaaatgcattgagaaccaagaacagcaaagctgagtgaggggtcagggtcttggtggctgggacctggacagcgatggccacaaagccagctggtgctcagtggcttctccctggggagcgagggcagcagtgggcagctgggttccagccacttccaggtggggggtagggacctcgccaataaaatgggatttaaattttgccaagaactctcactgtagagaaaatgggactgctgggatgagagcagaagcaaaatgcagttgggggggcccggccaggaggcacttgtgagggacttccggggaaggacgagttggagcaactcaggggaagcagttctgtctggggcacaaggaggagggaggccaggctggaggcaggagctcctgcagattcttgtctccacccctggatgcttccagagtggtttgccctaactgccactgccaccgcctcctcagctcctaggatgcccttgactttttgccattgggccacactcccctgctaacaggccaaatccgatgcttttctgaccacgtggcccagagactctggcagcgcagcttcagcctgtcctgacaccacccctgcccccgcacctccgcatcgtcctccttgccccgagacaatcccgttccctggatcccattcgtggcccactcccttccatgatctaccgctaggatataccagcgcccccgtgatccaaggtaagtcagattctgatcatcttgtgcaagcaactcacaagcagaggccctgtgcagtcgggagaaatctatacctttccccttggacatccaagagcaagttagatcccctaaaggccagctctcaccaataaccctgacatttccaatataaatgagagagaactctctgttttgagacaaggtcttgctatattgcccaggctggagtgcactgcaccatgacagctcactgcagcctcttaactcctgggcttaagcaatccttccgcctcagcatcccaagtagccaggactacaggcatgcgccaccgcaccctgctaatttttaaatttctttgtagagacggggtctcactgtgttgccaggctggtcttgaactcctaggctcaagtgatccgaccgcctccgcatcctgaagtgctagggttacaggtgtgagccaccacactggagagcggactcttatgtagccttcactgtaaaaattaagctagcttcccttgactcccaaaaccagaattcctagcataattttctcaagtcacaaggtgcgaaaatatatcaaattatttcccagtcaagaattcctacttttggccaggcgcagtggctcatgcctgtaatcccagcactttaggaggctgaggcaggaagatcacttgacgtcaggagtttgagaccagcctggccaacatgctgaaatcccatctctactgaaaatacaaaaattagctagtgtggtggcgcatgcctctaatctcagttacttgggaggctgaggcaagaagatcgcttgaacctgggatgtagaggttgcagtaagccgagatcgtaccactgtactccagcctgggtgacaagagactccttctcaaaaaataaataaataaaaaaagtgataaattggcagctggcgccagggacaggccatttcctgatgggccacacctatttcactaaagtgttaattgaatgcagattccagggagaatcaacttcccaggcatgtgcattaagagacaaagtggtggagtatgaccttcggggggcaccccacccaaaaacctcagatgggcatgcatacagtttcctaaacacactgcatgtgtttaggaactcccaagggtaaggagggtactgtacatgtgggcagcccaccctaagggaaggatcatgggaaaggggccagtctagaaagtcctaggatcaaggttaaacaccacacttgactttcatgtgcccacttaggtctcttccaagcgtactttcctttctttcctgttctaaagccttttaaaatacacttccactcctgctctgaaacttgcttcggtctctttttctgccttatgcccctcagtcgaattccttcttctgaggagccaagaattgtggttgctgaagacccatccggattcactgccagtgacttggacaccttccactgctaacactattaactcggacaccttccactgctaacacaataacactgggttattgtgtttatttggagagtgcgtatggtttaaggagatgtgtatgggtgccaggttgacaaggggtggacttgtgatggttaattctatatgtcagcttgtctgggccaccggatgcactcatatctggttacacattatttcctggcaggtctgtaagggtgttttcagaagaaagtggcatttaaattggtggtgcaagcagccatgcatgcatttctggagtagcttccctatagcttgcaggaaccagagcgggcaataagagccccatcctccaaatactggggatctgtgctctgattgatgatggctgctgttgattgtgaaaatgcagacaaagaggcaggcagccactgctgcaatctccactgctatgtttgcaacgtccctcctcaaccctcactgcctccctgggtacacaccttccaggaggtttttatggatcagcacctcctcctgccttcatttttcttttctccttcttgggaaccagatgtttaggactaggacattcaaaagcaaccatataccaggcacagtggctcatacctgtaatttcaacacttctggaggctgagggaggaagattgcctcagcccaggggtttgagaccaatctgggcaacatagcaagacaaaaaaaaaaaaaaaaattagccaggcgtggtgtgtgcctgtaatcccagttactagggaagctgaggcaggaggatggcttgagcccaggagttccaggctgcaagtgagctataatcgcgccactgcactcctgcctggatgaatttctctccactcaggatatcctccctcccagcaataatattgctaccattcattccagctatccataaaccataattactaaatacatgttgccataatttttttgaacaacctgtcatctattaaaaataataataaagattatattttattttactttattccttctctaatgcacttccctttttttagatccaaatttcctatatctttttccttctttttttgaaacagggtctctctgtgttgcccaagctggagtgcagtggtgcaatcacggctcgctgcatctcagccttctcagtagctgggactgcaggtgtgcaccaccatgcaggcaaactttttagttttttgtggagtcagggtctggctatattgcccaggctggtctctaactcttgggctcaagagatgctcctgctttggcctctcaaagtgctgggattacaggcatcagccatgcacctgagttttcatttctgttgtgtttttgacttcttacattttcttttagttttttgagatggagtctcactctgttgctcgggatggagtgcagtggcaggatctcagcttgctggaaacttcacctcctgggttcaagcaattctttggcctcggcctcctgagtagctgggattacagacatgcaccaccatgcctgcctttcttttttctttttttttttttgagatggagtcttgttctgtcgcccaggctggagtgcagtggcatgatcttggctcactgcaagctccgcctcccgggttcacgccattcttctacctcagcttcccgagtagctgggaccacaggcgcccaccaccatgcctggctaattttttgtattttagtagagatggggttttaccgtgttagccaggatggtctcgatctcctgacctcatgatccgcccgcctcggcctcccaataatttttgtatttttagtagagatgggttttcaccatgttgcccaggctggtcttgaactcctgacctcaagtgattcacccacctcggcctcccaaagtgttgggattagcggtgtaagccactgtgcctggccaacttctcacattttcttttcttttcttcttcttttttttttttttgagatggagtcttactctgttgcccaggcttgagggcagtggcatgatctcggctcactgcaacctccacctcctggattcaagcaatcctcccaccttagcttcccaagtagctggaattacaggtgcatgccaccacactagctaatttttgtattttttagtagagatggggtatcaccatgttggccaggctggtcttgagctcctgacctcaaatgatctgcccaactcagcctcccaaagtgctgggattacagacatgagccaccctgcctggccttcacattttctttagattatttcttagaacgtctacctctctgttcccattatcagtctgtaatctcatgttgtccactttttccattagagcccttagcatattaatcatagtcgtattccatttcttggtctgataattccaacatctctgccatatttaagtgtggctctgatgcttgctgtgtcttcgaactgtgttgttttttattatgccttgcaagtttttgttgagggctggtcatgctcaaccagtaaatggactttctgtgtgaagttttatgttggtctggttaggggttgtgccatgtgtgctgtttgttaactggaggtgtcagaggcttaaacctcctctagtgtcttagtttttgtctccctgttgtctttacggtttcctagagatttctcgaataagctctgagatgagcagttctttcacttgtatcccctgttattatacaggagccccgctgatgtggcagtaagatgtggggaaggagagccagagcaggctggggttagctatgttcccttcctccaggtttgttggtgtctggcaaaattatgattggcctggctgtgattttttttttttttgagggcaggccttgtaaaaaaaaaaaagactttttatttttattatttttaaatttatttatttatttattattttttttttttttgagacagaatcccaccctgtcgcccaggctggagtgcaatggcgcgatcttggctcactgcaacctccacctcctgggttcaagtgattctcctgcctcagcctccggagtagctgggattacaggtgtgaaccactgcgcccggcctcttatttttaggcagcatctcactctgtcacttaggctggagttcagtggtgccatcatagctcactgcagcctcgacctccggggttccagcaatcctcccacctcagcctcccgagcagttgggactacaggcgcaagccaccatgcccgggttttttttcttttaatttttatagagacaggggctcactttccatccaggctggaaaggatgccttttattattattattattattattattattattattattattttagatagagtctctgtcgcccaggttggagtgcagcggtgcgatctcggctcactgcaacctctgcctcccggttcaagctattctcctgccttagcctccggagtagctgggattactggctcccgccactacgcccggctaaattttgtatttttagtagagtcggggtttcgccatgttggtcaggctggtcaggaactcctgacctcaagtgatccgtccgcctcggcctcccaaagttcggggattacaggcgtgagccccggcgcccagccctggatgccttgtaaatcactactcacccaggaagagccagcgacaggccccgcccccgccaaacgcctggcgccctccgtcgcccggtttccatggtgacggggcgccaggctagggcggcctggccactgagccggggtgcagtggcagcgggagagtacctggcgatggcgatatgagcggtgcgggggtggcggctgggacgcggccccccagctcgccgaccccgggctctcggcgccggcgccagcgcccctctgtgggcgtccagtccttgaggccgcagagcccgcagctcaggcagagcgacccgcagaaacggaacctggacctggagaagagcctacagttcctgcagcagcagcactcggagatgctggccaagctccatgaggagatcgagcatctgaagcgggaaaacaagggtgagccggcgcggggccctaggccggccctgcctccccaggcacactcaacactgccgctcccgcagcacagaaacacagccatcaactccagcacacgcctgggctcagggggaacacaggacggtaagcccgccctgccacgctgagctgtcccctcctcccagagggagacccgcgtggcccccgggcagtgccgaacctgtctgcctgtctcctagggcagcagcatttcagatgctgactccgccagtcctctcctctccaagcagggtcagacacacctctgttaaaaactttatagaggggcccgggcgcggcggctcacgcctgtaattccagcactttgggagaccgagacgggcggatcgcgaggtcaggagatcgagaccatcctggctaacacggtgaaaccccgtctctactaaaaatacaaaaagttagctgggtgtggtggtgggcgtctgtagtcccagctactcgggaggctgaggcaggagaatggcgtgaacccgggaggcggagcttgcagtgagccgagatcttgccactgcactccagcctcggtgacagagcgagactccgtctcaaaaaaaacaaaaacaaacaaacaaaaactttatgcagggaaacgtgcccaagtgcgagtgcgcagctcagtgaatttcttccagctaaacacatcggatcaggagcaggcattcccccgccccccaccccgtccccgcttcgcctgtgcccatgctgtcctccggcggggccttcccaactgcacccttagctgctcctcacccccacgcccccctttctccctccctcctcccccacccctcctgccccgagcaggaggagggcccgccaggatgtgggatgtggctccgctcctcgctcttgcttcaggagggctccagcaagggcaccagtgcccgcggcctcctctcccgccccgttccctgctgctgccttacctgagtgccgagtatcccatcaccaggcgtctgttttgtcaggaccccgcttccccatgtcttcttgagcctcaggacgccccgttcctcatatcttcttgagcctccccccagtccctgatgcccacgggatcatgacaccgccatagccagggattccagagttcacctctgtacccctaaagcccaggttctgcctggcagtgtggacgccctccagtcatctcagatggatgaggctgatatgccaggtgggctctgcctagggaacaggcaggggccactcctggcctgacacccacgacaaaggcatggtggagagcgggatccatggggcagggcccaatcagccagtgggggggcaggactttcgggctcagcagccaggagaggacaggccaggcttcttctctcagaatggctgtggtttcacaagtgctgaggagagcgtgtgcttgctttcagatctccgttacaagctcataatgaatcagacatcacagaagaaaggtgagaactgggcccttcagttcctgatgggatgctcttgccacccaggggagcccctccagactgtccttgcccacctggctgcactggcccctgtatgccaacccagtggggacaggttctgggggacctggacagatgccgctacctctagccgtggctggatgatgttacgcagccaagcacagcacgtgctgctctcaggaagcccagggcctgaggtcattgcagtggggaggtgggcacagggtgctccccagacctccctcctccaagtagagctgaaatgggaaggaacccctgggacagcccctgccctgctagatctttgcctcagattgctgctgtggccaggcccaggatctccagccctatggctctgagtcctgacatgctgggggcccaggggctctggacacactccatccagggatcccttcctgccatctgggcagcaaccatggggacaaagggaggaagcagagtcctgtttccttgccacttgtccaaggcacttccccatcctgacagcggcccccacccagcccaggattctgggttgtggtctcgagctcacttcccgttatctttggggctggggctgacatcaggaggacatctgactggtggatggagccagcctgggaacatcgtagctggggcagtgcctagggctctcccttcccagagagacatggagaatggggttgagggagggcccttccctagccgctgtggcaactccagtgagctgttctgggcaaagtgtggcccaagtcggcagccccagccctgcagtgctggggacgctgacaggacacgggaagaggccatgctctccctcgggacctgctgttccatgtgtcccaagccctcctgctttccagatggcccctcaggaaaccacctttccagggcctctgctcccttgggcgctcgctgggtctgcatcaacggagtgtgggtagagccgggaggacccagccctgccaggctgaaggagggctcctcacggacacacaggccaggaggcaagcatgggcgtcttgcgggcggtagcgccgacactgtgcgctctcctgcagacagcctctccacgtcaagcttccagtctgtcaagtccatctctaattcaggtgagcaggctggcgtccatgtgctcacaggggtgagtgccccacggggaccccagcctgggtggcctctaggggtgctgaccaggtaccctctgctgggccaagctccaggatttggggatgaaggtgcagctgaaaagagccctgcaagctgaagccaccgggaggggattaagggggggtgggcaggaccagggaccagtcacagggcagtaaggcctcacagcaggcaccccagggtggggcagcaagcaggcaggaggggcctccccgggtggccgcctagggaggacgtaccctggctgcccagcagcagccctgcttggccgtgtccccgccacaagcaggcaggacacaggagggatggcactgcccacggagccctgggctcatggcaccttctgcagagtgaactgggagcagagagcctcttttccccactctgtgaggccgggcattttcctcagcccagccctaaccctcaccattcctgtgatgggcccaggtgctggacgggccttgtccttgccgaggaaacccatgcaggctgggtcccaagcctctcatgcagtttcattttacccaagatggggccagcttccactatgttgattgttctagagtgttcacaggcaccacccatgggcttcaccctcagaaccacattgattccccccagaaccctccaaggcaggcttccctttacagaggaggaaactgaggcagggacaggggcttcttgggtacacacagctgttcctggaaaacttgggcggtgactgttggtgcctgcagtgaccggccttgtagggtctgacttgcagccaactctcaaggcaaggccaggccccagcccggctccttcaacaagcaagattcaaaagctgacgtcccccagaaggcggacctggaagaggagcccctacttcacaacagcaagctggacaaggttcctggggtacaagggcaggccaggtaaggcttgggtgttcctggggtgcaagggcaggccaggtaagtcttgcctgcaccctaagggccccatgactacatttactaggctcacgccctgcagtcagccaactgctctgcccctagctccaggggcctgtctccagggctcatgatagcagatgacggccacgctctacccatcggtgggcgctgtcttcctcccttcccctgggggtcctgcagagtacctgtgctgacctgagcccacgaggggggccagtagcggcccctaaaccacttcctcagagaaaagtaaagtaatattctgaccaagagaaggcttgctgcccctggccacagcctgcctttggagggcactaggccagcaggtcagagtaggaacaggttctggtgatccctgagtttgcgggactgccctgagatgctacagagcgcacactttcagcagcgcaaccagcaggacaggctcctgcatctgcacatcccgagaccaggcctggagcaagatgccccactgggtgtggggtcacgatcaagagcagcctggcagctagctggtcacccactgaagcccatctgacccttagggcaccttcagaggggagaaagggacaggccccagtcccagagacccccacctggtgcccagagcaccctgctcagtgggagaggggaaccaggccctcttccccacattggcctgggcgctgcctggggaggtatgctgggtgggtggttagacgcacctgggcaggtcacctctggctgctgcaatgactgtttcagaaaggagaaagcagaggcctctaacgcaggagctgcctgtatggggaacagccagcaccagggcaggcagatgggggcggcggcacaccccccaatgatcctgccccttcccctgcgaaagcccaccacacttaggcagtgcgaagtgctcatccgcgagctgtggaataccaacctcctgcagacccaagaggtgaggccctgggtggtgggggtggccctgggcagtctggcaccgccacaggccccaccacgcccctgcccctgcctttcagctgcagcacctcaagtccctcctggaagggagccagaggccccaggcagtcccggaggaagctagctttcccaggtgagtgcctggtgcacccctgccccatccctgggggcctatccacagctcactgctgactcttccttcacccagggaccaagaagccacgcatttccccaaggtctccaccaagagcctctccaagaaatggtaagtcccacaggcatggggacagtggggcagccctgcagcctgggccccagggagggagtggggaagggagcgtggcagcgcagaagcggagctcgctgaggcctctctgtgcccccgtgagggccatgcaggccgcactccccacacactccactcgcgaggatgctcacggtgacagtccctctacccgcccccagcctgcttctgagcccacctgtggcggagcgtgccatcctgcccgcactgaagcagaccccgaagaacaactttgccgagaggcagaagaggctgcaggcaatgcagaaacggcgcctgcatcgctcggtgctttgagctacccgcatctggtcagtgccaggcccaccaacctgcagctggagattggctctctatagcatttcctgacacttccgctacttttaggcctggctaaattccaagatagataacactcaagatagataaagtacttgatctccaaactgacaaactgtttattttctagctgttattttgctatttggcatttacataaaagcacacgatgaagcaggtatcgccttacctgttgaaactgaaaataaagcttgtttatttccaagttgtgtgcctggctcctcactggctatcaggcgtcgcgtcccttcccagtcccagcccttggcctctagcccaggccccgctgtctgcagggacctgcctgggcagcccagtcttgcgcatcctgggacctgccagcggccctgggagacaggcattggcagagccatgcacatcctccctggaagcgggagggcttttctggtcccctggctgggctggagctgcgttccaaacacatgatcactctgtctcttgcccacactgcacactcctgggggcaagaggctgtcctctgctgccctgacacagcaccgcagacagggggtccagccaggctcgccctcctcctcggagggctgtctcgagtgccgtggccatgcccgcaccaccccttccgccacagcacagccatgtccatgtcgggggtggggtggccagcttagtgcccaccctcagccagccacacagtcttcccaggcccacggcccacttcgttggcattaatctcagcctcttcctgcttcagggagcagaactttgccaggaagctggggatgaaccaggccaaggaatgggacagtgcgtcagaaaggacagcccaggcccgggggcttacagcatcctaacaggacagcaggcccagtttcatggagtgggggacacagtgacgagtcctgacaaacacaggaataacaagattataacaatcacagatgatggggtggtatgcattaaaaggggcgactcttgtcaccaaagggctgctggccggtgcccagggcagtgggcaacatagggaggcactcactgcatggccccacccaccgccaggcccgcactctctgggtggcagtgcgtagtgttctgaggacgcgggtccagagggcctggctcttcatccttcacaatggggacacggccctcacacgagtgacacaggggtgccggcagcaggatcccccgtgcccctgctccctgggggccccacagtgctcagcaggaatgccgtgagggctgaggcccagctggtggtggggagctggcagcagccatgggccagccctccctccgataggcagcctgtgccctggacttaggggcttctctggggggtgccccacctgcccaaagcagcacagcctgaacccaaaactgtgagcagagaatccgatgtgtgtttgaagggtttcctatgtcctctgtgttctcctatagaaaggagcaggggtgtgttgagcacacacaagatgctctaacaacaccctgagcgcaccctcatgccgctgagatggacctggaccctggacaggccacggcgagcagcccgctccaccacgcccatctcagtctgtacaagtgcgggatttcatttggcagatccccaaggcaggagagaaaaggaggaaagctaacctaactccaggctctccatggacgccaacacctagaagtgtctgacaggaggcatgcgtccttggccccgatgaggctgcgggccatccctgcagtcctggctaagcagccgaggcaggccttgtggatgttccgggcacaggtgttgagcaaggtggtaaccgagtgcttttccaggctgtggctgcctggaggtcacgcagtggtgcaccgacctgaggaaggggttggtgcctgcaacagaacaatgggcatgagcctgaggcagctggtgctgcacccacggcctgcacacccggcagtgctctgggaagtggcagccccaggactggctgaagcaccaggaggagccctgcacgctgcctgcctggccatcgggctctcctcccagagcctcattgtcagtgaagaactgaatctgaatggggtttggagcccagggctttgcctctgacaaccttggggacccagccctggatctgctgtgtgccagctctgggctcagggacactgactcctgtcaacatgaccctctggagccacatcgcccagatggggccttgtgtgcccaagctactggtaggacctgtcagccccgctgagctccctggtggcctctgggaaaagcagggtctgtccttgagccgtaggctttctcacccctcagccactgaacacagccaccctggacaactgacctgacacaagatgaaggggcaaagggacacagaagaagagcagagacccctctctctcaggtgaccactggaaaacctgcgcctgcccgtgcagggggagtcagaagaggggagggaggggcctgagctgggaggggctggagtgcacagaggctgtccctgcagcactggaagcctggtgttccagaatgacccctgcctgccctgggcccccagacagctccctctgggtctgcccactgtatagccactgccggtccatccacagtggactctgggtagggtagtgggtgggcacatggtggcacacttgggaggtccttgtcgtttgcaacacacaaggctgtacctgtgacccagacaagccctggcctgccccgcctcacagccctgctcccgaacaggactcttcacacccagggaggttctggtgaggcttgcggcaaagcctggatgtctctctcactctcggatttccaaacctgaagccagagtgacctgcatcaatgggtgcccagaggtcctgcgtgccacctccctagggccccaggcaagcagagccgcatctgtgttggggaggtgagggtgcgtgggctccgggggtgggggcagaggcaatggccagtctgtcctgggcccagaagacagagcagacaacctgctggcacgtgaacataggatggggtctgggcactgtgccctccagcttggacagcagaggcctggggctgcagttgggggttgctctcttgcgccagcctggccctcctggacactcgcccagcttgcagatcaggcggacggtgccgttgttgctgcagtgagaagggtccaggtttatcatgcacttgggctccaacctggccaccaggccctggagcacactggggatgctctgctgctcatcgtcctcaagcctgtgcttctgggtgcataccactggggccttggggaggcaccatggtgaccacactgggcacacatggcccagggccaggcagccaccctctgcgaagcccagcccagctggatgtatgtgatgggtgggctgtggatggcaatcatggttggcatgaatatgcggtacagggaatggttgaagagggtgagcggatgttggccaggatggcatccaggagcagctggcataggtactgctgtttggtcagtggcaccgggggcagtggggcagtgggctggagcaggatgggcatggtcagcagcccggaacccaagggttccccatgtagggggctttcccaccccaggcccgtcctcactcacagccagattcccgaggcctctctctcagcctccacccttgtgcagacaatgttggctgatcctgggtatattccctacccccacagctttgtcctccccagagtccctgccccctctgggccaggtggcagaggcaccgatgggggccacagacagagagtgacttctcccatgttcccacccagcacagcaaacctggcctggaagaggcctggtgggcagggtctcaggtcaggcccagcccctacccagccaccctaaccttgaaggcccctcccagtggtgctcccaggagctcttgggggagccccaattcccccagggatgcccaggatcccactcaccactgccatgtcattcttgagttttcttttttttttttgagatggagtctcactctgtcgcccaggctggagtccagtggtgccatctcagctcactgcaagctccacctcccgggttcacgccattctcctacctcagcctcccaagtagctgggactacaggcgcccgccaccacgcccggctcatttcttttttgtatttttagtagagacggggtttcaccatgttagccaggatggtctcgatctcctgacctcatgatctgcccgcctcagcctcccataatgctatgattacaggcatgagccaccgcgccaggccaatatttttaaataaaaaattacaaagctgtttttcttaaaatgttacaataaaattgtatctgtgtataaaatgtatctattttatgtgtacgtgtgtgtgagtgtgtgtgtgagagagagagacagagagagacagagagagagagagagagagagagagagagagtgagttatgaattaggaagcatatgtaccaaaacattaggagtagttgagtaatgagattatgaatgattttcactttcttctttataccttatcaatcatcatctcaaaatttacaatcagaattcattataagctattgtttgtttatttatttatttatattcgcagtggcgcgatctcaactcactgtaacctcagcctcccagggtcaagcaatcctcccacctgagtctccggagtagctgggacaaatcaaagttaagttttcttcagtttaaaataacttgatataattatgttgtttagcctcatggtaaccagaaaacaaaaatcaataatagacaccctaaaaatgaaaagcaagcaattgaaacacactaccagaaaaaattatctcactacaaataaagacaggaaggaagggaaggaggaaagaaaagaaagaaagagagagatcagagagaagagaaagaggaaggaagagagaaagaacaaaagaaaaaagagagaatagcaaaacaaccagaaaacaagtaaaaaatggcagtagtatgtttttacctgttaataataaccttgaatataaatgaattaaattctccaattaagacagagtggctgaatgaattaaaagacaagacccaattatatactgcctacaagaaactcagttcacctatagacatacatagactgaaagtgaagacatgataaataatatcgcacacctgtggaaacaaaaaaagcaggagtagctctacttagattatatagactttcagtcaaaaaaagaaaaaaatgaagataattatataatgagaaaaagtaaacagcagcataacaattataagtgtatatgcaaccagcactcaagcaactaaacacagaagcaaatattaacagaccttaaaggatagactgcaatacaataacagaatgcctcaatgcttcgctataatcaacaccccactatcgtcaatggacagatcatccagacaacaaaacatcatcagttaaactgtactctataccgaatagacctaacatttgcacagctttccactccgcaactgcataatgcacattccactgaatagaatatggattattatccacaaaagactatgcattaggccaaaaaacaagtcacaacacatttttaaaaactgaaatcatggccgggcacggtggctcatgcctgtaatcccaggactttgggaggccgaggggggcggatcacaaggtcaggagacagagaccatcctggctaacacagtgaaaccccatctctactaaaaatacaaaaattagctgggcgtggcggcgtgcgcctgtagtcctagctgctggggaggctgaggcagtagaatgccgtgaacccgggaggtggagcttgcgtgagccaagatcgcgccactgcactccagcctaggcgacagagcgagactccgtctcaaaaaaaacaaaacaaaacaaaactgaaatcatatcgagtatcttttctgaccacagtggaatagtactagaaatcaataacaggaggaaaaactgtacaaatacatggaaattaaaatacatgctcatgaacaaccaataaataaatgaaaaaataaaaagaaaattaaaaatttattgaaaaaagaaacacaacataacaaatcctgtgggatgcagcaaaagcagttgtaagaggaaaagggcatagctataaatgcctacatcagagaaaatagaaagatctcaaatagccaacctgacagtacacctgaagtaatgagaaaaacgagaaaaataaaatactaaaattagcacaaaaaatatcataaagattagagaagaaattttaaaaatagaaacaaaaaatacaaaaagtcaatggaacaaagagctggattttttagaaaaaaatcaaaactgacaagctttaactagactaaggaaaagaaaaacaaaacaaaatgatagatgaaaaaggagacattacaatggataacacagaaatatgaaggattgtaagagattactaaaaacacctatataaaaacaagttggaaaatctaggagaagtgataaatttctggacacataacaaattcccaagatagaatgataaacaaaaaaccggaacaagatccataatgagtaatgtaattaaagcagtaataaaaagtcttccatcaaacaaaaacacaagaatcatggctttattgctgaattctaccaaacgttttttaaagagttaatacgaattttactcaaaatattccccaaaaaatgaagagaaaggaagtcttcgaaacttgttctatgaggacagcatgaccctggtacatggtacaaaaaccagaccaggaaacaacacaaaaagaaaaccacaggcattttaaatatccctgatgaacacagatgcaaaaaaattttcagcaaaatacttgaaaattgcacttgacaacacaataaaaagatgatctgccatgatcaagtgggattcatcccaggaatatgaggatgattcaataaaacacaaataaatgtgcgacatcacattcagcgaatcaagaacaaaaaccatataatcatttcagtagatgctgaaaaaaataaaaatcaacattccttcatgacaaaaactggacaacatgagtacagaaggaacatatctcagcgcaataaaggccatatatgacaaacccacagctaacatcataatcaatggggaaatgtttaaactcttcctctaaggcctggaacaagtgtggctacttttacgccacttttattcatcatagtactggaagtcctaagtggggcaattagacaagagaatgcaataaaaggcatccaaattggaaaaaaggaagtcaaactgtttgtttgcaggtgacatgatcatatatatacacagagaaagagagaaccctaaagattccacaaaatcacctactagaaataatttagtcaagttgcaagatacaatatcaacatataaaaatcagtagcacgcccatgcaccaatagtgaaatacctaagaaagaaatcaagaaagctatttcattaccaaaaaaattatatctagggataaacttaaccaaaaagacaaaagatcccacaatggaaactctaaaacacagatgaaagctattaaagcagacacaagtaaatggaaagatatcccatgtccatgcactagaagaatattgttaaaatgtctatatcacccaatgtgatctagagagtcaatgcaatccatgttaaattacaaaagacattcttcatagaaatagaaaaaaaaaatcctaaaattcacatggaaacgcaaaatacctcagatagacaaaagaatctggaataaaaagaaaagctggaggcatcacacctgacttcaaaacatactacaaatctgtagtaagcatggtaataccaaaacagcataatactatcaaaaaaaggggcgggggagaaacagaaacgaaggaatgacagacatagacaagtgaaacagaatagagaaatcagaaataaattcacccatttatggcgtactcatttttaacaaaggcaccaagaacacacattcgggaaggacaatctcttcaataaactgctaggataactcaacacccacatgtacaggaatacatctaggccgttatcttaccatatacaaaaatctactcaaaataaagatttaaatgtaggacctgaaactataaaactactagagaagaaaacataggataaatccttcatgaaactggttaggacaaggaattttcaaatagacatcaaaagcacaagcaacaaaagcaaagatgtaattacattaaacttgtcaaaagcacaagcaacaaaagcaaagatgtaattacataaaacttaaaagcttctgcaaagcacaggaagaaatcagtagaacgaagaaacaacccagagaatggaagaaagtatttgcaaactatgcatcagccaaggggctaatacacaaaatatataaataactactcaaaagcaaaaatacaaataatctgattaaaaaaaaatctaccccaaatctttgtctcccaccattattttcccaccttcttttcccgaccgcctttggcctcctccccctcgccacctgttttcttcctccatctaccccaaaactttttccccaccatttttccccaccgtcatttcgcaaagccttctctgctctctcactcaccacccttttccccatccacttacccactttccccactgtttttcccaccgtcttttccctttctccctggccaccttcttttcccccatcccactctcatcaccctcttttgctccttcatctaagcaaaaacattttctcccgtcttttcccaaacccttctcctcactcctgctgctcaccactttttcgcccttcatctacccgaaaactgttttcctcatcgtcttttcccccgctcctccttgccaccctctttcccttctctacccaaaaacatttcaccatagtcttttcgcaaagccttctccccactcctgctcacctccccttttccccctccatccaccccccagaatattccctactgtcttttcacagtcttccccccttcccactcgtcctcttctttgccctatcctgcttgccactctcttttttgccttccatctaccccaaactattttcccatttttttcccaactctctttccctgctccctctcgtcaccctctttcctcctcctcgttaccctctttccccccaccatctacccaaacactttttacccactgtcttttctttctccaccgtctttctttcctgcccactatctttttgcaaaaccttgtcttcctcccgctggctacccttttcccttcccccacctgttaccctcttttccccctctatctacccaaaaccttttctccccactgtcttttcacaaaaccttctctccctactgctcaacgctgtttctcccccccaccaccctctctttcctcctcccttgccaccctcttttcctcctccatctacccataaacattttacccaccatctttctgcaaaaccttccctccctcccgctccccaccccgtttttctccctccatctacccaaaaactttttttcccactatcttttccccaccgcctttttgcaacgcgctctcctgctcactatcctcttttccctttggcactaaccaccctctttaccccctccatctatcccaaaactcttttcctcctcttaccgcttccgccgcactgccgtctcggtcgcggttaccaccagtcgcagcgaggcgagccacggtgtagcggctccagcctccagcgtacggctggtgattacccattcctggtcctctaagccgggcactgagcagctccacaggaaaatacgggaacgtggaagagcctgacttcccttcagcagcagtcatataccgagattatatacaggaggattcctgactgcatgttctgattggatgagaaaaaccctccagggttacttggattggactttattatcatgttctgattggatgagagccagtcttaagacaaccaatcacagcatgaaaataaagtccaatcagagtaggcctagaggtttttctctcatccaatcagaacatgtagtctgggaaccacatgtgcgtaacctcagtaggtaaagcatgcggaggtggcgtcaggtcatttcaggctcttaagtgtgggcgtttggtatccggcatggctgctacctgtttctggctggagcctcggacactggctcactgcagttggtggtgtcgacagtggtaggagggcaaccagtagcgggagcttctcctgccaggcaggaagacgagtagaagggagcggcatgctggaggctggagcctgagcccctggggctcgccttgctgtgtttggtggtgacgtgggacactgcagctcggccagagtggtagaaatgtcctggtgtaggtgagttatccggggatgtactgcccgcctgtgggggcaggggttgggtgtcctattggggctcactacccgaggctgcactgcctgtgtcagggggctggttgggggcactctccgaggttgcattgctggcggtggaggggggcggttttggctggctgtccggggctacactgcccgtggtggcgggggtgatgggggggaggcaggttgtgtgcactaacgtgtactgccggtggcgggggaggggttaggggcactattttctgctgcactgcccggggcagggagtggttttggtggttatttggagctacaatgctggcagcggggggtggtttaggagtgttgtcgggtgctgcactgcccttactcagggtgcgctatcaggagctgcgctacctgtggtggggtgggggcggcgattttgaggcattgtcttgtgcagcaacacctgtggctgggtcaggttgtgggcactatcgggtgctacactgcctgtggagggggtggttggggggggtattggggttacactgcctgcaactggcacagggtgtggtgggtgtgctgtccgggggctacactgccggcggcaggggtcaggttagggttgctatgggggctatactgccagtggtgttggtcggctgcagaggtggtggggacagcaacagccatggtctccttgctccttcgggtgactcttctcttttccagactccagagttcctcctggtgcaatcttgagcagggcagggcccccacacccactgcggttctccggcctgcacctcccgcccacaccccatgctctgtgttggggagaccacctgggactaccgggtggggattagtgggcatcgcgggggactgtgggaacagggcactgtgggtggaggtgtcaggaatgggaaccagcagttgagtggggagggctggctgggtctgagtttctcctactcctgctccccaaggagtgcagccctggtgggcccagcaatttctggccagttgcacctggatgggggtggtttcagcataggcactcacacccgccccagttcctggccaccttttgccagaaagagaggctggacttcggtgggtgggtctgagtgccttagctgaagctggtccctgccacccagtggccagcatgacaaggtgaggctctaacgttaccactccctgcatcccattctaggcttttctggctttgcccgtctagctgctccaagccaggctggaggaggaggagaaggaatcacctgtggtacgctggagcctgcatgtggcgtgactctgcagctcgcctcgtgtgactgatggcagccacggagactgcagctcgacaggagtggtaggagggtgcccgcgggggcaaggtggtaggaaccttgtagggtgggctgctgcattgatggcgacagcaattgtattggcattggtgctagtggtggtagtggcagaaagtctggggactgggaagggggagtaggagcactgcagggcccagtccgacctggggtggggacgaacctgctggtgctgtaccatgggcctcggtggcagtggtggaggtgcacttagggaaaggagtcctcccccttctcttgcaatctctggagggtggcctccttctgagccaggcatgagtggcagcattgtctcattcttaacaaaatttagggggtgactgtgtgtgtgtgtgtgtggctttttttttttttttttttttttttttgagacagagtctcgctcttttgcccaggctggagtgccgtggtgtgatctcggctcactgcaagctctgcttcctgggttcacaccatcctcctgcctcagcctccctagtagctgggactacaggcgcctgccaccacgcccagccaattttatttttgtatttttagtagagatggggtttcacagtgttagccaggatggtctagatcacctgacctcatgatccacccaccttggcctcccaaagtgctaggattataggcgtgagccaccactcccggccattttttccttcttttttgttgtgatggtcttggactttttcaaattttgtgaattggggaggggataaaaggtgtcataataggccttctaattcctgcacctgttctttttgcttattctagtctgtgtattcttctcatcttcttgttcctcttcattttcttttgctgctgcttctatttcatgtttctattcttgtttctcctcctctttttgttttctttatgccaagcaatggccttaacaaacaacaagccaaaattgtcactgtgttgtatttttaaaataactggtcctttactatgttttagggatgaggaaaaaaatcagttgtataattagttacttgaatagctatgctttcacgattgtgttaaaccacttatgcctagtgttccattattggaatggtaagcatgagggagttaattacatcctactgctcaaggtcattagaaggtttgatttttcactcgtgcaaaaattcaaaaaattgcagcctctggcataaatgggctaatgcgttgtaagtagttattcagagaatcaaaaaatgaagcatcacataaaaatattggtggcaaacagccatttcatctctctcacatatttggagctatgcaggagtcacggggtaagttctaatttatgagattattaagtgaactgtattgccttcattttctctgccaccatttcctttgttttctttctttttttttttttttttttttgagatggagttttgctcttgttgcccaggctggagtgcaatggggcgatctcggctcactgcaacctccgcttcctgggttcaagcaagtctcctgcctcagcctcctgagtaactgggattgcaggcatgtgccaccatgcccagctaactttgtgtttttaatagagatgggttttctctatgttggtcaggctggtcttgaactcccgacttcaagtgatccacctgtctcagcttcccaaagtgctgggattggaggtgtgagccaccacgcctggctctctgccaccattttcaagagtattgtcatctgcctgagcaaacctggttcatcaccacctctttgcaagagaaaaaggaagtcgggagaattgtgtgtaattttttttttttttgagatgaagtctcgctcttgtcccccaggctgtagtacgatggcccgatcttggctcactgcaacccctgcctcctgggtgaaagcagttctcctgccttggccccctgagtagctgggattataggtgcctgccaccacgcccggctactttttgtatttttagtagagatggggtttcaccctgttggccaggctggcctagaacacctgacctcaggtgatccacccacctcggtcagctgtgtgtattttaaggcaaagattcacaaccaaaaacaaggctttattaacttttgtatctaagaacctgcagtgtcgagccctcttttattcctagtattactactttaggtgtgaactttttatctttattttactgattcctctagaagtttatgcattttcttgactgctttaaagacaatctatattgtatcatttttcaagcccgtagaaatgtgtaaggcctataatttgaacacttgttatttttaaggttatgagcatgtaatataccgttgatacatggaagaatatgtataaataccactagataggttatattgaagagataatatctaaatttttgtccagagttgatggggtgcagttttgtaggtgtgtttctcaatacattgtgtccatgttttaaagcatatagaaatttgaatactgtttaacctcatatagtccttgtttataggtttaatatttctaaagactaaagacgtcacagctccctttaagattcagtaatactaataaaatttgagatatacagggttagagtccaacaaattcaaaggaaaattgttgaattatatagccgtagagcaggaatgaaacccaggttctaagttccaagggggccatgagctaccatacaggtgcatcagtgactgggcatagaggtggcaaaattatttaagagagtgagctgtggatcctaattatgtgaacatgaatttttgaactgcatggtgcctcagtttatccatcattacagtggggacagtagtaagtttttctttttctgctcagttgtctgaattattaccccagtctctcttgttgccactcttgatgcccacatgagaggatctaaggtaatttctgacaacctgggactccttaaggaaaaacagaaggttccacaaaccccattttaggagaaacctctgttttcctcatggaaccccaagaactttaagcagacaggtccctctcaaaaatctaaggctctgctctgttttgccttgcgttgtctgacctttttggtttggatgggcatcagaaattagtaggggagagagatctaaagaaaattgtggatatgaagatgtatttattgtaagaaatgttatgaaggaaagaaatgttatatgagagaggatcttatatggcaaattgttgtcctaaagtagaatgactaattacaaaagagggaaatacaggacaggtcagataaatttaatcatgttgtagatagtctgtggaagttgtgttatgattcataaagtgggaaagaaaaactgaacaactgctagatcttttcttgtctagaagtgttgtgtatgtgatgtatatataatggctcggcttaaaagaaaatgaaagctcttaaatattttgtaagaaaaatagaagctctaatgccttttatttcatgtgacttcagtaatctttgggaaataaagatggtgttaaagtcagtggtaaaataaaaatatcttcaaaatttatccatttggtctaacttaagtcagaggttaagttttagaagtgctttaatgtcataaattgatgctttgactttggaaaatagttttgtttacctggtttggagccgttagatttctaggtaaggtctccggacaggtggagttagccatgtctcctagctatgctggaaggagtcagattttatctgcagttctgtcttgtatcctagactctgcacctggtatgcaattaaaacttactgctgctgctaatctctgggttccatttaaaatccttccatcacatgaatactatcccctgtactaaatttttccacaattaaatacttagaatcatttttgctgacttgacccaaccattagtgatatattttaaaaatgcttataatgtgtcacaatatatttagcaaatgtagggaatgacatttttactttcttcagcatttacatagcatttatgtagcaatgctatctcaagtatttttaatcatttaaatgttagataatagatatgttttttattccttttttctatgtaaataaatataattatgagatatttagtaaatagtatcaattgcatatgtcacttacagaatatacaatgaaattaggattatttttatacactacatcatatttacttgttggctttccaataacttagaaatagtattctggattaactgtgtgactcatgagagagggagtttgtgcaattatagtctttacaaatttttactagatttttcaagacttatgctagaactttgagaacaaggtaataaataagcatatctgttaatatcacctttggtcaactcttggctagacccaatgataatgtaggaattaacataatttttctactaaaggtgttggatttgttccgagagaccacagttcaaagtcattgacaaagaaagtttaaaaattgttagattaaaaccttttagtgtgtttgaaattgttttgtagaaaaatatcctggtttgcattgataggtttttttttaaataacaactagaccaagagaaagggagagtagtgataaatgtccatgttttcgagttgaaaagtaacaatcagtgtattacaacagatggatttgatgtcaaattacaaatgctgagaacgttatatgtaactgacttgccagagtaattatacaaggcaaagaaaggaaaggcatctaaataggaagggaaggggtgagattgtctgtgttttagactccacaaaaaacccattacagctgataaacactatattcaataaagttgagagttacaaaattaacattggcttctatacaccaatgacaaactgttatctgaaaaataaggtaattccatttataatagcaacaaaacaaatatataagtaaatcaaagagcaaggagtaattttaatgaaggatgtgaacgatttgtatactgaaaattatagcacattgatgaaagaaattgaaagtgacaaatagaaaaagtcctatatcaattgaaaaaattaatattgtcaaaatttcaatgctactcaaagcattctacagattaaatgcaaccgctgtcaaattccaatgtcattcttcacagaaatagaaaaattagtcctaaaatctgaatggaaccacaaaagatgctgaaaaacgaaagcaatctttagcaaaaagaacaaacctggaggcatcagactacctaatctttgacaaagcaaacaagaaaatgggaaaggacaccctattcaacatatgctgctggtataattggcaagccacatgcagaaaaatgaagccgttcttcaaaggttaaatacagaattaccacatgactcagtaaattcactcctatgtatacaccaaaaataaattaaaacaaatgccttacacataaaagtatttatagcaacaaaaagtaggaaacaacagaaatgtccatgaattttggagtggattaataaaatgtggtctgtccataaaataaaatattacttggcaatgaaaaagaaaaacatattaatacatgctccaaaaaggatgaacattgaaaacatgataaatgaaagtagtaagtcacacgtaactatattattatgattccacttacatgaaatgtccagaataggcaaatccttccagaataggcaaattcttaggtaatagatggatgattgcctagggctgggagaggtttaaaggaagaatggggaaaatagggaaagattgctaatgggtgcaaagtttcttttaaggagcataaaaatgttctaaaatcatggtgattgtttaactagttaatacacttaaaaaactgaattttatactttaaatgagtgaattaaataatatataaattgtatctcaatgaacctgtgaaaaaagttaaaaaatatgtggtatgcataaacaaaaagttcttcattttatttcctagggttttggggaaaaggtggtgtttgattatatgagtaagttctttagaggtgattcaggagattttggtgcacccaacacctgtgcagtatacactgtatacactgcaccggggcaggagctggctgcaatcctgtggccccaaatgcccccttgctgatggcctcgtgttctgggtgcggagcaaagaggagcaggtgttgaaggcacctcaggcaggccctgggctcggtgggcgtcttgtgctccgcgattttgaggccatttgcagccagctccgtcagcccgagctcccagctgcagctgctgccatgcacaacagcaccgccaggagtgtcctgggggcttttttcaaaggaggctgtcagcatcctcaagttccagacgcttagccccagtcctcctttaagaggctttttttttttcaccagaggcttctcagtggcttgaaagctcagctgactcccacgaagtttgccggaactcaaggctgtcagtgacattcgtggcgccaagacttaagcaggcgcgttgcatgcatcggccagtgtctgtgccacgtgccctgacgccacctgagataagcacgccgcacgcgcacgcgcacgccgcgcgcacacgcccggcagcagcttggctggcttgtaacggcttgcacgcgcacgccgcacgcgcataacggtttggccggcctgtaacggcttgcacgcgcacgccgcacgcgcgtaacggcttggctggcctgtaatggcttgcacgcgcatgctgcacgcgctttaacggcttggctggcctgtagcggcttggcttggctttgcgttcttggcttggcttggcgttggtagcttggattgacgtttcctgcttggattgacgttttctctctcgtgttcctttgctgggcttgaccttttctctgctgggtttggcattcccttgggtgggctgggtgttttcttgggggcgggggttggccctttctggggtgggcgtggggtcgcccagggggggcgtgggctttcctcgggtgggtgtgggttttccctgggtggggtgggctgggctggaatcccctgctggggttggcaggttttggctgggattgacttttctcttcaaacagattggaaacccggagttacctgctagttggtgaaactggttggtagacgcgatctgttggctactactggcttctcctggctgttaaaagcagatggtggttgaggttgattccatgccggctgcctcttctgtgaagaagccatttggtctcaggagcaagatgggcaagtggtgctgccgttgcttcccctgctgcagggagagcggcaagagcaacgtgggcacttctggagaccacgacgactctgctatgaagacactcaggagcaagatgggcaagtggtgccgccactgcttcccctgctgcagggggagtggcaagagcaacgtgggcgcttctggagaccacgacgactctgctatgaagacactcaggaacaagatgggcaagtggtgctgccactgcttcccctgctgcagggggagcagcaagagcaaggtgggcgcttggggagactacgatgacagtgccttcatggagcccaggtaccacgtccgtggagaagatctggacaagctccacagagctgcctggtggggtaaagtccccagaaaggatctcatcgtcatgctcagggacactgacgtgaacaagcaggacaagcaaaagaggtaaccaggcctgggctgggaggaggtgggacgtggggggatgatggggacataccctcctggcgggggaggagggggacctggctttctcgcctccgcaggcctcacaccaccctggatgtggaaacctcagagaggtcagggtacagggccctttatgaacagcaacacaaaaacaaaactttagctgatttccaatcataatttccctcatagaacactaatagactgttttaaagtgatttaacttgcaaaattaattaagtcaatgcagcagattatttttaatctacagattttaaaacaatgttctatacattatagaaaagtgtatattgagaactaagaacgaagccccataacacatcaacttcagggctaaatattcttcaaataaaatccagtatggattttatatcaatgtacagtatgtaaatatgttctttactgaggaaccttagaaggaaactgaaatgggaagatggttcctgtgcttgaacaggaagattgaattttcttaagatgtgagctttttggctgggcgcggtggctcacgcctgtaatcccagcactttgggaggcgggcggatcatgaggtcaggagattgagaccatcctggctaacacggtgaaaccctgtctctactaaaaaatataaaaaaaattagccgggtgcggtggtagacgcctgtagtcccggctactcaggaggctgaggcaggagaatggcgtgaacccgggaggtggagcttgcagtgagccgagatagtgccactgcactccagcctgggcaacagagcgagactccgtttcaaaaaaaaaaaaaaaagatgggagctttttctatttatcacttttacctaagccaaataaaaatagcaaagttttagcgtttttaaattacacatgctgtcttttattattgtgataaattaattttttgtaacagaatggaaaaaggctgcttttccagatatcaaaatgtgcatgttatttatttccacaaattgtttactaacagctgaaaagacatcaatgaatacaacagaataggaaatttagaaatacccaaatatatgttaagaatttagcgcttgataatggtggtgttttttattatttgaaaaagatggattgttcataattcatgtttggagaaaactagctagatttttatgtcacaaaaataagagtatagattaaaaaatttaaatatacaaaaagagaaacataccagaagagaaacatacaaatgcctatttatatatgcagatatatatttatatatatacttttttttggatggagtctgactctgctgccccggctggagtgcagtagtgcgatctcggctcactgcaacctctgcctcctaggttcaagcaattctctgcttcagcctactgagtagctgggattacaggcgcctgccaccacgcctggctaattgctttgtatttttagtagagatggggtttcaccatcttggccaggcaggtcttgaactcctgccctcgtgatccacccaccttggcctccccaagtgctggggttacaggcgtgagccactgtgcctggccatatttgcagacataataaaataagctcattttaaaattgggcaaagtactttttttgcatatctaccagtgacctatgcacataggaaaagatagcgttcctggtagaagaaggaatgtaagttagaagaggaatgaaatactgttttctatttaagttagaagaggaatgaaaggccgggtacagtggcttacgcctgttaccccagcactttaggaggctgaggcaggtggatcatgaagtcaggagtttgagaccagcctggccagtgtggtgaaatcccatctctactaaaaatacaaaaaattagctgggcatggtggcacgcacctgtaatcccagctacttagaaggctgaagcaagagaattgcttgaaccagggaggtggaggttgcagtgagccgagatcgcgccactacattccagcctgggtgatagagtgagactccatctcaaaaaaaaaaaagaaaaaaaaaaggaatgaaatactgttttttatccacaaagtttgtgaggatgaagaacagtggtacttacgtagtcatttaaagtttaaattgctacagcttttcaaacaaacactttagtggtaagaaccacatttttaaaatgttatgctttttactcatcaattccattatactgaaatatctttaccaaatagaggtctgtttttcttagtattgattaaaatagcagtgtatttagaagaacccatataaagatttcatggacaaatttcagtgcatccataggatggactaatatgtaactattgaggttgtcagtagatacagagatatgtcgacatgcaaagatgtactttgctataacaagtgagaaaaaaaatcggttacacatatacacgaacagaatctgctcgtgttagctgaaaacatgtagaaaatataatcaaacttgtttctggggatttgtaagtgaagtttttccctttctcttatctgtgatttctgcaatgaacatctgaagttttagttaaagttcattagtaatgcaataatccttgggaagagaaggaatatgcttcttgcatagatgcaaataatttgtcacattctattatttatttttctatctgtggttggctatcttccgtgaacttttaccctcttcagaagtagagggattgtgtttacctgttcctgtagattttattgtatatacattttattacataattatcttttcattagatatagattaacatgtaaaaagtatgaattaatcattttagttgagttatatgtttatgaaaattaaaatagcaaatgtaagtgattattactattgcaaatgtattgcattactcgacaggagttttctttgaaaatattgaactcccaagctgtgttcatccattctttcaatccatttagtcatcagacataagccagacacctattatgtggcaggcatattctactatctctcaggatccttccacctttgaaaacttcatgtttatctgctgggcttgagcaagatgagagatttaaaattggtgcattaggacttaatctcaattgaagcttttcctccctcctttcaaacagaagcatttctgaaggtagaaaatagtaaaagacaacccttaactgcccttttgaaaatttataagtcttggagaaagactgttttagttgttttaagaactaaaatgtggtacataaacagcatggaatactatgcagccataaaagaaggaacgagagcatgtcctttgcagggacatggatggtgttgaaagccattatccttagcaaactaacaaaggaagagaaaaccaaatattgcatgttcttacttataggtgggagctaaatggtgagaacacacagatacctagagggaagcaacacacactggggcctattacagggtgaagggtgggaggagggaaggaagcaggaaatagaatgaatgggtactgggcttaacacctgagtaatgaaataatctgtacaaccaacccccttggtgcacgtgtttacctgtgtaacaaacctgcacatctggcacgtgtacccctgaatgtaacagttgaaaaaagctccacaaatcgtttcataaatccgttttaaaaggagaaaatttataacagtcttaaatcctaatatgaatgattggaaatatctgatgtacatacattgtataaatctaagtattgacaaaaatgagcccatgctattcatttgaattccaggttttctttggcttaaagtttattgaaaaccaaagtaagaattggtttattttagaaatttgtttttgttttcacctcagctttcttattccatagttcttttaagaaccaaaatttatctaaatgctggtcatctgactggaaccgccccggacctgttataacatattctacttcatgtaagacaccagggattgtgtgatgccccattattttatgcctcaataagagaattatttaaatgccgccaattatagtaaatcatgaattgtaagtggtatttcagtggagacaacatggagacaatgatcatctcagaatcactaaaatacaatgttagctgtaatatttaaaacacacctgaaagtgtaggtataattgtatcatctcaattcaaatgttgtctttagtggtattagtaaaaattaaaatatctaacaattattgagctgttatttgtgttaggaactattctatatcttttgtgcagagtctcatttaagcattagagtggtttcctgtgagaaagctactattttcattcctattttattgatgaggaaactgagaccccaaaaggctaagcaacagccaggaagtgacagagcttcaagtaggattccagcccacgttgaatgtcatccaagggctatgctctttatattcatataggctgccctttcattaatacagcgagcaatgagagataataaatagtgtgcttttttcatgggaaagttaaatgtttgttttgaaggcagagtaatagcaggctattcagtgtttgcagttacatgaatcattgctatggctgtagctagtgcactacaatttcctaaaaagtcttctcactctcataggactgctctacatctggcctctgccaatgggaattcagaagtagtaaaactcctgctggacagacgatgtcaacttaatgtccttgacaacaaaaagaggacagctctgataaaggtatgcagtagccaactatatcagcatgaggtgggtttgatttcaatacatggcataaaaatgagttttctcctttaaatataactagttggtgaaagctgtggaatgttattttgaattcctaagatttgtaatttgtttttggtctaatactgacaggccgtacaatgccaggaagatgaatgtgcgttaatgttgctggaacatggcactgatccaaatattccagatgagtatggaaataccactctgcactacgctatctataatgaagataaattaatggccaaagcactgctcttatatggtgctgatatcgaatcaaaaaacaaggtatagatctaccaattttatcttcaaaatactgaaatgcattcattttaacattgacctgtgtaagggccagtcttccatatttggaagctcaagcataacctgaatggaaatattttgaaatgacctaattatctaagacttcattttaaatattgttactttcaaagaagcattagagggtacagtttttttttttaatgcacttgtggtaaatactttttttgaaaacactgaatttgtaaaagataatacttactatttttcaatttttccctcctaggatgttttttccctattgaatgtaaaatggcaaaatttgccctgaaataggttttacatgaaaactccaagaaaagttaaacatgtttcagtgaatagagatcctgctcctttggcaagttcctaaaaaacagtaatagatacgaggtgatgcgcctctcactggcaaggcttaagatatttctgattgctcatgagccagaagtggaaagcaaaaaagagagcaatcagaaatatcaaggccaatttggaaattagataatggagggaaaagaccatgaagaggttgtgtgtgtgtgtgttgttgttgttgttgactgatttgtttcctttgcatggtgagacaaggttctcttcaattttagagaatgacagttttcagtttgggagagggagttagtgggttgtaaactacctagagatcaattttaggaggcctctgaggaaccagattggcagtgaataggtggtaatgtaagaggaaacccttgagcagagggaatatcaagtaattaactgacttagtatcctattctggtagaaatggccaatgagagtctcagctctgctttcaaatctagagtgtctggatgggaaggtggaagataaacaagtaacaagatcaagttggattttgagttgactagaccctgttcttctcttaccggggaaaatcatgtggtgttttcagcaaatgggtctctctcctactccttactctttttggccaaatcttcaaatgagaaagggaattggtcatgtgggtgagaaatgagactgaagtaattgtctgttgcactagctttcagctagaattgtgcatcctagtaacctgaggaaaatgtttaaataacaagtctaggctttttctgaatattttgatacagtaagtctaataaagcctggatatgtatatttgaaaatgtttccttgaagccaggcatggtggtgcatggctgtagtcccagctgctagggaggctgaggtgggaggattgcttgagctcaggagttcgagtctagcctggtcaacataatgagaccctgtctctaacaacaacaacaacaacaacgacaacaacaacaattttctcaaaatctggatacactcctgcttaagaaccactcaatacataaatgtaatatgtaaattcttatgtctcagaaacttaaggtatctctagaagagttggggttggatatgtgctgatttctttaaatctttccaataacattaatctgacttttttttttttttgagatggggtctcactctgtttcccaggctggaatgcagtggtgtgatcacagctcactgcagcctcgacctctccaagctcagatggtcctccaacctcagttttttttttttttttttcagtagagatgaggtttttgccatgtttctcaggctggtcttgaactcctgggctcaagcgattcacccacctcagcctccccaaatgctaggattacaggtgtgagccaccattcctgacctagtctgacttttatctctgtggttgagacattaaaatgaatattattggtagtatgtatcagcttacagaataataccttttccttcctaccatcagttattcactgccgttcagaaggtctttagaaatttgctgtgagtagtctttcaataagtagaggatggccctctcaggattttgtgtctctttgttcagtcattcaagtacttaggtcagtaagtcgttaagagcagagttttctcaattagaatagtagcaaattctaaaccattttttgtcaattgacgctgtattatggactatccagtgtgtctcttaagtatgtagagctttggcataatcagcatggcagttttaaacactaaaaaccatggagttattaagaatacagataggaattctgttaatttagtttcagtagtcctatgaactgatggtttagttaacaatctgggaaaattaaatacaaatagattttaaataaataaatgttggaaaattttttcaaatgggcagtatgagttttaatagcaatttttgttgcatgttggaggttgaacttttggtaaaacatgaaactaaagaaatattttacatgcagattcttgctttatacacaatttgtcttagggttgaggatatagagacaaaagatacagcccctgccctcaagaagctttttgtttagatgggaaaaatattatcatccaataacaccaagccaaatgctggtttagaagcaaagagccttggaagcagtaaatgtttaaagtgagtttttgagatgattagagttactgtggtgaggcagagaaggggtgtttccaagggaaggagcagcgtgtgggaaagcacagaagagtgagaaggaagtgactacattttatttacttctatgcgtgtaagtccataagatcttatataaagtttccacttcggttgaggaatatgtacttttttgaattacataggtttttgctttatattgttttacagcatggcctcacaccactgttacttggtgtacatgagcaaaaacagcaagtcgtgaaatttttaattaagaaaaaagcgaatttaaatgcactggatagatatggaaggtatagttctttcttttaatctgtgtgttgtagatggacagcagtcactcaagtcataaatattaaattaataagattaatgtatacttactgggatgtagtgatcagtatcaacacaaatcagttaggtagaaaaacaattacttgggctgggcaacataaagaacagttttagtaggattcatcttctcttattatattgactgatgttatttgttatgtgacatttttggttacatgatcttatgttagcttaaaggatttcatattaattttatgaagtttgaactttaacttttagtttactttatgactcagtattgaacttcttaaccctttctaatagtttttaacctgtgtcttacatgcttttccattaaatatgctgtattaaacataaataggggttgaaaatccttttgtctttttaatgactttgctttaagttgctttctttgaagaatattagtgttagcttatccctacataacaattaattgctgttcccacatactgtgggttcaacagcttttttccttttgtttttccagtgtattttgatgtttttatttttaattggtatggagagagggagtgaagatagttttaagtggatacacttttcctttaatgaagacaagccgtacgtgggtgataaagagaaaagagctaggctttggattcacacaagactgggtttaattagtaactttcttacttgctaggtgtgtgaccttggggacgttatttaccaccaaatatgttgtcatatatgaaaagtaggagaatatatccttcaaagttggctgtgcataagtaagaaagatatatgtggcatttaattcagtgcctagcacatggttattggcaccattaactgaaactcctatgactactattcttaccattattattactgctttgagcatgcagagagctcttatttctcttaccccctagctgattttctattacagcatgtcagtctagggaagctgtgacaaaatcttcacttaaatctttgtccacctcagataagtggccctaacattgtttcttgcccatcaaaggactttaaattagtagcttctgctatgcaataccccactgagataagagggttttttttggtcccttccttttaaccttggtggcattttacaaagatgaacacttgagcactcaggttgcttatgtcttttagtgcatgtaaatgtttgattctgcacagacaggcaagatgttacattggtaaagtatatcaaattagctttaaaaataactttattacagttcctatctctgtcattttagaactgctctcatacttgctgtatgttgtggatcagcaagtatagtcagccttctacttgagcaaaatattgatgtatcttctcaagatctatctggacagacggccagagagtatgctgtttctagtcatcatcatgtgtaagtgtttacattaaaaggctagttaatgctgaattgaggtttaaaataattataacagttgcatctcacatatcaggtgagatgtcatagtttggttcaggtagttttcgcgtggcagtgagttttagtcccctgcatcagccagaaatcagacaaaaagcaagacaagttagaagtaccaatgggtgcgggattctttatctcaggacttttaagatctttatccttagagatcccagcattgttcatttgatccaagtgtaacacctatgcagggataaaaaatagtgtcacatctttaatttttctgattagttatttgggtcttgaaatgtccagtttagcggaaagtcttgtactgtcttctggggactatgtcctacatactccttgaatttttcaagaaccgaaggagttcactaaatccaaggaagacagtcccttttatcaagtcagaaggaggaggaaaaaagacattgcaatcattctgttgtttccgttgattctgttgctgctttgtggccactcaaactggtcctgctgccttaagatgaatcagtagattcagatccctcaagtcttcatggcgattcatacagtgactttgatgttttttaattcccatacctatgcttatatgctcagccattgttcccagagcaccagccccctgctctggccgctgggcatcctgactttatccgcacacaaagtgaccaaattgacccttccccgcatattcagaacctaatgtggaacccacatcttagccaggaattagctgagaccttcatggtaagagatcctttgaggctgttgttggtcttttctctggcagatgttaggtgggcttgttctaaagggtgagaggggttcaaataatgtggcagaaggaggtcagtgtctgtttcctctttgctaccagatctgtactgtgaggcacctttatatcctgtatagaaccttgggcagtagaaagtcccgtatgaaccttgccctgagcagtggctcccagctgtggttggccccttgagtgatctgatttacatgataatgaaaattatcggagctacttccatctctagctcaagattttaagatattttcaaactctagctcacaggaagccattgaagagaattctcagaatctcaagtaggttagttggacttcacagagccaagccttgtccatgacgcatcactaatcatatgtaaaagtagggctttgtgcttgcttcggcggcacatatcctaaaattggaacaatacggggaaagttagcgtggcttctgcataaggaggcaggacagatctttgaagcattccatattttgtgcagtcactggaaggtcatttgactatttttgctgactagctctgaggaaatagtgtgaatcaaagcaaaatgggtgccacccaaatattgaaattgtgatttgtgctgcaaaaatagtcatgtaagatggtctatgagatgactttgagctgaataacatgttccgtgcaaaatatattgttagtatgtatgtcgaaaattacagaatgtcagcttgctacttctccgtggaaactaaaaaaaaaataaaagtagacttttggtctcccatgtcagctggaattgaacatcaatataaagcattatcctaacagacatctactggctgagagtttgagtctgtagagaaggatcgttggtccaagtcaggtcttaacatccattggtttttctgcccttggtgtgattgatcaactccgtaatagtggacaatcacattatctactttaatgagatatttatgaataaatttagttataaactatgacatagttgagatgccctgaattataagccataaagagtaggacaactaagaagcaaaattaggacttaataacattttctgaaaactacaacatttgcatattagaacctatgaacaaaatacgcattgggttttatttgggattccaagataattttagtcataaagtttaggaacagattattccattgctttactatttctctgagcatttaaaaaatgttatctcgttaaatctttataacaacctagtgaaataaggcagcaaagtcctcactttgttgaagaagacattgagcctaagagaagcaagttgtccaagaacaaatagctgttcattatggagctaggacttatgcagagttgggacactttctattatgtcatgctaatgctagctaatttactgggtcacagtgccctcgatttatgactatttcaccttacatttttttcttctttaattagaagcttaaagagaagtttgtagaatgtactcacaagtgggtgggataatactattaagttctgttattctgatattgtttgaaatactcttaataattctacatttggtaagtttttttatatcagtattaaaatagtaatttggtttattacatttttatacatagaatttgccagttactttctgactacaaagaaaaacagatgctaaaaatctcttctgaaaacagcaatccaggtaagacttgtgatagtgaattactttagtcagttgtccccaatctttttgacaccagggaccggttttgtggaagacaatttttccatgggctgggggagggtggggatggtttcaggattattcaatcatgttacatttattgtgctactttatattattatcacattgtaatatataatgaaataattatacaacttaccataatgtagaatccgtggaagctctgaacttatttttctgcaactggatggtctcatctgggggcaaagtgagacagtgacagatcatcaggcattagattctcatacgaagcacacaacctagatccctcagatgggcagttcacaacagggttcatgctccaatgagtatctaatgctatcactgatctgactggaggcagagtagaggctgtaatatgagccataaggagtggctgtaaatacagatgaagcttccctggcttgcctgctgctcacctcctcctgtgtggtgtggttcataatagtccatggactggtatgagtctgtggcctgggagttgaggacccctgctctgcgtggtcctaccatagataaaaaagtaaaagtaaggaatttttgatcacaaaagaacactgaagcacaagtcatgttacatatgcttgtcccaataagttctcactattactgacttcattcctcctcatttgaagttggaaagagatatatttactttgttggaacaagatgtgttcttctacctgctggttaattgtcatcataacagtaattttgttagaacaagatgctctgctaccatttgccaaaagattgtcataataaatatacaaattgcccaactctaggctcagcagattataataaaagcagaaaaacgtttcacgctaacaaaaatgctagtatgctacctggttgtggacacctaatacattatattgtccaaactgtgtgaggacacctttaatttagtcatctatttatcaaagagcttttgtaagttaggttttataagttgcaggagacaaagatggaatagatgtagttttgatctttaaggtgctcataatagagctgtctccatttcatttctgtgctttttcaacagaatttacaaagaaaacatttctatttatgttttcacttgtccacttaacatataactatcaaatgtcttttagatagtaaccatttttctaatgctatagaccacaaacaattaaaaatacagacaggagctttttgttatcattgtcattttcattattttactactttattcagtgcttactgtgtgctagatgcccactggaagcttataattatgatttattatatattgattatgtgccagacatatgtgatgaggaatgaaagttttgggaaaaagtaggtatgatttaaggtaagcatgcagagagagaagaatttttctaggtaaagaagcagaagaagaatgtttggcagaaggaacatgcagcgaggtcgtgtgtttgccagaaggaacatctaatgagattgcctgtttgggaggaagagcagcaagtgcaaaagacaagatgcttgagtgaacatggcagggtttctgagcagttcacttttgctagtaccaaaagtgtgtgatacgagaggttgggaatgaggtgaatacttagctaaggcaagtttatgatagactttttaatactatagaaatgagtaggtcttatcctgtgtgccatgggaaatttactgggtagaatgctttggactgcaaatactagatgaacagtggctaaaacagtaggaaccagagttgttttggttgttcattgatatcctaggatcccacttttccctctttcagctgtgctgttgacagtgttttattcacgtctcctttcatggttggctaatccgcagcagctccaaacatcttgttctcacaacacaacatcgcaagggctgcttttcttcacatgtgtcttttaaacagggagaaaacttagaagcatgcaaagggcttcctgtaacatttcactggctgggtcacaccacatgctcattcccaaaccaggcactgggaaggtaaatacctgattagcttagaataaacatttctgtttctgaggctgaggagggggattgggataataaatatcccaatagacttggatttcttcttcaagaaagaataaggaatggctattgatagggagccaacaatgtgtgctgcaggggctcattggagaaatttgagcaggggagtcacaagattaaatttgagtattaaggcattctggttatggtgtaaaacgggttagcaagctttttctgtaaatggccaggtgggcaatattttagactatgtggtctctgtcatatctacttaaccctgctgttgtctgctgttgtagtgtgaaagccaccatgattatatgtaagcaaacaggcatgactgagctcctataaaactttatttacaaagccataaggcagattggatttggcctgtggcctatagtttgctgggattgatggaaggttctttaccatgtaaagaaaccaggagacaaaggaagttttcgcagtagtcagctatagtttccttgtcatacatccttggagtagcatcaatgtattacaaggttttcacccgtccttagtgaaatacataaagttaggaatctcaactacttgttttaatatgttggcctttgtttttttggtgttatgcttttttcatttgttttgcttaattttttttatgtaagaaataacattaatagttggcaggcttttttttaaataaaagccattttgtaaatgtttgtgttcccagtggcagtgggaatataaagcagaggcagaagagaggtatcgtcaatatgatttagtgataattgaatgagaaaggcttgggggacagagagaaatgtcagataatttccaggtttccaggttgtacagtagtatttaacctggatgtgaggacgaaggaggaaattttctggtgaatacagaagagcaaagagcagcaggtcagcaggaatgactaatgtttttctatgcacgtttaatggaaaattcatgtaggatattttgaataggtaattggataaccagcatttataactcgcatcctactagtttgactctcactaataagacttgtcaaagatccaagaatctgaaagttgatgataaatgtccatgtgtatcaccatcaatgatcaaaagttagcatccacagacatagaatgggacagatgaacttaatggataaagatgaatatcggagttgttccttttagggaatgatactctccatgacctgtgtgagtcacagctgccagaaaagagcgagcaaggagcgtatgaaggcagcacagcaaattcagtcctagagtgccctgcttgacttcatgtcatagttctgacttctaaaaaatcattttctgtgaaatacactttgattttttccccgcttgcagcctgcaaccaaacagaatccctttagcagggcatttttgtgttcttcctttaaacaaagcaatatataaataacaaaatgaaaaaagagaaagagtgttttttgtataggctagtatttaacataaacttgagagtgagtaccaggattatagttagaatttacggactgggtaggaagactggatagaaatctaaagattgctgactcaaacacaatgtggtttctttgctttattgtcacagctctgaattcacaattattagttaaattcataggcactataactttagaaagcaccttcccaaaccaagtattaagtgatttattataatttctctgacttcttatagaattgactttccaagtgttcatgagaattattgagaaattgctacatagtatcatctcagctctgtccacatgagctatctgtcaccttgtcttaatgaataattgttcagtaggaatattggttttggcatttaaagtgatctatatctaaatgcagataggatcagggaccactcttgaacattaatgtccaggcatcttaaaattacacataaggctttcataatctgacttctgccccactctccatctttaggccttttccctgtgtgccctttctctggcattactgagctgctggcaatgccctactcactcatcctcctattgtaggcaaatactttcactctttcaggcctcgctcccgctcttgctgctgcgtggcatgccgtcaccctttcctgccctctaccccttttaatctggctagtctcaatatttaagtctctgcttgggcatgttttctagaaaagccatccctgacatgctttattttaattctttttaaaccctaatgcctaacatgtatttagcaggactcaataagaaatttctgagtaaaatgtgagtaagatgatgctcaagactgtcctctgcagtcttggagcagacgggacagacatgtggaggaataatgtacagttcaggtggtaaagatgcagtagaaaaatcagtgaagtactaaggcagcctcaaggaaggaggtacctgtttatctggggaaagacatgcagaatcaaggaagacgttctcatagcattgttttaaaagataaatataaggacaagtgtggtggctcacacctctaatcccagcattttgggaggctggagcaggcagatcacaaggtcaaaagatcaggaccatcatggccaatggtgaaaccccgtctctactaaaaacacgaaaattagctgggcatgatggtacatgcctgtaatcccagcttctccggagactgagacaggagaatcgcttgaaccagggagtgtcagaggttgcagtgagctgagattgcgccactgcactccagcctggtgacagagcaagaccctggctcaaaaaaaaaaaaaaaaagaaagaaagaaaaatgaaaataaatgtgtcagaatagtggaggaaaacattttagatattgggaagacattgtacactaataaaggtgtcagtagtaatttttgaaatcatttgtaaggtactattgttgcagaaagcaggaggcaggagagaccaagtgggtgaaacaggaggatttcatttaggttcgcaacagctcagcagatttgcatccgaaagctgagccctgaacaaagacagggcttggcttatgtaggcaagcttacagaagcagaacaaaggcagttaatcatatagtgacagttttgcaacctcagcaaagcttatgaccttgcagctgcattgaaggtcaacaggaacttgcgaatagtatgcatttgtaaaaacagctctgagtgaatgctgaggggaaggggagacagtaaaggattttgttttcttaaccttgctctgggatgtctggagcccatacctgtgggctctggcttctcaggcagggtcaccacgacctttcctgggccctgcttgttactatccttagagtcagactagctaagtgcgggaaaacttgtttctctttaaaactaaatttccttttctttacatttactgcttcactattaggaagtggagaacaacatactgtgttaccttagatgcttctactgtattttaaagttgtgtttctggtggttttgttcatttatgttgggtggatgaatttgtgagtgaatgacatcaggtgtctccccaagtggtttgttgaagtttgggagaattatttcctaaataactatttcatgaaaaactaaacactcaatttatgaaataaaatgttgtcttaaatctatttttataaaggcaatagtttttaactgttctaagtggttcattttaactgaatatatggatttctcaacagaacaagacttaaagctgacatcagaggaagagtcacaaaggttcaaaggcagtgaaaatagccagccagaggcatggaaaattttaaatttaaatttttgatttaatgttgttttctttgctttaataatattagatagtccaaatgaaattacctttcagactaggttttgagaatcaatagattttttaaagaattttttaatagattgttaaaatttattttaataaattcagtaatctcattaacagaagaatggattctaatttaacatttgatatttaacttaaaaaacataaccactataaaatttaaaatactcttataatatttttatttaaaatactctcatctgcctttttgattagcttatagctaatctttccttttggaatagaggcaaaaacaaattgcagaactttgttcttttatttttacaacacccgaacatgatgaagaaggtacatcaattattggattatgttattaagcaatagaattatgaacaatgtaactctgatggtccctgagctggattcatggttaaggagtaatcatggccagtgattgaaaatctgcagttttatattgtcagtcattgataccaagcttaaaaatatattctgccttatggtctctcgttgacctcagcgtttctgttcagggagggaaccaggtcataaaagcaacacagctgcctattacaagaatcatatcttgcagaatggaacctttggtgttagtgcacaaacacaataacattctaatttatttcagttgcagaaaattagtacatattgttaaaaaatctttatccactgtaattagtacacattagaatatattagaactggacttaagcagataatctggatacataacactatcatatgacagtatataatctcaattaaaatttgagaatttgcatctctttctgtttggtgttgatttcggctcctaataatttaaatggtgcctacaatccagttaggaatcttttagaaaagcacttcagtgcactgtaggggctcactagttagggtttcatgaggtaaactcttttcaagtgaggaaggttttgcaacactacaaatcatctgctgattcatttttggtagatttaacacataacaaattaagtttagtccaaacaaatggtgaccaaattaagtttgctggttcacgttttttttcttcattgggctaaggtgaattatttttcacatgttagtcagaagccagtaatgtggcagtagctaaacatagattaaaaagttaattcttaattttaattattatttatttaacagttaaattttaattttaattattttctaatttttcattgtccatacttgattagttaagaataaaatttttttaaaacatgcgctccaaaagaggacacatcacagaaacacaacaagcaaattaaccttctgtttttgcctctgcagaaaatgtctcaagaaccagaaataaataaggatggtgatagagaggtataccttcatattcaaatgtttctgttgaattagatttttacattatgttgtttaacaaaatgtagtaagtgtaggcatacatgatcctatcatgtaagtagcataaatcatcagtgaaaaagttaatatttaactcagaaagaattctgtacattgagttttcaagagatacaaagcctagagatattctttgattattatggaacaatcctgaatggtgctataaaatgctaggtaatgccactttaggagctttggaccaatcattttatctttcttggttttagtctgattatcaatagataatgtggctaaagaagatagctttttattctgtgtatcttccagctagaaaattgtatggctattcaatgtgaaatttggggagcatctcattttctggaattccatgcttgcacgtcagcagtttcactctgcttcttgtgttgtggcaaactttggctcccattttcagtgagcaccatcatgtttttgatatcccagcaaccaaatgaaaaaagaatgctcaaaggcagtggggtagaagaatatcttagcgcagaaaagggcaaacttcctttctattcctgaagccgcacagtgtgtcatcctctaaatctgactgtttaatgtaaaatctaggtggtaaagacggaagaggacacattttgcatctttgtctttttatttgtgtgttcccatgaatcaaatggggtaaatacatacataagattctgaagagtgattgagaataaaagcacaaaatgaaggagggccctttttgaattttggaaaattctgtttcattcattgaaacagaaatgaagcaaactttacaaaaatttctgtgatatattaatgagatgataattacatcttaaagttatatggtaatatttctatatatatgatcaaatttaagtgtgaaatatttttaatgactaaaataatgacaaactgagtcaattgatagaatcaattaaaaaggttctttttattcaataaagtgataactatccttaatatcccactcaaggttgaagaagaaatgaagaagcatgaaagtaataatgtgggattactagaaaacctgactaatggtgtcactgctggcaatggtgataatggattaattcctcaaaggaagagcagaacacctgaaaatcagcaatttcctgacaacgaaagtgaagagtatcacaggtaagcctatggcaacattgaatagaagataactatgtgctgtcaaactaaccctaatttgggctaatattcatgatgaacaaattttatagttttactaggatattcagccttgcccgttaatcagaaaaatgaaaatcagcgaacaatgagttaccatttttccaggcattaatttatttgaaaaataaccagtattggcaaatgtgagggaaaaggcattttcttttctttttagtgaacttttcttttagtttcaggggtacatgtgcagggttattatataggtaaagtgtatcatggagatttggactgcagattatttcatcagccacataataagcaaaacaccaaaagttagttttttggtcgtctccctcccgcgacgctccaccttcaagtagacccttggtgcctgttattctcctctttgtgtccatgagttctcattatttagttcccagtaatgagtaagaatatgtggcatttgatcttctgttcctgcattagtttgcttaggataatggcctccagctccatctgtgttgctgcaaaagaaatggtttcattgtaaaagacatttcatacactgctggtaaatacattttgaacattaatttagtagcatattcacaaacacatgtataacaataaggatatataatatatgtaaaggatatttgtgtagatttgttacatatatacttatgtataaggacatttattacagcattattatatcaaaaagatggatccttatcaataggaaattatcattatcaaaagtaaatcattaccaataggaaatagctcaattttcatacccagaaattaatgtagtatgcaaccattttttacaagtgaggttagatctagagtatactgattatttcacaattaaagtgtatttaaagcatcagtgatgacatcttaagagttcttgttaaaattcttgtaatatttcctgtgttgcaaatggaagctacatgctatattgatgctgtaccttgttagcaacaagattgctagtcattaaatttttattgtcagtgcctgagtgctgaaatattggaccctcaatctgaatattgccaagggattgtacatggggatctgtatttaatataaacgtttcagtatattgggtaaaacttttattaaaatatatcaaaggatctttcatctgctaaaccaggagttggccagctttttctgcaaagagccagttagtaaatattttaggctttgtggactatataaattaatttatttttgagacagggtctcactctgtttcccaggctggagtgcagttgtgtgatcatggctcactgaagtcttgactttctgagctctagtgatcttcccacctcagcctctctactagttgggaccacaggtgtgcaacatcacacccagctaattgacactgtggactgtaaagtgaataagcatggctgtgttccaaaatacttgacttacaaaaacaggcagtgggctggatttggcccaaggtgctaatttgctgacccttgtgctaaaaggaaggtgctgcaagtgaagtgactcctatttgtaaaagtgccctgcgtgtgtgaaattatccttcctttgagaaaaggatatatttcagtattcacctcaccatatttttccacagtgacttcatataattttaaaaatttcatttataaaataagattattttctgcatttctcccactttattcctgttaatagaacccagtattttgctgtgatcaattattttgtatatttgatgagtatcagttttcctagaattggctgattttatcaagcaagaaatactctccttgaaacttttagtatttcttggtctttatgtataagcatgaataaaatgataatcagcttatgtgtaatctagaaatgttcaaggcgacttttagttctatagttttaagaatttaacacctcagtctggcatttttaatgccacatgtgtataatttttataacctttaaaatatataattgttatataaaatttgaaaactacacctgttatataaaacttgaaactatttgtctattacttttccatgactgtggaagaaaattacaacattctcagtcatgactcctaagcatgatgtccttaaaagaactgtccacactcacgaactcaaattttcttttcattcactcttaatctcacaccggcgtcttcaatttcagcagtcctccaacattgtttttcctcaagattatcacaatttttttctgtaaattatgcatttttcttacacctcattttatttaatctgtcagcaatatttgagccagtggagggcgtctcctccctaacggcgtcttcacttggctttcaggtcctcactccctcaagcttttcctcctgcatttctagtccattcatcatggtctgttttgcttgctcctcctcatctttctccttttggacattgttgtttcccagggctcagtcctcaatcttctttctcctgactttttctttttctttttttgagacggagtttgactctgtcccccaggctggagttcagtggtgtgatctcagctcactacaacttctgcctcttgggtttaagtgattctcctgcctcagcctcctgagtagctggcattacaggtgtgtgccaccatgcctggctaatttttgtatttttagtagagacagcatttccccatgttggccagcctggtctcaaactcctgacctcaggtgatctgtctgccttggcctcacaaagtgttgggattacaggtgtgagccactgcacccggcccctcatgactttttctattgtgtatatgctagtgatttccaaatgtatgtctccagctcagatctctctccttaattccagattttttatatcagcctgcctacttgacgtctctatttggttagttattgggtatcacacacttgtcagatccaaaattgggctactgatgtccttcctgaaatctacacctcatgtagtctttcctactttggttaagggcaactcttccaattgctctgccaaaaatgtcattgtcattcttgactcatctgtccctctgacacctcatatctaatctttcagtaaatcttgtcaggtctacctgaagaatatgaccagaagccagtcatatcttgtacatctgagccactgtcatctgcagtcatgagtgtcatagactgggaattgatcgtcctggcttttaaaaacttcccttttcatcaattcttaactcagtggatgtatttaaaacataagtcaaattgtgtcattcctcttccccagcacttctgattgcctccttttcactctgagtatgggtcaaagttcctcctgattatctcccttgctctgcttcagccacactgaattcttgccatcccgtatctacccctagtgcttaaagactccagacacacctctgtgcttggcagttccctgtgtctgggatgcttttcccccagatatcctcctagcttaactctttccattccttcagttctttatttaaaaccgcctttctaagaagaagaagacaaagggtaaaaagaaacacattaaggaacatccactttctgaggaagaaccgtgtactacccagacgcatcatgcttaagatgcaattggcagcatacaaggaatgctctctaaggtaatcaaggcaaggttcaatgaaacaaagtgatttatcatctctaacttcaaacctatttgtatcttgacatcaacgctgttaaccttatgtcatcatttcttagagtctttgatatacaaataaaaggttttttgtattagaaaaaaaaaaccctttctcagcagggactcttctggccatctcaactttcccaccaccctccccatcaaacacataaacatttcattttcctgctttagtttttctcctctaacataccgtatattttgccttatctgtctgttgttattgtgtgtttttctcactgtcatgaatagggtttttatttttcactaccatatcttcactgcttagaaaaaggcttagcatattggatgtagctacctaataaatacttattaaataagtgaatggagtttatcctgtgtatattgtttgattgattctcactttgaaaatgtttgacatgggtcgggcatggtggctgacacctgtaatcaaagcactttgggaggccgaggcaggtggatcatgaggtcaggaggtggagaccagtgaaaccccgtctctactaaaaatacaaaaaaattaaccgggtgtggcagcatgcgcctgtagtcccagctgctatggaggctgaggcaggagaatggcatgaacccgggaggcggagcttgcagtgagccaagatcgtgccactgcactccagcctgggtgacagagcaagactccatctcaaaaaaaaaaaaaaaaaaaagtttgacatggttctttcctaacagttttgcctggtaattgtctgcatttttaaaatcgttttggctctttgtaataagctacattctttatattaatttttttatttagggagaaaagcccaatattgtggttattcactatttattctttaatagtaatcataattgtcattatggtaaactgagtcagaggaattgcaaactttactattttattttattttattttatttttttgagatggagtctcgctgtatcgcccaggctggagtgcagtggcgcgatctcagttcattgcaacgtgggttcatgcatttctcctgcctcagcctcccaaggagctggcccttgcataaggcggccacacaaatttttgaagcattctatattttgcgcagtcactggaaggtcatttgactgtttgctgagtagctttaaggaaatggtgtgaatcaaagcagaatgggtgccacaaaaacattgaaattgtgatttgcgcaataaaaatagtcatgtaaggtggtctgtgagatgacaccagagccaaataacgtgtggggtgttgtgtaccaaatatactgttagtatgtatgttaaaaattagagaatggcaacttacaacttcttcgtggaacctaaaaaaaaataaaagtagggttttcgtctcccatgtcagctggagatgaacatgtatataaagcatcatcgtaacaaacatctggctgagagtttgagtctgtagagaaggatcattggtccaagtcaggtcttgacatccattggtttttctgcccttggcgtgattgatcaactccgtaatagtggacaatcacattatctactttaatgagatatttatgaaaaaatttagttacaaactatgacatagttgagatgccctgaattagaagccataaagagtaggacaactaagaagcaaaattaggacttaataacatttcctgaaaactacaacatttgcatattagaacctatgaacaaaattcgcactgggttttatttgggattccaagataatttcagtcataaagtttaggaacaaattattccattgttttactatttctttgagcatttaaaaaaatgttatcgtgttaaatcattataacaacctagtaaaataaggcagcatagtcctcactttgtagaagacattgagcctaagagaagcagcttgttcaagagcaaatagctgttcattatggagctaggacttatttagagttgggacactttctattatgtcaggctaatgcaagttaatttactgggtcaaagtgccctcgatttatgagtatttcatcttacttttttttcttctttaattagaagcttcatgagaagtttgtagaacgtacgcataagtggatgggataatactgttaagttctgatattctgatattgtttgaaatactctaagaattttacatttggtaagtttccagatcagtattttaaaacagtaattttatttgttatatttttatacatagaatttgtgaattactttctgactacaaagaaaagcagatgccaaaatactcttctgaaaacagcaacccaggtaagacttgtgatagtgaattactttaggtcagttgtccacaatctttttggcaccagggaccggttttgtggaagacaatctttccatgggctgggggaaggtggggatggtttcaggattgttcagtcacattacacttattgtgctactttatattattattacattgtaatatataataaaataattatacaacttaccataatgtagaatcagtggaagctctgagctgatttttctgcaactagatggtcccttctgggggcaaagtgagacagtgacagatcatcaggcattagattctcatacgaagcacacaacctagatccctcagatgggcagttcacaacagggttcatgctcctatgagtatctaatgctatcactgatctgactggagacagagtagaggctgtaatatgaaccataaggagtggctgtaaatacagatgaagcttccctggcttgcctgctgcgcacctactcctgtgtggcgtggttcctcacagaccgtggactggtaccagtctgtggccggggagttgtggagccctgctctgggaggtcctaccatagatttaaaaagtaaaagtaaggaatttttgttcacaaaagaacagtgaagcacaggtcatgttacatatgcttgtgccaacaaggtctcactgttactgacttcattcctcctattttgaagttgaaagagatgcatttactttgttggaacaagatgtgttcttccaccggctggttaattgtcatgataacagtaattttgttagaagaaggtgctctgctaccatttgccaaaagattgtcataatgtacaattttcccaatgcaagggtcagcagattataataaaaatataaaaatgtttcacagtaacaaaaatgctagtatgctacctggatgtggacacctaatacatggtacaatccaaactgtatgaggacacctttaatttagctacctatttatcaaagagcttctgtaagttaggttttataagttgcaggagacaaagatggaatagatgtagttttaatctttaaggtgctcacaacagaagtgtttctatttcatttctgtggtttttcaacagaatttacaaagaaaacatttttatttatgttttcacttgtccacttaacaaataactgtcaaatgtcttttagatactaagcagtttttctaaggctacagaacacaaaaacagacaggagcttgttattattatcattgtcatttttattattttgctgctttattcagtgcttactgtgtgctagatgcccactggaatcttataattatgatttattatatgtgatattgattgtgtgccagacatatgtgatgaggagtgaaagctttagaaagaaaggaggcaggatttaatgtaagcatgcagagtgagaagaatttttcagggaaagaagcagaggaatgacatttggcagaaggaacatggagtgagatagtgtgtttgccagaaggaacatctaatgagattgcccgtttgggaggaagagcagcaagtgcagaagacaagatgcttgagtgaacatggcagggtttctgagcagttcacttttgctagtaccaaaagtgtgagataccagaggttgggagtgaggtgaatacttagctaaggcaagtttacgatagactttttaatactatagaaatgagtaggtcttttcctgtgggccatgggaaatttaccaggtagaatgctttggactgcaaatactagatgagcagtgcctaaaacactaggaaccagagttgtttttttgttcattgatatcctagggatcccacttgtccctctttcagctgtgctgttgacagtgttttattcacgtctccttttgtggttggctaatctgcagcagctccaaacatcttgttctcacaacacaacatcgaaagggctgcttttcttcacatgtgtcttttaaacagggagaaaacttagaagcatgcaaggggcttcctgtaacatttcattggctgggtcataccacatgctcattcccaaaccaggcactgggaaggtaaatatctgattagcttagaataaacatttctttctgaggctgaggagggggattgggatattaaatatcccaatagacttatgttttttctgccagaaagaatgaggaatggctattgatagggagccaacaatgtgtgctggagggggctcattggagaaatttgagcaggggagccacaagattaaatttgagtattaaggcattctggttatggtgtaaaatgggttagcaagctttttctttaaaggaccaggtgggaaatattttggactatgtggtctctgtcatatctacttaaccgtgctgttgtctgctgttgtagtgtgaaagccaccatgattatatgtaagcaaacaggcatgactgagctcctataaaactttatttacaaaaccataatgcagattggatttggcctgtggcctatagtttcctgggattgatgggagataatcatgcaaagaaaccaggagacaaaggaagcttttgcagtagtcagctatagtttccatgtcacacatccttggagtagcatcaatgtattacaaggttttcacccgtccatggtaaaataaataacgttagaaatctcagttactcattttactatgttggccttcgttatgcctttttcacttgttttgcttaattttttttcctgtaagaaataacattaatagttggcagttttcttttaaatagaagtcattttgtaaatgtttgtgttcccagtggcagtgggaatataaaacagaggcagaagagaggtatagtcaatatgatttagtgataattgaatgagaaaggcttgggggacagagagaaatctcagatgatgtacaggtttccagattgcacactagtatttaacctggacatgaggaaggagtaggaaattttctggtgaatacaggagagcaaagagcagcaggtcagcaggaatgactaatgtttttcgatgcatgtttaatggaatattcgtgtagggtattttgagtaggtaattggataatcagcatttataactcgcatcctactagtttgactctaagtaataagacttgtcaaagatccaagaatctgaaagtcagtaataaatgtccatgtgcatcatcgtccgtgacagaaagtcagcatccacagaacacagaattgggacagatgaacttaatagataaagatgaatatcggagttgttcctcttagggaatgatactctccatgacctgtgtgagtcacagctgccagaaaagaaagagcaaggagcgtatgaaggcagcacagcaaattcggtcctagagtgccctgcttggcttcatgtcatagttctgacttctaaataatcattttctgcaaaatgtgctttttgtttttccctcttgccgcctgcagccaatcagaatctttttagcaggacataatgaaaagaagtaagagaaagagtgtttttttgtaatgggatagtatttaacgtaaacttgagagtgagtaccaggattatacttagaatttatggactggatgggaagactggatagaaatctaaagattgctgactcaaacacaatgtggtttctttgatttattttcacagctctgaattcacgactgttaattgtattcatatgcattataactttacaaagcatcttccgaaaccaaatatttactgatttattataatttgtatgactttattatagaactgactttccaagtgttcatgagaattgtttagaatttgctacattgtatcatctcagctgtgtccacatgagctatctgtcaccttgtcttaatgaataattgttcactaggaatattggttttggcattgaaatgatctatatctaaatgcagataggacccgggaccacttttgaacattaatgttcaagcatcttaaaattacacataatgctttcataatctgacttctgccccactctccatctttagcccttttccctgtgtgccctgtctctggcattactgaactgctggcagtgccctactcactcatccttctattgtaggcaaatactttcactctttcaggagtcgctcccgctcttgctgctgcgtggcatgccgtcaccctttcctgccctctacaccttttaatctggctagtctcaatatttaagtctctgcttgggcatgtgttctagaaaagccatctctgacatgctttattttaattctttttaaaccctaatgcctagcatgtatttagcaggactcaataaaatatttctgagtaaaacaaagactgtttttacaaagatgatgtgcaagactctcccctgcagtcttggagcagaggggacagacgtggaggaataatgtacagtttaggtggtcaagatgcagtagaaaaatcagtaaagtactaaggcagcctcaaggaaggaggtacctgtttatttggggaaagacatgcagaatcaaggaagacttcatatagcattgtttcaaaagatgaaaataaggccaggtgtggtggctcacacctgtaatcccagcactttcagagggaatcacgaggtcaggagatcgagaccatcctgtccaatggtgaaaccccatctctactaaaaatacaaaaatcagctgggtgtggtggtgcttgcctgtaatcacagttactcaggagactgaagcaggagaatcacttgaaccagggtcgcggaggttgcagtgagctgatcacaccactgcattccagcctggtgacagaggaagactctagctcataaaaaaaaaaaaagaaaaaagaaaaaaagaaaataaatttgtcagaatagtggagagaaacattttagatattaggaagatgttgtacactaataaaggtgtcagcagtgattttggaaatcatttataaggtactattaggaagtggagaacagtacactgtgtcaccttatatgtttctactgtattttaaagctgtgtttctggtggttttgttcatggatgttgggtggatgaatttgtgagggaatttttgacatgtttgtttgtcttcaatctggtgacatctgctatctccccaagtgggtttttgaagtttttgagaattatttcttaaatgacaatttcacaaaagatgaaacactcaatttatgaaataaaatgaaatgtctcaaatctgtttttaaaaggcaatagtttttaactgttttaagtggttgattttaactgaatgtatggatttttcaacagaacaagacttaaagctgacatcagaggaagagtcacaaaggcttaaaggcagtgaaaatggccagccagaggcatggaaacttttaaatttaaacttttgtttaatgttgtgttttttttgccttaataatattagatagtccaaatgaaattacctttcagactaggctttgagaatcagtagattgtttttttaagaatcttttggccaggcaaggtggctcacgcctgtaatcccagcactttgagaggctgaggcaggtggatcacgaggtctggagatcgagaccatcctggctaacatggtgaaaccccgtctctagtaaaaatacaaaaacttagctgggcgtggtggtgggtgcctgtagtcccagctactcgggaggctgaggcaggagaatggcatgaacccaggagagggagcttgcagtgagccgacatccaccactacactccagcctgggtgacagagcgagactccatgtgaaaaaaaaaaaaaaaaaaattttaatagattcttaaaatttattgtaataaaatcagcaaccttattaacagaagaatcaatagattctaatttaatatttgatatttaacttcaacataacccactataaaatttaaaatactcttattttaaaatattcttatctgccttcttgattagcttatagctaatctctccttttggaatagaggcaaaaacaaatttcagaactttgtttgttcttttatttttacaacaccctaacatgataaagaaagtaacatcaatgattgaatcatattattaagcaataggaattatgaacaatgtaacactgattccctgagctggattcatggttaaagagtaatcatggcctgtgattgaaaatccacagttttatattgtcagtcactgataccaaggttaaggacatatcctgccttgtggtctctcgttgacctcagtgtttctgttcagggagggaaccaggtcataaaagcaacccaactgcctattacaagaaccatatcttgcagaatgggacctttggtgttagtgcacaaacacaataacattctaatttatttcagttgcagaaaatcagtagagattaaaaaattttatctgctgtcattagtacacattagaatatattagaactggacttacgcagataatctggatacataacactatcatatgacagtatataatttcaattaaaatgtgagaatttgcatttctttctgtttggtgttgatttcggctcctaatagtttaaagggtgcctacaatccagttaggaatcttttaaaaaagcacttcagtgcactgtaggtgctcactagttagggtttcatgaggtaaactcttttcaagtgaggaagattttggaacactacaaatcatctgctgattcatttttggtagatttaacacataacaaattaaatttagtccaaacaaatagtgagaaagttaagtttgctggttcatgtttttcttctccctttgtctaaggtgaattatttttcacatgttagaagccagtgatgtggcagtagctaaacatagattaaaaagttaattcttaattttaattattatttattttaacagtttaattttaattactttctaatttttattgtccatacttgattacttcagaataaaattattttaaaaacatgcactccaaaagaggaaatgtcacagaaatacaacaagcaaattaaccttctgtttttacatctgcagaaaagatctcaagaaccagaaataaataaggatggtgatagagaggtatacctttatattcaaacgtttgtgttgaattagatttttacattatgttgtttaacaaagtgtagtaaatgtaggcatacatgatcctatcatgtaagtagcataaatcatcagtgaaaaattaaatagttaactcagaattctgtacattgaattttgaagaggtgcaaaccctagagctattctttcattattatggaataatcccgaatggtgccataaaatgctaggtaatgccactttaggagctttagaccaattattttatctttcttggttttagtctgattatcaatagataatgtgcctaaagtagataatttcttattctctgtattttccagctagaaaattttatggctatcgaagaaatgaagaagcacagaagtactcatgtcggattcccagaaaacctgactaatggtgccactgctggcaatggtgatgatggattaattcctccaaggaagagcagaacacctgaaagccagcaatttcctgacactgagaatgaagagtatcacaggtaagcctatggcaacattgaacaggaggtaactttgtgctgccaaactaatcctaatttgagctaatattcatgatgaacaaattttatacttttattaggatattgagccttgcctgttaatcagaaaaatgaaaatcagcaaacaatcagttaccgttttttttccagtcattaatttatttgaaaaataaccagtattggcaaatgtgagggaaaagacattttcttctcttttcagtgaacttttattttagcttcggggtacgtgtgcaggtttattatataggtaaactgtatcatggaggcttggggtacagattatttcaccagccacatagtaaacaaaatactcgaaaggtagttttttggtcgtctccctcctgccatgctcctccctcaagtaggccccagtgtatgttattctcctctttgtgtccatgagttctcatgtttagttcccactaatgagtaagaatatgtggcatttgattttctgttcctgcatgagttttcttaggataatggcctccagctccatccgtgttgctgcaggggaaatggtttcattgaaaaagacatttcatatactgttggtaaatacattttgaacattaattgagtagcatattcacacacacatatataacagtaagcatatataatacatgtaaaggatatttgtatagatatgttatatgtatacttatgtataaggacatttattatagtattatgtaaaaaatttggagctagtctaattccttatcaataggaaatagctcaatttccatatccccaaaataatgtattatgcaaccatttttaaaaaatgaggttagatgtagggtatactgattatttcacaattaaaatgtatttaaagcgtttagtttgatgacacatcttaagagttcttgttaaaattcttgtaatatctgctgtgttgcaaatggaagctacatgctacattgacactgtaccttgttagcaacaagattgctagttactaaatttttgttgtcagtgcctgagtgctgaaatattggaccctcaatctgaatattgccaagcgattgtacatggggatctatatttaatataaacatttcagtatatttggtaaaacttttattaaaatacatcaaagaatctttgatctactaaaccaggagttggccagctttttctgcaaagagctagttagtaaatattttaggctttgtggactacatatattgattttcttgagacagggtctcactctgtttcccaggctggagtgcagttgtgtgatcatggctgactgcagcctcgactttctgggctctagtgatcctctgacctcagtctctactagctgagaccacgggtgtgcaacatcacacccagctaactgacactatggactgtaaagtgaataagcatggctgtgttccaagatacttgacttacaaaaacgggcagtgggctggatttggcccacaggtgcttatttgctgacccttgtgctaaaaggaaggtgctgctaatgcagtgacttttatttgtaaaagtgccctgcatgtgtgacattatcctccctttgagaaaaggatatatttcagtatttacctcaccatatttttccagtgacttcatatgattttgaaaactacatttataaaataagattattttctgcatttctcccactttattcctgttaatagaactcagtattttactgtgatcaattacttcgtatatttgatgagtgtcaactgtcctagaattggctgatttttatcaagcaagaaatattctccttgagagttttagtatttcttggtctttatgtataagcgtgaacaaaatgataatcagcttatgtaatctagaaatgttcaaagggcctttaaaaccttggtctgacatttctaaatgccatatgtgtataatttttataacctttagaatatataatggttacataaaatttgaaaactccacctggtatgtaaaatttggaagctactatttcttgtctatcacttttccatgactgtggatgaaaattacatcattctcagtcatgagcgttaagtatattgtccttaaagaactgtctacactcatgaactcaaattttctttccattcactcttgatctcaatgccggtaagtcttcaatttcagcactcctccagaactatttttcctcaagattatcactaatttttttctgtactaaatctaggcatttttcttacacctcatttaatctgtcagcaatatttgagccaatggagggcatctcctccctaatggcgtcttcacttggctttcaggacctcactccctcaggcttttcctcctgcctttctagtccgtttatcatggtctgttttgcttgctgctcctcatctttctccttttggacattgttgtttctcatggctcagtcctcaatcttctttctcataatttttttttttttaagacagagtctcactttgtcccccaggctggagttcagtggcatgatcttggctcattgcaacctctgcctcatgagttccagcacttctcctgcctcagcctcctgagtagctgggattacaggtgtgcaacaccacgcctggctaatttttgtatttttagtagagacaggtttccccatgttggccaggctggtctcaaactcctgacatcaggtgatctgcccgccttggcctcatgacatgttgggattacaggcgtgagccaccgtgcctggcccctcgtgactttttctactgtgtatatgctagtgatttccaaatgtatgtctccaacttagatctctttccttaattccagatctctatatcatcccacctacttgacatctctatttgattagctgttgggtatcacacacttgtcagatccaaaattgggctactgatgtccttcctgaaatctacacctcatgtagtctttcctactttggttaacagcaactcttccagttgctctgccaaaaacctcagtgtctgatctttctctctcagtcaagatcttcttgactgatctttctctctgtcctgacacttcacatctaatctctcagtaaatcttgtcaggtctatctgaagaatatatccagaggccagtctatcttgtacatctgagccaccgtcatctgcagtttagatgagtgtcatagacgggaattgatagtcctggtttttaaaaacttcccctttcatcaattcttatctcagtggatgtatttaaaacataagtcaaatgttgtcattcctctgcccctgcccttctgattgcctcctatttcactctgagtatgtggcagagttcctcctaataactgaaaggcagtaaaccatctggcatgttacctctcctgctgaaacttctgtttcttatctctattgctgtgtttcagccacactgaacttgttgctattcctgacctatccctagtgcttaaagactccaggcacacctctgcacttagcagttccctgtgtctggaatgctttttccccagatattcttctagcttactgtttccattacttcagctctttacttaaaatcccctttctaagaagaagaaaaagggtaaaaagaaacccattaaggaataaccacttcctgaggaagaaccatgtaccagcacgattcctaatccagagaaaatgaagaaaatgaaaaaaaagagagataatgaggactaacagaaaggaattaggattgtatcatcaggacgcgtcaggcttgagattcaattgggaacataccagggatgctctctaacgtaattgagggaaggttcaatgaaacaaagtgatttatcatctttaacttcaaacctgtttgtgtcttgacatcaactctgttaacatcatcattttttagagtctttgatgtacaaataaaagtttctttgtattaaagaaaaatcctctttctcagcagggatttttctggccatcccaactttcccaccacccttcccatcaaacacataaagatttcattttcctgctttagtttttctcctctaacgtactgtgtattttgccgtatctgtctgttgttattgtgtgtttatctcactctcatgaatagggtttttattgttcattaccatatcctcacttcctagaaagaggcctagcctatcagacgtagctacctaataaataggtattaaatgaatgaatggagtttatcccgggtatattgtttgattgattctcacttaaaaaatgtttgacaaggttcattttaacaattttgcctggtaattatatgtattttaaaaattctttcggctttttataataagctacattctttatattaatattttttcacttagggagaaaagcccaatattgtggttattcactattcttttactggtaatcatgataattgcaattatggtaaaatgagttagagaaattacaaactttactggtattttatttatttagagacagagtctcgctctgtcacccaggctgcagtgcagtggtgtgacctcggctcactgcaacctctgcctcctgggttcaagcgattctcctacctcagccccctgaggagctgggattacaggtgcgtgccatgacacccggctaatttttgtatttttagtagagacggggtttcatcctgtttgtcaggctggtcttgaactcctgacctcaggcagtccacccacctgggtggattacaggtgtaagccaccacatccggccactagtattttagtttttttagggtggtaaatgtaatggactcacaaattctttccaagggattatggaccttcggtatttgaaataaaaagacagttggaattttttgcttccgatagtaagactatactggtcaggcactgtctattctgatggagcagctgttgctgcttggctgtctttcagaagcaagctgctcacactgatattggttggtgagcacagccagtggtcggtcattgatcgattgactagattttgaactggctctggctggcttcttgttaccatggctacaggtcaattctttcctaagtttgagtcaactttaaccagaaattttctgttcaaaagttgccttccattaactatgttcaaaaagaaactttttaatattccagaattgtggatttaaagttttggttatgatgacttggttaataatagctctcacgaagatttttttttttgatacatcatcttaaccagaagtgttctctgtataatttatttcttaaaaataagtgttttgtttttgtttttggtatttttagaagcttttgctcaagtcctaacataatctccagtaggagattttagtctctttgtcagttcatgtatgtatgtgatagtcatattctgtgtttttaaattccttttcttgttcacttttttctctgtacaataatagtgatattgttatacatttttatctcattaaaaagttaacaattttctgctggcaaatctagctttttctatattttgactgaataggttaaaagtgaagaaaatttacgagatcattttattttcaaacaaaatcataagtaataaaaattgctattttgaattataaataatgacatttagatattttaaaaataaggataacccccccccccccccgcaatagtttggctttgtgtttctatgcaaatctcatgtcaaattgtaattcccaggtgttgaggaaagaccagctgggaggtgattggctcatggggtcggtttcctccatgctgttcttgtgatagtgagtgagtcctcacaagagctgatggtttcataaggggctctttgcgcttcacttctctcttctctctctcctgccgccttatgaagaaggtgcctgcttccccttccccttctaccatggttgttaagtttcctgaggcctccccagctatgcgtaactgtgaatcaattaatcctctttcctttatgaattacctagtctcaggtatgtatgtgtgtgaattacccagtctcagctatgtacatatgtatatgtgtgagtgtatatacacacatacatatatatgatacatgtatgtgatgtgagatatatatatatgtatatacgtccattttctttattccacgcatcagttgatggacactggttgattccatatctttgcatattgtgaattgtgctgcagtaaacatatgtatgcgggtgtccttttgagagtatgatttcttttattttatgtagatatctggaaatgagaatgctggataaaatggtaggatctacttttagttctttgagaaatctccatactgttttccatagatttgtatgaagttgcattcccaccagcagtgtatcactgttgtcttttcaccgcatccacaccaacatctgttgttttttgatttctaatagtggccattctggctgcagtgaggtgatatctcactgtggttttattgtacatttccctgatgattagagatatttagcgtgtttttatatgcttgtttaccgtttgtacatcttcttttgagaaatgtctattcatgtaatttggccactttccaatggaattatttgcgtttttcctgttgatttgtttgagtttcttgtaggttatagatattagtcctttgttagcatcataattttcaaaattttcccattgtataagttgttgttttactctgatgattatttcttttgctgtgctgaagctttttagtttaattaggtcttatttatttattttcatttttgttggatttgcttttagggtcttcctcataaatcctttgcctaggccaatgttttcaggtcttaggtttaggcctttcatccatcttgaattaatttttgtatatggtgagagatagagatccagtttcattcttccacatgtggctatctttttttcccagcaccatttattgaataacctgtactttctccagtgtatgtttttgtatccttgctcagagatcacttggttgtagtggctttatttctgagttgtctgttctgttccattgatctatgtatctgtttttatacgagtaccacgctgcttctgttactgtggtcttagagtatcatttgaagtcaggtaatgtgatgccaacatatttgttccttttgcttggtatgtctgttgctgttcaggctcttttgtggttctacatgaatgtcagcattttaaaataattctgtgaagaatgacattggtactttggtaggtattgtatagaatgtgtagactgctttgggcactatggtcattttcactatatcagttctttcagtccatgaacataggatgtattttcatttgtttgtgtcatctgttattttctttggtggtgttttccagttatctttatatagatcactcacttttttcattaagtatattcctaggtacttcacacctttttgcagccactgtaaaagggattggattgttgatatgaactctcagcttggttgtagttgatgtatagtggtgctactgattggtattcatttattttgtaacctctgagactttactgaattcatgtatcaaatctaggagtgttttgtaggagtctttagggttttctaggtatgggataatatcattggtgaagagatagtttgacttcctcttttccaatttggatgccctttatttctcttgcccaattgctctgcctaggacttcccagttttattcttaatatgcatgaaataaaagtaaaatggaaagtgcttaacgatgagtttatttcacatctctctctcatacacagataaaattatttcaaagtcctatgttaaaaacataatattagaccctgtcttgttctaaaaggaatttctaagttgtttataaaatacataggaatcaagaatataagtagaaagtgtttccaaacaataaacataaagagtatgggttacaggacacagaccattagcagtccctggcctgttaggacctgggccacacagcaggaggttcgaggcaggtgagcaagtgaagctccatctgtttacagccactctctgtcgctcgcattaccgcccaagctcctcctcctgtcagatcagcggtggcattagattgtcctgagtgtgacctgaaccctgttgtaagttgctcatgcagggggatataggttgttcactccttatgagaatcgaatgcctttctgatctgtcaccatcttccgtcacccccaaatgggaccatctagttgcaggaaaacaagctcagggctcccactgattctacattttggtgagatataattatttcattatatattaataataatagaaataaagtgcacaatgaatgtaatatacttgaatcatcctggaaccatcccccacctcaggttcctggaaaaattatcttccacaaaagcagtccctggtgccagcatggttggggacacctggttaacagatgtgagacccctttgccttgtcttggaataatgtgcagatatgcattgtgtgaatgacatctgatggcgccatcttgccctgtacatcattttagggacagctccagtatttcatgaaaattaaaatttcttctagtgacgaacaaaatgatactcagaagcaattttgtgaagaacagaacactggaatattacacgatgagattctgattcatgaagaaaagcagatagaagtggttgaaaaaatgaattctgaggtattttctttagttattttcaaatgtttttatatgtgtgtatattttaaagaactgtattttggaaatataaaggatttttaagtcatatatatgtgtgtatatattctatatatcctttgtcatatatctatatacgtacgtataggataaagccatgttcttaattcaccttcatttgcctgcaacagttgagtagtgacctgcacaatggcctcaatccaaaggagaagtatttgatatttttcataagaattgatgatctttccacatcagaaataagttttgctactgaaaacagattttctcgtttttggacattagttttttaaaaaatgttaatagagaagtcaattgattatttttactgataagaaagtaggaaatgtatagctgggtcagaggccacattgtggataccattatccttacttttgcagagaggaacagtttgctccaagtagtttctcatttcaatgtaaagaggtttgaaaagaatgacatgccatgatacacatttagtaataatttattgataagtattttctttctggagaaatagttcagtatatttcccctatttcaccattactactgtttcaaacattataaagaggaaataaaagttaccgcaatggcaaataatctcatgatttctaagaaaatctctataagttgtatcttatttaccattcatattttgaaacaaaaggtttcttttgtatttatatatttacaccacagaagtaagtgtggttttgtggaggatcactagaagtagcatcagcagacctggggaaaatcctgcatctgtgtatattttttgacctctccttttaagaatcgcgatcttaaatgagttcagtattatatgtagaagagcaatgcctagatacagatgtgtacagtgtagaagggtacagtgcttatttgacagttataaataaatgtaattcttataactgagtatagaaatattagaaatgtagaatatcggtaaaacgttcttaagtaaaagaaacttaaagaactttgagaaattgcttctgtccaaatatatgcatagctaaggctcttaggatggtgtggttgataggttagatatcagagtgtaaacctaatcttaaaaatgtagtcaaattattaatcttatattttatgcctctgggttttttgtaactcagaaaaaaggctttttttaattctgggattcttaaaaatcctctagtgatttatttttcatcgtctttaaataaatatttaaacttttaggaatttaggagcagattcctaaaagtttaaatatttatttaaagacgatgaaaaataaatcactggaggaatttacactcttattaggtttgaagttttgtccaatttttttccagttaaatatccactatggggattctttcattatacaaatacatgttatttttgaatttcagaagaaatcatgatatgtcaatctattgagtgctaactaaaagttctttttgtttacttagctttctcttagttgtaagaaagaaaaagacatcttgcatgaaaatagtacgttgcgggaagaaattgccatgctaagactggagctagacacaatgaaacatcagagccagctaagagaaaagaaatatttggaggatattgaaagtgtgaaaaaaaggaatgataatcttttaaaggctctacaattgaatgagctcaccatggatgatgataccgctgtgctcgtcattgacaacggctctggcatgtgcaaggccggctttgcgggcgacgatgccccccgggctgtcttcccttccatcgtggggcgccccaggcagcagggcatgatggggggcatgcatcagaaagagtcctatgtgggcaaggaggcccagagcaaaagaggcatcctgaccctgaagtaccccatggaacacggcatcatcaccaactgggatgacatggagaagatctggcaccacaccttctacaacgagctgcgtgtggctcccgaggagcaccccgtcctgctgaccgaggccaccctgaaccctaaggccaaccgcgagaagatgacccagatcatgtttgagaccttcaacaccccagccatgtacgtggccatccaggctgtgctgtccctgtacacctctggccgtactactggcatcgtgatggactctggtgacggggtcacccacactgtgcccatctatgaggggaatgccctcccccatgccaccctgcgcctagacctggctgggcgggaactgcctgactacctcatgaagatcctcaccgagcatggctataggttcaccaccatggccgagcgggaaatcgtgcgtgacatcaaagagaagctgtgctatgttgccctggacttcgagcaggagatggccacggcggcctccagctcctccctagagaagagctacgagctgcccgatggccaggtcatcaccatcggcaacgagcggttccgctgccccgaggcgctcttccagccttgcttcctgggcatggaatcctgtggcatccatgaaactaccttcaactccatcatgaagtctgatgtggacatccgcaaagacctgtacaccaacacagtgctgtctggcggcaccaccatgtaccctggcatggcccacagaatgcagaaggagatcgctgccctggcgcctagcatgatgaagatcaggatcattgctcctcccaagcgcaagtactccgtgtgggtcggtggctccatcctggcctcgctgtccaccttccagcagatgtggatcagcaagcaggagtatgatgagtcaggcccctccattgtccaccgcaaatgcttgtaggtggactctgacttagttgcgttacaccctttcttgacaaaaccaaacttctcagaaaacaacatgagattggcatggctttatttgttttcttgtttcattttttgttttgttttttattggcttgactcaggatttaaaaaccggaatggtgaaggtgacagcagtcggttggaggaagcttcctccaaagttctacaatgtggccaaggactttgattgtaccttgttcttcttttcaatagtcattccaaatattgtgagacgcattgtttcaggaagccccttgccctgctaaaagccaccccacttctctctaaggagaatggcccagtcctctccctagttcacacaggggaggtgatagcattgcttttgtgcaaattacataatgcaaaattttttgaatcttcgccttaatactttttaattttgttttattttgaatgatcagccttcgtggcccccctcttttgtaccccaacttggggtgtatgaaggcttttggtctccctgagagtggctggaggcagccagggcttacctgtactctgacttgaggagagttggataaaagtgcacaccttaaaacaaattgaggaagcacagtatttcagtacagtggacagcttagcatgttgacaactgagaataaaatgctcagttctgaactggacaatgtaagacacaacgaggaaacactggaaatggaaattcaattacgtcattgtagactggctgctgctctacatgattgtgaccaaagtcagatagctgaaagagacttctttccagagaacaagacatgaacaggtttatttacagaagacaatgaattctcatttatctcacctaaaagagaacagattctttctcaacaagtctaatgaagacagtaaaatcaacaggctaaaaattaagctccacgaaacaagataaaactctgagagaaaagacggggcaggccgccatctttcccgttcaggcaacgtagtcattccagcctgcgggctttggagagtacaaaccgacgagggacagaagagatcccacagcacagcatagctgctttaccaaatcatggccagactgcttctgtaagcaggcccctgatcctgttccacctcactggacaggacctcccaactggggcctccagctacccccaccagcattccttggccaatggaaatgtgaaatgttcctgggacagagctcccggagaggggcaggcccccacctttgctctttcggtgactagccattctggcctgcgggctttggagagcccaagctgacaaggggtggaagaggtacctcagcacagcacagccacgctacaaaaacgtggccagactcttgtttacgtcagtccctgatcacatttctagtcagcgggtgaagtctttcaaccagggtctctggctaccttgactgctgttctctggccgacagaggtctcaggcctccctgagtcagagctcccagggggaggaccagattgtcatctttgctgtttgggcgacccagccatttcagccttagggcttcagagtgtctgaggcggaccaggggctgaagtgaacccccagcacagcacagctgctctataaaaacgtggccagacttttttttttgaagcaagtccctgttctcgttcgtcctgactaggtaagacttctcaacttgcctccagccacatcttataggtgtgttcagattggcaacaggttcgtacctcagtggtacagagcttccagaggaaggggcagactatcatcttccgtggaaaatacaaggcaattagggactggaggggacccccagcataccacagcagccctacagaaaagtggccgggctctctacttgatgggcagatcctcctggcctgggtctctagccagcccaccactggagctatcaagccagtagcaactctgcagttcctgggacagagcttccaggagcaaatgaaatcctttctgccactgcctctgcagtggaactgcccttgctaccctcagaagatgcaagggtgcaaagaccctaagtgccctatcaacacctccaataagctgcagttgacccaaagaacaagccagtccatctcccacgggtaccacacaccctccactactcatcaccagacagggaaccctggcttgggcccacagcacagaccctccatcctgggctgattacactaagtgattgctaactcacatgtctctgggatggagcacccaggaaacaagcaaagtggtggagcagcaagtcaggtgatgtggagcccagaggtcagggatggctgtctctctagggtccacttgcccttgtgagacactttatcccagcactttaggaatgctgaggtcataccagccacatcttatatgcaagattgcccagcagagatcaggtccgagagttccctttttaaaaaaaggagacttgcttaataaaagaagtctagccacgtttgtgtagagcagctgtgctgtgctgggggttcacttttgagagagttctcctctgagacctgatctctggaggctgggcagtcttgcacttgagatggggctggtctgatctcagcactccttagtctgctcgcctctcccatggccccagcctggccacacctgcttacggggcactcgtagatgcccacaccatagcttccatgctagtggactgtaccatatcagtggagagctgcagcaaggtggcctctagagccacgcaccagcctgcacattgcctctccatacggcagccctttatttggaaacttcctaaatcactttgctgtgtgtgtttacacgggtgggttttgctttacttgccctgagagcacacgggagtgcagcacacaccccaacccacatcaactgccattaaagaaaagaaatttcagcccagaatttcatgtccagcaaaattaagcatcataagtgaaggagaaataagatccttttcagacaagcaaatgctgagggaatttggtatcaccagatctaccttacgagagctcctgaaggaagcactaaatatggaaagaaaagatcatcacctgctactacaaaaacacactgaagtacacagtccaatgatgctaaaaagcaagcacatatgtaagtctgcaaaataaccagctgacagcatgacgacaggataaattccacacataccattactaaccttaaatgtaaatgggctaaatgctcccattgaaagacacggggcaagctgggtaaagaaccaagacccactggagtatgccgtcttcaagcaacccatctcatgtgcagtgccatacataggctcaaaataaaggaatggagaaaaatatttcaagcaaatggaaaacagaaaaaaggtgttgcactcccagtttctgacaaaacagactctaccaataaagataaaaaaagagaaggacattacaaaggtggtcctgacctttgataaatctcattattgcttgataccaacctgggctatttgtattgcccaaaccaataggataatttgctgaggttgtggagcttctccctttcacagagtccctgatctccgaaaatttggttgagatgtaaggttgattttgctgtacaactccttttttgaagttttactcatttccaacaaggaaggcaagttttcctgcttccattgacaaaggagagcaggcacctcctttcctgagtttcagcttgcttctgacagggaaggtgagtgtaagtttttccagcttctaagatggcagagaatgatcacccagtctgagccttatttccaggtaagtagctgaattagagttttgtcttaaaatttttgtttaatgactaaaatttaagattacccaccagctgcttttaattcctccttaccattagaacactcagttaatcatatgaattgtgcatttgtttgttttgcttaactctttttgtttatgtttggggttttattgttgtttcacttttctcccatctcttcctgacttggtcaaatccaaaggaatgttccaaattgtggggagcaaggcatctgaattggctaaaactcctgtggctgcaaaacaaaaacaaaacaaaaaaaaaacacaaaaaacaaaaaacaaaaaaaaatccagttggaaattttttaaaacttttttttaatttttaaattttattattatactttaagttttagggtacatgtgcacaatgtgcaggtttgttacatatgtatacatgtgccatgttggtgtgctgcacccattaactcgtcatttaacattaggtatatctcctaatgctatccctccctcctcccccaccccacaacagttctcggtgtgttatgttacccttctgtgtccatgtgttctcattgttcagttcccacctacgagtgagaacatgaggtgtttggttttctgttcttgtgttagtttgctgagagtgatgatttccagcttcatcgatgtccctgcaaaggacatgaactcatccttttttatgggtgcatagcattccatagtgtatatatgccacattatccagtctatcattgatgggcatttggattggttccaagtgtttgctgttgtgaactgtgccgcagtaaacatacgtgtgcatgtgtctttatattagaatgatttattattttttcagtatatacccagtaatgggattgctgggttaaatgtatttctagttgtagatccttgaggaattgtcacactgtcttccacaatggatgaactaatttatactaccaccaagagtgtaaaagcgttcctatttctccacgtcctctccaacatctgttgtttcctcatcttttaatgttggccattctaagtggtgtgagattgtatctcattgtggttttgatttccatttctctaatgaccagtgatgatgtggtttgcttcacatgttctttagctgcataaatgtcttatttgggaagtgtctgttcatatgttttgcccattttttgatggggttgttttttttcttgtaaatttgtttaagttctttgtagattctgcatattagctgtttgtcagatggatagattgcaaaaattttctcccattctgtgggttgcctgttcattctgatgatagtttcttttgctctgcaaacactctttagcttaattaggtcccatttgtcaattttggcttttgttgccattgcttttggtgttgtagtgatgaagtctctgcccatgcctatgtcctgaatggtattgcctaacacaaggacatttctgtgcctgagtgccataccacccaaagtgatttatagattcagtgctatccccatcaagctaccattgactttcttcacaaaattagaaaaactactttaaatttcatatggaaccaaaaaagagtctacatagccaagacaatcctaagcaaaaagaacaaagctggaggcatcacagtacctgacttcaaactattctacgaggccacagtaacggaaacagcatggtactggtaccaaaccaggtatatagaccaatggaatggaacagaggccacagaaatgacaccacacatgtaaaaccaccagatctttgacaaaactgacaaaggtaagcactggggaaaggattgcctatttaataaatggtgttaggaaaactggctagccatatgcagcaaactgaaactgggccacttccttacactttatacaaaaattaagaagcataaaagagttaaacgtaagacctaaaagcaaaaaacctagaagaaaatgtaggccaccaacctcaggggaatgtacttgtagtgaaatgcatggtacaaacacgcattccctacttccttgagtgggtgaggttggtggctggtccatctgctccaagtggacccttacagatgtggctggttgctctttgagccagcttggccttgcccggcatgcacaagcatcagtgaataactgtgctataaatggagccacatagaagaaatgagcagcaggctcaagaccatggtgtgcactgcctttggcgctccagtccgtgcctcagggatggtatggcactgcgagcttcttgtttgccaagaggcagaccacaggccatcttgaggaggactttatgttcaagggcagaaaacagccaggattaccacccaggggacttggccttctgtggccgtggccagacttagaatttgtgtcaaggcagggcaagctcactcggagcagcgtgttggtacctggggcctgtgcatgccaggcaaggccaagctggctcaaagagcaaccagccacctctgcaagggtgcgcctggaccaggtggactagccaccaacctcacccacagaaggaagcagggatgaccaggttacgagagcctgagtagctgccacctgagggctgatggagcagaggcctgaggaaaatcagatggcacgtttaactgtttaatggatcttaagttaatttttctataaagcagatgtcaccagtccatgcctcagagctcatatggcactgcagacacagaagtccgagtcacatgggtggcaatcatcgctactttcagcacttgaagcatcaagtcctcctcaagacggcctgtggtctgcctcttggccctacctttagggtagaagaaccgatgtaccatgtttggcagcaagtgaggttggtggctggtccggttgctcctggcacacccttgcagaggtggctggttgctctttgagccagcttggccttgctcgggatgcacaagcctcagtgcaactactgtgctacaaacggagccaaagaggaaatgagcagcaggctcagcagcagggtgtgcgctgtctttggggctccagtccatgcctcagggctcatatggcattgcaggcttcttggttgccaagaggcagaccacaggccgtcttgaggaggactttatgttcaagtgcagaaagcagccaggattaccacccaggggacttggccttctgtggccctgtccagactttgaatttgtgtcaaggcaggagaagctcactcggagcaacgtgttagtacctggggcctgtgcatgccaggcaaggccaagctggctcaaagagcaaccagccacctctgcaagggtgtgccaggaccaggtggaccagccagcaacctcagctactcaaggaagctgggatggccagtttcctacagcctgagtggctgcctcctgataactgatggagcagaggccttaggaaaagcagatggccctgtggccctacctttagggtagaagtactgatgtgccatgtgcagcagcaagtgaggttggtggctggtgcagcggctcctggcacaccctcgcagaggtgactggttgctctttgagacagcttggccttgcccggcatgcacaagcctcagtgcaacaactgtgctacaaatggagccacagagaggaaatgagcagcaggctcaggagcagggtgtgcgctgcctttggggctccagtccatgcctcaggggtcatatgacactgtgggcttcttggttaccaggaggcacacaacaggccgtctcgaggaggacttcatgttcaagtgcagaaagcagccaggattaccatccaggggactcggccttctgtggccctggccagactcagaatttgttccaaggcaggactagctctctcagagcagcgtgtcagtagctgggacctatgcatgccaggcaaggccaagctggctcaaagagcaaccagccacctctgcaaaggtgtgccaggagcaggtggaccagccaccaacttcagccactgaaggaagcacggatggccaggttccaacagcctgattggtgcttcctgatggctgatggagcagaggccttaggaaaagcagatggccctgtggccctacctttagggtagaagtactgatgtgccatgtgcagcagcaagtgaggttggtggctggtgcagcggctcctggcacaccctcgcagaggtgactggttgctctttgagacagcttggccttgcccggcatgaacaagcctcagtgcaacctctctgctacatatggagccacagagaggaaacgagcagcaggctcaggagcaggctgtgcgctgcctttggggctccagtccaagcctcgagtggtatagcactgcgggcttcttggttgcctagaggcagaaaacaggccgtcttgcggaggactttatgttcaagtgcagaaagcagccaggattaccatccaggggactcggccttctgtcgccccggccagaccttgcagaggtggctggttgctctttgagccagcttcacctcccttgcatgcacaggccccaggtactaacacgctgctctgagtgagcctgtcctgccttggctgccacctaattgctgatggagcagaggccttaggaaaagcagatggccctgtggcccaccttcagggtagaagaactgatgtaccatgtccggccactagttggtgactggtgcacctgctcctggcacacccttgcagaggtggctggttgctctttgagccagcttggccttgcccggcatgcacaagcctcagtgcaacaactgtgctacaaatggagccacagagaagaaaggagcagcaggctcaggagcagggtgtgcgctgcctttggggctccagtccatgccttgggtcatatagcactgcgggcttcttggttgcctagaggtggaccacaggccgtctggaggaggactttatgttcaagtgcagaaagcagccaggattaccatccaggggactcggccttctgtagccctggccagaccttgcagaggtggctggttgctctttgagccagcttggcctccctggcatgcacaggccccaggtactaacacggtgttctgagtgagcttgtcctgccttggctgccacctaattgctgatggagcagaggcattaggaaaagcagatggcactgcggcccacctttagggtagaagaactgatgtaccttgtccggccgctagtgggtgagtggtacaactgctcctggcacacccttgcagaggtggctggttgctctttgagccagcttggccttccccggcatgcacaagcctcagtgcaacaactgtgctacaaatggagccacagagaggaaacgagcagtaggctcaggagccgggtgtgtgctgcctttggggctccagtccatgcctcgggtcgtatggcactgtgggcttcttggttgccaagaggcagaccacaggctgtcttgaggaagactttatgttcaagtgcagaaagcagccatgattgccacgcaggggactcggccttttgtggccctggcgagacttagaatttgtgtcaaggcagaagaagctcactcagagcagcgtgttagtacctggggcctgtgcatgtccgggaaggccaagctggctcaaagagcaaccagccacctctacaagggtgcgcctggaccagttggaccagccaccaacctcacccactgaaggaagcagggatggccaggttcccacagcctgagtggctgccacctgagggctgatggagcagaggcctgagtaaaatcagatggcatgtttaactctttaatagatcttaggttaatttttctataaagcagatgtcactagtccacgtctcagagcttgtatggcagtgcagaccacagaaggctgagtccccttggtggcaatcctggctgctttctgcacttgaacataaagtcctcctcaagacggcctgtggtctgcctcttggccctgcctttagggtagaagaaccaatgtaccatgtcccgcagcgagtgaggttggtggctggtctggctgctcctggcacacacttgcagaggtggctggttgctgtttgagccagcttggccttgcctggcatgcacaagtctcagtgcaacaactgtgctacaaatggagccacagagaggaaatgagcggcagagttaggagcaggatgtgcgctgcctttggggctccagtccatgcctcgggtcgtatggcactgcaggcttcttggttgccaagaggcagaccacaggccctcttgaggaggacttcatgttcaagtgcagaaagcagccaggattaccatccaagggactcggccttctgtggccctggccagactcagaatttgtgccaaggcaggacaagctcactcggagcagcgtgtcagtagctggggcctatgtatgccaggcaaggccaagctggctcaaagagcaaccagtcacctctgcaagggtgcacctgtggtctggaggttggtggctccctgtgttagtcctccaagcccatattttccttctgcagtgccctcgcagaggtttctgaagaggctctgcctctgcagcatgcttctgcctggaaacagtgggaggtagttttgcagggtggaagccttcaccagtggttaagcaccatcttcatgatgctgacctcgtgatagtgagttctcatgagatctgcttgtataacatgatgtggcacctctttcctctctctgtcttgctccttctcctgctgtatgaaacatctccttgccccttggtcttctagtatgattgggaggcacctgagtcctcccagaagcagaagccactgtgcttcctttgcagcctgcagaaccgtgagccaatgaaacctcttttctttttgatcatacagaaggttagtactgtgaagtgaagctatgaaattccttcagggccttttccctatgaaatgccttcaaggccttttccccattgtcttggcaagcagcactcagcttcttttcatgcaaatatctgaagcctgtgtgaattttttccctgaaaatggacttttcttcttttaccacattgccaggctgtgacacagagagctgataatgtagaagcaggttcagtagtgggtaaaagacagaggtcgggagagttgggaaagcttaaaagacagcaagatgaggaaaagcttggacccctgtagagaattgttaaatacttgtgatcagaaggctgacagaaggatggacagtgaaggccagacttaaaagttctcggataaaaatcaggaatttcctgtgaataggagccaaagctacatttgatttgcccaacagaggctgcacagtgaccttgccctggagatctgtgaatctatgaacttgggggtgatgatttaggatgtatcttgtggaatgaacatctaagcagcatagctcaagaggtgtcctgtctgcgtcgaacagcctgtgttgttatgtatgagctaagaaatgacctcaagttggatcttctacttaaatgagaagcggagctcagaagtttggaaaatttgcagcctagtcaagtggttaaaaagaaaagctgattttcagggggaaaattcaataaggcttagagtatttgcataaaaaggaggttagtgcaagtgtccaagacaaagggtaaaaggccttgaaggcatttcagagacctttgcagtagcctttgctctcacaggccctggggcctagcagagaagaatggtttactgggccagctccatggccctgctgctgtgtgcatcctcaggacactgctgcctgcatccctgcagccccagctccagccatggctgaatgatgcacaggtacagcttgggtcactacttcacatgtggctccaagccttgatggcttccacatagtgttaaggcagcaggtgaagagagcaagagactagaggctttggagcctcttgtctggactccagaggatgtagagaaaagcctgggtgtccaggcagtagcctttccaagaggctcatggaaaacctctgctagggcagcaaagaagggccatgtagggttgaagccccctcacagggaggctccattctccaaaccccagattcatagacccaccaacatcttgcaccctcagtgtggaaaagctacaggcattcaaaacagccctgtccatgagaggcagctgtgggtgctgaacgctgcaaagccacaggtgcagatctgcccaaggccttgggagcccagccctcacagccctgtgccatggatgtgggacaaggattcaaaaaggatgattttggagctgtcggattgaatgactggcctgctgggttttggatgttcatgtatcctgtgagtcccatctgtgttttgtttttctttctggcattttttttttctactggctgggaatgcttactcattgcctgtacaatcattgtgccttggaattagataacttgctttataattcagaaacccaggggcagatcggactgtagccttgtctcagacgagactttgggctttggacatttgagtaaatgctggaattatttaagatttgggggcctgtagggcaggtatcattgtattctgcaatgtaagaagcaggagatttcggggaccagggacagaataatatgatttggctctgtgtccctaccaaagctcgtggaattgtaatgggcaaagttaaaggtggggactggtggaaggtgatttaatcatggtggagagtggagtttggaaggtgggggtggttgggagcattgtgggggattgtgttggggttgtggggaaaggcagagatggggggcagattcttcacaaatggttaaacaccatctttgtaatgctgtccttctgacagtgagtttgcttcatggtttaggagctttgagatttgaatactggcctgctgggttttggatgtgcattgggcctgtggtcccatttgtgttatttttctgggaaatttcttccctttggagtgagaaagcttacccaatgcctgtaccatcatcgtaccttaaaagaactccattttaagttcagggactcattggcagaagagaccgtagccttgtatcagataagactttgaacttcttacatttgagttaatgctggaatgagttaaggcttttgtaaacatttgaaaaggcatgactgtattttactctgcgagaatgacatgagattcggcggagggggtcaaggtcagaataatatggtttggctgtgtttccctagaaaaactcatgtggaattgcagtcccgaatgttggaggtggagcctggtgggaggtgatttaatcatggatggggggtgggtggggttggagggaaaaagaggtgggtagcgtggtgaggagtaagctcgctgtagggtggtgggagggtgggggtagtaggaaggaggagtagcctgctgcagaggcagaggctcgtggaaaacctctgctaggactgtgcacctgtggctttgcagggtgtagcccccgtggctgctctcatgggctgggctggtgttgagtgccggtagcttttccatactgaaggtgcgagctgttgatgggtctatgaatctgggctctggaggatggtggcctcctgcataggggctccaagcccatatttttttctgcactgccatagtacaggttttccaagaggctcaggctctgcctcaggcttctgtccggaaacagtagggggtggaggtgggttggggacggatccttcactaatgattaagcaccatcttcttgatgctgacctcgtgatagtgagttctcatgaacttcctcatagtgttaagccactggttgtatggagcatgagcctaggggcttgggaggctctttatagattttggaggatgtatggaaatgtctgggtgtccaggcgaaagccttcctaaaaggcagaacctcataagaaacctctactagggcagtacagaaggaaaatatggggttggagcccccacactggaggccaccatcatgaagaccgcagattaatagaccccgcaacagcttgtaccgtcagtgggtaaaagctacaggtgctcaacaccagcccagcccatgaagacagccgtggggcataaaccctgcaaagccacaggtgcagagctgcccaaggccttgggagcccagcccttacatccctgtgccctggatgtgggacaaggtttcaaaaagggtaattttggagctgtaggattgaatggccttctgggtttggagtttcatggggtctgtaagtcctttctgtcttttgttctttctggcaaaattattccttttggctgggaatgcttacccattgcctgtataagcattgtaccttggaagtagttaactttctttatatttcagaggctcatgagcctaagggtctgcagccttgtgttagatgagactttaagctttgaacatttgtataaatgatgaaatgatataagactttgggggactgtagggaagctatcattgtagtttgcaatgtgagaaggacatgagatttggggagccagggacagaataataaaatttagctctgtgtccctaccaaaattcgtgtggaattgtaatggggaatgttaaaggtggggcctggtggaaagtgatttaatcatggtggagtgtgggggttgggaggtgggggatagggagaattgggagattatgttgggggtgaggggtgaaaagtgatggtggctccttcacaaatgattagacacaatctctttatttctgtccttgtgatggtgagttctcttcatgattttggagcagtgagattgaatggatactggtctcctgggttttggacttgcattggtcctgtggtcccatctgtgttattttgctcggaaatttcttcctgttggactgagaaagcttacccaatgggtgtaccatcattgtgtcttaaaagaacacccttttaaattcagggacttataggcaaaagggactgtaggcttgtctcagatgagacgttgaaccttttacatttgagttaatgctggaatgagttaagacttttggcaacttttgaaaaggtgtgattgtattttgctctgtgagaaggacatgagattcggaagggtcagggtaagaataacatggtttggctgtgtttccctacagaaactcatgtgaattgtaatcttgaatgttggagatggggcctggtggaaggtgatttaaccatggatgggagggggttggagttggaaggaaataaagtggatagtgttgggaggagtgggttgtcagtagggtggtgagaggatggtgggtattaggaagggggagtagcctgctgcagagtcacatcctcatggaaaacctctgctagggcagtgctcctgtggctttgcaggctttagcccccatggctgctctcatgggctgggctggtgttgagtgcttttgcatactgagggtgcgggctgttggtgggcttatgaatctggggtctggaggatggtggcctcctgtgtgggggctccaagcccatatttcccttctgcactgccatagtagaagtttcccaagaggctctgcctctgcaggaggcttctgcctggaaacagtaggcggtggtgtgtgtggtggatccttcaccagtggttaatcttcttgatgctgatctgatagtgagttctcatgtgatctggttgtataatgggctgtggcacctcttttctctctgtgtcttgcttctatttctgccatatgaaacatctcattgccacttggccttctgatgtggttaggaggggcctgcctgatcagtgtgggcctgctcagtggacctagtcagttgggacttggtcagtgaggcctgtttagtgggaacctggccagcaggggtctgcttagggagggtctcattagggggatccagtagtgggggttttggcaagtggggacctactggcagccagttgcttggtgtctggtcagtgcagacctaggctgtggggcttgaccggtggcgacctggtcagctgggcttagtggtggcctggtcagcatgggctgggtcactggtgaccaggtcaaggggtgctattcagtggaggactggtcacatgggacctagtcagcagggcctggtgggcgtgtcctcatcagtgagtcccttgtcagtggggccctgatcagggcagcctggtcagtggaacctcatcagtgggggcctggtcagtgatgacttggtcagtggtggcttttgtaccactggtctacggggtgacccggtcagcggggacctgagtagtcggtgcctgttcagtggggcctactcactagggtcccagtcaggggcatctggtcacctcaggcctggttagtaggggcctgatcagtggcagcctgttccctggaggcctggtcagtggggcctcatctgtgggtccaggtagtggggtcatgatcagtggaatctgatcagtgaggccttgtcagtaatgacctagtcagtgaggccttgtcagtaaggacctggtccttgaggccttgccagtgaggccttgtcagtaaggtcctggtcactgaggccttgtcaataaggaccttgtcagtgaggccttgtcagtggggccttgtcagtaaggacctggtcagtgaggccttgtcaataaggtcctggtcagtgaggtcttctcagttaggacctggtccatgaggccttgtcagtggggccttgtcagtaaggacctggtccgtgaggccttgtcaataaggtcctggtccgtgaggccttgtcaataaggtcctggtctgtgaggccttgtcagtaaggacctggtccgtgaggccttgtcagtaaggacctggtccgtgaggccttgtcagtaaggacctggtcagtgaggaattgtcagtaaggagctcatccatgaggccttgtcagtgaggccttgtcaataaggtcctggtcagtgaggccttgttagtaaggactttgtcagtgaggccttgtcagtgaggcctggtcagtaaggacctggtcagtgaggccttgtcagtgaggcctggtcagtaaggtcctgttcagtgaggccttgtccgtgaggctttgtcagtaaggtcctggtcagtggagtcatggtcattgttggcctggcagcgggggtcttgttagtgggtcctggtgatgggggtctaatcagtgagggtgtggtcagggaagacctgatgtgtggggtctggtcagcaggaacctggtcagtggggactgctgagcgctgcttggagaagccaggtgcattgcacgttatcgagggccctctggacagctgggatggcccagtgatgcccaatggcccggtcaaaagtagacaaagcagttgtttggatggacctgggagatgttgctcagagattctgacaggacaaaggtgaagaaagggtcagagtggctggagagatggtcacagtctatgggctgcacaggatggagaaagccagggaacagggagggtgggcagtgcggtgcagggagaggcaggtgcatggtgggacgtcagaccctgtgaaggctgtgagggtgtcaggtgggttgggctccaggtgcaccctcagtgcactgggtgggtatcaccccaagctccctggaccccagccaggtgatgtggtcactccctgggggactgctctcaggccccggctacctaccctgggcagcgctgtcccatctcaggactggactttctcagatcctgtagagggcacagactccacccaggaggggcaaccgcatggtgcagcctgagctctccatgggcctggagcatcccctgccagccttgtgctccccattctcccaggtcccacttttccagtgtcagccagcagggatgccccatcctcccttccccatgtgtctcctaggctgaaacttgtggcagattgggacagggatggtgcttccctcaggcccatttggggaggggactggctcccagactggtgcaggtcctcagctctgcctcgactggcttagagtgagatggatcagtcagttccctgaaggtgaagataagagactgtccctgctgttgggaggctagtctagggatggaggacttaacaggtcctcccaggctgtgaggcctgggcagcactgtcttgtcttaggactcagaaagtccagtcctgagatgagacagtgctgcccagggcgggtggctgggacctgacaacattcccccagtgattgaccacatcacacgtccagggtccagtgagcctggcctcagacgtgcccagtacactgaaggtgcacctggagcccactccacctgatgcctccacagccctcgcagggtctgacctcccagcatgcacctgcctctccctgcaccccagctgtccaccatgcctgttccctgacttcctccatcctgtccagcgggatgggatggacatggggacagcctgtgtgcacatttcgtggcgagtgggaatgacacaccatctctgggaggcaccatggttcctggcaaacccgatcccaagactctgtccttgaggtggttttaccaaatcccaaacccagaactgcggttgtggctcaggggtcagctcctgctagtgccaggacactactgggaggctgggacccgaccaaagcccatggtgtttctggcctgagaacaaggtgtcttgggaccataaggccaggccaccaatggccattgggtcataggggctcagccccaatctttgtctttccctggctccttctgattcagtcccatcagggccctggatcccaagactcagcatccaaggtcccctccaggaatcctggcagctcagcatactttatcctgtttcatctgagagcaaaaatgtaaaattggatgcacagaaaagtgactcaaagtgcttaatgactagaagaaatctaggagcagcaagaaggtaatgtggagggagggacctccatgaccggtgtctgcagagccaggggtacaggcacccagtgctgtggcctggcaccacctgcctctcagagggtgggtggcacactccttaaccagaggacagcaggcctggtcaccagcttttctacctgtccctgtaagcatcacattgctggaggaaaatctcatgccagagcttggaccatccctagctcgggttaggggttgtcccttggtgacctaaatgaaaaaacaggtccagaacagagttcctgatgctggacactcattcagtctttgaatcgtgggaggggaggcctggtactaggtagacctaacctctttgaggaaccacagagcccaaggctggaaacctccagaatcctccaccccctgatcctccctggggacccctgtggcctgtctcactgagcactcttccatctgtagatgtctgggctgctgtacaagggagtcccctttcaggtgtggtgctagacatggtcactcctgctggatgtctaggtggtagaaaccaaggacctagggaaataccagctacagcctttccccgctcatccagagcaggacaaacaggccaggcggtgtcaggagcccaggtctccagctggagggaacgtcaaccctgcggtgggagtaggggccctttgcacatcctaggcacagatggtaatgtagacaccacaggtaagctgggcttggtacctacccctccccggattcagaaagaaaccaaacaaggagctttgtgcggaatgaaacctcctttcctcccagaagcactgctgactgtttggtggttgccatttgtggcagtgagcctttgtttgttctgaggttgggctggtttctcctcttggtcctgccctacagatcataaaggagaacagcaagaggtccccagcaaacatccacagatggccttggaagtaagtcaccttgtgagaaacatgtcatgttctgggagggataaggcatcaagtaaggcctatggggttggaggatccctgggcaggtggggcaatccttggggtcttcccatgggaatagggaggtcctgaggcagaggcaggggttccacaggaggagtcacagagctaccaagggctctcctgtgccaggaagcagtcaacaccatgaactgaacacctgctgggctccaagccctggtccaggctggggcatgtggggccaggaggcagctcagagagggaggcagagagaagtgtgctgagagggcacccatttctgggtgtaatgtggtccagagattttggctgggaagggcttccagagtttcatatctgttacggagctgcttcctctccctagcctcaccctgcaggaatgccagtgaacatactgctgacatcttggagctcagtaccctcatagtgtaacggcgtcagtagatctgcctgtgcttggacttcctgtactacccattcctgaggggcgatgcttctgcagggcctgtgacttggtgcacaacttcagacaccatcatcttgcagcagcaccgcaccctcactagccagggtgttgatgacttcctcaaggccaaggccacattcaaggcttcggactttattgatgcgcttgtgctgagcaaggtggcttctccaggatcttaattcaggaggtagaatggagcttgagatcaagtgtctgatcaaggtacttgaacttgatctggagggctctggggagccatggaaggtgctggataaaggagtgacagtcagctatgttttcgagatgactatagaaggctgcctggaaggagtgaacaagagccaggagaccagggagggagcttgtggggcaggtctggagatggcaaaggagggatcctgcttggatgaaaggtcttcagggactgtctcaggttacacacaggtgtcctcagagctagtgtgttcagagtcttgcctccaggatgaaaatgggaaggagttgtcagacgaggacatataaatggaggctggcatattcatgagtgctggtggtggtcccggtgtgggactactgtgggaacaggggtctctccatccagggatatgatggatggaccctacatcactccattctgcccttcctttccctcctcccattctccccaaagcctcagtgtatgggcgctgttcatcctctggtgctgaagcagccaagagacccaagtctgcctggctgcctcttaggatatgacagcagagccagtggcctctactagatcctgtacaacctcacaaaacacccagacatcgggagtgctgccagcctgtgatgcaagagtcctaatcctgaagacattgaatggtgggtgcagggcctcatggcctgttccccagcccctctcattggctctgctccaggtggtgaagggggaaaatgtttttgtcaattctgtcatgattgcctagcaagaaaaggagcagaacccagaagcagtaaaatcagttagtaaaatcagttttcttttctgaactacatttctaccatctgtaattgaggggaattccttcgactccacgagattgcttggagaatgactgacagtgtatgtagagcaggtgccagccagcaggcgtttggtgtccagagcactcttcccccttgattttctgcctatattttcattttgttcccaagaccctcactccccttaattttgcttttccctctgattcccaccttatcttctatcccatggattcaccaggatgtaagtgggtaacagtcatctatgcatgtatgtgtatgtgtgtatgttctctgttggtgttggagtatggtgtgtgtgtgtgtgtgtgtgtgtgtgagaatgtgtgtgtgtgtgtgtgtgtgtgtgtgttggggtcactgagggactgaaactctccacaccaggctgtggtcctgctcactgctggaggcgctgtcagggctcttgccattgaccctccaggtctcattcttgcagtgcaggcagggcattctggaggaatcatgtccttggacgaagccctgaggagtgactggtgtgtattggtggataaataatccaactcccttgctctgggtgggatgactctgaggcacatgttctatgctgtctctcagaggtacccggcagggctgagtcctggctgcccacagtggaaactttcttgatgaaggtccctttaactgctacattccattcctgtctcagttccccactcctctactggtgttgcctgcaattagtactctaaggaagaactgacagtagaattactgtcctggagtcatctccagataaaatttttatacttgaatctttgcctcaggatctacttccaggaaacttaaactaagacacacatttttctttcctccaatcttcatagacctgtcattctgctgtttttaccaaaaaggatcatgaggatcagagaggaaaagtcacttgcccaaagtcacacagctgaacagtggtggagttcaactttgaccatgggctgtctggccccaaggtgtatgcttgcttctctcccaagagactcctttcttatcaggctcaaatgaatgaaaggaggatgttaaaggtaggatctctgaagcctgtgccagtggaaccgcagctcatggctggcacctgtgttctcattcttacctcattgagagtaaagtttattgagtttattgaatttaagtatctttagtgagatcatatattattagtaagaactgggaccaaacagattttctgactctaaaagagagattttcacagaaacagatatatacctataagtatacagacacgcatacacacatttctttactgctcataaaaattagtccttattagaatgtgggatgtataaatgtaagagaattttcatgttaaaattgacagatacatttttaaattgtcctaaaagaaatttaattatttttcttttagaattttccattattaatgttatttttatggaaaactatataactttattgataatacatacgataaccctttgtttttcacattgaaaatacagtgtattttgcaaataactaagtcctaattttgtattaaaatttaaaattttcaatctttttttattatattttaagttctgggatacatgtggagaacatgcaggtttgttacataggtatacacatgacatggtagtttgctgcacccatcaacctctcatctacattaggcgcttatctacatttggtgctacattatgatatggagaaataggaacgcttttacattgctggtgggagtgtaaattagttcaaccattgtggaagacagtgtagtgattcctcaaggatccagaaccagaaatacatttgacccagcaatcccattactgagtatatacccaaaggattataaatcattctactataaagacacgtgcacacatgtgtttattgcggcactgttcccaatagcaaagacttgaaaccaacccaaatccccatcagtgatagactggataaagaaaatgtggcacatatataccatggaatactctgcagccataaaaaaggatgagttcctgtcctttgcaggaacatagatgaagctggaagccatcattctcagttaacgaacacagaaacagaaaaccaaacaccgcatgttcactcataagtgggagctgaacaatgagaacaaatggacacagggaggggaacatcacataccggaacctgtcagtgggtggggggctagaggagtgggggagggatagcattaggggatttaataattttaaaattcaattctgttgaaatgtttactccaagaagcaatgtgtttttgagagctaatcctgatatattcaaatcttaacgacttaagttgatggagtggacttcttttaaattagtgattccctaatttacctgacagttggaatttccaggccatgttgaaaatacagattatccagactcttacttctgcagatgtatttagtggttctaaaatggcacccaggagtctggatttttcctaggggccttgtgtaattcacactgatgaccaggcaagtttgggaaattgtgccttaaggagatttttcattaagcagtcttcatttgaaaagaggatcatttatcttctaataccccatgcttcctctttctcctgctctctttgtctcctgttgtttttcagttcctagaagctttaattgaatgaaagttcctagtagatctgtacctactaaaaaccacacttctgaagctacgtggccaccagaagacacagctagtctgccatgtaaaaaaggaaaggtggcgtgtgccctgaaggcgcaggggtgagaggcagggaaatggagaccccaacagccagcatcagtggccctcatcacagccctccaggagatatcaaaggaggtcagaccttggacagtagtcttgacttcctgctatagaacacattgttaacactgaaaaagatgatctgttctaggggaatggtgaaagctgactctagcacttgtgctttttgtttgtttgtttgagatttgagtcttgctcttgtttcccaggctgcagtgcaatgcgtgatctcagctcactgcaggctctgcctcctgggttccagctattgtcctgcctcagcctcccgagtagctgggattccaggtgcccgcaagtgtgcctggctaatttttatgtttttagtagagatgggatttcgccatcttggccaggctgttctccacctcctgaccttggccaaagtgttgggattacaggcataagccattgtgccaggcctgtgtttttaatttcatcatggcacagcttcaaaatgacatctaagtagctgacataaaaatgaaaattctgtgtactttgatattagcaggcttcaaatacaaaccagaatatgagtaaattgtttcttagccaaactgaataattttcacattggcaggccattggtaggctattcagcatctggctcatgtttgggccaagctgggtgcctcatgaaagtctggagggaaatacagagggcaattctccataagcattaggctcagaaatatcctgcttcccagtgaaaagtttaagttgactttcacgccaccttcatcaaaccgaggttggtcaacacttttcaccactgccactggacactttgacttccaattttgctacctgctgtacaaagatacccccttctttcctttcctgcattctccctacaaccttgtctttttttgagtacatccactctgtctcatcattgtccatctctgcttcccaaaaccttatatagtacagaaattggccaatcaagattaccatgtggaagagcatccatttactgaataaggtagctggctcctgggggcagaaaagtgattagacacagagataaaaatcataatccctgctctcctggactcactgtctagtgggcagacagacagatgaacaaatacaacttgataactacaacaatcaagtgaattatataatttagactggagaaagagagcaaacgttatcataagacagtgtgtccactacttactctctaacagatttactcataaaccactaggcttgccaacttgttgaatgagataaaattggagggtcaggggatgggaaatgcatatgcttcttagtatttcattccataaatacataaataatgaaaccattaatgatatcatcatgttgattcaataagtttgaaagcaaaattgatagtaatctttggaagaaattgtatatgtgtctatgtatgtacatacatagacatacatattgctttctgaaattttcaatgactttatgcttcttctgaagcaattcgagtttagaatttgagacccctgggtcaaaacatcttcttgagtatactgaagaacatttggattaatttcagacacgtattttggtttatatagtgttttttatccatggtaacttatttactgtttaaaaacatattgagaacaaacagcagcagcaaccctgaattgagtgaaagtcctgagaagggctttgcccaatcagtgcatgtgcatgtttacactaatctcctgtctgaacctaggaactggctgaatgagctagcagatggcctgtctggctgcattataaatgataatttatttttatttgttttactcattcattcattttgagacagggtctcactctgtcatccaggctagagtatggtgacacaattatggctcactgcagccttcaactctcaggctcaagcaatcctcctgcctcagcctcctgaatagctgggactacaggtgcacaccaccatgcccagctaatttttaaacctttttttgtagagactgggtcttattatgttgcccagggtggtctcaaactcatggactcaagcgatcctccttccttggcctctcaaagtgctgggattacaggcttgagctaccacatatggccagaaatgacattttaaaaccaactattacgtacacagatatatgatgctctccgattgttttatctaaaaataggagtgccttaggtttattggctcattttgttttatcaaacagcttaaatgcagttgtgaatatttttgtttaccctcataacctgagaatttagaatgaatataattattgccattttcacaagtaaaattggaacgagaatataaaacagtggaatacaggtgcagctaagagtagcagaggtaagccatctatagaatattaaaatttcttccatgtgtgttgttagaaatctataaaaccaaaattttttcatgcctggaatcccagcactttgggaggccaaggcgggcagattacctgaggtgaggagttcgagactagcctggccaacatggtgaaaccccgtctctactaaaagtacaaaaattagccaggtgtggtgggcacctgaaatcccagctactcaggaggctgaggcaggagaattgcttgaacccggggagtggagattacagtgagccaagattgtgccactgcactccagcctgggcgacaagagtaagacttcatttcaaaacagcaacaacaacaacaacaaacaacaagaacaacaacaacaaaacccaacattttctaatacaaggaaactcactaagctttataaggcccccatccctccaagtattatcctttccggctgccccaacattaatgacagcaactggtactagccatctgacatacaatcccagtacatttttgggctctgatgttttctaggaattttttctttacagacttattgaaattaagctctattccctgaagacctggttttcataaactgtatagttttgcagttgaggggaaatggtcttaggtcagtcaatccactgttgacacagaaatgaaatgaggtaacggtaggattattgttaagattgagagatgtatgaatcatcctatgctctttcccagcccccaggtgaccatataattaaaatatccttgatatttctatgtagtttaagtttttaattctagaacccctcaatctttctctattcaacttaaatgaaaagacatttagtatccagaagacactcagcactcagaagcaaaggaagaaaaatggaaacataatggtcaatgcccatgttctaccccaggatcaaatgaggcattaatattgagagttgtgattctgaatgagtcttaacctcatgcacatcttagttgttcaaaaatgtgtcatgtcagcaaggtggtaacagtgaagattttctacaatcaaaatagtatagttatgttctgttgtataaaatagaaataaatggaccacattgatcagaaaaatcaccctagcatattccgacagcttaatttttaaaaagctatgttatattaacaatttaaccacatagatctgtcagctagaatacatgttgcatatttaattgacaataattttttttgctctctcagagagttgtagatagattttggggttatcaaatggttctgactttttttttttttttttttgagatggagtcttgctctgtcaccaggctggagtgcagtggcaagatctcagcttactgcaacctttgctccctggtttaagtgattctcctgcctcagcctcctgaatagctgggatcacaggcgcacgccaccatgcccagctaatttttgtatttttagtagagacggggtttcaccatgttggccaggatggtctcaatctcctgacctcgtgatctgcctgccatggcctcacaaaatgctgggattacaggcatgagccactgtgcccggctggttctgacatatttttaagaaagtttaggctgatgctatggtaactcagttattgatgatacatgtcatcttgtaagtaggagtattcaatattcatggttttgaagcaatttcagagtactataatcaccacacttgacagacttcagaattcattgaaaatctctggaaataattagcaatttgttatcatagactctaaacagaccttcaaaatgcattacatagatttggtaaaacagattgtcagtctgccaaaagtaatcactttggtttgatttgcattgaatgaaaattttaaaatgtaaagctaaactcttcaggatcactgcactgggtttctataggacaacactgaacagggctgccactgaaacaccttggatgagagcctcaaactccctgaacacagcttccagcacactactgggagaatgattgggactcaaaaatatgtgcaatgaatgtacttacgaccttgttggaaaggaagtaaagaggttgtatgtaattacctcaaattaatactgctacagataaagtagtaattagcactgacaaagtaatagtagcgctaaagtagtattaatagccctacactagtagtagtaataaagtggcatcgctattgattctatccgtctacctacctacctgtctggataggagggcataggatttgactgtaagattgggcttagcagtgaaaacaaaagagctcatgcatgctttggacaggtatgcactcaaacttggtatcatgtgactgttctgaattccagaaccaaggctaaaaggtggaagttttgtttgactgtgtctcacattttccttttggtctattgtctaacacatgttgctagaattctttttattctgactaccttttatgtatagacaacgccattattgacgagatcactcccaagcggattggagattgtcccaatacttagacctatagcaaggccttgggagaaatggtggtgcagcaggagagcaggaacctaaccattgccatcctaaggccctccattgtgcggagcaacgtcgcaccagcttttcctggtaagcccacttacctggattctgtgttttgctttcaaactaaggttcttctagcccaattattttctgatgtctttcttcttcttcttctcctcaggatttatagctaagtgcagccaatcaaatattaacccattatactagggcaaaattccactttgggatcatgtggctcttctggcagttaccctatcttagagttggatggagatcttaaattagcttctaactactctagtctcaaaattcccatgggtgatgttttcatatcttggctttcccactgtagcttaaacatatcagaatgttcttgatggccaaattggtagtattgcaactaactgccagggttacaaagctcacagaaggatctcagtttgggaatactaattttttttaacctcagtatactgctatgtccttttgaaagtactttgaattataccatcttcttcaaagcaagtccgtgaagctttgggggcagatacattccagctccaccacttactggcaatgcaacatttttgagttgttctttcggtagaacaggacagatactactttggagagttggtttaatgagacaatgtatgaggagtgctcagtctctagtaaaaggtagatccctactaaaaggctcaacgaatattagtcccttcatcttctcctcccgtatatctggtctcccattatatgcatttcgacttttatactgtaaggtaaagagacggcttagggcttaggaaacaaactcaaaaagacatttccctctaactactcttaagcataatctttcctgtaacatctttaagcaagtcaacacacattattaccaatcatgttttcaatgttctcctaaggaggctaaaatcaggaatgaggtgaagtatggttggtgaattgcttagcagatcatgatataaggacaaggtccattgtagcccttaagttgtcataaaacaccacaaaaccattcatgactcctgctacaaacaacaatcctggccaaattttaattcttgcacaaactgcagaaaatagccaacgctttgccattgatctttgggatcttaataatactgaattagatagttctatcggctggagtctcagtgcccttgtttgaaatttattaagattagacggcagatgttccaaagatacaacaattttctgaaatgtgtaaccaatggaaattttctttcttttagggttgggttgataatctaaatggatgtagctgactcattattgcggtatgtatagggatgaggaagtaactgtaatgtagtggaggaatagtaagaaaattcttagtgctggcttagcttcattgatccaaaaacataaatgctactttactatcaattgaagcatattatttcaattattctggttataatatggaggcaggatgaaattgtttttattcttttagaattttttttatcaggaaaacagaggtaaagtgctatcaattactatttaagagttctattttgaaaagtgagaattaaggatttttcttttctttttaaaaaaaacttttttaaaaattaaaaataaaagaagcaaaagtcttaggaaaatgaggcaagtagccctgccactctatgtacagtaataacaatatctgtcccagttattatgtacaatattataaaaaatgtcgcagacagtacaaattaaggcccttatttctcaaaggacatcaagtcttatgccccgtgggagggaagatgccacttaattattgcaccattttgaaaaacagaactcgtcaaggcggatagtggtcttttcccacctggtcttcagtcacagatgggggctggggcggtggcgccgtcttcgtcgtcgaggtagaccctatggagccacaccgccctgccgagttctctgggtgcaataaataccagtcacagtttgggagggggccgcgtgcaggtggggaggcctctgggctgcatgtgccgcttcctgtgcagggcaaggtgggcagagcgcaagaaggcgtggtggcaccaatggccctgaaatggccggtggcccctgtgcttgtggtggtggagcgttagcttgtctgagtgagcacacttccagctgcttccatcccagtggcagtggtcgggcttgtcacttgtgtgctgcgcagatgtgcctgcaggggagaattc

SEQ ID NO: 89 - DNA, 85660 bp, human
ttgcaaacgttacttaataccaagtatgattttctatatgaaaaggagaataataatatgtcctttaaggactattgtgttgaagtaacattatgaatgtcaacagcatttaattcagggtctcatacattcttatcagcatcattaactaaacttagcatgactactactaatatcattattcctaatattgttttaagccttcagattgctgtcacttgtctgacttctagctgattttgaagtataaactattatgtcagactaaggaagaaatagataatttttcatttaaatctttgcctcttttagattagtgaacagagcataatttcttgcctctcaaaggactttatattagccaattctagtgtgccatatccccgtgggacatgaatctttttgctccttccttttagccttggtggtgatttacaaggataaacacttgagcactcaagatacttatgtttgttggtacatgtaaatggttaattctacaatgacaggcacatattaaattggttctgctcataataatgaagttatctctttgttattttagcacagccctcatgcttgctgtatgtcatggatcatcagagatagttggcatgcttcttcagcaaaatgttgacgtctttgctgcagatatatgtggagtaactgcagaacattatgctgttacttgtggatttcatcagtaagtgtttacgtttagaggctaggtgagattttatagtttgtttcaggtagtttttgaattacagtgagttcacttcatcagccagaaactaggcaaaaagctagactagttagaaggagtattgggtccaggattctttattttaggactttcaacaactttatccctagggatcctaatgttgtctacttgatttttctaattagttatttgggtcttgaagtgtccactttagcagaaaacctgatagtgtcctctgggggctgtcttccatatctaaatacttgaattttttaaaaaaaatctaaggagttccctaagtccaaggaagacattctttttgtgtaagtcaaaaggattgggggcagaaatcaccattctctttattttgttgtttccattgattctgttgttgcattgttgccactgaaactgcccctgcagtctagtaatgattgacctttgtgaccaggatgcccttactaacacagatccctcagtcttcatggtgatccatatgtagatttcaaagttattacagttttttaaagttcacatacatattctcagccattgtttccaaagtaccagcaccctgctctggcagctagaacttttagctttagccacacacgtagtgagcaaattgaccctgctcctccactcaaaacctgatgtgaaacccacatcttagcctggacttggcctagaccttcagggtaagttatcctttgaatgacttttttctattttctcttgcaaatattaattgtgatagtttaaaactgtaagtcaggttgaagtaatgttataggaagaaattagagacccatttttattttgttaccagatctatatccctggcactttatatcctgtgtagtgccattttgtaagtagcagaaggtcttatcttattccgtaagatcccttgtcatctttccaaatttgtagtgggttccaacttgtggttataccctcaagtgattcttttttcctaaaagtaaaaatctcccatgctacttgcatctctatctcaagtttttaaaatattttcaaattctgcattaccatgaagccattcaatagacttcactctatctcaagtaagttggttagatttaacagagctaagcctcatccatcattgatcagtcttcaggtataaaagtagggatttgtgctggcttcagtggtacatatactaaaattgaaacaatgttgagaagatcagcatcgtccctgcacaaggatgacacacaaatctgtgaagtgttgcatatttcttgcagtccccaaaaggacatttgactactttctaactagctccaaggaaatgatgtgtcaaagcaaaatgtgtgacaccaagtattgcaattgtgattttcatacaaaaaatatttatgtaaggtgatgtatgaaatgagatttggtgagtaacacataggatcttgtgtgcagtatgctgttagtaggcatctcagaaatgagaaaataccaacttgcatcttctttgtggaacttacaaaaaacagtggtgggttttgtcttccacagcagctggaaatgatcatagttactaagcatcattctatcaaagatttgttagttcaggtttaaggaggtaaataaagagtagcaatagtccaagccagatgctgacatctattagttttctgcccttggtgtgattgatgagctcagtaatagaggataatcaggttatccaatttgatgaattaatatatttataaataaatttcattacaaattataaaatagcttagatgccttgaattacatgccacaaagaatagaacatctaataaccaaaagtaggaattaataacagaaaactgcaacatttgaacattataacctatgaagaaacactttttttaaaaaattaattttttgtagagacagggtctccctatgttgcccagggttgtcatgaacttctgggctcaagcaattctcctgtctcagcatcccaaagtgcttgcatcacaggcgtcaggcactgcaccaggccaacacattgggttttattgggattttaaaacagtttcagcaattaggttcaagaacaaattatttcattgcttcactatttctttgagcatttttaaaatgttatcttgttagatctttataatagtctagtgaaatagggctctaaaatcctcatttttagaagacattgagcctaagagaagcaacttgttcaagaaaaaatacccgttggtaaccatgctaggactttttctgagttagggagattttccattaagccaagctaactctagttaatttactgagttatactgccctaaatgcatgagtatttcatcttactttatttcttctttaattagaagcttaataagttcatagagcttacaaacttaaagtctatggaataagtaatgttctgatgttagttctgatattgtctgaaatgctctaagaacttaataaatttggtaaatgttttttataaagtcagtgttaaaatggtaattttatttatcacatttttatacatagcattcatgaacaaattatggaatatatacgaaaattatctaaaaatcatcaaaataccaatccaggtaagacttctgatagtaaactacccttggtggtgctatcataagattatggaagtgttgatcacaaaaaggtaattcaaaaagcaatgtgtaaatagcatgtgtttacatatatacatatatgtgggtgtgggtgtgtgtatgtgtgtgtgtgtgtgtgtatatatatatatatatatagctttgatttgattttttggtttataattcagaattagttaagaatttagttgtaggcagtttataatctcaaaaaatgttacctgaaaaaatatttgtttaattatggtccctaaatcctatataatacttttgtataaataacgcaagttttaagtttgtatattttatgtttcctcaactgtcctaacaacttaggcttgttataaaatgtataacccttggggtgattgatgagctcagtaacaggggatgatcaggttatccaatttaatgaattaatatatttataaataaattttattacaaattataaagtagcttacatgccctgaattacaagccacaaataattgaacatctaatcatgaaaagtaggaattaataacagaagccaagatggctgaataggaacagctccagtctacagctcccagcgtgagcaacgcagaaagggatgggtgatttctgcatttccaactgaggtacccggttcatctcactggacagtgttggaaagtgggcgcaggacagtgggtgcagcgcactgagcatgagccgaagcagggcgaggcattgcctcacctgggaagcgcaaggggtcagggaattccctttcctagtcaaagaaaggggtgacagacggcacctggaaaatcgggtcactcccaccttaatactgtgcttttccaatggtcttagcaaacggcacaccaggagattatatcccgagcatggctcagagggtcctacccccatggaaccttgctcattgctagcacagcagtctgagatcaaactgtaaggtgacagtgaggttgggggagggacgcttaccattgccgaggcttgagtaggtaaacaaagaggctgggaagctcgaactgggtggagcccaccgcagctcaaggaggcctgcctgcctctgtagactccacctctgggggcagggcattgccaaacaaaaggcagcagaatctgcagacttaaatgtccctgtctgacagctttgaagagagtagtggttctcgcagcacccagcacgcagctggagatctgagaacggacagactgcctcctcaagtgggtcccagaccctcgagtagcctaactgggaggcacccccaagtaggggcagactgacacctcacacgcccgggtactcctctgagacaaaacttccacaggaatgatcaggcagcaacatttgctgttcaccaatatccgctcttctgcagcctccactgctgatacccaggcaaacagggtctggagtggacctccagcaaactccaacagacgtgcagctgaggatcctgactgttagaaggaaaactaacaaacagaaaggacatccacaccaaaaccccatctgtacgtcaccatcatcaaagaccaaaggtagataaaaccacaaagatggggaaaaaacagagcagaaaaactggaaactctaaaaatcagagggcctctcctcctccaaaggaacgcagctcctcaccagcaacagaacaaagctgatggagaatgactttggtgagttgagagaagaaggcttcagacgatcaaactactctgagctaaaggaggaagttcgaacccatggcaaagaagttaaaaaccttgaaaaaaaattagatgaatggctaactagaataaccaatgcagagaagtccttaaaggacctgatggagctgaaaaccaaggcacgagaactatgtgacgaatgcacaagcctcagtagccgattcgatcaactggaagaaagggtatcagtgattgaagatcaaatgaatgaaatgaagtgagaagagaagtttagaggaaaaagaataaaaagaaatgaacaaagcctccaagacatatggggctatgtgaaaagaccaaatctacatctgattggtgtacctgaaagtgacggggagaatggaaccaagttggaaaacactttgcaggatattatccaggagaacttccccaatctagcaaggcaggccaacattcaaattcaggaaatacagagaacgctgcaaagatactccttgagaagagcaactccaagacacataattggcagattcaccaaagttgaaatgaggaaaaaatgtcaagggcagccagagagaaaggtcgggttacccacaaagggaagcccatcagactaacagctgatgtctcggcagaaacgctacaagccagaagagagtgggggccaatattcaacattcttaaagaaaagaattttcaacccagaatttcatatccagccaaactaagcttcataagtgaaggagaaataaaatactttacagacaagcaaatgctgagagattttgtcaacaacatttgaatattataacctgcaggggtctgtcctgcagatggttgagacacataaccagatactgatattcagtgaaagagcagcaaggattccgagctgattacagacaccaaggaagatgctgtaaagagtcagcagccacagccctggctagctggccctgtgggcatttattagtaaagttttaatgacaaaagctttgagtcaacacacccgtgggtaattaacctggtcatccccaccctggagagccatcctgcccatgggtgatcaaaggttagtcttaagaccacataagtaaacaagctatttagataaactactctaccttcctttgtatccactttaagctatttatgcaaggtaagaattaggctgctttcagccataaccatatcttgagacttttacaaaaccctctggccttccaagaagatttgtgtctatgtcctataacttcatattaaaatgtttctcaccagcctgactgaactcccacaataacctatgaagaaacatggttttattgattgattgattgattgattgtagagacatggtctccttattttgcccaggttgatattgaacctctggtctctatttaatttttacaataaatggtttgcatttagtaaatgagaattaattacagttgagtcttgagcaatatgagagttagggtgctgatcccccatgcagctgaaaatctgcttcatatgaaaatctctttcttttgactcctccaaaactttactagtagtccactgttgacctgtagccttcctgaaagcacaaacagtcaattaactcatagtttctattttatatgtactatatagtgtattcttacaataaagtgagctggaggaaagaaacttttataagaaggaaaaatatattcacttttgagtggaagtgaattattatacataaagatcttaattcttattgccttcatgttgagtagtctgataaggaggaagcagaggagagatttgtcttgctatcttgcagtggcaaaggaaaagaaaaatctgtctgttagtgggctcctacagtgaaaacccatattcaagcatcaactgtgtgacatagtgactcgtgtcactaaaaaagtaactatctttagaatttgaaaacttaataatgctttcctggtgccataaacaaatgtcaacaagaattacaaaacttagccagggtgcctcagtaccaataggaaattattttccaaagatacctactgaatgcagaagtcagaaaagcaattatttgttgagaagcgcaggttatgttacatagtcttgtaccaacaaggtctcactattatcaacttcatttcctccaagttgaaaccaaataagatatatttacttcattaaaacaagatatgttgttctatctgctggataattagggtgttaatagtaatttgttacaacaagatactctgtttctaatagccaaaatattatcattataaatattcaaatagcttaactctaggctcaacaaattataatgaagttagaaaaatttttcataataacaaaaatgctactatgatacctaaatgtgacataatgcattgtacaatatgaattatatgggcatatctttattatatatttattaaaggacctctgtaagttaggttttgcaagttgcaggagacaaggatagaatacatatagtttgggtctttaaggtgctcataaaacagtagagctgtccttattgaatttctgcatttttccaacagaatttcgtaaaaaatgtttttttattcatttatccacttgtccacttaacaaataattgttaggtatctttaaggtactaagcatcttccttgttattatataattggtcattttatattatttactactttattaaggtattaagcatttttcttgttattattgtctttttttattatttgttactttatttagtgcttactctgtgccagaacccctttggaagcttataattattacttattatgtcatgtcgttactgtattcagcatgtgtcagaccttttatatccaaggtgaagaattaaagctttaaaaagtttggtagtgtccaggcacagtggctcactcctgtagtcctagcactctgggaggccaaggcagatgtattgcttgagctcaggagtttgagaccagcctgactaacatggtgaaacccgtctctactaaatatgaaaaattagctgggtgatatggcccacgtctgtaatcccagctacttgggagactgaggtaggagaatcgcttgaggtaggagaattgcttgaattgcattgagctgagatcacaccactgcactccagcatgggtgactgagtgagactctgtctcaaaaaaaaaaaaaaaaaaaaagagaaaagaaaagtttggtagtatttaaggaaagcatacagaatgagtagaagtttgccaggtgaagagtcaggaggatgatatttagtagaaggaaaatttaaccagattgtgtgtttggcagaaggaacatctgcaggaacacctgatgaggctgcacccttggcggaaagaacacctgacacagctgaaagcttggtggaaaaaacacctgatgaggctgcacccttggtggaaagaacacctgacacggctgaaagcttggtggaaaaaacacctgatgaggctgcatccttggtggagggaacatctgacaaaattcaatgtttggagaaagcgacatctggaaagttcgaacagtcagcagaagaaacacctagggaaattacgagtcctgcaaaagaaacatctgagaaatttacgtggccagcaaaaggaagacctaggaagatcgcatgggagaaaaaagaagacacacctagggaaattatgagtcccgcaaaagaaacatctgagaaatttacgtgggcagcaaaaggaagacctaggaagatcgcatgggagaaaaaagaaacacctgtaaagactggatgcgtggcaagagtaacatctaataaaactaaagttttggaaaaaggaagatctaagatgattgcatgtcctacaaaagaatcatctacaaaagcaagtgccaatggtaagatgctagagcgaactttgtaaggtttattggcacttcaggttccctagtgaaaaaagtgtgatatgggagtagttgggaatgacttgaatatctaaataaggcgagcttaggcaacactttttaatagtataggaataagtagctcttattctgtaggtcctggaaaaattctcacaattcttctggccgtaaatgctagatgaactaactaacaatggctaaaaccataggaaccaaagttgtttggtggtacagggatatcataggatcacacttttttttttttttttttttttttttttttgtgagacagagtctcgaactgtcccctgggctggagtgcaatggcacgagtgatctcggctgactacaacctccgcctcccaggttcatgtgattatcctgccttagcctcccaagtagctaggattacacgtgcccaccaccacacctggataattttttgtatttttagtagagatagggtttcactatattgaccagactcaaactcctgacctcatgatatgcctgccttggcctccaaaagtgctgggaattcaggtgtgaaccactgtgcccagccaggatcccacttatggttggctagttagcaacagctccaataatcatgctctctcaagacaatatttaaaggctggaagggctgcttttgttcacatgtttcttttaaatagggagaaaacttggaagcttgcagtaatcttcctgtaacattttattggctggattacaccacatgcccatatctaaaccagtcactaggaaagcaaatgtaattactgtgattagcttagaataatgatttctctttttgagatgggattggggtaatggaatactgaatatctaaagaaacttgtgtttctgcagcaagaaagaaaaaataatgcttatgcataggaagccagcaatgttttctgcagggattcattggaaaattttgagcaggggagtcacaagattagatttgagtatcagggcatactggttatggtataaggcagagattggcaaacttttcctgtaaagtgacagatagggaatatgttaggccgctgggcatggtggctcacacctgtaatcccagcactttgggaggctgaagcatgcggatcaccaggtcaggagttcgagaacagtctggccaacatggtgaaacctcatctttactaaaaatacagaaagtagccaggtgtggcggcatgcacctgtaatcccagctacctaggaggctgaggcaggagaatcacttgaacccaggaggcagaggttgcagtgagctgagatcgtaccactacactctagcctgtgtgacagagcaacattccatctaaaaaaagaaaaaaaaatgttaggccatgtggtctctataacagctattgaactctgcattgtagggtgaaagcagtcatagataatgtgtgagcaaacaggcaagattgtattccaataaaactttgtataaaaagccatttggtaagctgaatttggcctgtggcctatagtttgctggcccttcatatagaagaaagatggagggtaattacataaaaagatttaaagacgaaacttttgtagtagttcatgctatagtcttttttttttttttttgtcaccaatctgtggcctagtatcaatctattatgaaagtttgacccatccagggtaaaaagagtcaagttcagaagctcaatttacacatttaaatacgtaggtctttctttgccgttattttattttgatttcttttactgaattttttttaacttaaaaataaccgtagtaattggtagggtttctttgtccctgtgaaagccatcagttaaggggctgtgtctaactaggaaatttgagtgaattcacttcagaagaatttagaatatttctgttatatatttgttttctgtctcatggctgtcagtattccttgggccttgcatggaccttgatttaattctatgaaatgtgggaaatttagttacttaaggcaattgtttttcctataaatttttaatgtttctccaaggaggcacaagttgattctgaaggtattgttgcattaaggaaagactatctcattgtaattaaagcagtttttaatttatatgcaatacaagttctttaagcttatttatctaaaacacatacacaccaaatatactgtcatggcatattgaaatttaaaagggttggacatatattttgctaatatcaaaaagttaatattttgaagcatcttatttattgataaataatcttttttgaagaaccatataataaagattagtcaatcaaactacataatactaaaaaataaaagaaagtaatgagagctgacaggtaattaaagttatctgagtgaacagcaaatatctgacatagtatgcttcacgggagaagaggataggacttcttttgtgaagaaataattaatacaaattaatcaaatttttaatttttttgcattctaatgaatataaaattaatttttacatttttaagattgcagctctaaccatttgactatagaagtggttgtggttatctaccaatagaatatacaggctatagaagtaagtccacagattcatgaatgaaaatagatttgtatatgttttaaaaatttataataagaaatgttatcatgaatatatctgtgattaactttttatagatcagaggttcccatcagaatccaaacaagaggaagatgaagaatattcttgtgattctcgggtattgtgtattattgatgttattctctaaaaatattaatattgagtgatgtgaaaatataaagtcagaggctttgacttggttttcttaccactgcctatgctcagaagaaattctgatatttctaaaaacatacttggctgggcacggtgactcatgcctgtaatcccagcacttcggaaggcccgaggtgggaggatcacctgagttcaggagtttgagaccagcctggctaatatggtgaaaccctgtctgtactaaaaatacaaaattagttgggtgtggtggcatacccctgtaatcccagctactgaggaggctgaggcaggagaatcacttgaacccgggaggtggaggttgcagtgagctgagatcgtgccattgcactcagatcgggccattgcactccagtctgggtgacagagtgagactctgtctccaaaaaaagaaatacttaaaaaaaaatgtgtgctgagtagattactgcttggtagtgaaattctgctaattttcagaattagtttcagacacttgtgtagtacagtgtaaaaaataaggcttagaattcaccagtaattcacatgagttctgaggtgaaatttgatcaaaagctaaagaattactccgagttcatctttgggcaattatatgactcagtggtatatctagacgtacaaaaattttaattggattcatagaaaaacagtttaagctgcagttttgtacaacttgaaacataaaagataatgcttggattggaaattattgacgtatctgtattgtccagtatagatctgaaggaacatttcaggagacaaacatgcataaagaataggaaaccagtgggactataataacaacaatttggttgagtagtattttaaaaagtagaaattatttttacagatagaactctttgagtccgtttgtggtagccatgcttatagcaacataagtatcaaataatagtgatatagtccaaggtcacaactgtggaaagacacaggaagtgcctgacctcttagatccaagcccctgctgcacagtggtaacagaaatgagatgacaacagacaagcagactttatctaatttgtccatagacaaaaaaagactttctaatacttgattgctttcatttaaacataacataaatataagttttccttactactttttcattttattcaagtactttttccatcagcagatattttatcaaaaacatgttgattattttaagcccatttacctaaataaagcagcttctacaatgttctgagtgcactttatatgacttattcttaacattcttttcatgccatgcatcatttttaacactaaataattctattatcatgaataatttcagtgtttaatgcagtatgtatttgcggcagcgctcctctggagggggcaccagctgcagggagtctgtcccttgcagacccctgacccggggacagatgaataaagtacattgacacacagatattcttctctgccagtccagctgagggcgtctgagtggcttacagactccctgttgagttctgtaaacagttgtgactatggccctgatcagctagtcagactcgcatttattcagtatgattaattaacaaaagcttgagtcaatacccttagagggtaattgacattgtggacttcctggggtagtcctgagtaaaaagcacttaagcacctgtggtacatcaaaggtgagtcttaagattatatgagtaaacaagctagctaggtaagctactctgccttcctttatgactactttaatttgtttaactaaaggtaaagatcaggttgccttcaaccatatctattaccaaagttatgcaaacttctcggccttccaagaagatttgtgtctatttatataactacctctaatattttttccaccagtctgattgaaccccaacatgtgttatggcaggtagcaagtgtattgtacttgtttgatgtgccttcttgatttttggtgaggaagataaagtaagatatttttaagtgagctactttttgaaatatgcagatgagtgaaaatttttgtcaatgttatttatttttcatgctcagtaaccgagtcaatagctgcatgaagataacagataagttttgatatagatagcatgtgtgagggtttctatcaaaatgtcatgattttcaatatgtgcctatcctcaaatcaaattgcctttgaagtcagaaattgagtttaaaaaatattttaattaggaattttcatgcttatgattttaaaactgaaattatctattgatactacttttaacagagtctctttgagagttctgcaaagattcaagtgtgtatacctgagtctatatatcaaaaagtaatggagataaatagagaagtagaaggtaagaacgattttttatttgaaaagtcttttaaccatatgtttgtctaaatgcatgatgactgatatactctaatagccaaataaaattacccactaaatacataacatcgaaaagagaggagtaaaatgataagttatgtatcttttcaggtgttggcaacaggaatatgtagagagtgcaaaaaaaacaaccgaattatttgtttgaatccaagatactctaggacatgacgaaagtcaggaaacaagtaagggaaaaggagtaaaaagggaatgaagactaaagaaggcagagagagtacagggagttcatgaaggaacaagaagcaggtttatataatggaggtggcaaaataaaatattttttagaaaagatagaccatgcttagaaagatggcaagaatataaaatgatcttccagtaccaaaacttgtccgaaaattatagctaaagttttctcacttttcctgtctttctcactactgggaaggcattaggaatggaattatctgagcatgcagaattgtgttttatttgcaataggtgagtattaacaaaaatgcataggtgtgcatctataaaatttatcatatacactcagtatagacaaatacttatgaaacattagaaaatcagctgaataccttgttaatacacagtatcattcagcataattgagtttctaaattttaataagttctcaggcgatgctgataccagtggtacttggagctcattctgagtagcaagaggagaggccattcatgggaaaaatgtggaagaagagcaattggataaaaggataagacaaaacagggtcaagagaaagcattatattccttttatttctgagtatgcttttagccagagaagaagaataaaaagataaagaaacaaattataaatttaagttactgtcactaaacaaggagaaaggaagtgatggaaaaataaatttgaaacagtgttggatggaaaaaatcagttctaggaagttagagtttttttaaatttttttaaaaatttttttagcagaggcattctaggcaggtaacgggacaaacaagagagaaggtaggtaggtaattttggagactctgagaaggacagttcagtgaactcgcttcagatgtgtttagattatttgcactccagaaagtaaattatggacactccagagactaccagaagcaggagtcttactagaattggagaaaccaaagtgactcattaggtttctatgttacaatcaggcatgacaaatatatatttggttgatgttagctattcaagtgagatatatttgaaactaagaacattggctttatattttaagaaagtgagttgttttggtaaaattgtcattccacaaaaatttattatagactaatgatacgccaaatcagatgaattgtaggagctaaaaaaatactgaatttatacttgaataataagattgctttttaaaataaatattgtggtgactttataatataaaaagttatttatgtttaatacatctattgcaattaatttttatatacatatgtcaatattgaaagcttattatagattatttccatgatgagttttatacatcatcttgcatgagtggatcaagaagcattcagatgtataaactggaggatacagaaatgtaggcatattattacaccatatggttatggaaaacaatgaatatttatatttagtattatgaactaagtatatatccaagctgatcaattcataacacttcactgatgagatgtcaattctacattcagctgaactctcatcataactatgtacttttccaaagataggccatattaaagaacatgatgaatagaataacataaatgattctaatatgtttcattaagtacgtggtgtagcattccatgttcagctttgacatttattttctcatatcaaccctttttacacgtgaaacacaatctcgctttgaagtcttaactgcacgatctatgaaacctatatttatattttcttcagtgtattcctgtcgtgtgtgtgtcctaaacaaaccaaaagaaaactttccaaatctaaagtattcgttctcaaagtcgaccaagaggaatcagttacatactatgactgcattcatttggttggcattgtcatacttacttatgattgatgataaatctcttttgctttttagagcctcctaagaagccatctgccttcaaggtatttagttttattatttcattttgaatgacttattaattatgtatttgtgaagtatacattctttattaatcattttgcttccaaccccatttagcctgccattgaaatgcaaaactctgttccaaataaagcctttgaattgaagaatgaacaaacattgagagcaggtaaatttttcaatttaactatgcaaagacgaatatttcaatattggacattttgatggtctttctatccccaatgctttatttttttcaactttgatgaaaacatttgatctagataatgccaatactggtattgatgtttgaaaacctgatattacaagaacagtaatttttaatatattttttaaaaatgtagccttaatctcagatgtttctacttttgtatcccgaaactgtaatgtttactattttgaacttctgtaattcttaaagattcaagtaggtgaattttgaaactcttaacttcttttagttcttcgaagcttgattcaaattccactgtttacttcggggatcacgtcttttactgatataacacttgtgttttaatattaattaccttgtttccagtgttgtcactttgggaatcctaagaaaatcagtaactcggattagtgaactctgtgtgtttgtgtgtgtgtgtgtctgcgtgtgttcctgtgtgtgtctgtttatgtgtttggtacctttaccttgtaaagatgaggaaagtaattagtcatttctttgtgaatatttgataagcacactttttcctaaaactgtaattctgaagcatttgggtttagagtctttttacactaacctactttttaaaactattcttatgcatgtttaaacattttacaacatgtgtgcgtggtcatatttaatatgtacaatttttttcaacttctaatgcatacatggttgtacggtgtaatgttcagaaacattcttttttgatcagcatcataatttttagtgacatccataaaggacacaattaactgtgttttttaatattaggttgtttataaaattccattgtatgacagtaccatagttaattgcacaatttttatgctgataagtaataaataaaaatgaaaacattcagatttcagactctttcaaaagttagtgttatctgctgattttttaggtattagaaactaaaagtaaaatatttaaagtatttccttgtgcaatcatacactccactaagaatttgaactgtgccccacagcttcttagagctatggtgtggcaacacgttagatctctgaaacgatccagggtacgcttctaaaaatgagtgaacatggtgacttaccaaagtgtgatttgagtttcttgtaccctctgcatgaaatgtgaacattagggatgctaagatcagaagttaaacttacttttgaaaaccaggtatacagttgatggatgtcaaatgagaaacgtatgcatagtgatgaaacggtcttttaagttttagttgtgcatgtttgctttttttcctcaacctgattcaaatagttgtaatttgtactttgtactgataaagaaaactggacattatttttggtataaattcattttctgtctcatgttcctgagaactcgtcaagtcttgagtgggccttgattttatcctataacatgtgggaacgttagattacttaaggcaattatattttcctatacatttctgatgtttctccaaggtgtcacaaactgactctgaagatattgttgcattagggaagaagtatgtcattgtaattgaagcagttgttaatttatattcaataaaagtttttaagcttatcttcctaaaacatatacacacccaaaacacacccaatacactgtcatagaatattgaaatgtaaaaggattggacatatagttgactaacatcaaaaagttaatactcctaaaaaatcttattcattgataaatcatctttgtggaagaactgtgtaatagagattgctgagtcaatcaaactaagtaatactaccaaatataaatttacaaagaaaagagagatgataggtaattaaagttttctgaatgaacagcaaatataggacatactgtgtttcagaggagaagaggatatgactgctttatgaagaaataatccatacaagttaatcaaatttctatttttctgcattgtaatgaaataatgttaattttcagactttttagatttcaattctaatggaatgactatagaagtagttattgtaatcgacaaaaagaatatacgggccacagaggaaaaaccacagattcgtgaatgaaagtagatttgtatatgtttttaaaatttatagtagagaaatgttgtcatgaatgtttctgtgattaaccttttatagatccgatgttcccaccagaatccaaacaaaaggactatgaagaaaattcttgggattctgaggtactatgtgttattgatttttttttaatattagtattgcatgatatgaaaacataaaggcagaggcttaggctttattttctcgcctcttcatatgtcaccccgaaattatttttgatatttttcagaatatgcttaatggagaatctatgtgctaagtagattactgcttcatagtgaaattctgggaatttgtacgattaatttaagaagccaaagtggtatagtgtaaaaactaaggcttagaattcactagaaattcacatgggatctgaagtacatttgtctaaaagtgaaagaattacactgattccagctatgggcaaatatatgattctgtcttacatctagatgtacaaaagttctaattggatgcatagagagacattttaaactgcagtattgtaaaagctgggacctgaaaagttaatgcctgggacttgaatgtattgacatatgtttattgttcagtatagatctgaaggaacatttcagcagaaagaaaagcatgaggaataggaaaccttgtgggagtataataacaagagtattggttgagtagtcttttgaaaaatataaattatattttcacaaatagaactccttgagtcctcttatggcagtcaagctgcagcagcatgagcgtcaaataatagtgatgtattttaaggtcacaactgtggaaagacatgggaagtatctgacctcttagaaacaagcagctgctgcctggtggtaacagcaaaaggtggaagtcaataggtagattttatctgatattttattagacaaaaagactttaacacttgtttgctttcatttagatgtgacgtaatttttttcttactgcatttacattctattcaagcactttttctatcaccataaattttgtctaaaacatgttgcttattttaagcccctgtttaccgaaaggaagcagctttttcatattctgtgcatacattctgtgacagactctaaaagttctcttcatgacatgcatcatttttaacactaaacagttctgtgatcatgaatattttaaatgtttaatgctgtatgtgtcctggcaagtagcaaatgtgttgtattttgtttgaaatgtcttatttatttttgatgaggactagaacataagttagatatttttaagagagatactttttgaaatatgcacgagtgaatttttttatcaaggtgatttgtttttcctgctcagtcactgagttaatggccacatgaatgtaaagatcagcttgatgtagagagggttgtgtgaggttttctatcaaaatgtgttggttttctatatgtatctgcacttaagtcgaattgtttgcaaaggaggaaattgtgtttttaaaaaagaatttaattaggaaattttgatactcttcattactaggatttatccattgaaattatttattgctattacttttaacagagtctctgtgagactgtttcacagaaggatgtgtgtttacccaaggctacacatcaaaaagaaatagataaaataaatggaaaattagaaggtaagaaccgttttttatttaaaaatcagttgaccgaatatttctctaaactgatgaggagggatatcctctagtagctgaagaaaattacctcctaaatgcaaaccatggaaaaaaagagaagtgcaatggtcgtaagttgtatgtctcatcaggtgttggcaacagactatattgagagtgctgaaaaggagctgaattattagtttgaattcaagatattgcaagacctgaggaaaatgagaaaataagaaagaagaatgtagtaatagagtaactgaagactgagaaagacagaacgtacagagagtacatgaaggaacaagaagcaggtttatatagtggaggatgataaaatgaaatgattctttaggaaaagattgggtattgttagaaatttgggaagaatataaagtgaccttctagtatcaaaatttaccagagaattacagcaaaaatattctgactcttaatgtctttctcactggtgggaagccattagggatggaagcacctgaccatggagagctgtgttctatttgcaataggtgagaataagcatatctgcatggccacacatgtatataatttgtcatatacactccgtatagaccagaagttttcaaactttagaaaatcagctgaaaaccttgttaacaattcacactgagattcagccgacttgggattctgcatttttaaaaagttctcaggtgatgctgatgctggtggtccttggaccttgctctgggtagcaagaggagaggccttttgtgggaaaaatgtggaagaacaattggatagaaggtcaagacagaaaagggtcaggagagagcagtatggtgttctcaattttgaggatgtttttagtcaggggagaagaagaaaggggcaatgcaatagaaattatagatggaagatactaccactaaacaaaaggaaagagtgggaaaaaaattttcacaggtgttgaatgggaagaaccaagagcacaagtctagagattatttttgctaaagcagagagattgtgaggcaggaagcaggggacaagagagaagccagctagatgattttggattttctatgaatgaaaatggagtgagctcacttcagaagcatttagaaaagttttactgcagaatgctagagtgtgggtgctctggagactactggaatcatgaggattactagaattggactaaacctcagtgactcattaattttctttattactatgaggcatcaaatatatttatgtatatatgtatgtatgtatgttgttgatattcacaatttgaataagatatacttgaatgtaagaaccttggctttatttttaaggaagcaaattgtgttggtaaaattgccattttataaaacctcattagcaaataacatgatacacgaaaccagactaattttaaaaaccataaaattctgaatttatgacttgaatacttaaattgttgttattcataggtattttactgattttaacatagaaaatgttattaatatttaaaagtctattgcaactaaatttaaatgaaatacagcaatattgaaagcttattaaatttgctattcctgatgaagtttgtacatcttcctccatgagtggatcaagaaatattgagatggctaagctggaaattacaaaaatgttggcatactattatagcatatggttataaaaatcaatgaatatttatatttagtattatgctttaagtatatatccaagctgatcaattaattacactttcactgatgagatgtcaattctacatttagctgaactctcatctgaactgtgtggcttctcaatgacaggacatattaaagaacatgatgaatgtttgtaatgtaatgatataaattattataatgtgttgcattaaagacacatggtgtagcattctacgttcagcttttgcatttattttctcagtgtcatgatttgctcctgtgattccagatcagttttctccttatcagtcagcatatacatattggtattaacactttttgcaaaaatcatatataaatggttgtagaatgttgttgtcattctacagcaactgtttagtttttgttcagcgattagcttgtttttcatttattttaagatttcaggccagtcgtggtggctcacgcctgtaattccagcactttgggagggcgacgcgggcggatcacaaggttaggagatccagaccatcctggccaatacggtgaaaccccatctctactaaaaatacaaaaaattagccgggcgtggtggtgggtgcctgtagtcccagctactctggaggctgaggcaggaaaatggcgtgaacttgggaggcggagcttgtagcgagccaagatctcgccagtgcactccagtctgggcaacagagtgatacgctatcaaaaaaaaaaaaaaatcatagtgtgcatttttactttacaccacctttaattagacgctattcatggtgactaaagagcaacataaatataattacggatgtcaacaagcctttgtttaagtgtatgtcctacttcatgctatactagaaattaaaagtgaagtactgaaaatccaagtacgtacagccatacatttcaatagccattacaaatgcggctcatgaatctttcgagctatgccatgtgacctgtcctgattctaaacaactccagtgtacccctttataaaaataaaagtaataaaaaaaaactaaggtggtattttcctacccggttcctctgcatgaattttgaacttcagggatgatcagatcacaatttagaaccagagttttcaaccttacataggattcttaagtgccaagtgataaatggttccttgatgatgagctcacctttactgtttcatctctgcgtgttttgcttttttatcttgtcttagaaaatttaatgccagttattttctttataaaggaaaatattttggttacatattcatttcctgtctcatgacactctgctttctttgcacttagtgaggatgtgtatttctcacagttttacctaaaacaagcagatgaattcatggtatcatttcttatatatgaattttggaggtttcttcagtgcttcagaagtaacttttaaagatattaatgaatagagaatgaccttctcattgtaattaaagcaggtttttatttaaagcgtattgaatacaaatttaagcactttttttctaacacacatacatgcaatatggtcatgcatatttagccttaaaatggttagacataaattttgtgtgttttagagagtttatgtccctgaagtgtcttcattattgatcaatgatctctcaagggaaacaacatatatagttgatatttctaaatatattaaagcatgtcatatggaaatatttgaggagtagagaaaaatgagtgattaaacatgtttgattaatattatttgaagacatgagatctatttctatctcaatgaaatgtctgtgattttcttgaagctaagttttttataagagcttattcataacaatagtttaacacctggcacggtgtaacaaatagaattagttttagaaacaaaactattgtaggattcattccttgaacactaaaactgttattttgaataagcattattgtaacattgaaatatgcaggtgaatgatatgtaaaaagtctctagaagtttgcttcttgtttgtaatacatccatataagatgttttctcgctctgtcatccaggctggagtgcagtggcatgatcttggctcattgcaacatctgcctcctgggttcaagtgattctggtacctcaggcacccaagtagctgggtttacaggcatgtaccaccatacctggctgatttttgtaattttagtggagacagtgtttccaaaatagtgattttggccaggctggtttggaactcctggcctccagagatccgccctcctcagccttccaaattgctggaattacaggcatgagccactgcacctggcctgtattggtttgttgatgggtacgcttggacctttttgtataagtggatcaggaaattttaagaggactaaactagagaaccctatgaatgtgaaaatactcgtatttcacagaggtacaaaaatgaatatatttattaacttttattctataagtagatatgctgctaaatttagaaccttctctgcaatgataagtacattgtacctttgaactctcatcacaactttgcaattcctaaactccaggacaaattgaagaacataatagctacatgtatatattgacataagtgattctgacgtgtttccttaacaatatgccatagcattctcccattagcttagtcatttattctgttttggtggggtgtggcatgacttggtcatcttattaaatacaacttctttccttatacggccgctttctcttactgatagtaggatatttctgctttagttattgtcaccttaaatatattttcaatgttgaaatcctcacagcatgtttgatgaaatctagttttcaaattttcttaggtatatttctgtcacgttggcatgataacaaatacaataacccaaaagaccccaaaacatagtgtaatccgttttgcaatccaagcatgaggattcatcttcatgttcacactgtgtgaatgttcggtaggctttgtcaggcttgcatataatcaattatgtatgtcccttttcttatagagtctcctaataaagatggtcttctgaaggtaataacttttatatttttgtcttgagtatcaactacatattttatgaagtatacattgtatattaattgttttgtttccaaacccatttaggctacctgcggaatgaaagtttctattccaactaaagccttagaattgaaggacatgcaaactttcaaagcaggtaaattttgtaattttaattttactctggaaaggagaatattaaaatatttgaaatgctgtgagacttttcattcccaatgttgttttctattcaaaatttgatgggatattttgatacaataatgccaatgtgagtatttctgtttgagaaaatgccatttacaagcataagatttagagatttagaaaaaaaattctgctttacctcatgtggttctactttaatatcctgatagtgtaaagtttccagtttgcaatttctgtacgtgcttggttttaaggcaggtgaattttgagactgaaatatttgcagtggttcaaaggctgattggaattccgatctttacttagaagaaagtttcacttgctgacatgacagttgtgagtgttgccactcagagaatattaagaaaatcagctttttttttgattagctgactgtgtgtgtgacttttaattttaaaaaatcagtcaagcaatcattacttgatgactctttgctagacatagtgttttagaagagtgactataaagcatttggccttggtgtcttttaatgctactgtaatgaattgcctagagatacaaaacagcctgaattagtttttgctgtcattcccatgcacgtttaaaacattttacaacaggctgtgcatggtgacacgtgcctgtaatcctagcattttgggagacaaaggcgggcagataagttaaggtcaggagtcccagaccagcctggtcaaattagtgaaaccccatctctactaaatataacaaaaaatagccggttgtgatggtgggtgcctgtcatctcagctactctggaggctgaggtgggagaatcgcttgaaactaggaggcaaaagttgcagtgagccgtccttttgccactttagcttgagtgacagagtgagactccatctgaaaaagcaaagaaataaaaaaacaaaaactcacaacatgtatgtggttatagaccatgtgtacaatttgttttcatgtcttatacgctgtgtgtagaatttgttttcatgtcttataggctatatgtagaatttgttttcatgtcttaaattttcttctatttttgtttgtatttttttcttcagtttttttgttttgttttgtttttgtttttttgagatcacccaggctggagtgccatggcgcgatctcggctcacgcaagctctgcctcccgggttcacgccattctcctgcctcagcctcccgagtagctgggactccaggcgcctgccaccatgcccggctaatgttttgtgtttttagtagagacagggtttcagtgtgttagccaggatggtctgatccggatctcctgaccttgtgatccgcccacctcggcctcccaaagtactggaattacaggcgtgaggcaccgtgcccggccgatgtctgaaattttcaataaatggttgtacactgtacatattgtcctggaacttccttgtttgcttatcgaatattttagatggacccaatacaattagctctgctttgatttaaggcctcatagtttgccatgttaattctaaaacacatttaattagaccgtcttttctagccactaaaagtcgacattaaatggcagctgatatcacagagcttttactgaagtgggggtatttactgcttcatccactgttagaaattaaaatgaaaatatttaaaatacacgaattggaaaagtcctacttttcaatacgcattacaactgttactcattaaccatgaaaattatgacattgtgacatgtcatgagtctgaaaaattttagtttacactttttagaaaatatcagtaaataggagaaataggaaaaacattatgtgaactattaggcccccggtgtatttgttgaacttcagagatgctcagatcagaagttagaacaaaaattttcaaaaatgcataaggttctaaagtgccaaataacaaatgggctcttgtatatgaaataatgtctgaagttgcacctctgagtcttttttctctttctcttttttttaaaaaaatataaatcaaacaaaatcaaaattttgtttataatgaaaagaattggttacagattcattctgtgtctcatggcactgtgctctctttttccctagagaggatctagacttttcatctgtttacatgggaagaagtagttcagtttatggtctcatttctcatataaattgtgaaaattctccacggcttcacatgctagttcagaagatattgatgccttgagaataaagcatctggttgtaactccagcagttttatatttaaagtatacctaatataattgtcaaccacattactttagaaaacataaacaaaagatagttacacatatttattttaaaaatggtgactgggtgcggtggctcacgcctgtaatcccagcacgttgggaggccgtgactggcagatcacgaggttaggagatcgagaccatcctgactaacaaggcgaaaccctgtctctactaaaaatacaaaaaaattagtcggacgtggtggcacgcatttctaataagttctcaggtgatgctgatgctggtggtccttggagcttgctctgagtagcaaaaggagaggcctttcatgggaaaatagtggaagaagagcagttggatagaaggtcaagacataacatggtcaggagaatgcattatattgcttttatttctgggtatgcttttagccagagaagaagaagaaaaagataaacagaaattatagatttaagatactatcactaaacaaagagaaaggaagtgttgaacaaataaatttgaaacagtgttgtatgggaagaattctacagagttagaggtttttttttattttctgtttttattttttatttatttatttttttagttgaagcattctaggcaggtgacaggggacaaacaagaaagaatgtaggcaggtaattctggagactctgagaagaacagttgagtgaactcacttcagttgcatttagattatttgtactccagaatgtagactgtgggcactccagagactaccggaagcaggagtcctactagaattgggggaaccacagtgactcattaggtttctctgttacaatcagggaagacaaatatatattttgttgctgttcgctatacaaatgacacatattgaaaactaagaacattggctttatgtttaaggaagtgtgttgttttggtaaaattgccattccacaaaaaatttattatagactaatgatacaccgaaccagacgaattgtaggaactgagaaaatactgaatttatacttgaataataagattgctttttaagataaatattgtggtgacttaacaatataaaaaagttatttatgtttaattcatctattgcaataaatttttatataaatatgtcaatattgaaagcttattatagattatttccatgatgagttttacacatcttcttgcatgagtggatccagaagcattcagacagctaaagtagaggatacagaaatataggcatatgattacaccatatggttatggaaaaaaacaaatattcatatttagtgttatgacctaagtgtatatccaagctgaacaattcataacatttcagagacaagatgtcagttctacattcagctgaactttcatcataactatgtacctttccgaagataggctatattaaagaacaggatgaatggaataatataagtgattctaatgtgtttcattaagtacgtggcgtagcattccatgttcagctttgacatttattttctcatatcaaccctttatacacatgaaacacaatctcgcttttgaagtcttaactgcatgatctgagaaacctgtatttatattttcttcagtgtattcttgtcgtgtgtgtgtcctaaacaaaccaaaagaaaactttccaaatctaaagtattcattctccaattggagcaagaggagtcagttaaatactatcacggcattcatttgtggttggcttgtcatatttacatatgattgatgataaatctcttttgctttttagagcctccggggaagccatctgccttcgaggtatttagttttatgatttcattttgaatgacttattatctatgtattttgtgaagtatacattctttattaatcattttgcttccaaccccatttagcctgccactgaaatgcaaaagtctgtcccaaataaagccttggaattgaaaaatgaacaaacattgagagcaggtaaatttttcaatgtaactatggaaagaccaatatttcaatattggacattttgatggtctttctgtacccaatggtttatttttttcaactttgatgaaaagatttgatctagataatgccaatactggtattgatgtttgaaaagctggtattacaagcacagtaattttcaatatattttttaaaaaaatgtagccttaatctcagatgtttctacttttgtatcctgaaactgtaatgttttctattttgaacttctgtatttcttaaggattcaagaaggtgaatgttgaaactccaatttctttttttagctcttcgaagcttgattcaaattccacggtttacttcggggatcccatctcttactgatataacccttgtgttttaacatgaattaccttgtttccggtgttgtcactttgagaatcctaagaaaatcagtaactcggactagtgaactctgtgtgtttatgtgtgtgtgtgtctgcgtgtgtgcctgcgtgtgcctgtgtgtgtgtggtacctttaccttgtaaagatgaggaaaggaattattcatttctttgtgaatatttgataaacacactttttcatgaaactgtgattctgaagcatttggctttagagtccttttacgctagtaccatttattgcctggaagtaaccaatattctaaactgtttttagaaactcttcttatgcatgtttaaacattttacaacatgtgtgcgtggtcatatttaatatgtacgatttttttcaacttctaatgtgtacatggttgtagagtgtagtgttcggcaacattcttttttgatcagcattataatttttagagacatccgtaaaggacacaattaattgtgtttttaaatattaggttatttataaaattccattgtatgacagtaccatagttaattgcacaatttttatgccgagaagtaataaataaaactgaaaacatgcaaatttgggagtctttaaaaagttagttttatctgctgatttcttagtcattagaaattaaaagtaacatatttaaagtatttccttgtgcaatcatagactccactaagaatttgaactgtgccccacagcttcttagagctatggtgtggcaacacgttagatctctgaaacgatccagggtacgcttctaaaaatgagtgaacatggtgacttaccaaagtgtgatttgagtttcctggaccctctgcatgaattgtgaacatgagctatgctgagatcacaagttaaatttacttttgaaaccaggtatacagttgatggatgtcaaatgataaacgtatgcatagtgatgaaacggtcttttaagttttaggtgtgcatgtttgcttttttcctgaacctgattcaaatagttgtaatttgtactttgtgctgataaagaaaactggacgttatttttggtataaattctttttctgtcttatgcccctgagaactcgtcaagtcttgagtgggccttgattttatcctattacacgtgggaacattagattacttaaggcaattatattttcctatacatttctgatgtttctccaaggtgtcacaagctgactctgaagatattgttgcattagggaagaagtgtgtcattgtaaatgaagcagttcttaatttatatgcaataaaatttttaaagcttatcttcctaaagcatatacacacgcaaaacacacccaatacactgtcatagcatacggaaatgtaaaagggttggaaatatagttgactgacatcaaaaagttaatattcctaaaacatcttattcattgataaatcatcttttttgaagaactgtgtaagagagattgctgagtcaatcaacgtaagtaatactaccaagtaaaaagtaacaaagaaatgagagatgataggtaattacagttttctgaatgaacaggaaacctagggcatactgtgtttcacaggagaataggatatgactgctttgtgaagaaataattcatacaagttagtcaaatttctatttttctgcattctaatgacataatgttaattgtcagagtttttagatttcaattcttttggaatgactatagaagtagtcattgtaatcaacaaaaagaacatatgggccacagaggaaaatccacagattcgtgaatgaaagtagatttgtatatgtttttaaaatttttagtagagaactgtgctcatgaatgtatctgtgattaaccttttatagatgagatactcccatcagaatccaaacaaaaggactatgaagaaaattcttgggatactgaggtactgtgtgttgttgatttttttaaatattagtattgcatgatatgaaaacataaaatcagatgcttagtctttattttctcacctctgcatgtgtcaccctcaaattatttttgatgtttttcagtatatgcttaatggagaatctatgtgctaagtagattcctgcttcacagtgaaattctgggaatttgtacgattaatttaagaagccaaagtggtatagtgtaaaaactaaggcttagaattcactagaaattcacatgggatctggagtacgtttgtctaaaagcaaaagaattacactgagtcgagctatgggcaaatatatgattctgtcttatatctagaggtagaaaagttctaattgtattcatagagagacactttaaactgcaatattgtaaaagttgggacctgaaaatttaatgcctgggacttgaacatattgacatatctttattgttcagtatagatctgaaggaacatttcagcagaaagaaaagcatgaggaataggaaaccttgtgggagtataataacaagagtattggttgagtagtcttttgaaaaatataaattatattttcacaaatagaactccttgagtccccttatggcagtcaagctacagcagcatgagcgtcaaatcatagtgatgtattttaaggtcacaacggcggaaagacatagaaagtatctgacctcttagaaacaagcagctgctgcctgatggtaacagcaaagggtggaagtcaataggtagattttatctgatattttattagacaaaaagacgttaatacttgttttctttcatttagatgtgacataacttttttcttactgcatttacattctattcaagcactttttctctcaccaaaaattttgtcagaaacatgttgcttattttaagcccctgtttaccgaaagaaagcagctttttcaaattctgtacatacattctatgacagactctaaaagttctcatcatgacatgcatgatttttaacactaaacagttctatgatcgtgaatattttaaatgtttactgctgtatgtgtccaggcaagtagcaaatgcgttgtattttgtttgaaatgtcttatttatttttgatgaggactagaacataagttagatatttttaagagagttactttttgaaatatgcacgagtgaatttttttgtgaaggtgatttgtttttcctgctcagtcaccgagttaatggccacgtgaacgtaaagatgagcttgatgtagagagggttatgtgaggttttctatcaaaatgtgtttgttttctacatgtatctgctcttaagtcgaattgtttgcaaaggaggaaattgtgtttttaaaaaagaatttaattaggaaattttgatactcttcattactaggatttatccattgaaattatttattgctattacttttaacagagtctctgtgagactgtttcacagaaggatgtgtgtttacccaaggctgcgcatcaaaaagaaatagataaaataaatggaaaattagaaggtaagaaccgttttttatttaaaaatcatttgaccaaatatttctctaaactgatgaggaaggatatcctctagtagctgaagaaaattacctcctaaatgcaaaccatggaaaaaaagagaagtgcaatggtcataagttatgtgtctcatcaggtgatggcaacagactatattgtgagtgctgaagcggagctgaattactagtttgaattcaagatattccaagacgtgaggaaaatgagaaaataagaaagaagaaagtagtaatagagtaaatgaagactgagaaagacagagcatacagagagtacatgaaggaacaagaagcaggtttatgtaatggaggatgataaaatgaaatgattgtttaggaaagatcggatgcaggggacaagagagaagccagctagatgattttggattttctatgaaggaaaatggagtgagctcacttcggaagcatttagaaaagttttactgcagaatgttagagtgtgggtgctctggagactactggaatcatgaggattactagaattggactaaacctcagtgactcattaattttctttattactatgaggcgtcaaatatatatatatatagatgtgtgcatgtatgtatgtttttgatgttcacaatttgaataagatatacttgaatgtaagaaccttggctttatttttaaggaagcaaattgtgttggtaaaattgccattttataaaacctcattagcaaataacatgatacacgaaaccagactaattttgaaaaccataaaactctgaatttatgacttgaatacctaaattgttgttattcgtaggtattttactgattttaacatagaaaatgttattaatatttaaaaatctgatgcaactaaatttaaatgaaatacatcaatattgaaagcttattaaatttgccattcctgatgagatttgtacatcttcctgcatgagtggattaagagatattgagatgactaagctagaaattacaaaaatgttggcatactattccagtatatgggtatgaaaatcaaggaatatttatatttaatattatgctctaagtatatatccaagctgatcaattcataacactttttctgatgagatgtcaattctacattcagctgaactcttatccgaactgtgtggcttctcaatgacaggacatattaaagaacatgatgaatgtttgtaatgcaatgatataaattattataatgtgttgccttaaagacacatggtgtagcattctacgttcagcttttgcatttattttctcagtgtcatgatttgctcctgtgattccagatcagttgtctccttatcagtcagcatatacttattggtattaacactttttgcaaaaatcatatataaatggttgtacaatgttgttgtcattctacagcaactttttaatttttgttcagcgattagcttgtttttcatttattttaagatttcaggccaggtgtggtggctcacgcctgtaatcccagcactttggtaggccaaggtgggtggatcacgaggtcaggagatccagaccatcctggctaacatggtgaaacctcacctctactagaaatacaaaaaatcagccgggcgtggtggtgggtgcctgtagtcccagctactccggaggctgaggcaagagaatggcgtgaacccgcgaggcggagcttgtggtgagccgatattacattattgtaacattgaaatatgcaggtgaatgatatataaaaagcctctggaagtttacttctagtttttgatactttcacgttacgtgttttttcttttttagagacagagtctcgctctgtcatccaggctggagtgcaatggcatgatcttggctcactgccacctccagctcctgggttcaagtgattctggtacctcagtcacccaagtagctgagattacaggcatgtgccaccatacctggctgatttttgtaattttagtggggacagtgtttctaaaatagtgattttggccaggctggtttggaactcctggcctccagagatccgccctcctcagccttccaaagtgctggaattacaggcatgagccactgcacctggcctgtattggtttgttgatgggtacgcttggacctttttgtataagtggatcaggaaattttaagaggactaaactagagaaccctatgaatgtgaaaatactcgtatttcacagaggtacaaaaatgaatatatttattaacttttattctataggtagatatgctgctgaatttagaacattctctgcaatgataagtacgttgtacctttgaactctcatcacaactttgcaattcctaaactccaggacaaattgaaggacataatagctacatgtatatattgacataagtgattctgacgtgtttccttaacaatatgccatagcattctcccattagcttagtcatttattctgttttggtggggtgtggcatgacttggtcatcttattaaatacaacttctttcctaatacggccgctttctcttactgatagtaggatatttctgctttagttattgtcaccttaaatatattttcaatgttgaaatcctcacagcatgtttgatgaaatctagttttcaaattttcttaggtatatttctgtcacgttggcatgataacaaatgcaataacccaaaagaccccaaaagctagtgtaatcccttttgcaatccaagcatgaggattcatcttcatgttgacagtgcgtgaatgttcggtaggctttgtcaagcttgcatacaataaattatatatgtcccttttcttttagggtctcctgttaaagatggtcttctgaaggtaataacttttatatttttatcttgagtattaactacatattttatgaagtatacattgtatattaattgttttctttccaaacccatttaggctaactgcggaatgaaagtttctattccaactaaagccttagaattgatggacatgcaaactttcaaagcaggtaaattttgtaattttaattttactctggaaagaagaatattaaaatatttgaaatgctgtgagacttttcattcccaatgttgttttctattcataatttgatgggaaatttcgatacaaataatgccaatgtgagtatttctgtttgagaaaatgccatttacaagcataagatttagagatttagaaaaaaaattctgctttacctcatgtggttctactttaatgtcctgatcggataaagtttccaatttgcaatttctgtacgcgcttggttttaaggtaggtgaattttgagactgtgaaatatttgcagtggttcgaaggctgattggaattctgatgtttactcagaagaaagtttcacttgctgacatgacagttgtgagtgttaccactctgagaatcttaagaaaatcagttttttttttaatagatgactgtgtgtgtgtgtgacttataattttaaaaaatcagtcaagcaatcattacttgatgactctttgctaggcaccgcattttagaagagtgaccccaaagcatttggccttggtgtgttttaatgctactgtaatgaattgcctagagatacaaaagagcctgaagtagtttttgtcattcccgtgaacgtttacaacattttacaacaggccttgcacggtggcacatgcctggaatcctagcagtttgggagacaaaggcgggcagataagttaaggtcaggagtcccagaccagcctggtcaaactagtgaaaccccatctctactaaacataacaaaaaatagccggttgtggtggtgggtgcctgtcatctcagctactctggaggctgaggcaggagaatggcttgaacctaggacgcagaagttgcatgagccgagcttgtgccactttagcctgagtgacagagtgagactccatctgaaaaggtaaagaaataaaaacacaaaactcacaacatatgtgtggttatagactatgtgtagaatttgttttcatgtcttataggctgtgtgtagaatttgttttcatgtcttaaattttctttttttttgtttgtatttttttcttcagtttttttgtttggttttgtttttgtttttttgagatcacccaggctggagtgccatggcgccatctcggctcacgcaagctctgcctcccgggttcacgccattctcctgcctcagcctcctgagtagctgagactccaggtgcctgccaccacgcccggctaatgttttgtatctttagtagagacagggtttcagtgtgttagccaggatggtctgatccggatctcctgaccttgtggtccgcccgtctcggcctcccaaagtactggaattacaggcatgaggcaccgtgcccggccgatgtctgaaattttcaataaatagttgtacgctgtacatattgtcctggaacttccttgtttgcttatcgaatattttagatggacccaatacaattagctctgctttgatttaaggcctcatagtttgccatgttaactctaaaacacatttaattagactgtgtttcctagccactaaaagtcgacattaaatggcagctgatatcacagagcttttactgaagtgggagtatttactgcttcattcactgttagaaattaaaatgaaaatatttaaaatacacgaattggaaaagtcctactttccaatacgcattacaactgttactcattaaccatgaaaattatgacattgtgacatgtcatgagtctgaaaaattttagtttacactttttagaaaatatcagtaaataggagaaataggaaaaacattatggcccccggtgtatttgttgaacttcagagatgctcagatcagaagctagaacaagaattttcaaaaatgcataaggttttaaagtgccaaataacaaatgggctcttgtatatgaaataatgtctgaagttgcacctctgagtcttttttctctttttcttttttaaaaagatataaatcaaacaaaatcaaacttttgtttataatgaaaagagttggttacagattcattctgtgtctcatggcactgtgctctctttttccctagagaggatctagacttttcatctgtttacatgggaagaagtagttcagtttatggtctcatttctcatataaactgtgaaaattctccacggcttcacatgctagttcagaagatattgatgccttgagaataaagcatctggttgtcactccagcagttttatatttaaagtatacctaatatagttgtcaaccacattactttagaaaacataaacaaaagatagttacacttatttatttaaaaaatggtgaccgggtgcggtggctcacgtctgtaatgccagcacgttgggaggccgtgactggcagatcacgaggtgaggagagcgagaccatcctgactaacgcggtgaaaccctgtctctactaaaaatacaaaaaattagccggacgtggtggcacgcacttctaataatttctcaggcgatgctgctgctggtggtccttggagcttggtctgagtagcaaaaggagaggcctttcatgggaaaatagtggaagaagagcagttggatagaaggtcaagacataacagggtcaagagaatgcattatattgcttttatttctgggtatgcttttagccagagaagaagaagaaaaagataaacagaaattatagatttaagatactatcactaaacaaagagaaaggaaatgttgaacaaataaatttgaaacagtgttgtatgggaagaattctacagagttagaggtttttttttaatttctttttttttttttttttttttttagtagaagcattcaagtcaggtaacgggacaaacaagaaagaatgtaggcaggtaattctggagactctgagaagaacagttgagtgaactcacttcagatgcatgtagattatttgcactccagaatgtagactgtgggcactccagagactaccagaattgggggaaccacagtgactcattaggtttctctgttacaatcaggcatgagaaatatatatattttgttgatgttagctattgaaatgagatatattggaaactgagaacattggctttatgtttaaggaagtgtgttgttttggtaaaattgccattccacaaaaatttattatagactaatgatacaccaaaccaggcgaattgtaggatctgaaaaaatactgaatttatacttgaataataagattgctttttaagataaatattgtggtgacttaacaatataaaaaaagttacttatgtttatttaatctattgcaataaatttttatataaatatgtcaatattgaaagcttagtatagattatttccatgatgagttttacacatcttgcatgagtggatcgagaagcattcagatggataaagtagaggatacagaatcataggcatatgattacaccctatggttatggaaaaaaacgaatattcatatttagtattatgacctaagtgtatatccaagctgatcaattcataacacttcagtgatgagatgtcagttgtacattcggctgaactctcatcataactatgtacctttccaaagataggctatattaaagaacaggatgaatggaataatataagtgattctaatgtgtttcattaagtacgtggcgtagcattccatgttcagctttgacatttattttctcatatcaaccctttttacacgtgaaacacaatctcgctcttgaagtcttaactgcatgatctgtgaaacctgtatttatattttcttcagtgtattcttgtcgtgtgtgtgtcctaaacaaaccaaaagaaaactttccaaatctaaagtattcattctccaattggagcaagaggagtcagttagatactatcacggcattcatttgtggctggcttgtcatatttacttatgattgataataaatctattttgctttttagagcctcccgagaagccatctgccttcgaggtatttagtttcatgatttcattttgaatgacttattaactatgtattttgtgaagtatacattctttattaatcattttgcttccaaccccatttagcctgccattgaaatgcaaaagtctgttccaaataaagccttggaattgaagaatgaacaaacattgagagcaggtaaattttttaatggaactatgcaaagaccaatatttcaatattggacattttgatggtccttctatccccaatgctttatttttttcaactttgatgaaaagatttgatctaggtaatgccaatactggtattgatgtttgaaaagctggtattacaagcacagtaattttcaatatctttttttaaaaatgtaggcttaatctcagatgtttctacttttgtatcctgaaactgtaatgttttctattttgaacttctgtatttcttaaggattcaagaaggtgaatgttgaaactccaatttctttttttagctcttcgaagcttgattcaaattccacggtttacttcggggatcccatctcttactgatataacccttgtgttttaacatgaattaccttgtttccggtgttgtcactttgagaatcctaagaaaatcagtaactcggactagtgaactctgtgtgtttatgtgtgtgtgtgtctgcgtgtgtgcctgcgtgtgcctgtgtgtgtgtggtacctttaccttgtaaagatgaggaaaggaattattcatttctttgtgaatatttgataaacacactttttcatgaaactgtgattctgaagcatttggctttagagtccttttacgctagtaccatttattgcctggaagtaaccaatattctaaactgtttttagaaactcttcttatgcatgtttaaacattttacaacatgtgtgcgtggtcatatttaatatgtacgatttttttcaacttctaatgtgtacatggttgtagagtgtagtgttcggcaacattcttttttgatcagcattataatttttagagacatccgtaaaggacacaattaattgtgtttttaaatattaggttatttataaaattccattgtatgacagtaccatagttaattgcacaatttttatgccgagaagtaataaataaaactgaaaacatgcaaatttgggagtctttaaaaagttagttttatctgctgatttcttagtcattagaaattaaaagtaacatatttaaagtatttccttgtgcaatcatacactccactaagaatttgaactgtgccccacagcttcttagagctatggtgtggcaacacgttagatctctgaaacgatccagggtacgcttctaaaaatgagtgaacatggtgacttaccaaagtgtgatttgagtttcctggaccctctgcatgaaatgtgaacatgagctatgctgagatcacaagttaaatttacttttgaaaccaggtatacagttgatggatgtcaaatgataaacgtatgcatagtgatgaaacggtcttttaagttttaggtgtgcatgtttgcttttttcctgaacctgattcaaatagttgtaatttgtactttgtgctgataaagaaaactggacgttatttttggtataaattctttttctgtcttatgtccctgagaactcgtcaagtcttgagtgggccttgattttatcctattacacgtgggaacattagattacttaaggcaattatattttcctatacatttctgatgtttctccaaggtgtcacaagctgactctgaagatattgttgcattagggaagaagtgtgtcattgtaaatgaagcagttcttaatttatacgcaataaaagttttttaagcttatcttcctaaagcttatacacacgcaaaacacacccaatacactgtcatagcatactgaaatgtaaaagggttggacatatagttgactgacatcaaaaagttaatatttctaaaacatcttattcattgataaatcatcttttttgaagaactgtgtaagagagattgctgagtcaatcaatgtaagtaatactaccaaataaaaagtaacaaagaaatgagagatgataggtaattccagttttctgaatgaacaggaaacctagggcatactgcgcttcacaggagaataggatatgactgctttgtgaagagataattcatacaagttagttaaatttctatttttctgcattctaatgaaataatgttaattttcagactttttagatttcagttctattggaatgactatagaagtagtcattgcaatcaacaaaaagaatatacgggctacagaggaaaaacctcagattcgtgaatgaaagtagatttgtatatgtttttaaaatttttagtagagaactgtgctcatgaatgtatctgtgattaaccttttatagatgagatactcccatcagaatccaaacaaaaggactatgaagaaagttcttgggattctgaggtactatgtgttattgattttttaaatattagtattgcatgatatgaaaacataaaatcagatgcttagactttatgttctcacctctgcatgtgtcacccccaaattatttttgatatttttcagtatatgcttaacggagaatctgtgtgctaagtagattcctgcttcacagtgaaattctgggaatttgtacgattaatttaagaagccaaagtggtatagtgtaaaaactaaggcttagaattcactagaaattcacatgggatctggagtacttttgtctaaaaggaaaagaattacactgagtccagctatgggcaaatatatgattctgtcttatatctagaggtagaaaagttctaattgtattcatagagagacactttaaactgcaatattgtaaaagttgggacctgaaaatttaatgcctgggacttgaacgtattgacatatctttattgttcagtatagatctgaaggaacatttcagcagaaaggaaagcatgaggaataggaaaccttgtgggagtataataacaagagtattggttgagtagtcttttgaaaaatataaattatattttcgcaaatagaactccttgagtccccttatggcagtcaagctgaagcagcatgagcgtcatatcatagtgatgtattttaaggtcacaacggcggaaagacatagaaagtatctgacctcttagaaacaagcagctgctgcctggtggtaacagcaaagggtggaagtcaataggtagattttatctgatattttattagacagaaagactttaacacttgtttgctttcatttagatgtgacataatttttttcttactgcatttacattctattcaaacactttttctatcaccaaaaattttgtcaaaaacatgttgcttattttaagcccctgtttaccgaaagaaagcagctttttcaaattctgtacatacattctatgacagactctaaaagttctcttcgtgacatgcatgatttttaacagtaaacagttctatgatcgtgaatattttaaatgtttactgctgtatgtgtcccggcaagtagcaaatgtgttgtattttgtttgaaatgtcttatttatttttgatgaggactagaacgtaagttagatatttttaagagagatactttttgaaatatgcacgagtgaattttttgtgaaggtgatttgtttttcctgctcagtcaccgagttaatggccacgtgaacgtaaagatgagcttgatgtagagagggttatgtgaggttttctatcaaaatgtgtttgttttctacatgtatctgctcttaagtcgaattgtttgcaaaggaggaaattgtgtttttaaaaaagaatttaattaggaaattttgatactcttcattactaggatttatccattgaaattatttattgctattacttttaacagagtctctgtgagactgtttcacagaaggatgtgtgtttacccaaggctgcgcatcaaaaagaaatagataaaataaatggaaaattagaaggtaagaaccgttttttatttaaaaatcatttgaccaaatatttctctaaattgatgaggaaggatatcctctagtagctgaagaaaattacctcctaaatgcaaaccatggaaaaaaagagaagtgcaatggtcataagctatgtgtctcatcaggcattggcaacagactatattgtgagtgctgaagaggagctgaattactagtttaaattcaagatattccaagacgtgaggaaaatgagaaaataagaaagaagaaagtagtaatagagtaaatgaagactgagaaagacagagcgtacagagagtacatgaaggaacaagaaacaggtttatgtaatggaggatgataaaatgaaatgattgtttaggaaaagatcgggtatggttagaaatttgggaagaatataatgtgaccttctagaatcaaaatttaccagagaattacagcaaaaatattctgactcttaatgtctttctcactggtgggaagccattagggatggaagcacctgaccatggagagctgtgttctatttgcaataggtgagaataagcatatctgcacggccacacatgtatataaattgtcatatacactccgtatagaccagaggttttcaaactttagaaagtcagctgaaaaccttgttaacaattcacactgagattcagccgacctgggattctgcatttttaaaaagttctcaggtgatgctgatgctggtggtccttggacgttgctctgggtagcaagaggagaggccttttgtgggaaaaatgtggaagcagagcaactggatagaaggtcaggacagaaaaggtcaggagaaaacatcatggtcctcttaattttgaggatgtttttagtcaggggagaagaagaaaggagcaatgcaatagaaattatagatggaaggtactaccactcctcaaagtaaaaggaaagagtgggaaaaaataattttaacaggtgtcgaatgggaagaatcaagagcacaagtctagtgattatttttgctaaagcagagagattgtgaggcaggaagcaggggacaagagagaagccagctagatgattttggattttctatgaaggaaaatggagtgagctcacttcggaagcatttagaaaagttttactgcagaatgttagagtgtgggtgctctggagactactggaatcatgaggattactagaattggactaaacctcagtgactcattaattttctttattactatgaggcgtcaaatatatatatatatatatatatatatatatatatatatatatatatatatagatgtgtgcatgtatgtatgtttttgacgttcacaatttgaataagatatacttgaatgtaagaaccttggctttatttttaaggaagcaaattgtgttggtaaaattgccattttataaaacctcattagtaaataacatgctacacgaaaccagactaattttgaaaaccataaaactctgaatttatgacttgaatacctaaattgttgttattcgtaggtattttactgattttaacgtagaaaatgttattaatatttaaaagtctgttgtaactaaatttaaatgaaatacatcaatattgaaagcttattaaatttgctattcctgatgagatttgtacatcttcctgcatgagtggattaagagatattgagatgactaagctagaaattacaaaaatgttggcatactattcctacattatgggaatgaatacccatattccaagtatatgggtatgaaaatcaaggaatatttatatttaatattatgctctaagtatatatccaagctgatcaattcataacactttttctgatgagatgtcaattctacattcagctgaactcttatccgaactgtgtggcttctcaatgacaggacatattaaagaacatgatgaatgtttgtaatgcaatgatataaattattataatgtgttgccttaaagacacatggtgtagcattctacgttcagcttttgcatttattttctcagtgtcatgatttgctcctgtgattccagatcagttgtctccttatcagtcagcatatacttattggtattaacactttttgcaaaaatcatatataaatggttgtacaatgttgttgtcattctacagcaactttttaatttttgttcagcgattagcttgtttttcatttattttaagatttcaggccaggtgtggtggctcacgcctgtaatcccagcactttggtaggccaaggtgggtggatcacgaggtcaggagatccagaccatcctggctaacatggtgaaacctcacctctactagaaatacaaaaaatcagccgggcgtggtggtgggtgcctgtagtcccagctactccggaggctgaggcaagagaatggcgtgaacccgcgaggcggagcttgtggtgagccgatattacattattgtaacattgaaatatgcaggtgaatgatatataaaaagcctctggaagtttacttctagtttttgatactttcacgttacgtgttttttcttttttagagacagagtctcgctctgtcatccaggctggagtgcaatggcatgatcttggctcactgccacctccagctcctgggttcaagtgattctggtacctcagtcacccaagtagctgagattacaggcatgtgccaccatacctggctgatttttgtaattttagtggggacagtgtttctaaaatagtgattttggccaggctggtttggaactcctggcctccagagatccgccctcctcagccttccaaagtgctggaattacaggcatgagccactgcacctggcctgtattggtttgttgatgggtacgcttggacctttttgtataagtggatcaggaaattttaagaggactaaactagagaaccctatgaatgtgaaaatactcgtatttcacagaggtacaaaaatgaatatatttattaacttttattctataggtagatatgctgctgaatttagaacattctctgcaatgataagtacgttgtacctttgaactctcatcacaactttgcaattcctaaactccaggacaaattgaaggacataatagctacatgtatatattgacataagtgattctgacgtgtttccttaacaatatgccatagcattctcccattagcttagtcatttattctgttttggtggggtgtggcatgacttggtcatcttattaaatacaacttctttcctaatacggccgctttctcttactgatagtaggatatttctgctttagttattgtcaccttaaatatattttcaatgttgaaatcctcacagcatgtttgatgaaatctagttttcaaattttcttaggtatatttctgtcacgttggcatgataacaaatgcaataacccaaaagaccccaaaagctagtgtaatcccttttgcaatccaagcatgaggattcatcttcatgttgacagtgcgtgaatgttcggtaggctttgtcaagcttgcatacaataaattatatatgtcccttttcttttagggtctcctgttaaagatggtcttctgaaggtaataacttttatatttttatcttgagtattaactacatattttatgaagtatacattgtatattaattgttttctttccaaacccatttaggctaactgcggaatgaaagtttctattccaactaaagccttagaattgatggacatgcaaactttcaaagcaggtaaattttgtaattttaattttactctggaaagaagaatattaaaatatttgaaatgctgtgagacttttcattcccaatgttgttttctattcataatttgatgggaaatttcgatacaaataatgccaatgtgagtatttctgtttgagaaaatgccatttacaagcataagatttagagatttagaaaaaaaattctgctttacctcatgtggttctactttaatgtcctgatcggataaagtttccaatttgcaatttctgtacgcgcttggttttaaggtaggtgaattttgagactgtgaaatatttgcagtggttcgaaggctgattggaattctgatgtttactcagaagaaagtttcacttgctgacatgacagttgtgagtgttaccactctgagaatcttaagaaaatcagttttttttttaatagatgactgtgtgtgtgtgtgacttataattttaaaaaatcagtcaagcaatcattacttgatgactctttgctaggcaccgcattttagaagagtgaccccaaagcatttggccttggtgtgttttaatgctactgtaatgaattgcctagagatacaaaagagcctgaagtagtttttgtcattcccgtgaacgtttacaacattttacaacaggccttgcacggtggcacatgcctggaatcctagcagtttgggagacaaaggcgggcagataagttaaggtcaggagtcccagaccagcctggtcaaactagtgaaaccccatctctactaaacataacaaaaaatagccggttgtggtggtgggtgcctgtcatctcagctactctggaggctgaggcaggagaatggcttgaacctaggacgcagaagttgcatgagccgagcttgtgccactttagcctgagtgacagagtgagactccatctgaaaaggtaaagaaataaaaacacaaaactcacaacatatgtgtggttatagactatgtgtagaatttgttttcatgtcttataggctgtgtgtagaatttgttttcatgtcttaaattttctttttttttgtttgtatttttttcttcagtttttttgtttggttttgtttttgtttttttgagatcacccaggctggagtgccatggcgccatctcggctcacgcaagctctgcctcccgggttcacgccattctcctgcctcagcctcctgagtagctgagactccaggtgcctgccaccacgcccggctaatgttttgtatctttagtagagacagggtttcagtgtgttagccaggatggtctgatccggatctcctgaccttgtggtccgcccgtctcggcctcccaaagtactggaattacaggcatgaggcaccgtgcccggccgatgtctgaaattttcaataaatagttgtacactgtacatattgtcctggaacttccttgtttgcttatcgaatattttagatggacccaatacaattagctctgctttgatttaaggcctcatagtttgccatgttaactctaaaacacatttaattagactgtgtttcctagccactaaaagtcgacattaaatggcagctgatatcacagagcttttactgaagtgggagtatttactgcttcatccactgttagaaattaaaatgaaaatatttaaaatacacgaattggaaaagtcctactttccaatacgcattacaactgttactcattaaccatgaaaattatgacattgtgacatgtcatgagtctgaaaaattttagtttacactttttagaaaatatcagtaaataggagaaataggaaaaacattatgtgaactattaggcccccggtgtatttgttgaacttcagagatgctcagatcagaagctagaacaagaattttcaaaaatgcataaggttttaaagtgccaaataacaaatgggctcttgtatatgaaataatgtctgaagttgcacctctgagtcttttctctctttttcttttttaaaaagatataaatcaaacaaaatcaaaattttgtttataatgaaaagagttggttacagattcattctgtgtctcatggcactgtgctctctttttccctagagaggatctagacttttcatctgtttacatgggaagaagtagttcagtttatggtctcatttctcatataaactgtgaaaattctccacggcttcacatgctagttcagaagatattgatgccttgagaataaagcatctggttgtaactccagcagttttatatttaaagtatacctaatataattgtcaaccacattactttagaaaacataaacaaaagatagttacacttatttatttaaaaaatggtgaccgggtgcggtggctcacgtctgtaacgccagcacgttgggaggccgtgactggcagatcacgaggttaggagatcgagaccatcctgactaacacggtgaaaccctgtctctactaaaaatacaaaaaattagccggacgtggtggcacgcatttctaataatttctcaggcgatgctgctgctggtggtccttggagcttggtctgagtagcaaaaggagaggcctttcatgggaaaatagtggaagaagagcagttggatagaaggtcaagacataacagggtcaagagaatgcattatattgcttttatttctgggtatgcttttagccagagaagaagaagaaaaagataaacagaaattatagatttaagatactatcactaaacaaagagaaaggaaatgttgaacaaataaatttgaaacagtgttgtatgggaagaattctacagagttagaggtttttttttaatttctttttttttttttttttttttagtagaagcattcaagtcaggtaacgggacaaacaagaaagaatgtaggcaggtaattctggagactctgagaagaacagttgagtgaactcacttcagatgcatgtagattatttgcactccagaatgtagactgtgggcactccagagactaccagaattgggggaaccacagtgactcattaggtttctctgttacaatcaggcatgagaaatatatatattttgttgatgttagctattgaaatgagatatattggaaactgagaacattggctttatgtttaaggaagtgtgttgttttggtaaaattgccattccacaaaaatttattatagactaatgatacaccaaaccaggcgaattgtaggatctgaaaaaatactgaatttatacttgaataataagattgctttttaagataaatattgtggtgacttaacaatgtaaaaaaagttacttatgtttatttaatctattgcaataaatttttatataaatatgtcaatattgaaagcttagtatagattatttccatgatgagttttacacatcttgcatgagtggatcgagaagcattcagatggataaagtagaggatacagaatcataggcatatgattacaccctatggttatggaaaaaaacgaatattcatatttagtattatgacctaagtgtatatccaagctgatcaattcataacacttcagtgatgagatgtcagttgtacattcggctgaactctcatcataactatgtacctttccaaagataggctatattaaagaacaggatgaatggaataatataagtgattctaatgtgtttcattaagtacgtggcgtagcattccatgttcagctttgacatttattttctcatatcaaccctttttacacgtgaaacacaatctcgctcttgaagtcttaactgcatgatctgtgaaacctgtatttatattttcttcagtgtattcttgtcgtgtgtgtgtcctaaacaaaccaaaagaaaactttccaaatctaaagtattcattctccaattggagcaagaggagtcagttagatactatcacggcattcatttgtggctggcttgtcatatttacttatgattgataataaatctattttgctttttagagcctcccgagaagccatctgccttcgaggtatttagtttcatgatttcattttgaatgacttattaactatgtattttgtgaagtatacattctttattaatcattttgcttccaaccccatttagcctgccattgaaatgcaaaagtctgttccaaataaagccttggaattgaagaatgaacaaacattgagagcaggtaaattttttaatggaactatgcaaagaccaatatttcaatattggacattttgatggtccttctatccccaatgctttatttttttcaactttgatgaaaagatttgatctaggtaatgccaatactggtattgatgtttgaaaagctggtattacaagcacagtaattttcaatatctttttttaaaaatgtaggcttaatctcagatgtttctacttttgtatcctgaaactgtaatgttttctattttgaacttctgtatttcttaaggattcaagaaggtgaatgttgaaactccaatttctttttttagctcttcgaagcttgattcaaattccacggtttacttcggggatcccatctcttactgatataacccttgtgttttaacatgaattaccttgtttccggtgttgtcactttgagaatcctaagaaaatcagtaactcggactagtgaactctgtgtgtttatgtgtgtgtgtgtctgcgtgtgtgcctgcgtgtgcctgtgtgtgtgtggtacctttaccttgtaaagatgaggaaaggaattattcatttctttgtgaatatttgataaacacactttttcatgaaactgtgattctgaagcatttggctttagagtccttttacgctagtaccatttattgcctggaagtaaccaatattctaaactgtttttagaaactcttcttatgcatgtttaaacattttacaacatgtgtgcgtggtcatatttaatatgtacgatttttttcaacttctaatgtgtacatggttgtagagtgtagtgttcggcaacattcttttttgatcagcattataatttttagagacatccgtaaaggacacaattaattgtgtttttaaatattaggttatttataaaattccattgtatgacagtaccatagttaattgcacaatttttatgccgagaagtaataaataaaactgaaaacatgcaaatttgggagtctttaaaaagttagttttatctgctgatttcttagtcattagaaattaaaagtaacatatttaaagtatttccttgtgcaatcatacactccactaagaatttgaactgtgccccacagcttcttagagctatggtgtggcaacacgttagatctctgaaacgatccagggtacgcttctaaaaatgagtgaacatggtgacttaccaaagtgtgatttgagtttcctggaccctctgcatgaaatgtgaacatgagctatgctgagatcacaagttaaatttacttttgaaaccaggtatacagttgatggatgtcaaatgataaacgtatgcatagtgatgaaacggtcttttaagttttaggtgtgcatgtttgcttttttcctgaacctgattcaaatagttgtaatttgtactttgtgctgataaagaaaactggacgttatttttggtataaattctttttctgtcttatgtccctgagaactcgtcaagtcttgagtgggccttgattttatcctattacacgtgggaacattagattacttaaggcaattatattttcctatacatttctgatgtttctccaaggtgtcacaagctgactctgaagatattgttgcattagggaagaagtgtgtcattgtaaatgaagcagttcttaatttatacgcaataaaagttttttaagcttatcttcctaaagcttatacacacgcaaaacacacccaatacactgtcatagcatactgaaatgtaaaagggttggacatatagttgactgacatcaaaaagttaatatttctaaaacatcttattcattgataaatcatcttttttgaagaactgtgtaagagagattgctgagtcaatcaatgtaagtaatactaccaaataaaaagtaacaaagaaatgagagatgataggtaattccagttttctgaatgaacaggaaacctagggcatactgcgcttcacaggagaataggatatgactgctttgtgaagagataattcatacaagttagttaaatttctatttttctgcattctaatgaaataatgttaattttcagactttttagatttcagttctattggaatgactatagaagtagtcattgcaatcaacaaaaagaatatacgggctacagaggaaaaacctcagattcgtgaatgaaagtagatttgtatatgtttttaaaatttttagtagagaactgtgctcatgaatgtatctgtgattaaccttttatagatgagatactcccatcagaatccaaacaaaaggactatgaagaaagttcttgggattctgaggtactatgtgttattgattttttaaatattagtattgcatgatatgaaaacataaaatcagatgcttagactttatgttctcacctctgcatgtgtcacccccaaattatttttgatatttttcagtatatgcttaacggagaatctgtgtgctaagtagattcctgcttcacagtgaaattctgggaatttgtacgattaatttaagaagccaaagtggtatagtgtaaaaactaaggcttagaattcactagaaattcacatgggatctggagtacttttgtctaaaaggaaaagaattacactgagtccagctatgggcaaatatatgattctgtcttatatctagaggtagaaaagttctaattgtattcatagagagacactttaaactgcaatattgtaaaagttgggacctgaaaatttaatgcctgggacttgaacgtattgacatatctttattgttcagtatagatctgaaggaacatttcagcagaaaggaaagcatgaggaataggaaaccttgtgggagtataataacaagagtattggttgagtagtcttttgaaaaatataaattatattttcgcaaatagaactccttgagtccccttatggcagtcaagctgaagcagcatgagcgtcatatcatagtgatgtattttaaggtcacaacggcggaaagacatagaaagtatctgacctcttagaaacaagcagctgctgcctggtggtaacagcaaagggtggaagtcaataggtagattttatctgatattttattagacagaaagactttaacacttgtttgctttcatttagatgtgacataatttttttcttactgcatttacattctattcaaacactttttctatcaccaaaaattttgtcaaaaacatgttgcttattttaagcccctgtttaccgaaagaaagcagctttttcaaattctgtacatacattctatgacagactctaaaagttctcttcgtgacatgcatgatttttaacagtaaacagttctatgatcgtgaatattttaaatgtttactgctgtatgtgtcccggcaagtagcaaatgtgttgtattttgtttgaaatttcttatttatttttgatgaggactagaacgtaagttagatatttttaagagagatactttttgaaatatgcacgagtgaattttttgtgaaggtgatttgtttttcctgctcagtcaccgagttaatggccacgtgaacgtaaagatgagcttgatgtagagagggttatgtgaggttttctatcaaaatgtgtttgttttctacatgtatctgctcttaagtcgaattgtttgcaaaggaggaaattgtgtttttaaaaaagaatttaattaggaaattttgatactcttcattactaggatttatccattgaaattatttattgctattacttttaacagagtctctgtgagactgtttcacagaaggatgtgtgtttacccaaggctacgcatcaaaaagaaatagataaaataaatggaaaattagaaggtaagaaccgttttttatttaaaaatcatttgaccaaatatttctctaaattgatgaggaaggatatcctctagtagctgaagaaaattacctcctaaatgcaaaccatggaaaaaaagagaagtgcaatggtcataagctatgtgtctcatcaggcattggcaacagactatattgtgagtgctgaagaggagctgaattactagtttaaattcaagatattccaagacgtgaggaaaatgagaaaataagaaagaagaaagtagtaatagagtaaatgaagactgagaaagacagagcgtacagagagtatatgaaggaacaagaagcaggtttatgtaatggaggatgataaaatgaaatgattgcttaggaaaagatcgggtatggttagaaatttgggaagaatataaagtgaccttctagaaccaaaatttaccagagaattacagcaaaaatattctgactcttaatgtctttctcacttgtgggaagccattagggatggaagcacctgaccatggagagctgtgttctatttgcaataggtgagaataagcatatctgcatggccacacatgtatataaattgtcatatacactctgtatagaccagaggttttcaaactttagaaagtcagctgaaaaccttgttaacaattcacactgagattcagccgacttgggattctgcatttttaaaaagttctcaggtgatgctgatgctggtggtccttggacgttgctctgggttgcaagaggagaggccttttgtgggaataatgtggaagcagagcaactggatagaaggtcaggacagaaaaggtcaggagaaagcatcatggtgctcttaattttgaggatgtttttagtcaggggagaagaagaaaggagcaatgcaatagaaattatagatggaagatactaccactcctcaaagtaaaaggaaagagttggaaaaaataatgttaacaggtgtcgaatgggaagaatcaagagcagaagtctagtgattatttttgctaaagcagagagattgtgaggcaggaagcaggggacaagagagaatccagctagatgattttggattttctatgaaggaaaatggagtgagctcacttcggaagcatttagaaaagttttactgcagagtgctagagtgtgggtgctctggagactactggaatcatgaggattactagaattggactaaacctcagtgactcattaataatctttattaatatgaagcatcaatatatagatatatatatatagatgtatgtatgtatgtatgtttttgacgttcacaatttgaataagatatacttgaatgtaagaaccttggctttatttttaaggaagcaaattgtgttggtaaaattgccattttatgaaacctcattagcaaataacatgctacacgaaaccagactaattttaaaaaccataaaactctgaatttatgacttgaatacctaaattgttgttattcgtaggtattttactgattttaacatagaaaatgttattaatatttaaaagtctgttgcaactaaatttaaatgaaatacatcaatattgaaagcttattaaatttgctattccttgatgagatttgtacatcttcctccatgagtggattaagagatattgagatgagtaagctagaaattacaaaaatgttggcatactattccagtatatgggtatgaaaatcaaggaatatttatatttaatattatgctctaaatatatatccaagctgatcaattcataacactttttctgatgagatgtcaattctacattcagctgaactctcatccgaactgtgtggcttctcaatgacaggacatattaaagaacatgatgaatgtttgtaatgcaatgatataaattattataatgtgttgccttaaagacacatggtgtagcattctacgttcagcttttgcatttattttctcagtgtcatgatttgctcctgtgattccagatcagttttctccttatcagtcagcatatacatattggtattaacactttttgcaaaaatcatatataaatggttgtacaatgttgttgtcattctacagcaactttttaatttttgttcagcgattagcttgtttttcatttattttaagatttcaggccaggtgtggtggctcacgcctgtaatcccagcactttgggaggcaaaggtgggtggatcacgaggtcaggagatccagaccatcctggctaacatggtgaaaccccacctctactagtaatacaaaaaatcagccgggcgtggtggtgggtgcctgtagtcccagctactccggaggctgagacaaaagaatggcgtgaacccgcgaggcggagcttgtggtgagccgatattacattattgtaacattgaaatatgcaggtgaatgatatataaaaagcctctggaagtttacttctagtttttgatactttcacattacatgttttttcttttttagagacagagtctcgctctgtcatccaggctggagtgcaatggcatgatcttggctcactgcaacctccacctcctgggttcaagtgattctggcacctcaggcacccaagtagctgagattacaggcctgtgccaccatacctggctgatttttgtaattttagtggggacagtgtttctaaaatagtgattttggccaggctggtttggaactcctggcctccagagatccgccctcctcggccttccaaattgctggaattacaggcatgagccactgcacctggcctgtattggtttgttgatgggtatgcttggacctttttgtataagtggatcaggaaattttaagaggactaaactagagaaccctatgaatgtgaaaatactcgtatttctcagaggtacaaaaatgaatatatttattaacttttattctataagtagaaatgctgctaaatttagaacattctctgcaatgataagtacatttgtacctttgaactctcatcacaactttgcaattcctaaactccaggacaaattgaagaacataatagcaacatgtatatattgacatcagtgattctgacgtgtttccttaacaatatgccatagcattctcccattagcttagtcatttattctgttttggtggggtgtggcatgacttggtcatcttattaaatacaacttctttccttatacggccgctttctcttactgatagtaggatatttctgctttagttattgtcaccttaaatatattttcaatgttgaaatcctcacagcatgtttgatgaaatctagttttcaaattgtcttaggtatatttctgtcacgttggcatgataacaaatacagtaacccaaaagaccccaaaagctagtgtaatcccttttgcaatccaagcatgaggattcatcttcatgttgacagtgcgtgaatgttcggtaggctttgtcaagcttgcatacaataaattatatatgtcccttttcttttagagtctcctgataatgatggttttctgaaggtaataacttttatatttttatcttgagtattaactacatattttatgaagtatacattgtatattaattgttttgtttctaaacccatttaggctccctgcagaatgaaagtttctattccaactaaagccttagaattgatggacatgcaaactttcaaagcaggtaaattttgtaattttaattttactctggaaagaagaatattaaaatatttgaaatgctgtgagacttttcattcccaatgttgttttctattcataatttgatgggaaatttcgatacaaataatgccaatgtgagtatttctgtttgagaaaatgccatttataagcctaagatttagagatttagaaaaaaaattctgctttacctcatgtggttctactttaatgtcctgatcggataaagtttccaatttgcaatttctgtacgcgcttggttttaaggtaggtgaattttgagactgtgaaatatttgcagtggttcaaaggctgattggaattctgatgtttacttagaagaaagttccacttgctgacatgacagttgtgagtgttaccactctgagaatcttaagaaaatcagttttttttttattagatgactgtgtgtttgtgtgacttataattttaaaaaatcagtcaagcaatcattacttgatgactcttgtctagacacggtgttttagaagagtgaccccaaagcatttggccttggtgtcttttaatgctactgtaatgaattgcctagaggtacaaaagagcctgaattagttcttgttgtcattcccgtgaacgtttacaacattttacaataggccgtgcacggtggcacgtgcctgtaatgatagcagtttgggagagaaaggcagacagataagttaaggtcaggagtcccagaccagcctggtcaaattagtgaaaccccatctctactaaacataacaaaaagtagccggttgtggtggtgggtgcctgtcatctcagctactctggaggctgaggcaggagaatggcttgaacgtaggaggcagaagttgcatgagccgagcttgtgccactttagcctgagtgacagagtgagactccatctgaaaaagcaaagaaataaaaacacaaaagctcacaacatatgtgtggttatagactatgtgtagaatttgttttcatgtcttataggctatgtgtagaatttgttttcatgtcttcaattttcttccttttttttgtttgcatttctttcttcagtttttttgttttgttttgtttttgtttttttgagctcgaccaggctggagtgccatggcgccatctcggctcatgcaagctctgcctcccgggttcacgccattctcctgcctcagcctcccgagtagctgggactccaggcgcgtgccaccacgcctggctaatgttttgtatttttagtagagacagggtttcagtgtgttagccaggatggtctgatccggatctcctgaccttgtgatgcgcccgtctcggcctcccaaagtactggaattacaggcgtgaggcaccgtgcccggccgatgtctgaaattttcaataaatagttgtacactgtacgtattgtcctggaacttccttgtttgcttatcgaatattttagatggacccaatacaattagctctgctttgatttaagacctcatagtttgccatgttaactctaaaacacatttaattagaccgtcttttctagccactaaaagtcgaaattaaatggcagctgatatcacagagcttttactgaagtgggagtatttactgcttcatccactgttagaaattaaaatgaaaatatttaaaatacacgaattggaaaagtcctactttccaatatgcattacaactgttagttgttaaccatgaaaattatgacattgtgacatgtcatgagtctgaaaaattttagtttacacttcttagaaaatatcagtaaatagaagaaataggaaaaacattatgtgaactattaggcccccggtgtatttgttgaacttcagagatgctcagatcagaagttagaacaagaattttcaaaaatgcataaggttttaaagtgccaaataacaaatgggctcttgtatatgaaataatgtctgaagttgcacgtctgagtcttttttctctttttctttttcaaaaagatataaatcaaacaaaatcaaaattttgtttataatgaaaagagttggttacagattcattctgtgtctcatggcgctgtgctctcttccctagagaggatctagacttttcatctgtttacatgggaagaagtagttcagtttatggtctcatttctcatataaactgtgaaaattctccacggcttcacatgctagttcagaagatattgatgccttgagaataaagcatctggttgtaactccagcagttttatatttaaagtatacctaatataattgtcaaccacattactttagaaaacataaacaaaagatagttacacatatttatttaaaaaatggtgaccgggtgcggtggctcacgcctgtaatgccagcacgttgggaggccgtgactgacagatcactaggtgaggagatcgagaccatcctgactaacacagtgaaaccctgtctctactaaaaatacaaaaaattagccggacgtggtggcacgcatttctaataatttctcaggcgatgctgctgctggtggtccttggagcttgctctgagttgcaaaaggagaggcctttgatgggaaaaattggaagaagagcagttggatagaaggtcaagacataacagggtcaagagaatgcattatattgcttttatttctgggtatgcttttagccagagaagaagaaggaaaagataaacagaaattatagatttaagatactatcactaaacaaagagaaaggaagtgttgaacaaataaatttgaaacagtgttgtatgggaagaattctacagagttagaggtttttttatattttctatttttttttttttttttttgtagtagaagcattctaggcaggtaacaggggacaaacaagaaagaatgtaggcaggtaattctggagactctgagcaaaacagttgagtgaactcacttcagatgcatgtagattatttgcactccagaatgtagactgtgggcactccagagactaccggaagcaggagtcctactagaattgggggaaccacagtgacacattaggtttttctgttacaatgaggcatgacaaatatatatattttgttgatgttagctattgaaatgagatatattggaaactaagaacattgggtttatgtttaaggaagtgtgttgttttggtaaaattgccattccataaaaatttattatagactaatgatacactgaaccagacgaattgtaggaactgtaaaaatactgaatttatacttcaaaaataagattgctttttaagataaatattgtggtgacttaacaattaaaaaagttatttatgtttaattcatctattgcaataaatttttatagaaatatgtcaatattgaaagcttagtgtagattatttccatgatgagttttacacatcttcttgcatgagtggatcaagaagcattcagatgaataaagcagaggatacagaaatataggcatatgattacaccctatggttatggaaaaaaatgaatattcattttaagtattatgacctaagtgtatatccaagctgatcaattcaaaacacttcagtgacgagatgtcagttgtacattcagctgaactctcatcataaccatgtacctttccgaagataggctatgttaaagaacaggatgaatggaataatataagtgattctaatgtgtttcattaagtacgtggcgtagcattccatgttcagctttgacatttattttctcatatcaaccctgtttacacgtgaaacacaatctcgctcttgaagtcttaactgcatgatctgtgaaacctgtatttatattttcttcagtgtattcttgtcgtgtgtgtgtcctaaacaaaccaaaagaaaactttccaaatctaaagtattcattctccaattggagcaagaggagtcagttagatactatcacggcattcatttgtggctggcttgtcatatttacttatgattgatgataaatctcttttgctttttagagcctcccgagaagccatctgccttcgaggtatttagttttatgatttcattttgaatgacttattaactatgtattttgtgaagtatacattctttattaatcattttgcttccaaccccatttagcctgccattgaaatgcaaaagtctgttccaaataaagccttggaattgaagaatgaacaaacattgagagcaggtacatttttcaatgtaactatgcgaagaccaatatttcaatattgaacattttgatggtctttctatccccaatgctttatttttttcaactttgatgataagttttgatctaggtaatgccaatactggtattgatgtttgaaaagctggtattacaagcacagtaattttcaatattttttttaaaaaatgtagccttaatctcagatgtttctacttttgtatcctgaaactgtaatgttttctattttgaacttctgtatttcttaaagattcaagaaggtgaatgttgaaactccaatttctttttttagttcttcgaagcttgattcaaattccacggtttatttcggggatcccatcttttactgatataacactcgtgttttaacttgaattacctcgtttccagtgttgtcactttgagaatcctaagaaaatcagtaactcggattagtgaactctgtgtgtttatgtgtgtgtgtctgcgtgtgtgcctgtgtgtgcctgtgtatgtgtgtggtaactttaccttgtgaagatgaggaaaagaattattcatttctttgtgaatatttgataaacacactttttcatgaaactgtgattctgaagtgtttggctttagagtctttttacactagtaccatttattgcccagaagtaaccaatattctaaactgttttttaaaactattcttatgcatgtttaaacattttacaacatgtgtgcgtggtcatatttaatatgtacaatttttttcaacttctaatatgtagatggttgtgcagtgtaatgttcggcaacattcgtttttgatcagcattataatttttagaaacatccgtataggacacagttaactgtgtttttaatattaggttatttataaaattccattttatgacagtaccatagttaactgcacaatttttatgctgataagtaataaataaaaatgaaaacatgcagatttctgcatctttaaaaggttagttttatctgctgattttttggttattagaaattaaaagtaaaatatttaagatatttccttgttcaatcatgcattccaccaagaatttgaactgtgccccagagcttcttagagatatggtgttgcaacacgttgtatctctgaaactatccagggttcacttccaaaaatgagtgaaaacggtggcttaccaaagtatgatttaagtttcttcgacactctgcatgaaatatgaacgttagctatgctgtgatcacaagttaaatttacttttgaaaatcaggtatacaattgatggatgtcaaatgatatacgtacccttaatgatgaaatggtcttttaagttttagttgtgcatgtttgcttttttctttaacctgattcaaatagttgtaatttgtactttgtgctgataaagaaacttcaacgttatttttggtataaattcattttctgtctcatgttcctgagaactcctcaagtcttgagtgggccttgattttatcctataacatgtgggaacgttggattacttaaggcaattattttttcctatacatttctcatgtttctccaaggtgtcacaagctgactctgaaggcattgttgcattagggaagaagtatgtcattgtaattaaagcagttcttaattcatatgcaataaaagttttttaagcttatcttcctaaaacatatacatacccaaaccacacccaatacactgtcatagcatattgaaatgtaaaagggttggacatatagttgactgacatcaaaaagttaatattcctaaaaaatcttattcattgataaatcatcttttttgaagaactgcgtaatagagattgctgagtcaatcaacgtaagtaatactaccaaataaaaatttacaaagaaatgagagatggtaggtaattaaagttttctgaatgaacaggaaatataggatataccttgtttcagagtagaagagtatgtgactgctttgtgaagaaataattcatagaagttagtcaaatttatatttttgtgcattctaatgaaataatgttaattttcagacttttcagatttcaattctaacggattgactataggaagtagtcattgtaatcaacaaaaagaatatacgggctacagaggaggaaccacagattcgtgaatgaaagtagatttgtatatgtttttaaaatgtatagtagagaaatgttctcatgaatgtatctgtgattaaccttttatagatcagatgttcccttcagaatcaaaacaaaagaaggttgaagaaaattcttgggattctgaggtactatgtgttattgatttttttaaatattagtattgcatgatatgaaaacataaaatcagatgcttagactttattttctcacctctgcatatgtcacccgcaaattatttttgatatttttcagaatatgcttaatagagaatctatgtgctaagtagattactgcttcacagtgatattgtgtgaatttgtaggattaatttaagaagcccgtgtggtacagtggaaataataagagttagaattcactagaaattcacatgggatctgaagtacgtttgtctaaaagcaaaagaattacactgagtccagctctgggcaaatatatgattctgtcttatatctagatgtacaaaagttctaattggattcatagagagacagtttaagctgcagtattgtaaaagttgggacctgaaaagttaatgcctgggacttgaacgtattgacatatctttattgttcagtatagatctgaaggaacatttcagcagaaagaaaagcatgaggaataggaaaccttgtgggagtataataacaagagtattggttgagtagtcttttgaaaaatataaattatattttcacaaatggaactccttgagtccctttatgacagtcaagctgcagcagcatgagcgtcaaataatagtgatgtattttaaggtcacaacggcggaaagacatagaaagtatctgacctcttagaaacaagcagctgctgcctggtggtaacagcaaagggtggaagtcaatagacaggtagattttatctgatttgtaattggacaaaaagactttaatacttatttgcttttatttagacatgacataatttttttcttactgcatttgcattctattcaagcacttttttatcaccataaattttgtcaaaaacttgttgctgattttaagcccttgtttatcaaaataaagcagctttttcaacattctgtacatacattctatgacagactctaaaagttctcttcatgacaggcatcatttttaacattagacaattgtatgatcatgcatattttacatgtttaatgcagtatctgttatggcaagtagcaagagtattgtattttgtttgaaatgtcatatttattgttgttgaggacgacaacatgttagatatttttaagagagatacgttttgaaatatgcacgattgaattttttcgtgtatatgatttgtttttcctgctcagtcaccgagttaatggccacatggacataaagatcagcttgatgtagagagggttatgtgaggttttctattaaaatgtttttttttatacgtgtctgctcttaagttgaattgcttgcaaaataggacttttttttaaaaaaagattttaattaggaaattttgataatcttcattattaggatttttccattgaaattatttattgatattacttttaacagagtctccgtgagactgtttcacagaaggatgtgtgtgtacccaaggctacacatcaaaaagaaatggataaaataagtggaaaattagaaggtaagaaccatcttttatttaaaaggtcatttgaccaagtatttctctaaaatgatgaggaaggatatgctctaatagctgaagaaaattacctcctaaatgcaaaccatggaaaaaaagagaagtgcaatggtcataagttatatgtctcatcaggtgttggcaaaagactatattgagagtgctgagaaggaggtgaattattagtttgaattcaagatattccaagacctgaggacaatgagaaaataagaaagaagaaagtagtaaaagaggaaatgaagactgagaaagacagagcgtacagagagtacatgaaggaacaagaatcaggtttatataatggaggatgatgaaatgattgtttaggaaaagattgggtgtggttagaaatttgggaagaatataaagtgaccttctagtaccaaaatttaccagagaattacagcaaaagtattctgactcttaatgtctttctcactggtgggaagccattagggatggaagcacctgaccatggagagctgtgttctatttgccataggtgagaataagcatgtctgcatggccacacatgtatataatttgtcatatacactccgtatagaccagaaattttcatagaaaatcagcgggaaacctagttaacaattcacactgtgattcagctgacttgggattctgcatttttaaaaagttctcaggtgatgctgatgctggtggtccttgtaccttgatctgagtagctgaggagaggccttttgtgggaaaaatgtggaagaatagcaattgaatagaaggtcaggacagaaaagggtcagaagaaagcattatgttacttttatttttgagtatgtttttagccagcggagaagaagaaaggggcaacgcaatagaaattgtagatggaagtaaagcagtcctcagcaaatgtaaaagaacagaaattatcacaaactgtctctcagaccacagtgcaataaaactagaactcaggattaagaacctcactcaaaaccacttaactacatggaaactgaacaacctcctccagaatgactactgggtacataacgaaatcaaggcagaaataaatatgttctttgaaaccgatgagaacaaaggcaccacataccagaatctgtgggacacatttaaagcagtgtgtagagggaaatttatagcactaaatgcccacaagagaaagcaggaaagatctaaaattgatacactaacatcacaattaaaagaactagagaagcaagagcaaacacattcaaaagctagcagaaggcaagaaataactaaaatcagagcagaattgaaggagatagagacacaaaaaatccttcaaaaaatcaatgaatccaggagctggttttttgaaaagattaacaaatatgatagactgctagcaagactaataaagaagaaaagtgagaagaatcaaatagacacaataaaaaatgataaaggggatatcaccaccgatcccacagaaatacaaactaccatcagagaatactataaacacctctatgcaaataaactggagaatctagaaaaaatggataaattcctggacagctacaccctcccaagactaaaccaggaaggagaatccccgaatagaccaataacaggctctgtaattgaggcaataattaatagcctaccaaccaagaaaagtccaggactggacagattcccagccaaattctaccagaggtacaaggaggagctgataccattccttctgaaatgattccaatcaatagaaaaagagggaatcctccctaactcattttatgaggccagcatcatcctgataccaaagcctggcagagacacaacaaaaaaacagaattttagaccagtatccctgatgaacatcaatgcaaaaatcctcaataaaatactggcaaaccgaatccagcagcacatcagaaagcttatccaccatgatcaagttggcttcatccctgggatgcaaggctggttcaagatatgcaaatcaataaatataattcatcatataaacagaaccaaaaacaaaaaccacatgattatctcaatagatgcagaaaaggcctttgacaaaattcaacagcacttcatgctaaaaactctcaataaactaggtattgatgggatgtatctcaaaataataagatctatttatgacaaacccacagccagtatcataccgaatgggcaaaaactggaagcattccctttgaaaactggcacaagacagggatgccctctctcaccactgctattcaacatagtgttggaagttctggccagggcaatcacacaggagaaagaaatgaatggtattcaataaggaatagaggaagtcaaattttccctgtttgcagatgacatgattgtatatttagaaaaccccattgtctcagcccgaaatcttcttaagctgatataagcagcatcagcaaagtctcaggatacataataaatgtgcaaaaatcacaagcactcttatacaccaataacagacaaacagagaaccaaatcatgagtgaactcccattcacaattgcttcaaagagaataaaatatctaggaatccaacttacaagggatgtgaaggacctcttcaaggagaactatacaccactgcttaacgaaataaaagaggacacaaacaaatggaagagcattccatgcttatggataggaagaatcaatatcgtgaaaatggccatactgcccaaggtaatttatggattcaatgccatccccattaagctaccaatgactttcttcacagaattggaaaaactactttaaagttcatatggaaccaaaaaagagcctgcagtgccaagacgatcctaagccaaaataacaaagctggaggcatcacactacctgacttcaaactatactacaaggctacagtaaccaaaacagcatggtattagtaccaaaacagagatatagatgaatggaacagaaccacacatctacaaccatctgatctttgatgaaccagacaaaaacaagaaatggggaaaggattccctatttaataaatggtgctgggaaaactggctagccatatgtagaaagctgaaactggatcccttccttacaacttatacaaaaattaattcaagatggattaaagacttaaatgttagacctaaaaccataaaaaccctagaagaaaacctaggcaataccattcaggacaaaggcatgggcaaagtcttcatgactaaaacaccaaaagcaatggcaacaaaagccaaaattgacaaatgggatctaattaaactaaagagcttcttctgcacagcaaaagaaactaccatgagaatgaacaggcaacctttttgcaatctacccatctgacaaagggctaatatccagaatctacaaaaacttaagtttacaagaaaaaatcaaacaccccatgaaaaagtgggcaaaggatatgaacagacacttctcaaaagaagatatttatgcagacaacagacacatgaaaaaatcctcatcatcacctgtcatcagagaaatgcaaatcaaaaccatgatgagatatcatctcacaccagttagaatggcgatcattaaaaagtcaggaaacaacaggtgctggagaggatgtggagaaataggaacgcttttacactgttggtgggagtgtaaactagttcaaccattgtggaagacagtgtggtgattcctgaaggatctagaactagaaataccatttgacccagggatcccattactggttatatacccaaaggattataaatcgtgctactgtaaagacacatgcacacatatgtttattgcagcactcttcacaatagcaaagacttggaactaacccaaatatacatcaatgatagactggattaagaaaatgtggcacatatacaccatggaattctctgcagccataaaaaaggatgtgttcatatcctttgtagggacatggatgaagctggaaaccatcattctgagcaaactatcgcaaggacagaaaaccaaacatcacatgttctcacccataggtgggaattgaacaatgagaacacttggacacaggaagtgcaacatcacacacgggggcctgtcatggggtggggggaggtgggagggatagcattaagagaaatacctaatgtaaatgacgagttaatgggtgcagtgcaccaatatggcacatgtatacatatgtaaaaaacctgcacgttgtgcagatgtactctagaacttaatgtataataacaacaaaaaaaatttccacccgtacttacacgcttgtggaagacattggagatggaattacctgactatgtgaagctgtgttttatctattattggtgaatataagcatatatgagcagccacacatgtatatattttttcagtattgtctagaaggtttcaaactttaagaaatgagctataaaacttgttaacagtgcacattctgattcagcagacttgaggtcatgcaattttaataaattctctgatgatgctgatgctggtggttcttggactgtacactgagtagcgaaggtatagagttttcattgaaataatttggaagaagagcaattggatagaatgtcaagacataacagagtcaagggaaagcattattttgattttatatctgagtatgtttttagccagaagaggagaagaaagaggttaaaaagcaaaaacagaatttatagatgcacgatattacttctgaacaagagaaagaaactggtaggaaaatttattttaaaaagtgttgaagggaaagaatcaagaccacagatccagatccggagattattttgctaaagaatagcaattgtgaggcatgaagtgggaggggggaagaagctatgaacttaattttgaggtttctgagaaggaaacttgagtgaattcacttcagatgcatttggaatgtttgcactccagaagatgagattgtgtgtgctctggagagtattggaagaaggaggtattactagatttggcgactcccacagtgactcattactcttctctgttactttcaggattcatagagatatgttttgttgatattatttatttaagtgagataaatttgaatatgaatccattggcttttttttgtaaaatttctgctttataaaatctgttagaaggctgggcccggtggctcatgcctgtaatcccagcactttgggaggccaaggcgggcagatcacttggggtcagcagttcgagaccagcctggccaacatggtgaaacccagtctctactaaaaatacaaaaattaattagctgggcttggtagcacatgcctgtaaacccagctactcaggaggctgaggcaggagaattgcttgaacccagggggcagagactgcagtgagctgagattgctccactgcactccagcctgggtggcagagtgagactcccatctcaaaaaaaataaaaatgaaaaaataaaaatttcttagagactaacatgataaatcagactgattttagaaacaaacaaaaaatgctaaatttattacttgaatactaaaactgatttttacataaatattatactgatttcaaaataaaaatggttatacttaattaatatttaacaattaagttgttgaatacatatttcaatattgaaagttttttatacattattttctttatgagttttatatgccctcttacatgaggggatcaaaaaacattcagatggataagtgagaggatgcaaaaaaatgtaggcataaaattacaccatgtgtatggaaaacaatgaatattttatttaccattattttctaatatacatccatactcataaattcattatactttcgttgatgagacatcaattttacattcagctaaactctcattgtaactgtgtaccttctcaattataagataaattaaagaatatgatgaatgtttgtagtttactgatataagtgattctgaagtgtatcttgtacttcagaaactctcacaatttataaccagtatatgttagccatgtgtgagattcatagatgccatatgataaatgggctctaaatgattaaatggcttgtaaaattttccttgtgtgttctttgcttttttcttttgtcacttaaaaattttaaactcaatgtttttgtttatgaaggaaaatagttacatacttactttttgtcacctgggactctgcacacttttgatatagtttgaacctagacttttttttttaacttgtgaaaaattaggtcacttatggctattatttttatataaatttctaatatatttatccaaaatttcacaagtacattcttaatatattggtgcattagaaagagactatatcattatcattataatagttttttgaaaatatattcaataaagtgttttaaagtccatgtctgtataattcattgacagaatagtctcatacctctttaaactttaaatgactagacataaatttcattgatagaaaagtatatatatctttgaagaatcttattgatgaataatttatcctggaaaacaaactatgtcataaagtttgctgagtcaattaaagaaagtaatagtaaaatattaattcctgaaagaaaatgagggttgatatttaattaaacatatctgggtaaatggtgactatcagacatagtgtgttttttgtggggaaaggaagacaagagactgctttttagaagaaaaaaaaattcatacgagctctttatgagtcttaggtcagggttttgataaggaagcagagccattcatgaccactcaggacatagaactgatgctgcattttctctttcctctgggagcctctttgcaattcactaagagcttttcctgagacccttgtcttggctggtgtctgtgcccctcattagggtatccaaacatcaagccagttttgtcagtccagtccagcttgtcagtgtagttttgctcagcttcgtccttctttctggagctgagtagagaattcagggcactgggcattacacagacttgccgataacctgaggtaacagagagttcctaccggtaaaacaaagacaaagcatattcttatccgcttctgccacagtacattctaacctgtggtcaccatttgttggctgggaggttgcactgcacaccccattacaaaatttgctgactcaagtgtggatagggaaatgatgtaggcttcttgagacctccactttctcatttctgcagcaaattgagggccttctcacctgcccagtatgctgccatgagaactagcatttgggaaaaccaccatactaagtttacatataaccaaaagaatcatacagaggctgactcccctgacagcactcagaaacaaattgcattcttcagcatatgttaaagccacaccctccaaggtgataacatgtctcaagcaagtgaaagtaaacttaaatagaagaagtgactctcttttcagattggaaagatgagccaaaagtctgaaagctgaaaaaacacaggatatcagcacacgactttattcctcaaccctttggcaaccactgatctttttactctatctatacataattttgttttctggataatgtcatttttagaaatatgcatttaagtcttttcatgtcttttcatagcttgatagttcatctcttttgagtccttagtaatattccattgtatgactatagcacagtttatacactcatctttctaaggttatcattattgcctcacaaattggttgaatttttgcaatcatgttcataaggcagttctcttgtttgtaataatatcatctggttttgatttagagtaatgctagcattatacaatgagttaaaatgtactgtgtattaggctatttgtgcattgctatagagaaatacctgagattgggtaatttatgaggaaatagatttcattggcgcacagttctacaggttgtacaggaagcataacgtggggctctgcttctggggaggcctgtggaagcttacaattatggtggaacgtgaggaagaacttgcacatcacatggccagagcaggagcaagagagagaggtgggaggtgccatgcatgtttaaatgaccaatattcatgaggatcactattgtgagtacagcaccaaaggcacggtgctaaattatttgtgagaaatcttccctcatgatgcattcacctctcaccaagctttactgccaacattggagatgacatttcaatgggagattttggtggggacacacatccaaactatgtcatactttcttttattttaggagtagagtttgtttagaattatacctctttttttctaaaatatttggtagaactcaccagtgtaggattcttttcctggacttttcaatgtaggaagacttttaactatgacttaatcttcttttatatatttagggcaatgcaggttaactattttttcttgagatattatattttttttgacactgactagatcttattttcctattgttgacatcatttatattttggcaaaagagagttggatcttttacataaaaatttcctttgaacctttgccagaatcagtatactgtcattcctagttaaaaaggttttcttatattataatttaagttctaggttacatgtgcagaacatgtgggtttgttacataggtatacacattccatggtggtttgctgtacccatcaacccatcatctacattaggtatttctcctaatgctacccctcccccagcccctcatccccaacagtccctggtgtgtgatgttcccctccctgtgtccatatgttctcattgttcaactcccacttatgagtgagaacatgcagtgtttggttttctgttcttgtgctagtttgctgagaatgatggtttccagcttcatccatgtccctgcaaaggacatgaattcatatccttttttatcactgcatagtattttatggtgtatatgtgccacattttctttatccaatgtctatcattgatggacatttgagttggttccaagtctttgctattgagaatactcccccaataaacctatgtatgcatgtgtctttataatagaatgatttataatcctttgggtatataaccagtaatgggatcgctgggtcaaatggtatttctggttctagatccttgaggaatcgccacactctcttctacaatggttgaactaattttcactcccaccaacagtgtaaaaacgattctatttctccacatcctctccagcatctgttgtttcctgactttttaatgattgccattctaactggcgtgagatgatatcttattgtggttttgatttgcatttctctaatgatcagtgatgatgagctttttttcatatgtttgttggctgcataaatgtgatctttgaagaagtatctgttcatatcctttgctcactttatgatggggttgtttgttttttccttgtaaatttgtttaagttcttgtacattctggatattagccctgtgtcagatggatagattgcaaaaattttctcccattctgtaggttgcctgttcattttgatgttagttgcttttgctgtgccgaagctctttagttgaattaagtcccatttgtcaattttggcttttgttgccattgcttttagtgttttagtcatgaagtctttcccatgcctatgtcctgaatgatattgcctaggttttcttctaggctttttatggttttaggtcttatgtttaagtgtttaatccatctttttttttatctagaattttttttattattatactttaagttttagggtacatgtgcataacatgcaggttagttacatatgtatacatgtgccatattggtgtgctgcacccattggctcatcatttaacattaggtatatctcctaatgctatccctcccccaccccacaacaggccctggtgtgtgatgttccccttcctgtgtccatgtgttctcattgttcaattcccacctatgagtgagaacatgtggtgtttggttttctgttcttgtgctagtttgctgagaatgatggtttccagcttcatccatgtccctacaaaggacatgaactcatcattttttatggctgcatagtattccatggtgtatatgtgccacattttcttaatccagtctatcattgttggacatttgtgttggttccaagtctttgctattgtgaatagtgctgcaataaacatatgtgtgcatgtgtctttatagcagcatgttttataatcctttgggtatataaccagtaatgggatggctgggtcaaatggtatttctagttctagatccctgaggaatcaccacactgacttccacaatggttgaactaatttacagtcccaccaacagtgtaaaagcattcctatttctctacatcctctccagcacctgttgtttcctgactttttaatgatcgccattctaactggtgtgagatggtatctcattgtggttttgatttgcatttctctgatggccagtgatgatgagcattttttcatgtgtcttttggctgcataaatgtcttcttttgagaagtgtctgttcatatcctttgcccaatttttgatggttcaccactcctattcaacatagtgttggaagttctggccagggcaatcatgcaggagaaggaaataaagggtattcaattagggaaagaggaagttgaattgtccctttttacagacgacatgattgtatatctagaaaaccccatcgtctcagcccaaaatcttctcaagctgataggcaacctcagcaaagtctcaggatacaaaatcaatgtgcaaaaatcacaagcattgttatacaccaataacagacaaacagacagccaaatcatgagtgaactcccattcacaattgcttcaaagagagtaaaatacctaggaatccaacttacaagggatgtgaaggacctcttcaaggagaactacaaaccactgcccaatgaaataaaagaggatacaaacaaatggaagaacattccacgcttatgggtaggaagaatcaatgtcgtaaaaatggccatactgcccaaggtaatttatcgattcaatgccatccccatcaagctaccaatgaccttcttcacagaattggaaaaactactttaaagttcatatggaaccaaaaaagagcccgcattgtcaagtcaatcctaagccaaaagaacaaagctgcaggcatcacgctacctgacttcaaactataccacaaggctacagtaaccaaaacagcatggtactggtaccaaaacagagatatagaccaatggaatagaacagagtcctcagaaataacgtctttaatccatgttgagttaatttttgtgtaaggtgtaaggaaggggtcgagtttcagttttctgcatatggccagctagctttcccaacaccatttattaaatagggaattatttccccattgctttttttttcagttttgtcaaagatcagatggtaattttcacatttcaatgaatttgtcaattttgtctaagttgtcatgtttattcacataaattttatattattaccatgtatttttaagtatctgtagaatcttaaagaatgtcacattaactcattttcaaacttggtaatttgtagtttttgttttcaagagctagtttggctagatagacttttgcttttattgatcctctcaaagtgtttggtgttgttcaagttttctatcatttttgcgttttattcgttccatctctgatgttttagttctctttgcctattatttaaaaaatctgtagttgggagcttatcggcaacataaattgaattctcaccattgtggaagctgcacagttcaagatcaaggtatcatcagatttgatgtctgaataggtaccacttcatgattcatcgatgcactgtttttccctgtgtcttcacatggcagaaagggtaaagaggctctctggggttatttctatcagggcattaatccatttatgaagggttggccctcatgacctaattacatcccaaaactcctacctcctaataacatcacctctatgatttgattttagcatacgaaaattgcaaggacaaaaacattcaaaccagtacagatattcatcatttcctttcctctgcttccactgagtttacttttttcttcttcttctttttttttttttttttggtttcttaaggtaaaagtcaaagttatttatttgagagtcttctaatgtagacattcagtgctgaaaattttaatttaagtgctgttgtagatgcattttacaatatctaatgtgttgtgttttcatttgaaattagttcaaaatactttctaattttcctttttattatttttgaaccaacccagggttatttggatgtatgttacttaatttccaatagttgagttgtttcaggtacttttttattattgatttttaatttgattgcttttgggtcaggtaacatgttttgtatgacttaaatccttcttaatccttttattaagatatgatctagaatatagtctttgagtacttctgaagaatggtgttaagtctatcacattcttgccgattttctgcctctcattttaccacttgagtaaggggtttagaaattttagaccacacttgggcatttgtccatttcttttgcagttctatctttgtatcatgtattttgaaggatttatgttattatgcacataaatatttaggactgttatgttttcttgattaagtgaaccctttgtcactataaaatgactttgtttattgctggtaatatttttgctatgaaatgtactttggtattaatacaaccacccttcctcagccttcttttgtcaagtgttagtgtggtatatgttgtttcatctttaatcaatttttgtctttatatttaaagtttatttcttataggcattacacaggtagatcttacttttatatccattctgacaatctgcttttgagcagaggtttttagaccactttaatttataatgtaattattgatatgataaaagtttgctgtcatcatgctgtttgatttctattagttccagatcttctttgctttgcttttgttctttttctgcttccttcagactagtttagtaatttttatgatttagttttataggtatatatatatatatatatatgtataacttttttagttattgatctagttttatatttctttatgatttagtcatatctttttgtggtataagtttatttggttattaggtataactctttgctttgctgtcttagtggtttttttaggatttatagtgtatggatttacctcatcacaacccatcttcaagtaatattatatcatatcatagatggtataagaaattacaattgtgtattttcatttcttctcttccagccagatcccagcctggatctgtgggattgtagctttcattaagtttagaatgtatttagccaatttttcctcaaatgtttttttctgtctctcctcctacctgtttggagacttatatattacctgctgggagtttgctcatagttctctagtgtctcaaatttgtgaatcttttctttcctgatggcaaatcaatctatgctcatatctactcagtgtagctttcatctccggcattgtaacttgtatctctacaagtgcaatttagtttttaaaagtatattctattgctctacttatcttcttgatttttattgtagaatagagttgagttacttataaacagcttgatctttttcatttttgctttttatggtatgagctaactccccacacctgaggggcaaggcctttctgactattcattttcctgtgaagtctgagtcttccaggtaaatctataaaaatagacactcttcttgccactatgtgagaaccaggtgtgattttctctaattttgtaagacccccccgacccagcttggtcttaggtagttttctgactcacatgcaatctttattattttgctaaatagtaggaggggtttccaggggtttcttgctattgcttctgtatctctctcccctcctcagtgttttgttctgtaatgtctgtctgctttggttttaccagactctaagcttcatcactgtaaccaagagagtctgataggttccacctcagttttttcttcctgtgtcacgtcttggaatctctgtcaaagcaggaagctgaaacattcataaggtttgctttccattttttttctgtttttcagggattatcatcttctttgcctaatgtccactgtctaaaaaaattgtttaatgtattttgtgtgttttctttttagttattttagctaagaagaaaaatcaatacccattgttctcttttggctggggtcagactacactagagcttcagcacatgccacacactggctaaaatgcttttcttccccctttgctcaactgcttccttttcaatctttattccacagtgtagccatacattcctcaggggaattttctgtgggcctagtatatatcttattcttagcaatctgttttcttacagtatctatctgaatttataattgtcacttttctggggctttgcttttcagtatattttaagctaaacaagaggagaggttttttttttttctgtttaatctgcagagcttagtataatgccttccacgtggtaggcaatcaatatatatttgttcagtgtataagtgattgttaaaatatgcagccctttatatcccaaaagtactaatatattttatttgtatctcctccttgagacagattcaactagcctatcaaaaatcttggatacagttcattcttgtgaaagagcaagggaacttcaaaaagatcactgtgaacaacgtacaggaaaaatggaacaaatgaaaaagaagttttgtgtactgaaaaagaaactgtcagaagcaaaagaaataaaatcacagttagagaaccaaaaagttaaatgggaacaagagctctgcagtgtgaggtgtgatttcctagttttaaataaatatttcagctatttatactaaaactacgtaggatactttttgtaatagctgacttaccttctgaggttttactggagaaaaaaatgtctgtcttgtagagtgtcaaattctttaaataatacaagttcttaaatgtgaatacttctgctgataattaaatgcatatttatttaaatcacagttttaatggctatatagaaggccattatttggaaatcccattacttacttaacaaattaagttttttgatttttaactttctgtattataaatgctgcaaggcataactgcatgtaaaactttttctacttttctaattattgacttgaaataaattcttcaatataaaaatatttggttaagttataggaagtttttaagaagttatttgttcattacttctaaattgttctcaagaaaatgtatattcattacagttcaacaaagagagcgtgaaatggccattcttctttccccaagactcgatttctttcaattatacacttttaatcttaatgtgcatggaatataaagaaaatgtataataatttattagtttattcaacatctcttgctgtcctacataaaatgaattcagagttctatgttaaaaatgcatactatttttattctttaattaaatattatattctgtcttgttctaaaagagatttaaaatttgttgataaaatatataataatcaacaaggtacgtttaaagcattactcaaaaaagaaacaaaaggcatatgggataacagatattagactctttaacctaatctgagattttaatattaaaattatttttagggatacttgccatattttagaagtatgaatatttatgtctaacagatctgtaaacttgttcataaaaagtaatcagttaactttaatcagtcactttaatcagttaactctaaataatctgtcctcattgaggagtaattttgactgcgtgagatttttaaaaaataattttcaacttataaatttactggatagcttccaatattcttttctataacagtcgttgcagttaatagtagcagaaactttctaactggaagatgttcttttctcgctatttttcaagtatgtatgtcatttggaagagtttggagtaataaatagctgagaactaggaagaaaaaaattcaagaatataggaattttattggactaataacccaatataggaagaaatagatctcaaaatgaattctattttctaacaaaatgaattttaagataagtatgtttaatggcagattgactttaaaccaagaagaagagaagagaagaaatgccgatatattaaatgaaaaaattagggaagaattaggaagaatcgaagagcagcataggaaagagttagaagtgaaacaacaacttgaacaggctctcagaatacaagatatagaattgaagagtgtagaaagtaatttgaatcaggtaaatcaatctctgataaaaattttatatttctaactttatttcatcaatattacttttaatatccctttgatttagtatgtattattcaggtctaaatcaaacaaaaatgttgtcttaaaattaactatcacatttgtagctacagttatttattataagttatgacatccaagtagtatcttatttcagtacaaagagcttttgaaaacaatgataatccctaccatatagttagtgataatttattggtaagtagtttgttcctagcaacatagtttagtgtttttcctagttaacatttaatactgactcaaacattatcaagaggaagcaaaagttacagccatagtaaataagctcatggttttctatgtagggctctctagattttatcttctttcttttgttttgaaatagaagacttcttttatatttacatatttacccagtagaatgaactgagatttggtgaagtcctggatgtagactcagaagactgggagaaaatcctacaacttgtttatatttttaatcttttcctttcagaattgtgataactaaaagtgcttgttacaatgtctcaacttatcaaacattcataaatataattcttaaaattaactatattttttaaaaagatagaatatctagagaatattatcaggaaaaaggaactgaaagagctctgggaaattttatctgtctaaatatatgcagcactaagattcttagtatatgttagacatcagagtgtaaacccaattttcgtatgtagtcaaattgatttatcttttattttatgcttttgagcttgttgtaattcagggaaagttttttttttcaattccgaggttcttaaaaactctctagtcatttcttttgtactttcatgaattcgttgtcttcaaataaatgtttgaactttggggaatttatgctctatagcatttaaagttttgattcagtggttcttcaactgatacctacttataaaaaccctttcattgtataaatgtacaagttattctttagtttcagaggaaccatgatatgccattttattgagtgctagctaaaagtttattttgttttatttaggtttctcacactcatgaaaatgaaaattatc

SEQ ID NO: 90 - DNA, 2726 bp, human
acccagaagaccgtgccttgcctggaagtcctgcctgtaggcctgaaggacttgccctaacagagcctcaacaactacctggtgattcctacttcagccccttggtgtgagcagcttctcaacatgaactacagcctccacttggccttcgtgtgtctgagtctcttcactgagaggatgtgcatccaggggagtcagttcaacgtcgaggtcggcagaagtgacaagctttccctgcctggctttgagaacctcacagcaggatataacaaatttctcaggcccaattttggtggagaacccgtacagatagcgctgactctggacattgcaagtatctctagcatttcagagagtaacatggactacacagccaccatatacctccgacagcgctggatggaccagcggctggtgtttgaaggcaacaagagcttcactctggatgcccgcctcgtggagttcctctgggtgccagatacttacattgtggagtccaagaagtccttcctccatgaagtcactgtgggaaacaggctcatccgcctcttctccaatggcacggtcctgtatgccctcagaatcacgacaactgttgcatgtaacatggatctgtctaaataccccatggacacacagacatgcaagttgcagctggaaagctggggctatgatggaaatgatgtggagttcacctggctgagagggaacgactctgtgcgtggactggaacacctgcggcttgctcagtacaccatagagcggtatttcaccttagtcaccagatcgcagcaggagacaggaaattacactagattggtcttacagtttgagcttcggaggaatgttctgtatttcattttggaaacctacgttccttccactttcctggtggtgttgtcctgggtttcattttggatctctctcgattcagtccctgcaagaacctgcattggagtgacgaccgtgttatcaatgaccacactgatgatcgggtcccgcacttctcttcccaacaccaactgcttcatcaaggccatcgatgtgtacctggggatctgctttagctttgtgtttggggccttgctagaatatgcagttgctcactacagttccttacagcagatggcagccaaagatagggggacaacaaaggaagtagaagaagtcagtattactaatatcatcaacagctccatctccagctttaaacggaagatcagctttgccagcattgaaatttccagcgacaacgttgactacagtgacttgacaatgaaaaccagcgacaagttaaagtttgtcttccgagaaaagatgggcaggattgttgattatttcacaattcaaaaccccagtaatgttgatcactattccaaactactgtttcctttgatttttatgctagccaatgtattttactgggcatactacatgtatttttgagtcaatgttaaatttcttgcatgccataggtcttcaacaggacaagataatgatgtaaatggtattttaggccaagtgtgcacccacatccaatggtgctacaagtgactgaaataatatttgagtctttctgctcaaagaatgaagctccaaccattgttctaagctgtgtagaagtcctagcattataggatcttgtaatagaaacatcagtccattcctctttcatcttaatcaaggacattcccatggagcccaagattacaaatgtactcagggctgtttattcggtggctccctggtttgcatttacctcatataaagaatgggaaggagaccattgggtaaccctcaagtgtcagaagttgtttctaaagtaactatacatgttttttactaaatctctgcagtgcttataaaatacattgttgcctatttagggagtaacattttctagtttttgtttctggttaaaatgaaatatgggcttatgtcaattcattggaagtcaatgcactaactcaataccaagatgagtttttaaataatgaatattatttaataccacaacagaattatccccaatttccaataagtcctatcattgaaaattcaaatataagtgaagaaaaaattagtagatcaacaatctaaacaaatccctcggttctaagatacaatggattccccatactggaaggactctgaggctttattcccccactatgcatatcttatcattttattattatacacacatccatcctaaactatactaaagcccttttcccatgcatggatggaaatggaagatttttttttaacttgttctagaagtcttaatatgggctgttgccatgaaggcttgcagaattgagtccattttctagctgcctttattcacatagtgacggggtactaaaagtactgggttgactcagagagtcgctgtcattctgtcattgctgctactctaacactgagcaacactctcccagtggcagatcccctgtatcattccaagaggagcattcatccctttgctctaatgatcaggaatgatgcttattagaaaacaaactgcttgacccaggaacaagtggcttagcttaagtaaacttggctttgctcagatccctgatccttccagctggtctgctctgagtggcttatcccgcatgagcaggagcgtgctggccctgagtactgaactttctgagtaacaatgagatacgttacagaacctatgttcaggttgcgggtgagctgccctctccaaa

SEQ ID NO: 91 - DNA, 2641 bp, human
gcagtgtgaggcaatcgctctatccttgaccccttcctttgcacagtgagtgatggcgtttttatctcctgatgatgatgcacagccttcagcgggggacatttaagacgcagaacaccaggtccaggctgcagctgcgggactcagaggcgaagcttgaggggctcaggaaggacgaagaaccacccttgagagaagaggcagcagcagcggcggcagcagcagcggcagcgaccccaccactgccacatttgccaggaaacaatgctgctagcgacattcaagctgtgcgctgggagctcctacagacacatgcgcaacatgaaggggctgaggcaacaggctgtgatggccatcagccaggagctgaaccggagggccctggggggccccacccctagcacgtggattaaccaggttcggcggcggagctctctactcggttctcggctggaagagactctctacagtgaccaggagctggcctatctccagcagggggaggaggccatgcagaaggccttgggcatccttagcaaccaagagggctggaagaaggagagtcagcaggacaatggggacaaagtgatgagtaaagtggtcccagatgtgggcaaggtgttccggctggaggtcgtggtggaccagcccatggagaggctctatgaagagctcgtggagcgcatggaagcaatgggggagtggaaccccaatgtcaaggagatcaaggtcctgcagaagatcggggggccccgtgactttgtgagcgtgcgctgtgccaagcgccgaggctccacctgtgtgctggctggcatggccacagacttcgggaacatgcctgagcagaagggtgtcatcagggcggagcacggtcccacttgcatggtgcttcacccgttggctggaagtccctctaagaccaaacttacgtggctactcagcatcgacctcaaggggtggctgcccaagagcatcatcaaccaggtcctgtcccagacccaggtggattttgccaaccacctgcgcaagcgcctggagtcccaccctgcctctgaagccaggtgttgaagaccagcctgctgttcccaactgtgcccagctgcactggtacacacgctcatcaggagaatccctactggaagcctgcaagtctaagatctccatctggtgacagtgggatgggtggggttcgtgtttagagtatgacactaggattcagattggtgaaagtttttagtaccaagaaaacagggatgaggctcttggattaaaaggtaacttcattcactgattagctatgacatgagggttcaggcccctaaaataattgtaaaactttttttctgggcccttatgtacccacctaaaaccatctttaaaatgctagtggctgatatgggtgtgggggatgctaaccacagggcctgagaagtcttgctttatgggctcaagaatgccatgcgctggcagtacatgtgcacaaagcagaatctcagagggtctcctgcagccctctgctcctcccggccgctgcacagcaacaccacagaacaagcagcaccccacagtgggtgccttccagaaatatagtccaagctttctctgtggaaaaagacaaaactcattagtagacatgtttccctattgctttcataggcaccagtcagaataaagaatcataattcacacaaacatcagtctttgttttaatattgtactgttaaaaaaatctatgcagctgggtgcagtggctcacgcctgtaatcccagcattttgggaggctgaggtaggcggatcacaaggtcaggagatcgagaccatcctggccaacatggtgaaaccccgtctctactaaaatacaaaaaattagctgggtgtggtggcgcaaacctgtagtcgtagctacttgggaggctgaggcaggggaatcacttgaaccccggaggcggaggttgtagtgggccgagattgtgccactgcgctccagcgtgggcgacagagtgagactccatctcaaaaaaaaaaaaaaaaaaatctatgctagtagattacaacttcacactagaggagttctggacaaagcttttaattagtcaaactaaattaaggctcattaaaaggaaaggaactactgggaaattatgcaattcaataatttagactctgttaccaggatctttcataaaaatttaatttccataatcataacctaaatgagttcttaaagaattctataagcaatagctgattaatgggccctggaagatgaagattataactgtttatttacctaattaaaaggaaaggcagtgccaaatatgagaggataaacaatattagttaacatttctgttatttatgatgccaattagtagtaagataattccacagctgtcaactttgtttggggctggcaacttctctgcttaaacaggctaaaagtttagtattctgggagaagtggctggaagaaggggtaatatggtgaaagcaaattccctttcccaggagtcaagagaatttatgtgaggtggctcccgcctgtaatctcagcactttgggaggccaaggcaggcaatcacctgagggcaggagaccagcctgactaacatggagaaaccccatctctactaaaaatac

SEQ ID NO: 92 - DNA, 2908 bp, human
cgggcgccgcaggagcgagtgagctgggagcgaggggcgaaggcgcggagaagcccggccgcccggtgggcggcagaaggctcagccgaggcggcggcgccgactccgttccactctcggcccggatccaggcctccgggttcccaggcgctcacctccctctgacgcactttaaagagtctccccccttccacctcagggcgagtaatagcgaccaatcatcaagccatttaccaggcttcggaggaagctgtttatgtgatccccgcactaattaggctcatgaactaacaaatcgtttgcacaacttgtgaagaagcgaacacttccatggattgtccttggacttagggcgccctgcccgccttttgcagaggagaaaaaacttttttttttttttgcctcccccgagaactttccccccttctcctccctgcctctaactccgatccccccacgccatctcgccaaaaaaaaaaaaaaaaaaaaaaaagaaaaaaaaagaaaaaaaaagaaaaaaaattaccccaatccacgcctgcaaattcttctggaaggattttcccccctctcttcaggttgggcgcgtttggtgcaagattctcgggatcctcggctttgcctctccctctccctcccccctcctttcctttttcctttcctttcctttctttcttcctttccttccccccacccccacccccaccccaaacaaacgagtccccaattctcgtccgtcctcgccgcgggcagcgggcggcggaggcagcgtgcggcggtcgccaggagctgggagcccagggcgcccgctcctcggcgcagcatgttccagccggcgcccaagcgctgcttcaccatcgagtcgctggtggccaaggacagtcccctgcccgcctcgcgctccgaggaccccatccgtcccgcggcactcagctacgctaactccagccccataaatccgttcctcaacggcttccactcggccgccgccgccgccgccggtaggggcgtctactccaacccggacttggtgttcgccgaggcggtctcgcacccgcccaaccccgccgtgccagtgcacccggtgccgccgccgcacgccctggccgcccaccccctaccctcctcgcactcgccacaccccctattcgcctcgcagcagcgggatccgtccaccttctacccctggctcatccaccgctaccgatatctgggtcatcgcttccaagggaacgacactagccccgagagtttccttttgcacaacgcgctggcccgaaagcccaagcggatccgaaccgccttctccccgtcccagcttctaaggctggaacacgcctttgagaagaatcactacgtggtgggcgccgaaaggaagcagctggcacacagcctcagcctcacggaaactcaggtaaaagtatggtttcagaaccgaagaacaaagttcaaaaggcagaagctggaggaagaaggctcagattcgcaacaaaagaaaaaagggacgcaccatattaaccggtggagaatcgccaccaagcaggcgagtccggaggaaatagacgtgacctcagatgattaaaaacataaacctaaccccacagaaacggacaacatggagcaaaagagacagggagaggtggagaaggaaaaaaccctacaaaacaaaaacaaaccgcatacacgttcaccgagaaagggagagggaatcggagggagcagcggaatgcggcgaagactctggacagcgagggcacagggtcccaaaccgaggccgcgccaagatggcagaggatggaggctccttcatcaacaagcgaccctcgtctaaagaggcagctgagtgagagacacagagagaaggagaaagagggagggagagagagaaagagagagaaagagagagagagagagagagagagaaagctgaacgtgcactctgacaaggggagctgtcaatcaaacaccaaaccggggagacaagatgattggcaggtattccgtttatcacagtccacttaaaaaatgatgatgatgataaaaaccacgacccaaccaggcacaggacttttttgttttttgcacttcgctgtgtttcccccccatctttaaaaataattagtaataaaaaacaaaaattccatatctagccccatcccacacctgtttcaaatccttgaaatgcatgtagcagttgttgggcgaatggtgtttaaagaccgaaaatgaattgtaattttcttttccttttaaagacaggttctgtgtgctttttattttgattttttttcccaagaaatgtgcagtctgtaaacactttttgataccttctgatgtcaaagtgattgtgcaagctaaatgaagtaggctcagcgatagtggtcctcttacagagaaacggggagcaggacgacgggggggctgggggtggcgggggagggtgcccacaaaaagaatcaggacttgtactgggaaaaaaacccctaaattaattatatttcttggacattccctttcctaacatcctgaggcttaaaaccctgatgcaaacttctcctttcagtggttggagaaattggccgagttcaaccattcactgcaatgcctattccaaactttaaatctatctattgcaaaacctgaaggactgtagttagcggggatgatgttaagtgtggccaagcgcacggcggcaagttttcaagcactgagtttctattccaagatcatagacttactaaagagagtgacaaatgcttccttaatgtcttctataccagaatgtaaatatttttgtgttttgtgttaatttgttagaattctaacacactatatacttccaagaagtatgtcaatgtcaatattttgtcaataaagatttatcaatatgccctcaaaaaaaaaaaaa

SEQ ID NO: 93 - DNA, 3308 bp, human
aaaagatagatcctgctccaggagccgggaagcctcgccctggccagctgtgctgggcacctcccctgcctgcttcctggcccacttgcaggcaaggtgagggcatgcgaatggctgccactgcctgggcggggctccaagggccacccctccccaccctctgtcccgcagtgaggacgggactctactgccgagaccaggctcacgctgagaggtgggccatgacctccgagacctcttccggaagccactgtgccaggagcaggatgctgcggcgacgggcccaggaagaggacagcaccgtcctgatcgatgtgagcccccctgaggcagagaagaggggctcttacgggagcacagcccacgcctcggagccaggtggacagcaagcggccgcctgcagagctgggagtcctgccaagccccggatcgcagacttcgtcctcgtttgggaggaggacctgaagctagacaggcagcaggacagtgccgcccgggacagaacagacatgcacaggacctggcgggagacttttctggataatcttcgtgcggctgggctgtgtgtagaccagcaggacgtccaggacgggaacaccacagtgcactacgccctcctcagcgcctcctgggctgtgctctgctactacgccgaagacctgcgcctgaagctgcccttgcaggagttacccaaccaggcctccaactggtcggccggcctgctggcatggctgggcatccccaacgtcctgctggaggttgtgccagacgtaccccccgagtactactcctgccggttcagagtgaacaagctgccacgcttcctcgggagtgacaaccaggacaccttcttcacaagcaccaagaggcaccaaattctgtttgagatcctggccaagaccccgtatggccacgagaagaaaaacctgcttgggatccaccagctgctggcagagggtgtcctcagtgccgccttccccctgcatgacggccccttcaagacgcccccagagggcccgcaggctccacgcctcaaccagcgccaagtccttttccagcactgggcgcgctggggcaagtggaacaagtaccagcccctggaccacgtgcgcaggtacttcggggagaaggtggccctctacttcgcctggctcgggttttacacaggctggctcctgccagcggcagtggtgggcacactggtgttcctggtgggctgcttcctggtgttctcagacatacccacgcaggaactgtgtggcagcaaggacagcttcgagatgtgcccactttgcctcgactgccctttctggctgctctccagcgcctgtgccctggcccaggccggccggctgttcgaccacggcggcaccgtgttcttcagcttgttcatggcactgtgggccgtgctgctgctggagtactggaagcggaagagcgccacgctggcctaccgctgggactgctctgactacgaggacactgaggagaggcctcggccccagtttgccgcctcagcccccatgacagccccgaaccccatcacgggtgaggacgagccctacttccctgagaggagccgcgcgcgccgcatgctggccggctctgtggtgatcgtggtgatggtggccgtggtggtcatgtgcctcgtgtctatcatcctgtaccgtgccatcatggccatcgtggtgtccaggtcgggcaacacccttctcgcagcctgggcctctcgcatcgccagcctcacggggtctgtagtgaacctcgtcttcatcctcatcctctccaagatctatgtatccctggcccacgtcctgacacgatgggaaatgcaccgcacccagaccaagttcgaggacgccttcaccctcaaggtgttcatcttccagttcgtcaacttctactcctcacccgtctacattgccttcttcaagggcaggtttgtgggatacccaggcaactaccacaccttgtttggagtccgcaatgaggagtgcgcggctggaggctgcctgatcgagctggcacaggagctcctggtcatcatggtgggcaagcaggtcatcaacaacatgcaggaggtcctcatcccgaagctaaagggctggtggcagaagttccggcttcgctccaagaagaggaaggcgggagcttctgcaggggctagccaggggccctgggaggacgactatgagcttgtgccctgtgagggtctgtttgacgagtacctggaaatggtgctgcagttcggcttcgtcaccatcttcgtggccgcctgtccgctcgcgccgctcttcgccctgctcaacaactgggtggagatccgcttggacgcgcgcaagttcgtctgcgagtaccggcgccctgtggccgagcgcgcccaggacatcggcatctggttccacatcctggcgggcctcacgcacctggcggtcatcagcaacgccttcctcctggccttctcgtccgacttcctgccgcgcgcctactaccggtggacccgcgcccacgacctgcgcggcttcctcaacttcacgctggcgcgagccccgtcctccttcgccgccgcgcacaaccgcacgtgcaggtatcgggctttccgggatgacgatggacattattcccagacctactggaatcttcttgccatccgcctggccttcgtcattgtgtttgagcatgtggttttctccgttggccgcctcctggacctcctggtgcctgacatcccagagtctgtggagatcaaagtgaagcgggagtactacctggctaagcaggcactggctgagaatgaggttctttttggaacgaacggaacaaaggatgagcagcccaagggctcagagctcagctcccactggacacccttcacggttcccaaggccagccagctgcagcagtgacgcctggaaggacatctggtggtccttaggggagtggcccctcctgagccctgcgagcagcgtccttttcctcttccctcaggcagcggctgtgtgaaccgctggctgctgttgtgcctcatctctgggcacattgcctgcttccccccagcgccggcttctctcctcagagcgcctgtcactccatccccggcagggagggaccgtcagctcacaaggccctctttgtttcctgctcccagacataagcccaaggggcccctgcacccaagggaccctgtccctcggtggcctccccaggcccctggacacgacagttctcctcaggcaggtgggctttgtggtcctcgccgcccctggccacatcgccctctcctcttacacctggtgaccttcgaatgt

SEQ ID NO: 94 - DNA, 551 bp, human
accccatccgctggctctcacccctcggagacgctcgcccgacagcatagtacttgccgcccagccacgcccgcgcgccagccaccatgctaggtaacaagcgactggggctgtccggactgaccctcgccctgtccctgctcgtgtgcctgggtgcgctggccgaggcgtacccctccaagccggacaacccgggcgaggacgcaccagcggaggacatggccagatactactcggcgctgcgacactacatcaacctcatcaccaggcagagatatggaaaacgatccagcccagagacactgatttcagacctcttgatgagagaaagcacagaaaatgttcccagaactcggcttgaagaccctgcaatgtggtgatgggaaatgagacttgctctctggccttttcctattttcagcccatatttcatcgtgtaaaacgagaatccacccatcctaccaatgcatgcagccactgtgctgaattctgcaatgttttcctttgtcatcattgtatatatgtgtgtttaaataaagtatcatgcattc

SEQ ID NO: 95 - DNA, 1607 bp, human
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctggtccctgcctgcaaaa

SEQ ID NO: 96 - DNA, 1753 bp, human
cagccccgcccctacctgtggaagcccagccgcccgctcccgcggataaaaggcgcggagtgtccccgaggtcagcgagtgcgcgctcctcctcgcccgccgctaggtccatcccggcccagccaccatgtccatccacttcagctccccggtattcacctcgcgctcagccgccttctcgggccgcggcgcccaggtgcgcctgagctccgctcgccccggcggccttggcagcagcagcctctacggcctcggcgcctcacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgcggtttctggagcagcagaacaagctgctggagaccaagtggacgctgctgcaggagcagaagtcggccaagagcagccgcctcccagacatctttgaggcccagattgctggccttcggggtcagcttgaggcactgcaggtggatgggggccgcctggaggcggagctgcggagcatgcaggatgtggtggaggacttcaagaataagtacgaagatgaaattaaccaccgcacagctgctgagaatgagtttgtggtgctgaagaaggatgtggatgctgcctacatgagcaaggtggagctggaggccaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagcctggtaccagaccaagtttgagaccctccaggcccaggctgggaagcatggggacgacctccggaatacccggaatgagatttcagagatgaaccgggccatccagaggctgcaggctgagatcgacaacatcaagaaccagcgtgccaagttggaggccgccattgccgaggctgaggagcgtggggagctggcgctcaaggatgctcgtgccaagcaggaggagctggaagccgccctgcagcggggcaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggaccgcatccgccagtcgcaggagtgcccgcgactgagccgcctcccaccactccactcctccagccaccacccacaatcacaagaagattcccacccctgcctcccatgcctggtcccaagacagtgagacagtctggaaagtgatgtcagaatagcttccaataaagcagcctcattctgaggcctgagtgatccacgtgaaaaaaaaaaaaaaaaaaaaaaaaaaaaaaaaaaaaaaaaaaaaaaaaaaa

SEQ ID NO: 97 - DNA, 2276 bp, human
aagcccagcagccccggggcggatggctccggccgcctggctccgcagcgcggccgcgcgcgccctcctgcccccgatgctgctgctgctgctccagccgccgccgctgctggcccgggctctgccgccggacgcccaccacctccatgccgagaggagggggccacagccctggcatgcagccctgcccagtagcccggcacctgcccctgccacgcaggaagccccccggcctgccagcagcctcaggcctccccgctgtggcgtgcccgacccatctgatgggctgagtgcccgcaaccgacagaagaggttcgtgctttctggcgggcgctgggagaagacggacctcacctacaggatccttcggttcccatggcagttggtgcaggagcaggtgcggcagacgatggcagaggccctaaaggtatggagcgatgtgacgccactcacctttactgaggtgcacgagggccgtgctgacatcatgatcgacttcgccaggtactggcatggggacgacctgccgtttgatgggcctgggggcatcctggcccatgccttcttccccaagactcaccgagaaggggatgtccacttcgactatgatgagacctggactatcggggatgaccagggcacagacctgctgcaggtggcagcccatgaatttggccacgtgctggggctgcagcacacaacagcagccaaggccctgatgtccgccttctacacctttcgctacccactgagtctcagcccagatgactgcaggggcgttcaacacctatatggccagccctggcccactgtcacctccaggaccccagccctgggcccccaggctgggatagacaccaatgagattgcaccgctggagccagacgccccgccagatgcctgtgaggcctcctttgacgcggtctccaccatccgaggcgagctctttttcttcaaagcgggctttgtgtggcgcctccgtgggggccagctgcagcccggctacccagcattggcctctcgccactggcagggactgcccagccctgtggacgctgccttcgaggatgcccagggccacatttggttcttccaaggtgctcagtactgggtgtacgacggtgaaaagccagtcctgggccccgcacccctcaccgagctgggcctggtgaggttcccggtccatgctgccttggtctggggtcccgagaagaacaagatctacttcttccgaggcagggactactggcgtttccaccccagcacccggcgtgtagacagtcccgtgccccgcagggccactgactggagaggggtgccctctgagatcgacgctgccttccaggatgctgatggctatgcctacttcctgcgcggccgcctctactggaagtttgaccctgtgaaggtgaaggctctggaaggcttcccccgtctcgtgggtcctgacttctttggctgtgccgagcctgccaacactttcctctgaccatggcttggatgccctcaggggtgctgacccctgccaggccacgaatatcaggctagagacccatggccatctttgtggctgtgggcaccaggcatgggactgagcccatgtctcctcagggggatggggtggggtacaaccaccatgacaactgccgggagggccacgcaggtcgtggtcacctgccagcgactgtctcagactgggcagggaggctttggcatgacttaagaggaagggcagtcttgggcccgctatgcaggtcctggcaaacctggctgccctgtctccatccctgtccctcagggtagcaccatggcaggactgggggaactggagtgtccttgctgtatccctgttgtgaggttccttccaggggctggcactgaagcaagggtgctggggccccatggccttcagccctggctgagcaactgggctgtagggcagggccacttcctgaggtcaggtcttggtaggtgcctgcatctgtctgccttctggctgacaatcctggaaatctgttctccagaatccaggccaaaaagttcacagtcaaatggggaggggtattcttcatgcaggagaccccaggccctggaggctgcaacatacctcaatcctgtcccaggccggatcctcctgaagcccttttcgcagcactgctatcctccaaagccattgtaaatgtgtgtacagtgtgtataaaccttcttcttctttttttttttttaaactgaggattgtcattaaacacagttgttttctaaaaaaaaaaaaaaaa

SEQ ID NO: 98 - DNA, 7381 bp, human
tacagccccaaggtcgctccctctggggccctttcttccccattcttcccagcagcccaaagctctggtgggacaggggcagcccctggggagggaggagaggacccaggaacccggctaggagggtggcccacccatttccagtgtgacctgttcccattcccccatgtctcctcccatccctcccgccactcagctcaggctgatgagaagcagagcaacgggtgtatcggtgttttctttcctggtggggtagtggggtggggctgaggagagaaaagggtgattagcgtggggccccgccctcttttgtcctcttcccaggttccctggccccttcggagaaacgcacttggttcgggccagccgcctgaggggacgggctcacgtctgctcctcacactgcagctgctgggccgtggagcttccccagggagccagggggacttttgccgcagccatgaagggggcacgctggaggagggtcccctgggtgtccctgagctgcctgtgtctctgcctccttccgcatgtggtcccaggaaccacagaggacacattaataactggaagtaaaactcctgccccagtcacctcaacaggctcaacaacagcgacactagagggacaatcaactgcagcttcttcaaggacctctaatcaggacatatcagcttcatctcagaaccaccagactaagagcacggagaccaccagcaaagctcaaaccgacaccctcacgcagatgatgacatcaactcttttttcttccccaagtgtacacaatgtgatggagactgttacgcaggagacagctcctccagatgaaatgaccacatcatttccctccagtgtcaccaacacactcatgatgacatcaaagactataacaatgacaacctccacagactccactcttggaaacacagaagagacatcaacagcaggaactgaaagttctaccccagtgacctcagcagtctcaataacagctggacaggaaggacaatcacgaacaacttcctggaggacctctatccaagacacatcagcttcttctcagaaccactggactcggagcacgcagaccaccagggaatctcaaaccagcaccctaacacacagaaccacttcaactccttctttctctccaagtgtacacaatgtgacagggactgtttctcagaagacatctccttcaggtgaaacagctacctcatccctctgtagtgtcacaaacacatccatgatgacatcagagaagataacagtgacaacctccacaggctccactcttggaaacccaggggagacatcatcagtacctgttactggaagtcttatgccagtcacctcagcagccttagtaacagttgatccagaaggacaatcaccagcaactttctcaaggacttctactcaggacacaacagctttttctaagaaccaccagactcagagcgtggagaccaccagagtatctcaaatcaacaccctcaacaccctcacaccggttacaacatcaactgttttatcctcaccaagtggattcaacccaagtggaacagtttctcaggagacattcccttctggtgaaacaaccatctcatccccttccagtgtcagcaatacattcctggtaacatcaaaggtgttcagaatgccaatctccagagactctactcttggaaacacagaggagacatcactatctgtaagtggaaccatttctgcaatcacttccaaagtttcaaccatatggtggtcagacactctgtcaacagcactctcccccagttctctacctccaaaaatatccacagctttccacacccagcagagtgaaggtgcagagaccacaggacggcctcatgagaggagctcattctctccaggtgtgtctcaagaaatatttactctacatgaaacaacaacatggccttcctcattctccagcaaaggccacacaacttggtcacaaacagaactgccctcaacatcaacaggtgctgccactaggcttgtcacaggaaatccatctacaagggcagctggcactattccaagggtcccctctaaggtctcagcaataggggaaccaggagagcccaccacatactcctcccacagcacaactctcccaaaaacaacaggggcaggcgcccagacacaatggacacaagaaacggggaccactggagaggctcttctcagcagcccaagctatagtgtgattcagatgataaaaacggccacatccccatcttcttcacctatgctggatagacacacatcacaacaaattacaacggcaccatcaacaaatcattcaacaatacattccacaagcacctctcctcaggaatcaccagctgtttcccaaaggggtcacactcgagccccgcagaccacacaagaatcacaaaccacgaggtccgtctcccccatgactgacaccaagacagtcaccaccccaggttcttccttcacagccagtgggcactcgccctcagaaattgttcctcaggacgcacccaccataagtgcagcaacaacctttgccccagctcccaccgggaatggtcacacaacccaggccccgaccacagcactgcaggcagcacccagcagccatgatgccaccctggggccctcaggaggcacgtcactttccaaaacaggtgcccttactctggccaactctgtagtgtcaacaccagggggcccagaaggacaatggacatcagcctctgccagcacctcacctgacacagcagcagccatgacccatacccaccaggctgagagcacagaggcctctggacaaacacagaccagcgaaccggcctcctcagggtcacgaaccacctcagcgggcacagctaccccttcctcatccggggcgagtggcacaacaccttcaggaagcgaaggaatatccacctcaggagagacgacaaggttttcatcaaacccctccagggacagtcacacaacccagtcaacaaccgaattgctgtccgcctcagccagtcatggtgccatcccagtaagcacaggaatggcgtcttcgatcgtccccggcacctttcatcccaccctctctgaggcctccactgcagggagaccgacaggacagtcaagcccaacttctcccagtgcctctcctcaggagacagccgccatttcccggatggcccagactcagaggacaggaaccagcagagggtctgacactatcagcctggcgtcccaggcaaccgacaccttctcaacagtcccacccacacctccatcgatcacatccagtgggcttacatctccacaaacccagacccacactctgtcaccttcagggtctggtaaaaccttcaccacggccctcatcagcaacgccacccctcttcctgtcaccagcacctcctcagcctccacaggtcacgccacccctcttgctgtcagcagtgctacctcagcttccacagtatcctcggactcccctctgaagatggaaacatcaggaatgacaacaccgtcactgaagacagacggtgggagacgcacagccacatcaccaccccccacaacctcccagaccatcatttccaccattcccagcactgccatgcacacccgctccacagctgcccccatccccatcctgcctgagagaggagtttccctcttcccctatggggcaggcgccggggacctggagttcgtcaggaggaccgtggacttcacctccccactcttcaagccggcgactggcttcccccttggctcctctctccgtgattccctctacttcacagacaatggccagatcatcttcccagagtcagactaccagattttctcctaccccaacccactcccaacaggcttcacaggccgggaccctgtggccctggtggctccgttctgggacgatgctgacttctccactggtcgggggaccacattttatcaggaatacgagacgttctatggtgaacacagcctgctagtccagcaggccgagtcttggattagaaagatgacaaacaacgggggctacaaggccaggtgggccctaaaggtcacgtgggtcaatgcccacgcctatcctgcccagtggaccctcgggagcaacacctaccaagccatcctctccacggacgggagcaggtcctatgccctgtttctctaccagagcggtgggatgcagtgggacgtggcccagcgctcaggcaacccggtgctcatgggcttctctagtggagatggctatttcgaaaacagcccactgatgtcccagccagtgtgggagaggtatcgccctgatagattcctgaattccaactcaggcctccaagggctgcagttctacaggctacaccgggaagaaaggcccaactaccgtctcgagtgcctgcagtggctgaagagccagcctcggtggcccagctggggctggaaccaggtctcctgcccttgttcctggcagcagggacgacgggacttacgattccaacccgtcagcataggtcgctggggcctcggcagtaggcagctgtgcagcttcacctcttggcgaggaggcgtgtgctgcagctacgggccctggggagagtttcgtgaaggctggcacgtgcagcgtccttggcagttggcccaggaactggagccacagagctggtgctgccgctggaatgacaagccctacctctgtgccctgtaccagcagaggcggccccacgtgggctgtgctacatacaggcccccacagcccgcctggatgttcggggacccccacatcaccaccttggatggtgtcagttacaccttcaatgggctgggggacttcctgctggtcggggcccaagacgggaactcctccttcctgcttcagggccgcaccgcccagactggctcagcccaggccaccaacttcatcgcctttgcggctcagtaccgctccagcagcctgggccccgtcacggtccaatggctccttgagcctcacgacgcaatccgtgtcctgctggataaccagactgtgacatttcagcctgaccatgaagacggcggaggccaggagacgttcaacgccaccggagtcctcctgagccgcaacggctctgaggtctcggccagcttcgacggctgggccaccgtctcggtgatcgcgctctccaacatcctccacgcctccgccagcctcccgcccgagtaccagaaccgcacggaggggctcctgggggtctggaataacaatccagaggacgacttcaggatgcccaatggctccaccattcccccagggagccctgaggagatgcttttccactttggaatgacctggcagatcaacgggacaggcctccttggcaagaggaatgaccagctgccttccaacttcacccctgttttctactcacaactgcaaaaaaacagctcctgggctgaacatttgatctccaactgtgacggagatagctcatgcatctatgacaccctggccctgcgcaacgcaagcatcggacttcacacgagggaagtcagtaaaaactacgagcaggcgaacgccaccctcaatcagtacccgccctccatcaatggtggtcgtgtgattgaagcctacaaggggcagaccacgctgattcagtacaccagcaatgctgaggatgccaacttcacgctcagagacagctgcaccgacttggagctctttgagaatgggacgttgctgtggacacccaagtcgctggagccattcactctggagattctagcaagaagtgccaagattggcttggcatctgcactccagcccaggactgtggtctgccattgcaatgcagagagccagtgtttgtacaatcagaccagcagggtgggcaactcctccctggaggtggctggctgcaagtgtgacgggggcaccttcggccgctactgcgagggctccgaggatgcctgtgaggagccgtgcttcccgagtgtccactgcgttcctgggaagggctgcgaggcctgccctccaaacctgactggggatgggcggcactgtgcggctctggggagctctttcctgtgtcagaaccagtcctgccctgtgaattactgctacaatcaaggccactgctacatctcccagactctgggctgtcagcccatgtgcacctgccccccagccttcactgacagccgctgcttcctggctgggaacaacttcagtccaactgtcaacctagaacttcccttaagagtcatccagctcttgctcagtgaagaggaaaatgcctccatggcagaggtcaacgcctcggtggcatacagactggggaccctggacatgcgggcctttctccgcaacagccaagtggaacgaatcgattctgcagcaccggcctcgggaagccccatccaacactggatggtcatctcggagttccagtaccgccctcggggcccggtcattgacttcctgaacaaccagctgctggccgcggtggtggaggcgttcttataccacgttccacggaggagtgaggagcccaggaacgacgtggtcttccagcccatctccggggaagacgtgcgcgatgtgacagccctgaacgtgagcacgctgaaggcttacttcagatgcgatggctacaagggctacgacctggtctacagcccccagagcggcttcacctgcgtgtccccgtgcagtaggggctactgtgaccatggaggccagtgccagcacctgcccagtgggccccgctgcagctgtgtgtccttctccatctacacggcctggggcgagcactgtgagcacctgagcatgaaactcgacgcgttcttcggcatcttctttggggccctgggcggcctcttgctgctgggggtcgggacgttcgtggtcctgcgcttctggggttgctccggggccaggttctcctatttcctgaactcagctgaggccttgccttgaaggggcagctgtggcctaggctacctcaagactcacctcatccttaccgcacatttaaggcgccattgcttttgggagactggaaaagggaaggtgactgaaggctgtcaggattcttcaaggagaatgaatactgggaatcaagacaagactataccttatccataggcgcaggtgcacagggggaggccataaagatcaaacatgcatggatgggtcctcacgcagacacacccacagaaggacactagcctgtgcacgcgcgcgtgcacacacacacacacacacacgagttcataatgtggtgatggccctaagttaagcaaaatgcttctgcacacaaaactctctggtttacttcaaattaactctatttaaataaagtctctctgactttttgtgtctccaaaa

SEQ ID NO: 99 - DNA, 2323 bp, human
agctatgatcgcaacaccttggtggccatcgtggtgggtgtggggcgcctcatcactggcatggaccgaggcctcatgggcatgtgtgtcaacgagcggcgacgcctcattgtgcctccccacctgggctatgggagcatcggcctggcggggctcattccaccggatgccaccctctacttcgatgtggttctgctggatgtgtggaacaaggaagacaccgtgcaggtgagcacattgctgcgcccgccccactgcccccgcatggtccaggacggcgactttgtccgctaccactacaatggcaccctgctggacggcacctccttcgacaccagctacagtaagggcggcacttatgacacctacgtcggctctggttggctgatcaagggcatggaccaggggctgctgggcatgtgtcctggagagagaaggaagattatcatccctccattcctggcctatggcgagaaaggctatgggacagtgatccccccacaggcctcgctggtctttcacgtcctcctgattgacgtgcacaacccgaaggacgctgtccagctagagacgctggagctcccccccggctgtgtccgcagagccggggccggggacttcatgcgctaccactacaatggctccttgatggacggcaccctcttcgattccagctactcccgcaaccacacctacaatacctatatcgggcagggttacatcatccccgggatggaccaggggctgcagggtgcctgcatgggggaacgccggagaattaccatccccccgcacctcgcctatggggagaatggaactggagacaagatccctggctctgccgtgctaatcttcaacgtccatgtcattgacttccacaaccctgcggatgtggtggaaatcaggacactgtcccggccatccgagacctgcaatgagaccaccaagcttggggactttgttcgataccattacaactgttctttgctggacggcacccagctgttcacctcgcatgactacggggccccccaggaggcgactctcggggccaacaaggtgatcgaaggcctggacacgggcctgcagggcatgtgtgtgggagagaggcggcagctcatcgtgcccccgcacctggcccacggggagagtggagcccggggagtcccaggcagtgctgtgctgctgtttgaggtggagctggtgtcccgggaggatgggctgcccacaggctacctgtttgtgtggcacaaggaccctcctgccaacctgtttgaagacatggacctcaacaaggatggcgaggtccctccggaggagttctccaccttcatcaaggctcaagtgagtgagggcaaaggacgcctcatgcctgggcaggaccctgagaaaaccataggagacatgttccagaaccaggaccgcaaccaggacggcaagatcacagtcgacgagctcaagctgaagtcagatgaggacgaggagcgggtccacgaggagctctgaggggcagggagcctggccaggcctgagacacagaggcccactgcgagggggacagtggcggtgggactgacctgctgacagtcaccctccctctgctgggatgaggtccaggagccaactaaaacaatggcagaggagacatctctggtgttcccaccaccctagatgaaaatccacagcacagacctctaccgtgtttctcttccatccctaaaccacttccttaaaatgtttggatttgcaaagccaatttggggcctgtggagcctggggttggatagggccatggctggtcccccaccatacctcccctccacatcactgacacagctgagcttgttatccatctccccaaactttctctttctttgtacttcttgtcatccccactcccagcccctattcctctatgtgacagctggctaggacccctctgccttccttcccaatcctgactggctcctagggaaggggaaggctcctggagggcagccctacctctcccatgccctttgccctcctccctcgcctccagtggaggctgagctgaccctgggctgctggaggccagactgggctgtagttagcttttcatccctaaagaaggctttccctaaggaaccatagaagagaggaagaaaacaaagggcatgtgtgagggaagctgcttgggtgggtgttagggctatgaaatcttggatttggggctgaggggtgggagggagggcagagctctgcacactcaaaggctaaactggtgtcagtccttttttcctttgttccaaataaaagattaaaccaaaaaaaaaaaaaaaaa

SEQ ID NO: 100 - DNA, 741 bp, human
tcacgtgacccgggcgcgctgcggccgcccgcgcggacccggcgagaggcggcggcgggagcggcggtgatggacgggtccggggagcagcccagaggcggggggcccaccagctctgagcagatcatgaagacaggggcccttttgcttcaggggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctgaggcccccagctgccttggactgtgtttttcctccataaattatggcatttttctgggaggggtggggattgggggacgtgggcatttttcttacttttgtaattattggggggtgtggggaagagtggtcttgagggggtaataaacctccttcgggacacaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 101 - DNA, 2383 bp, human
cctggcgcagggactgctggaacctggctgtgcgcgctgtcgctttaagacagactctgccggcgccgtccggagccttagaaaccggccccggatcgcgagccggagccggagccggagccggggccggccgggctgctgaggcccgagcggcaggagcgcagcgcggagcgctgagccaggcgcccagtcgcgagaagctgccgccgcctctgcccgcccggcgccgcagccccgggcggtccatggggcgggcacggcgtcgctgcaggcgccggcagccctggagggcagccgcttaggcgctgcgctcttgtccccgcaggtcgcagccagggcggcggggcgcgcccagccccggcccctggagcgcccgccgcggtccccacctccatggacgccttcaaggggggcatgagcctggagcggctgccggaggggctccggccgccgccgccgccaccccatgacatggggcccgccttccacctggcccggcccgccgacccccgcgagccgctcgagaactccgccagcgagtcgtctgacacggagctgccagagaaggagcgcggcggggaacccaaggggcccgaggacagtggtgcgggaggcacgggctgcggcggcgcagacgacccagccaagaagaagaagcagcggcggcaacgtacgcacttcacaagccagcagttgcaagagctagaggccacgttccagaggaaccgctaccccgacatgagcatgagggaggagatcgccgtgtggaccaacctcaccgagccgcgcgtgcgggtctggttcaagaaccggcgagccaagtggcgtaagcgcgagcgtaaccagcagctggacctgtgcaagggtggctacgtgccgcagttcagcggcctagtgcagccctacgaggacgtgtacgccgccggctactcctacaacaactgggccgccaagagcctggcgccagcgccgctctccaccaagagcttcaccttcttcaactccatgagcccgctgtcgtcgcagtccatgttctcagcacccagctccatctcctccatgaccatgccgtccagcatgggcccaggcgccgtgcctggcatgcccaactcgggcctcaacaacatcaacaacctcaccggctcctcgctcaactcggccatgtcgccgggcgcttgcccgtacggcactcccgcctcgccctacagcgtctaccgggacacgtgcaactcgagcctagccagcctgcggctcaagtccaaacagcactcgtcgtttggctacggcggcctgcagggcccggcctcgggcctcaacgcgtgccagtacaacagctgaccgccccgccgcaccacgcgggccggcggccggagcggggaagggcgcgggcgcgggcgcggaggacgcacgcggggccccggctcgcaagccccagctcaccgcgccgcggacctcacacctgcgcagccccctcctcccacttcccactccgggttggttttgtgtttgcttttccggaccccactctgccctccaaaaagacaaaaaaaaaaaaaaaaaaaaaaaagcaaaaagacgtcggagaaaagtgccgcgaaaaaatggatgagttgcaatttctctcgggatggcgcgggtggtgtgtgtgttcccacgggccccggaggcccactccgcggagggcacgcggcgcggtaggcgagcgccgaggcccagcggccgggggaggacgacctcgtatcccgcgtccccgccgcgctggatccggactgagtggccgggcctgcggactggatgtgcggggcctggacttgcctaggatttcccgaccccgtacaaaccaagttgccctctccgagctaggcccggccgagagcgccttagctcgagtcggatccgtgttggggcgggcgttgggtttggggggacggtgcccccagcccaggatcgggcactcagtggagccgcacacggccccggcgcgcctggtagagcctcgctggccccgcgccccggagccctatattaaggccacggagcgacagcgggcagtgcgggcctggcgggaggtgggggaggtccatctcagaacaccccagccttgagcttagctgcaggcccaggccctctgctctgctcccgggctaggaggtggccctctgtctgggcgaacagccccctcctcaccgcccgccgtgcaagagtcgagccggcagagcaaggggcgcggccccagggccctgcgcccactttgcacacccgctctccggcccgcgcccctgtttacagcgtccctgtgtatgttggactgactgtaataaatctgtctatatcgacttgtccatgtacgtctattaaaaccatagtccgagcgtgctaagca

SEQ ID NO: 102 - DNA, 1314 bp, human
aattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatccgagagccaagatgccggcccacttgctgcaggacgatatctctagctcctataccaccaccaccaccattacagcgcctccctccagggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactaaaaaaaaaaaaaaaaaaaaaaaaaaaaaaaaaaaaaaaaaaaaaaaaaaaataaaaaaaaaaaaaaaaaa

SEQ ID NO: 103 - DNA, 476 bp, human; identical to SEQ ID NO: 1

SEQ ID NO: 104 - DNA, 476 bp, human; identical to SEQ ID NO: 1